(19) United States
(12) Patent Application Publication
(10) Pub. No.: US 2007/0054355 A1
(43) Pub. Date: Mar. 8, 2007

(54) Cbl-b polypeptides, complexes and related methods

(76) Inventors: Yuval Reiss, Kiriat-Ono (IL); Daniel N. Taglicht, Lapid (IL); Iris Alroy, Ness-Ziona (IL); Shmuel Tuvia, Netanya (IL); Haim Michael Barr, Kfar Sava (IL)

Correspondence Address: FISH & NEAVE IP GROUP;ROPES & GRAY LLP, ONE INTERNATIONAL PLACE, BOSTON, MA, 02110-2624, US

(21) Appl. No.: 10/547,849

(22) PCT Filed: Mar. 5, 2004
(86) PCT No.: PCT/US04/06619

§ 371 Date: Sep. 22, 2006

Related U.S. Application Data
(60) Provisional application No. 60/452,284, filed on Mar. 5, 2003.
(60) Provisional application No. 60/456,640, filed on Mar. 20, 2003.
(60) Provisional application No. 60/469,462, filed on May 9, 2003.
(60) Provisional application No. 60/471,378, filed on May 15, 2003.
(60) Provisional application No. 60/480,376, filed on Jun. 19, 2003.
(60) Provisional application No. 60/480,215, filed on Jun. 19, 2003.

(51) Int. Cl. A61K 31/675; C07H 21/04; C12P 21/06; C12N 9/16; A61K 31/5415; A61K 31/473; A61K 31/4035; A61K 31/445; A61K 31/18

(52) U.S. Cl. 435/69.1; 435/196; 435/320.1; 435/325; 530/350; 536/23.2; 514/80; 514/471; 514/290; 514/414; 514/638; 514/657; 514/326; 514/224.8; 514/602; 514/352

(57) ABSTRACT

The application provides novel complexes of Cbl-b polypeptides and Cbl-b-associated proteins. The application also provides methods and compositions for treating Cbl-b-associated diseases such as viral disorders.

### RELATED APPLICATIONS

This application claims the benefit of priority of U.S. Provisional Application No. 60/452,284 filed 5 Mar. 2003; 60/456,640 filed 20 Mar. 2003; 60/469,462 filed 9 May 2003; 60/471,378 filed 15 May 2003; 60/480,376 filed 19 Jun. 2003; and 60/480,215 filed 19 Jun. 2003. The teachings of the referenced Applications are incorporated herein by reference in their entirety.

### BACKGROUND

Potential drug target validation involves determining whether a DNA, RNA or protein molecule is implicated in a disease process and is therefore a suitable target for development of new therapeutic drugs. Drug discovery, the process by which bioactive compounds are identified and characterized, is a critical step in the development of new treatments for human diseases. The landscape of drug discovery has changed dramatically due to the genomics revolution. DNA and protein sequences are yielding a host of new drug targets and an enormous amount of associated information.

The identification of genes and proteins involved in various disease states or key biological processes, such as inflammation and immune response, is a vital part of the drug design process. Many diseases and disorders could be treated or prevented by decreasing the expression of one or more genes involved in the molecular etiology of the condition if the appropriate molecular target could be identified and appropriate antagonists developed. For example, cancer, in which one or more cellular oncogenes become activated and result in the unchecked progression of cell cycle processes, could be treated by antagonizing appropriate cell cycle control genes. Furthermore many human genetic diseases, such as Huntington's disease, and certain prion conditions, which are influenced by both genetic and epigenetic factors, result from the inappropriate activity of a polypeptide as opposed to the complete loss of its function. Accordingly, antagonizing the aberrant function of such mutant genes would provide a means of treatment. Additionally, infectious diseases such as HIV have been successfully treated with molecular antagonists targeted to specific essential retroviral proteins such as HIV protease or reverse transcriptase. Drug therapy strategies for treating such diseases and disorders have frequently employed molecular antagonists which target the polypeptide product of the disease gene(s). However, the discovery of relevant gene or protein targets is often difficult and time consuming.

One area of particular interest is the identification of host genes and proteins that are co-opted by viruses during the viral life cycle. The serious and incurable nature of many viral diseases, coupled with the high rate of mutations found in many viruses, makes the identification of antiviral agents a high priority for the improvement of world health. Genes and proteins involved in a viral life cycle are also appealing as a subject for investigation because such genes and proteins will typically have additional activities in the host cell and may play a role in other non-viral disease states.

Other areas of interest include the identification of genes and proteins involved in cancer, apoptosis and neural disorders particularly those associated with apoptotic neurons, such as Alzheimer's disease).

It would be beneficial to identify proteins involved in one or more of these processes for use in, among other things, drug screening methods. Additionally, once a protein involved in one or more processes of interest has been identified, it is possible to identify proteins that associate, directly or indirectly, with the initially identified protein. Knowledge of interactors will provide insight into protein assemblages and pathways that participate in disease processes, and in many cases an interacting protein will have desirable properties for the targeting of therapeutics. In some cases, an interacting protein will already be known as a drug target, but in a different biological context. Thus, by identifying a suite of proteins that interact with an initially identified protein, it is possible to identify novel drug targets and new uses for previously known therapeutics.

### SUMMARY

Described herein are novel associations between Cbl-b polypeptides and Cbl-b-associated proteins (termed “Cbl-b-APs”). In certain aspects, the application relates to the discovery of novel associations between Cbl-b proteins and Cbl-b-APs, and related methods and compositions. In preferred embodiments of the application, the application relates to the discovery of novel associations between Cbl-b and the Cbl-b-AP, POSH, and related methods and compositions. In certain embodiments, the application relates to an isolated, purified or recombinant complex, comprising a Cbl-b polypeptide and a POSH polypeptide. The certain further embodiments, the application relates to an isolated, purified or recombinant complex, comprising a Cbl-b polypeptide and a polypeptide comprising a domain that is at least 90% identical to a POSH SH3 domain. In certain embodiments, the application provides methods and compositions for identifying an antiviral agent. In certain aspects, the application relates to a method of identifying an antiviral agent, comprising identifying a test agent that disrupts a complex comprising a Cbl-b polypeptide and a POSH polypeptide. In certain embodiments, the present application relates to a method of identifying an antiviral agent, comprising identifying a test agent that disrupts a complex comprising a Cbl-b polypeptide and a domain that is at least 90% identical to a POSH SH3 domain. In certain aspects, the Cbl-b polypeptide is a human Cbl-b polypeptide. In certain aspects, the POSH polypeptide is a human POSH polypeptide.

The application additionally relates to methods and compositions for identifying an agent that modulates an activity of a Cbl-b polypeptide and a POSH polypeptide. In certain embodiments, the application relates to a method of identifying an agent that modulates an activity of a Cbl-b polypeptide and a POSH polypeptide, comprising identifying an agent that disrupts a complex comprising a Cbl-b polypeptide and a POSH polypeptide, wherein an agent that disrupts a complex of a Cbl-b polypeptide and a POSH polypeptide is an agent that modulates an activity of the Cbl-b polypeptide or the POSH polypeptide. In further embodiments, the application relates to a method of identifying an agent that modulates an activity of a Cbl-b polypeptide and a POSH polypeptide, comprising identifying an agent that disrupts a complex comprising a Cbl-b polypeptide and a domain that is at least 90% identical to a POSH SH3 domain, wherein an agent that disrupts a complex comprising a Cbl-b polypeptide and a domain that is at least 90% identical to a POSH SH3 domain is an agent that modulates an activity of the Cbl-b polypeptide or the POSH polypeptide.

The application further provides methods and compositions for identifying an antiviral agent. In one embodiment, the application relates to a method of identifying an antiviral agent, comprising identifying a test agent that disrupts a complex comprising a Cbl-b polypeptide and a Cbl-b-AP polypeptide and evaluating the effect of the test agent on a function of a virus, wherein an agent that inhibits a pro-infective or pro-replicative function of a virus is an antiviral agent. In certain embodiments, the Cbl-b-AP is POSH in certain aspects, the virus is an envelope virus, such as a human immunodeficiency virus (e.g., HIV-1, HIV-2). In certain embodiments, the evaluating the effect of the test agent on a function of the virus comprises evaluating the effect of the test agent on the budding, release, infectivity, or reverse transcriptase activity of the virus or a virus-like particle.

In certain embodiments, the present application relates to a method of treating a viral infection in a subject in need thereof, comprising administering, in an amount sufficient to inhibit the viral infection, an agent that inhibits the expression of or an activity of a Cbl-b polypeptide. In certain embodiments, the agent is selected from among an siRNA construct, an antisense construct, an antibody, a polypeptide, and a small molecule. In certain embodiments, the agent is an siRNA construct comprising a nucleic acid sequence that hybridizes to an mRNA encoding a Cbl-b polypeptide. In preferred embodiments, the siRNA construct inhibits the expression of a Cbl-b polypeptide. Examples of siRNA constructs of the application include an siRNA construct selected from among SEQ ID NOS: 59-64. In certain embodiments, an agent that inhibits the expression of or an activity of a Cbl-b polypeptide is a small molecule. For example, examples of small molecules include: 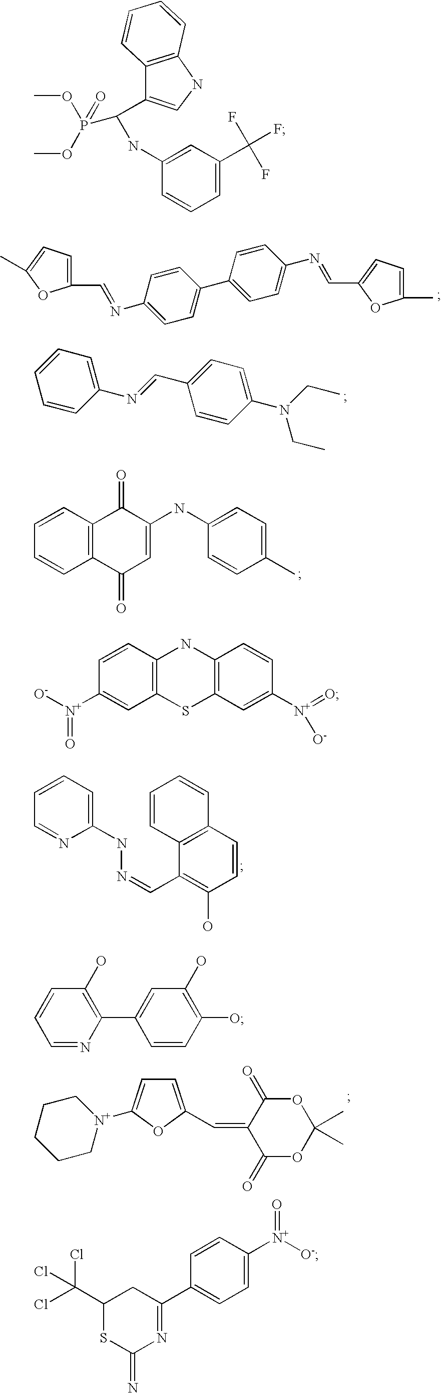 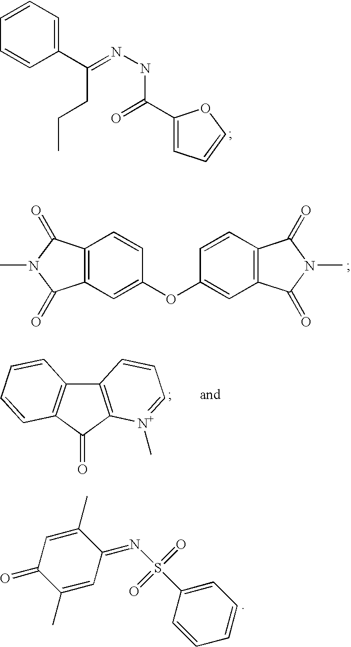
In certain further embodiments, the small molecule inhibits the ubiquitin ligase activity of a Cbl-b polypeptide. In certain embodiments, the subject is infected with an envelope virus. Optionally, the envelope virus is a human immunodeficiency virus (e.g., HIV-1, HIV-2). In certain embodiments, the subject is infected with a West Nile Virus.

The application further relates to the use of an inhibitor of Cbl-b for the manufacture of a medicament for treatment of a viral infection. In certain aspects, the application provides a packaged pharmaceutical for use in treating a viral infection, comprising a pharmaceutical composition comprising an inhibitor of a Cbl-b polypeptide and a pharmaceutically acceptable carrier and instructions for use. In certain embodiments, the viral infection is caused by an envelope virus, such as a human immunodeficiency virus (e.g., HIV-1, HIV-2). In certain embodiments, the viral infection is caused by West Nile Virus.

The application additionally relates to methods of identifying an antiviral agent, comprising identifying a test agent that inhibits an activity of or expression of a Cbl-b polypeptide and evaluating an effect of the test agent on a function of a virus. In certain embodiments, the application relates to a method of evaluating an antiviral agent, comprising providing a test agent that inhibits an activity of or expression of a Cbl-b polypeptide and evaluating an effect of the test a gent on a function of a virus. In certain aspects, the virus is an envelope virus, such as a human immunodeficiency virus. In certain embodiments, the virus is a West Nile Virus. In certain embodiments, evaluating the effect of the test agent on a function of the virus comprises evaluating the effect of the test agent on the budding, release, infectivity, or reverse transcriptase activity of the virus or a virus-like particle. In further embodiments of the application, the test agent is selected from among an siRNA construct, an antisense construct, an antibody, a polypeptide, and a small molecule. In certain embodiments, the test agent is an siRNA construct that inhibits the expression of Cbl-b and is selected from among SEQ ID NOS: 59-64.

The application further relates to a method of identifying an agent that modulates a Cbl-b function, comprising identifying an agent that modulates a POSH polypeptide and testing the effect of the agent on a Cbl-b function. In additional aspects, the application relates to a method of evaluating an agent that modulates a Cbl-b function, comprising providing an agent that modulates a POSH polypeptide and testing the effect of the agent on a Cbl-b function. In certain embodiments, testing the effect of the agent on a Cbl-b function comprises contacting a cell with the agent and measuring the effect of the agent on Cbl-b-mediated ubiquitination. In certain embodiments, testing the effect of the agent on a Cbl-b function comprises contacting a cell with the agent and measuring the effect of the agent on the budding, release, infectivity, or reverse transcriptase activity of a virus or a virus-like particle.

The application further relates to a method of identifying an agent that modulates a POSH function, comprising identifying an agent that modulates a Cbl-b polypeptide and testing the effect of the agent on a POSH function. In additional embodiments, the application relates to a method of evaluating an agent that modulates a POSH function, comprising providing an agent that modulates a Cbl-b polypeptide and testing the effect of the agent on a POSH function. In certain embodiments, testing the effect of the agent on a POSH function comprises contacting a c ell with the agent and measuring the effect o f the agent on P OSH-mediated ubiquitination.

In certain embodiments, the application relations to a method of identifying an antiviral agent, comprising forming a mixture comprising a Cbl-b polypeptide, ubiquitin, and a test agent; and detecting the ubiquitin ligase activity of the Cbl-b polypeptide, wherein an agent that inhibits the ubiquitin ligase activity of the Cbl-b polypeptide, is an antiviral agent.

In yet other embodiments of the application, the application relates to a method of identifying an antiviral agent comprising providing a Cbl-b polypeptide and a test agent; and identifying a test agent that binds to the Cbl-b polypeptide. In further embodiments, the application relates to a method of identifying an antiviral agent, comprising providing a Cbl-b polypeptide and a test agent; and identifying a test agent that binds to the Cbl-b polypeptide, further comprising evaluating the effect of the test agent on Cbl-b-mediated ubiquitination. In certain embodiments, the application relates to a method of identifying an antiviral agent comprising providing a Cbl-b polypeptide and a test agent; and identifying a test agent that binds to the Cbl-b polypeptide, further comprising evaluating the effect of the test agent on the budding, release, infectivity, or reverse transcriptase activity of a virus or a virus-like particle.

In other embodiments, the application relates to a method of identifying an agent with antiviral activity, comprising contacting a Cbl-b polypeptide with a test agent; and identifying a test agent that inhibits a Cbl-b activity. In further embodiments, the application relates to a method of identifying an agent with antiviral activity, comprising contacting a Cbl-b polypeptide with a test agent; and identifying a test agent that inhibits a Cbl-b activity, further comprising evaluating the effect of the test agent on Cbl-b-mediated ubiquitination. In certain embodiments, application relates to a method of identifying an agent with antiviral activity, comprising contacting a Cbl-b polypeptide with a test agent; and identifying a test agent that inhibits a Cbl-b activity, further comprising evaluating the effect of the test agent on the budding, release, infectivity, or reverse transcriptase activity of a virus or a virus-like particle.

In yet other embodiments, the application relates to a method of identifying an antiviral agent, comprising providing a Cbl-b polypeptide and a test agent; and identifying a test agent that interacts with the Cbl-b polypeptide. In certain embodiments, the application relates to a method of identifying an antiviral agent, comprising providing a Cbl-b polypeptide and a test a gent; and identifying a test agent that interacts with the Cbl-b polypeptide, further comprising evaluating the effect of the test agent on Cbl-b-mediated ubiquitination. In certain embodiments, the application relates to a method of identifying an antiviral agent, comprising providing a Cbl-b polypeptide and a test agent; and identifying a test agent that interacts with the Cbl-b polypeptide, further comprising evaluating the effect of the test agent on the budding, release, infectivity, or reverse transcriptase activity of a virus or a virus-like particle.

In additional embodiments, the application relates to a method of inhibiting a viral infection, comprising administering an agent to a subject in need thereof, wherein said agent inhibits the interaction between a Cbl-b polypeptide and a POSH polypeptide. In further embodiments, the application provides a method of inhibiting a viral infection, comprising administering to a subject in need thereof, an agent that inhibits the expression of or an activity of a Cbl-b polypeptide, wherein said agent inhibits the expression of or an activity of the Cbl-b polypeptide. Optionally, the agent inhibits the ubiquitin ligase activity of the Cbl-b polypeptide.

The application further provides Cbl-b nucleic acid and amino acid sequences. In certain embodiments, the application relates to an isolated Cbl-b nucleic acid comprising a nucleic acid sequence at least 85% identical to the nucleic acid sequence depicted in SEQ ID NO: 43. In further embodiments, the application provides an isolated Cbl-b nucleic acid, wherein the nucleic acid comprises the nucleic acid sequence depicted in SEQ ID NO: 43. In certain embodiments, the application provides an isolated Cbl-b polypeptide, comprising the amino acid sequence depicted in SEQ ID NO: 45. In yet additional embodiments, the application relates to an isolated Cbl-b nucleic acid comprising a nucleic acid sequence at least 8 5% identical to the nucleic acid sequence depicted in SEQ ID NO: 44. In further embodiments, the application provides an isolated Cbl-b nucleic acid, wherein the nucleic acid comprises the nucleic acid sequence depicted in SEQ ID NO: 44. In further embodiments, the application provides an isolated Cbl-b polypeptide, comprising the amino acid sequence depicted in SEQ ID NO: 46.

In further embodiments, the application relates to a method of identifying an anti-apoptotic agent, comprising identifying a test agent that disrupts a complex comprising a Cbl-b polypeptide and a POSH polypeptide; and evaluating the effect of the test agent on apoptosis of a cell, wherein an agent that decreases apoptosis of the cell is an anti-apoptotic agent.

In certain embodiments of the application, the application relates to a method of identifying an anti-cancer agent, comprising identifying a test agent that disrupts a complex comprising a Cbl-b polypeptide and a POSH polypeptide; and evaluating the effect of the test agent on proliferation or survival of a cancer cell, wherein an agent that decreases proliferation or survival of a cancer cell is an anti-cancer agent. In preferred embodiments, the cancer cell is a cell derived from a POSH-associated cancer.

In yet other embodiments, the application provides methods and compositions for identifying an agent that inhibits the progression of a neurological disorder. In certain embodiments, the application relates to a method of identifying an agent that inhibits the progression of a neurological disorder, comprising identifying a test agent that disrupts a complex comprising a Cbl-b polypeptide and a Cbl-b-AP polypeptide; and evaluating the effect of the test agent on the trafficking of a protein through the secretory pathway, wherein an agent that disrupts localization of a Cbl-b-AP polypeptide is an agent that inhibits progression of a neurological disorder. In certain embodiments, the Cbl-b-AP is POSH. In certain embodiments, the Cbl-b-AP is a POSH-AP. In further embodiments of the application, the application relates to a method of identifying an agent that inhibits the progression of a neurological disorder, comprising identifying a test agent that disrupts a complex comprising a Cbl-polypeptide and a POSH polypeptide; and evaluating the effect of the test agent on the ubiquitination of a protein.

The application further relates to a method of treating or preventing a POSH-associated cancer in a subject comprising administering an agent that inhibits the expression of or an activity of a Cbl-b polypeptide to a subject in need thereof, wherein said agent treats or prevents the POSH-associated cancer. In certain embodiments, the cancer is associated with increased POSH expression.

In further embodiments of the application, the application relates to a method of treating or preventing a POSH-associated neurological disorder in a subject comprising administering an agent that inhibits the expression of or an activity of a Cbl-b polypeptide to a subject in need thereof, wherein said agent treats or prevents the POSH-associated neurological disorder. POSH-associated neurological disorders include Alzheimer's disease, Parkinson's disease, Huntington's disease, schizophrenia, Niemann-Pick's disease, and prion-associated diseases.

Additionally, the application relates to a method of treating or preventing a POSH-associated viral disorder (e.g., HIV-1 infection) in a subject comprising administering an agent that inhibits the expression of or an activity of a Cbl-b polypeptide to a subject in need thereof, wherein said agent treats or prevents the POSH-associated viral disorder.

The practice of the present application will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Maizual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the application will be apparent from the following detailed description, and from the claims.

### BRIEF DESCRIPTION OF THE DRAWINGS

FIG. 1 shows human POSH coding sequence (SEQ ID NO:1).

FIG. 2 shows human POSH amino acid sequence (SEQ ID NO:2).

FIG. 3 shows human POSH cDNA sequence (SEQ ID NO:3).

FIG. 4 shows 5′ cDNA fragment of human POSH (public gi:10432611; SEQ ID NO:4).

FIG. 5 shows N terminus protein fragment of hPOSH (public gi:10432612; SEQ ID NO:5).

FIG. 6 shows 3′ mRNA fragment of HPOSH (public gi:7959248; SEQ ID NO:6).

FIG. 7 shows C terminus protein fragment of HPOSH (public gi:7959249; SEQ ID NO:7).

FIG. 8 shows human POSH fall mRNA, annotated sequence.

FIG. 9 shows domain analysis of human POSH.

FIG. 10 is a diagram of human POSH nucleic acids. The diagram shows the full-length POSH gene and the position of regions amplified by RT-PCR or targeted by siRNA used in FIG. 11.

FIG. 11 shows effect of knockdown of POSH mRNA by siRNA duplexes. HeLa S S-6 cells were transfected with siRNA against Lamin A/C (lanes 1, 2 ) or POSH (lanes 3-10). POSH siRNA was directed against the coding region (153—lanes 3, 4; 155—lanes 5, 6) or the 3′UTR (157—lanes 7, 8; 159—lanes 9, 10). Cells were harvested 24 hours post-transfection, RNA extracted, and POSH mRNA levels compared by RT-PCR of a discrete sequence in the coding region of the POSH gene (see FIG. 10). GAPDH is used an RT-PCR control in each reaction.

FIG. 12 shows that POSH affects the release of VLP from cells. A) Phosphohimages of SDS-PAGE gels of immunoprecipitations of ³⁵S pulse-chase labeled Gag proteins are presented for cell and viral lysates from transfected HeLa cells that were either untreated or treated with POSH RNAi (50 nM for 48 hours). The time during the chase period (1, 2, 3, 4, and 5 hours after the pulse) are presented from left to right for each image.

FIG. 13 shows release of VLP from cells at steady state. Hela cells were transfected with an HIV-encoding plasmid and siRNA. Lanes 1, 3 and 4 were transfected with wild-type HIV-encoding plasmid. Lane 2 was transfected With an HIV-encoding plasmid which contains a point mutation in p6 (PTAP to ATAP). Control siRNA (lamin A/C) was transfected to cells in lanes 1 and 2. siRNA to Tsg101 was transfected in lane 4 and siRNA to POSH in lane 3.

FIG. 14 shows mouse POSH mRNA sequence (public gi:10946921; SEQ ID NO: 8).

FIG. 15 shows mouse POSH Protein sequence (Public gi:10946922; SEQ ID NO: 9).

FIG. 16 shows Drosophila melanogaster POSH mRNA sequence (public gi:17737480; SEQ ID NO:10).

FIG. 17 shows Drosophila melanogaster POSH protein sequence (public gi:17737481; SEQ ID NO:11).

FIG. 18 shows POSH domain analysis.

FIG. 19 shows that human POSH has ubiquitin ligase activity.

FIG. 20 shows that Cbl-b associates with POSH in vivo.

FIG. 21 shows that POSH knockdown results in decreased secretion of phospholipase D (“PLD”).

FIG. 22 shows effect of hPOSH on Gag-EGFP intracellular distribution.

FIG. 23 shows intracellular distribution of HIV-1 Nef in hPOSH-depleted cells.

FIG. 24 shows intracellular distribution of Src in hPOSH-depleted cells.

FIG. 25 shows intracellular distribution of Rapsyn in hPOSH-depleted cells.

FIG. 26 shows that POSH reduction by siRNA abrogates West Nile virus infectivity.

FIG. 27 shows that POSH knockdown decreases the release of extracellular MMuLV particles.

FIG. 28 shows that knock-down of human POSH entraps HIV virus particles in intracellular vesicles. HIV virus release was analyzed by electron microscopy following siRNA and full-length HIV plasmid transfection. Mature viruses were secreted by cells transfected with HIV plasmid and non-relevant siRNA (control, bottom panel). Knockdown of Tsg101 protein resulted in a budding defect, the viruses that were released had an immature phenotype (top panel). Knockdown of HPOSH levels resulted in accumulation of viruses inside the cell in intracellular vesicles (middle panel).

FIG. 29 shows that siRNA-mediated reduction in Cbl-b expression inhibits HIV virus-like particle (VLP) production

FIG. 30 shows that siRNA-mediated reduction in Cbl-b expression inhibits HIV virus-like particle (VLP) production

FIG. 31 shows RT activity in VLP secreted from cells treated with control and Cbl-b siRNAs.

FIG. 32 shows the results of an HIV-1 infectivity assay in cells treated with siRNA against Cbl-b.

FIG. 33 shows RT activity in VLP secreted from cells transfected with indicated plasmids (empty and Cbl-b RING mutant).

FIG. 34 shows inhibitors of Cbl-b activity.

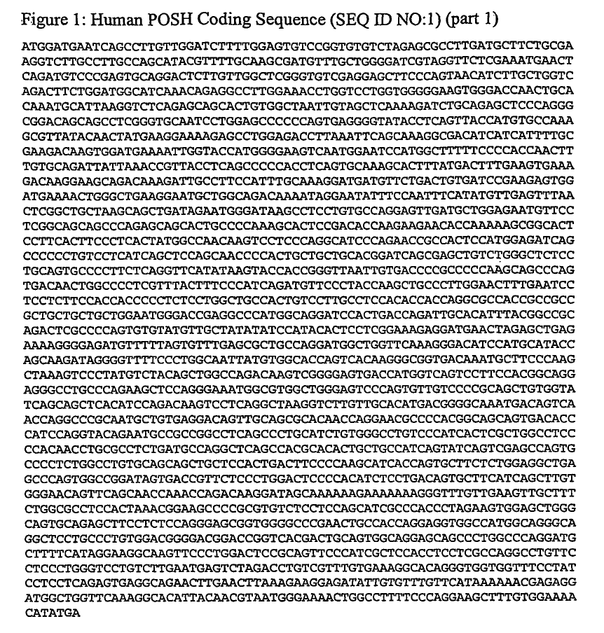

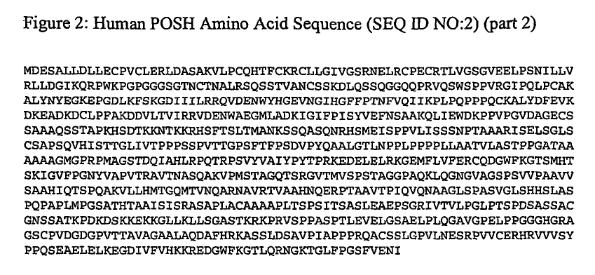

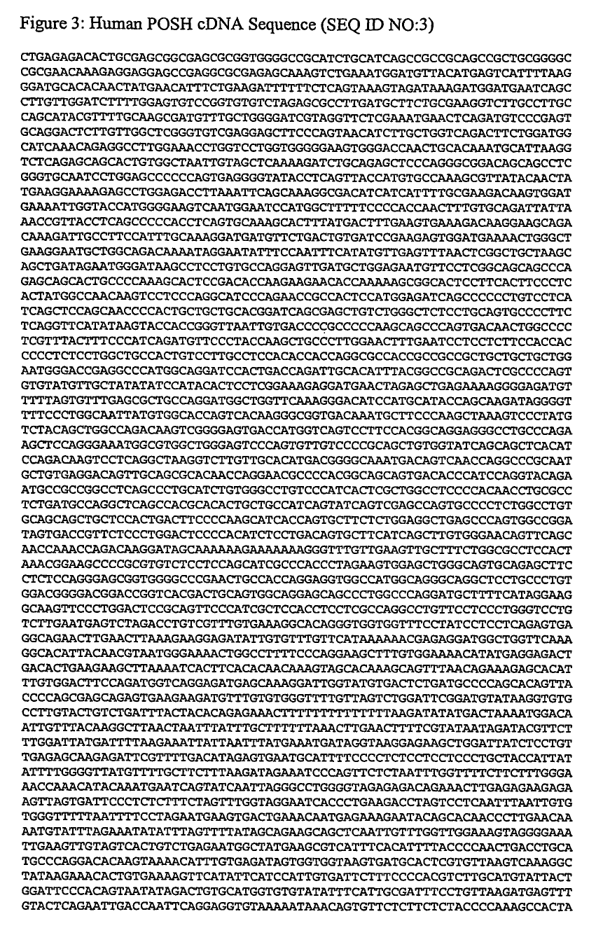

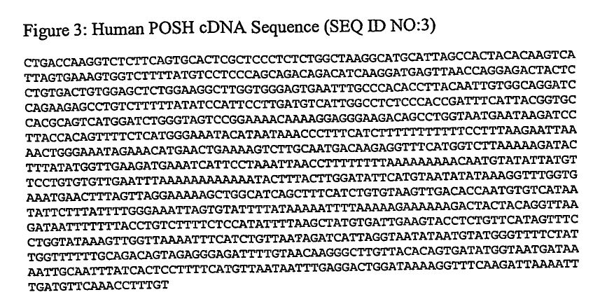

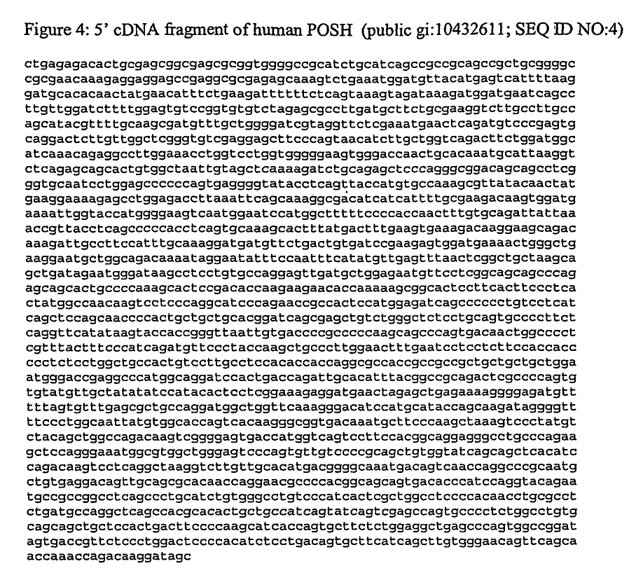

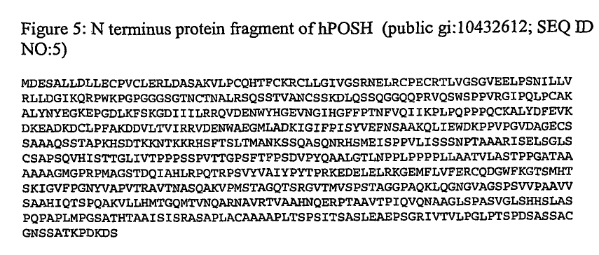

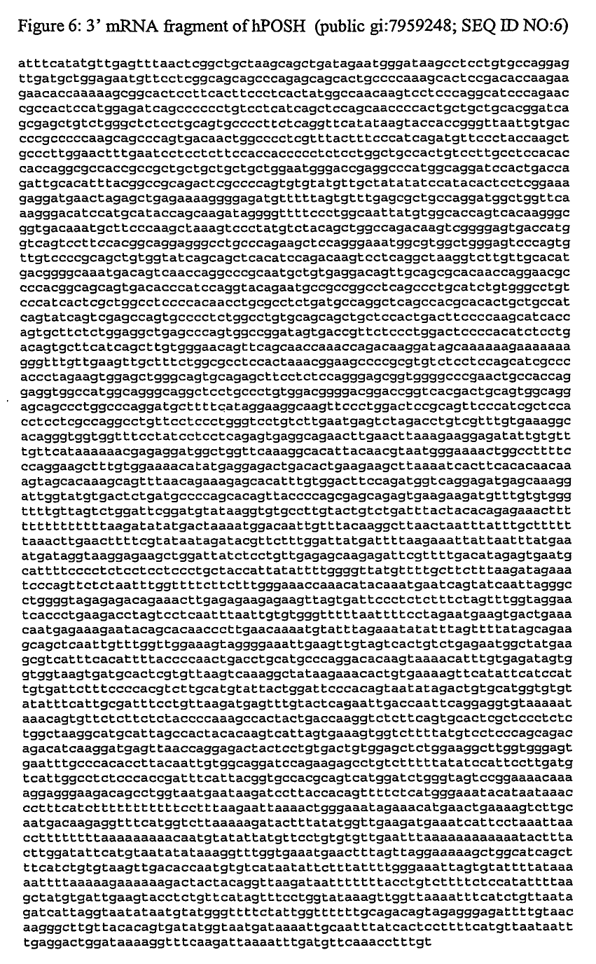

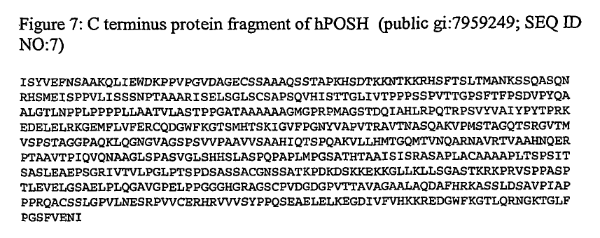

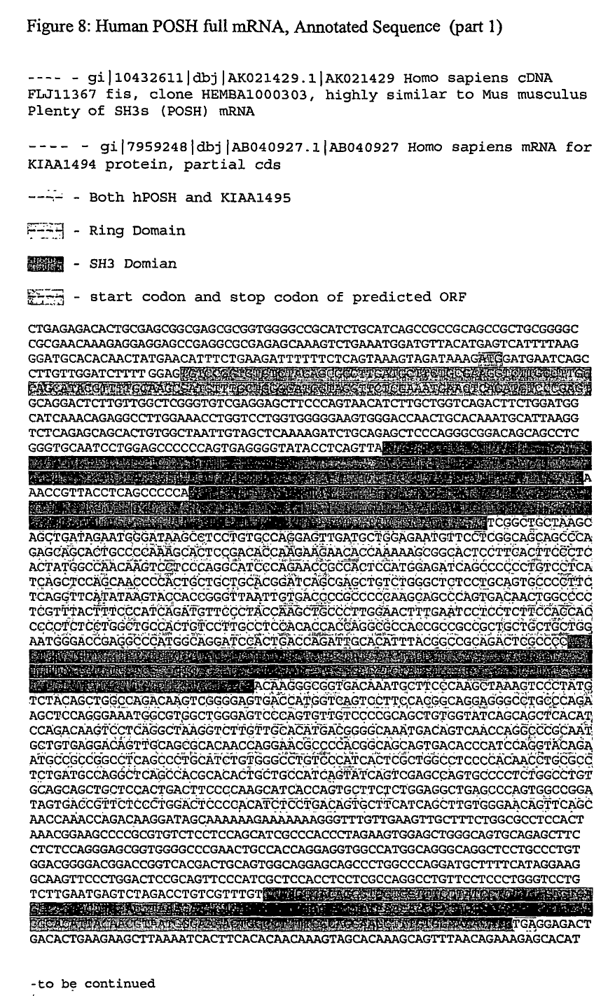

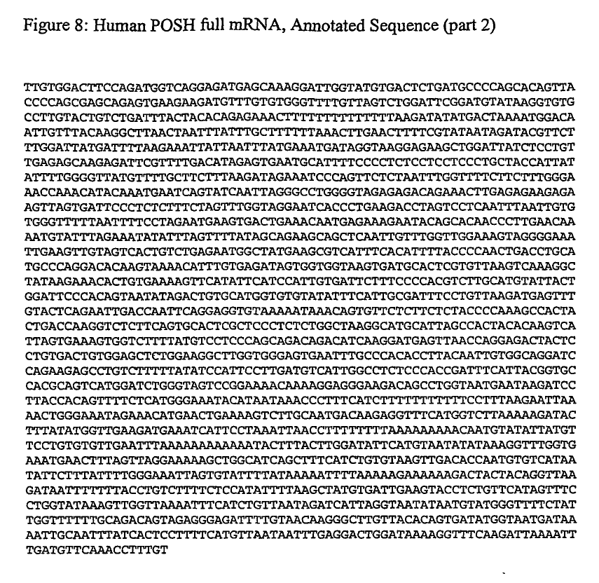

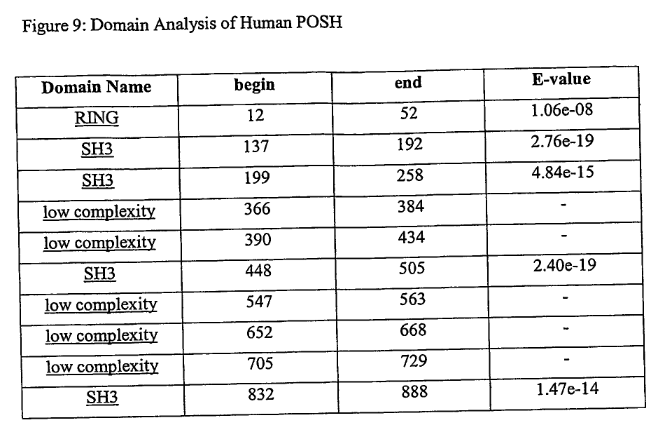

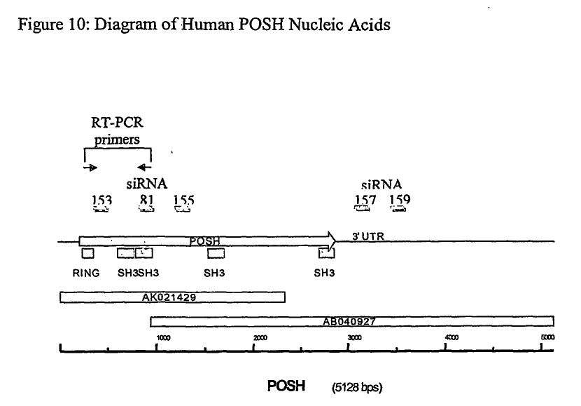

### DETAILED DESCRIPTION OF THE APPLICATION

### 1. DEFINITIONS

The term “binding” refers to a direct association between two molecules, due to, for example, covalent, electrostatic, hydrophobic, ionic and/or hydrogen-bond interactions under physiological conditions.

A “Cbl-b nucleic acid” is a nucleic acid comprising a sequence as represented in any of SEQ ID NOs: 37-44 and 51-54 as well as any of the variants described herein.

A “Cbl-b polypeptide” or “Cbl-b protein” is a polypeptide comprising a sequence as represented in any of SEQ ID NOs: 45-50 and 55-58 as well as any of the variations described herein.

A “Cbl-b-associated protein” or “Cbl-b-AP” refers to a protein capable of interacting with and/or binding to a Cbl-b polypeptide. Generally, the Cbl-b-AP may interact directly or indirectly with the Cbl-b polypeptide. A preferred Cbl-b-AP of the application is POSH. Examples of POSH polypeptides are provided throughout.

A “chimeric protein” or “fusion protein” is a fusion of a first amino acid sequence encoding a polypeptide with a second amino acid sequence defining a domain foreign to and not substantially homologous with any domain of the first amino acid sequence. A chimeric protein may present a foreign domain which is found (albeit in a different protein) in an organism which also expresses the first protein, or it may be an “interspecies”, “intergenic”, etc. fusion of protein structures expressed by different kinds of organisms.

The terms “compound”, “test compound” and “molecule” are used herein interchangeably and are meant to include, but are not limited to, peptides, nucleic acids, carbohydrates, small organic molecules, natural product extract libraries, and any other molecules (including, but not limited to, chemicals, metals and organometallic compounds).

The phrase “conservative amino acid substitution” refers to grouping of amino acids on the basis of certain common properties. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). Examples of amino acid groups defined in this manner include:
(i) a charged group, consisting of Glu and Asp, Lys, Arg and His,
(ii) a positively-charged group, consisting of Lys, Arg and His,
(iii) a negatively-charged group, consisting of Glu and Asp,
(iv) an aromatic group, consisting of Phe, Tyr and Trp,
(v) a nitrogen ring group, consisting of His and Trp,
(vi) a large aliphatic nonpolar group, consisting of Val, Leu and Ile,
(vii) a slightly-polar group, consisting of Met and Cys,
(viii) a small-residue group, consisting of Ser, Thr, Asp, Asn, Gly, Ala, Glu, Gln and Pro,
(ix) an aliphatic group consisting of Val, Leu, Ile, Met and Cys, and
(x) a small hydroxyl group consisting of Ser and Thr.

In addition to the groups presented above, each amino acid residue may form its own group, and the group formed by an individual amino acid may be referred to simply by the one and/or three letter abbreviation for that amino acid commonly used in the art.

A “conserved residue” is an amino acid that is relatively invariant across a range of similar proteins. Often conserved residues will vary only by being replaced with a similar amino acid, as described above for “conservative amino acid substitution”.

The term “domain” as used herein refers to a region of a protein that comprises a particular structure and/or performs a particular function.

The term “envelope virus” as used herein refers to any virus that uses cellular membrane and/or any organelle membrane in the viral release process.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present application. In comparing two sequences, the absence of residues (amino acids or nucleic acids) or presence of extra residues also decreases the identity and homology/similarity.

The term “homology” describes a mathematically based comparison of sequence similarities which is used to identify genes or proteins with similar functions or motifs. The nucleic acid and protein sequences of the present application may be used as a “query sequence” to perform a search against public databases to, for example, identify other family members, related sequences or homologs. Such searches can be performed using the NBLAST and XBLAST programs (version 2.0) of Altschul, et al. (1990) J Mol. Biol. 215:403-10. BLAST nucleotide searches can be performed with the NBLAST program, score=100, wordlength=12 to obtain nucleotide sequences homologous to nucleic acid molecules of the application. BLAST protein searches can be performed with the XBLAST program, score=50, wordlength=3 to obtain amino acid sequences homologous to protein molecules of the application. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used. See http://www.ncbi.nlm.nih.gov.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

The term “isolated”, as used herein with reference to the subject proteins and protein complexes, refers to a preparation of protein or protein complex that is essentially free from contaminating proteins that normally would be present with the protein or complex, e.g., in the cellular milieu in which the protein or complex is found endogenously. Thus, an isolated protein complex is isolated from cellular components that normally would “contaminate” or interfere with the study of the complex in isolation, for instance while screening for modulators thereof. It is to be understood, however, that such an “isolated” complex may incorporate other proteins the modulation of which, by the subject protein or protein complex, is being investigated.

The term “isolated” as also used herein with respect to nucleic acids, such as DNA or RNA, refers to molecules in a form which does not occur in nature. Moreover, an “isolated nucleic acid” is meant to include nucleic acid fragments which are not naturally occurring as fragments and would not be found in the natural state.

Lentiviruses include primate lentiviruses, e.g., human immunodeficiency virus types 1 and 2 (HIV-1/HIV-2); simian immunodeficiency virus (SIV) from Chimpanzee (SIVcpz), Sooty mangabey (SIVsmm), African Green Monkey (SIVagm), Syke's monkey (SIVsyk), Mandrill (SIVmnd) and Macaque (SIVmac). Lentiviruses also include feline lentiviruses, e.g., Feline immunodeficiency virus (FIV); Bovine lentiviruses, e.g., Bovine immunodeficiency virus (BIV); Ovine lentiviruses, e.g., MaediVisna virus (MVV) and Caprine arthritis encephalitis virus (CAEV); and Equine lentiviruses, e.g., Equine infectious anemia virus (EIAV). All lentiviruses express at least two additional regulatory proteins (Tat, Rev) in addition to Gag, Pol, and Env proteins. Primate lentiviruses produce other accessory proteins including Nef, Vpr, Vpu, Vpx, and Vif. Generally, lentiviruses are the causative agents of a variety of disease, including, in addition to immunodeficiency, neurological degeneration, and arthritis. Nucleotide sequences of the various lentiviruses can be found in Genbank under the following Accession Nos. (from J. M. Coffin, S. H. Hughes, and H. E. Varmus, “Retroviruses” Cold Spring Harbor Laboratory Press, 1997 p 804): 1) HIV-1: K03455, M 19921, K02013, M38431, M38429, K02007 and M17449; 2) HIV-2: M30502, J04542, M30895, J04498, M15390, M31113 and L07625; 3) SIV:M29975, M30931, M58410, M66437, L06042, M33262, M19499, M32741, M31345 and L03295; 4) FIV: M25381, M36968 and Ul 1820; 5)BIV. M32690; 6) ELAV: M16575, M87581 and U01866; 6) Visna: M10608, M51543, L06906, M60609 and M60610; 7) CAEV: M33677; and 8) Ovine lentivirus M31646 and M34193. Lentiviral DNA can also be obtained from the American Type Culture Collection (ATCC). For example, feline immunodeficiency virus is available under ATCC Designation No. VR-2333 and VR-3112. Equine infectious anemia virus A is available under ATCC Designation No. VR-778. Caprine arthritis-encephalitis virus is available under ATCC Designation No. VR-905. Visna virus is available under ATCC Designation No. VR-779.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA m ade from nucleotide analogs, and, as applicable to the embodiment b eing described, single-stranded (such as sense or antisense) and double-stranded polynucleotides.

The term “maturation” as used herein refers to the production, post-translational processing, assembly and/or release of proteins that form a viral particle. Accordingly, this includes the processing of viral proteins leading to the pinching off of nascent virion from the cell membrane.

A “POSH nucleic acid” is a nucleic acid comprising a sequence as represented in any of SEQ D NOs: 1, 3, 4, 6, 8, and 10 as well as any of the variants described herein.

A “POSH polypeptide” or “POSH protein” is a polypeptide comprising a sequence as represented in any of SEQ ID NOs: 2, 5, 7, 9 and 11 as well as any of the variations described herein.

A “POSH-associated protein” or “POSH-AP” refers to a protein capable of interacting with and/or binding to a POSH polypeptide. Generally, the POSH-AP may interact directly or indirectly with the POSH polypeptide. A preferred POSH-AP of the application is Cbl-b. Examples of Cbl-b polypeptides are provided throughout.

The terms peptides, proteins and polypeptides are used interchangeably herein.

The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “substantially free of other contaminating proteins”) is defined as encompassing individual preparations of each of the component proteins comprising less than 20% (by dry weight) contaminating protein, and preferably comprises less than 5% contaminating protein. Functional forms of each of the component proteins can be prepared as purified preparations by using a cloned gene as described in the attached examples. By “purified”, it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 85% by weight, more preferably 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above.

A “recombinant nucleic acid” is any nucleic acid that has been placed adjacent to another nucleic acid by recombinant DNA techniques. A “recombined nucleic acid” also includes any nucleic acid that has been placed next to a second nucleic acid by a laboratory genetic technique such as, for example, tranformation and integration, transposon hopping or viral insertion. In general, a recombined nucleic acid is not naturally located adjacent to the second nucleic acid.

The term “recombinant protein” refers to a protein of the present application which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

A “RING domain” or “Ring Finger” is a zinc-binding domain with a defined octet of cysteine and histidine residues. Certain RING domains comprise the consensus sequences as set forth below (amino acid nomenclature is as set forth in Table 1): Cys Xaa Xaa Cys Xaa₁₀₋₂₀ Cys Xaa His Xaa₂₋₅ Cys Xaa Xaa Cys Xaa₁₃₋₅₀ Cys Xaa Xaa Cys or Cys Xaa Xaa Cys Xaa₁₀₋₂₀ Cys Xaa His Xaa₂₋₅ His Xaa Xaa Cys Xaa₁₃₋₅₀ Cys Xaa Xaa Cys. Certain RING domains are represented as amino acid sequences that are at least 80% identical to amino acids 12-52 of SEQ ID NO: 2 and is set forth in SEQ ID No: 26. Preferred RING domains are 85%, 90%, 95%, 98% and, most preferably, 100% identical to the amino acid sequence of SEQ ID NO: 26. Preferred RING domains of the application bind to various protein partners to form a complex that has ubiquitin ligase activity. RING domains preferably interact with at least one of the following protein types: F box proteins, E2 ubiquitin conjugating enzymes and cullins.

The term “RNA interference” or “RNAi” refers to any method by which expression of a gene or gene product is decreased by introducing into a target cell one or more double-stranded RNAs which are homologous to the gene of interest (particularly to the messenger RNA of the gene of interest). RNAi may also be achieved by introduction of a DNA:RNA hybrid wherein the antisense strand (relative to the target) is RNA. Either strand may include one or more modifications to the base or sugar-phosphate backbone. Any nucleic acid preparation designed to achieve an RNA interference effect is referred to herein as an siRNA construct. Phosphorothioate is a particularly common modification to the backbone of an siRNA construct.

“Small molecule” as used herein, is meant to refer to a composition, which has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures comprising arrays of small molecules, often fungal, bacterial, or algal extracts, which can be screened with any of the assays of the application.

An “SH2” or “Src Homology 2” domain is a protein domain that binds specific phosphotyrosine (pY)-containing motifs in the context of three to six amino acids located carboxy-terminal to the pY, providing specificity. An invariant arginine in the SH2 domain is required for pY binding.

An “SH3” or “Src Homology 3 ” domain is a protein domain of generally about 60 amino acid residues first identified as a conserved sequence in the non-catalytic part of several cytoplasmic protein tyrosine kinases (e.g., Src, Abl, Lck). SH3 domains mediate assembly of specific protein complexes via binding to proline-rich peptides. Exemplary SH3 domains are represented by amino acids 137-192, 199-258, 448-505 and 832-888 of SEQ ID NO:2 and are set forth in SEQ ID Nos: 27-30. In certain embodiments, an SH3 domain interacts with a consensus sequence of RXaaXaaPXaaX6P (where X6, as defined in table 1 below, is a hydrophobic amino acid). In certain embodiments, an SH3 domain interacts with one or more of the following sequences: P(T/S)AP, PFRDY, RPEPTAP, RQGPKEP, RQGPKEPFR, RPEPTAPEE and RPLPVAP.

As used herein, the term “specifically hybridizes” refers to the ability of a nucleic acid probe/primer of the application to hybridize to at least 12, 15, 20, 25, 30, 35, 40, 45, 50 or 100 consecutive nucleotides of a POSH sequence, or a sequence complementary thereto, or naturally occurring mutants thereof, such that it has less than 15%, preferably less than 10%, and more preferably less than 5% background hybridization to a cellular nucleic acid (e.g., mRNA or genomic DNA) other than the POSH gene. A variety of hybridization conditions may be used to detect specific hybridization, and the stringency is determined primarily by the wash stage of the hybridization assay. Generally high temperatures and low salt concentrations give high stringency, while low temperatures and high salt concentrations give low stringency. Low stringency hybridization is achieved by washing in, for example, about 2.0×SSC at 50° C., and high stringency is acheived with about 0.2×SSC at 50° C. Further descriptions of stringency are provided below.

As applied to polypeptides, “substantial sequence identity” means that two peptide sequences, when optimally aligned, such as by the programs GAP or BESTFIT using default gap which share at least 90 percent sequence identity, preferably at least 95 percent sequence identity, more preferably at least 99 percent sequence identity or more. Preferably, residue positions which are not identical differ by conservative amino acid substitutions. For example, the substitution of amino acids having similar chemical properties such as charge or polarity are not likely to effect the properties of a protein. Examples include glutamine for asparagine or glutamic acid for aspartic acid.

As is well known, genes for a particular polypeptide may exist in single or multiple copies within the genome of an individual. Such duplicate genes may be identical or may have certain modifications, including nucleotide substitutions, additions or deletions, which all still code for polypeptides having substantially the same activity.

A “TKB” or “Tyrosine Kinase-Binding” domain is a phosphotyrosine-binding domain that comprises three structural motifs: a four-helix bundle, an EF hand, and a divergent SH2 domain. These three structural motifs together form an integrated phosphoprotein-recognition domain.

A “virion” is a complete viral particle; nucleic acid and capsid (and a lipid envelope in some viruses. A “viral particle” may be incomplete, as when produced by a cell transfected with a defective virus (e.g., an HIV virus-like particle system).

TABLE 1
Abbreviations for classes of amino acids*

| | | Amino Acids |
|---|---|---|
| Symbol | Category | Represented |
| X1 | Alcohol | Ser, Thr |
| X2 | Aliphatic | Ile, Leu, Val |
| Xaa | Any | Ala, Cys, Asp, Glu, Phe, |
| | | Gly, His, Ile, Lys, Leu, |
| | | Met, Asn, Pro, Gln, Arg, |
| | | Ser, Thr, Val, Trp, Tyr |
| X4 | Aromatic | Phe, His, Trp, Tyr |
| X5 | Charged | Asp, Glu, His, Lys, Arg |
| X6 | Hydrophobic | Ala, Cys, Phe, Gly, His, |
| | | Ile, Lys, Leu, Met, Thr, |
| | | Val, Trp, Tyr |
| X7 | Negative | Asp, Glu |
| X8 | Polar | Cys, Asp, Glu, His, Lys, |
| | | Asn, Gln, Arg, Ser, Thr |
| X9 | Positive | His, Lys, Arg |
| X10 | Small | Ala, Cys, Asp, Gly, Asn, |
| | | Pro, Ser, Thr, Val |
| X11 | Tiny | Ala, Gly, Ser |
| X12 | Turnlike | Ala, Cys, Asp, Glu, Gly, |
| | | His, Lys, Asn, Gln, Arg, |
| | | Ser, Thr |
| X13 | Asparagine-Aspartate | Asn, Asp |

*Abbreviations as adopted from http://smart.emblheidelberg.de/SMART_DATA/alignments/consensus/grouping.html.

### 2. OVERVIEW

In certain aspects, the application relates to the discovery of novel associations between Cbl-b proteins and other proteins (termed Cbl-b-APs), and related methods and compositions. In certain aspects, the application relates to novel associations among certain disease states, Cbl-b nucleic acids and proteins, and Cbl-b-AP nucleic acids and proteins. In preferred embodiments, the application relates to the discovery of novel associations between Cbl-b proteins and POSH proteins, and related methods and compositions. In further embodiments, the application relates to novel associations among certain disease states, Cbl-b nucleic acids and proteins, and POSH nucleic acids and proteins.

In certain aspects, by identifying proteins associated with Cbl-b, and particularly human Cbl-b, the present application provides a conceptual link between the Cbl-b-APs and cellular processes and disorders associated with Cbl-b-APs, and Cbl-b itself. Accordingly, in certain embodiments of the disclosure, agents that modulate a Cbl-b-AP, such as POSH, may now be used to modulate Cbl-b functions and disorders associated with Cbl-b function, such as viral disorders, and disorders of the immune system. Additionally, test agents may be screened for an effect on a Cbl-b-AP, such as POSH, and then further tested for an effect on a Cbl-b function or a disorder associated with Cbl-b function. Likewise, in certain embodiments of the disclosure, agents that modulate Cbl-b may now be used to modulate Cbl-b-AP, such as POSH, functions and disorders associated with Cbl-b-AP function, such as disorders associated with POSH function, including viral disorders, POSH-associated cancers, and POSH-associated neural disorders. Additionally, test agents may be screened for an effect on Cbl-b and then further tested for effect on a Cbl-b-AP function or a disorder associated with Cbl-b-AP function. In further aspects, the application provides nucleic acid agents (e.g., RNAi probes, antisense nucleic acids), antibody-related agents, small molecules and other agents that affect Cbl-b function, and the use of same in modulating Cbl-b and/or Cbl-b-AP activity.

In certain aspects, the application relates to the discovery that a Cbl-b polypeptide interacts with one or more POSH polypeptides. Accordingly, the application provides complexes comprising Cbl-b and POSH. In one aspect, the application relates to the discovery that Cbl-b binds directly with POSH. This interaction was identified by Applicants in a yeast 2-hybrid assay.

Cbl-b polypeptides contain an amino-terminal tyrosine kinase-binding (TKB) domain, which inlcudes three interacting domains comprising a four-helix bundle, a Ca²+-binding EF hand, and a variant Src homology 2 (SH2) domain. Cbl-b polypeptides additionally comprise a RING finger and a carboxyl-terminal proline-rich domain with potential tyrosine phosphorylation sites. Cbl proteins have a high degree of sequence homology between their tyrosine kinase-binding domains and RING finger domains. Further, Cbl-b is highly homologous to the mammalian Cbl and the nematode Sli-1 proteins.

This application provides four Cbl-b variants and shows that POSH interacts with one or more of these variants. In one aspect, a POSH polypeptide interacts with a human Cbl-b polypeptide (UniGene No.: Hs.3144). In another aspect, the POSH polypeptide interacts with an alternative human Cbl-b polypeptide (UniGene No.: Hs.381921) that may be a splice variant of Cbl-b. In yet another aspect, a POSH polypeptide interacts with a human Cbl-b polypeptide that is a splice variant represented by the amino acid sequence depicted in SEQ ID NO: 45, which is encoded by the nucleic acid sequence depicted in SEQ ID NO: 43. In yet another aspect, a POSH polypeptide interacts with a human Cbl-b polypeptide that is a splice variant represented by the amino acid sequence depicted in SEQ ID NO: 46, which is encoded by the nucleic acid sequence depicted in SEQ ID NO: 44. SH3 domains bind proline-rich sequences. Accordingly, in certain embodiments, a Cbl-b polypeptide of the application may interact via its carboxyl-terminal proline rich domain with an SH3 domain of a POSH polypeptide.

Cbl-b polypeptides have been shown to function as adaptor proteins by interacting with other signaling molecules, e.g., interaction with cell surface receptor tyrosine kinases, e.g., EGFR (Ettenberg, S A et al (2001) J Biol Chem 276:77-84) or with proteins such as Syk, Crk-L, PI3K, Grb2, or Vav (See, for example, Elly, C et al (1999) Oncogene 18:1147-56; Elly, C et al (1999) Oncogene 18:1147-56; Fang, D et al. (2001) J Biol Chem 16:4872-8; Ettenberg, S A et al (1999) Oncogene 18:1855-66; Bustelo, X R et al. (1997) Oncogene 15:2511-20). It has been demonstrated that Cbl-b polypeptides interact directly with the nucleotide exchange factor, Vav (Bustelo, X R et al. (1997) Oncogene 15:2511-20).

Cbl-b has been shown to function as an E3 ubiquitin ligase that recognizes tyrosine phosphorylated substrates through its SH2 domain and through its RING domain, recruits a ubiquitin-conjugating enzyme, E2 (Joazeiro, C et al. (1999) Science 286:309-312) Additionally, certain Cbl-b polypeptides have been shown to associate directly with the p85 subunit of P13K and to function as an E3 ligase in the ubiquitination of PI3K (Fang, D et al. (2001) J Biol Chem 16:4872-8).

Cbl-b has also been shown to be a negative regulator of T-cell activation. Cbl-b-deficient mice become very susceptible to experimental autoimmune encephalomyelitis (Chiang, Y J et al. (2000) Nature 403:216-220). Also, Cbl-b-deficient mice develop spontaneous autoimmunity (Bachmaier, K, et al (2000) Nature 403:211-216). Furthermore, Cbl-b is a major susceptibility gene for rat type 1 diabetes mellitus (Yokoi, N et al (2002) Nature Genet. 31:391-394).

Accordingly, in certain aspects, the Cbl-b-AP, POSH, participates in the formation of Cbl-b complexes, including human Cbl-b-containing complexes. Certain P OSH p olypeptides m ay b e i nvolved i n d isorders o f the immune s ystem, e.g., autoimmune disorders. Certain POSH polypeptides may be involved in the regulation of T-cell activation. In certain aspects, POSH participates in the ubiquitination of PI3K. In certain aspects, Cbl-b polypeptides participate in POSH-mediated processes.

The term Cbl-b is used herein to refer to full-length, human Cbl-b (UniGene No.: Hs.3144) as well as an alternative Cbl-b (UniGene No.: Hs.381921) composed of two separate Cbl-b sequences (e.g., nucleic acid sequences) that may be a splice variant. The term Cbl-b is used herein to refer as well to the human Cbl-b splice variant represented by the amino acid sequence of SEQ ID NO: 45, which is encoded by the nucleic acid sequence of SEQ ID NO: 43 and to the human Cbl-b splice variant represented by the amino acid sequence of SEQ ID NO: 46, which is encoded by the nucleic acid sequence of SEQ ID NO: 44. The term Cbl-b is used herein to refer as well to various naturally occurring Cbl-b homologs, as well as functionally similar variants and fragments that retain at least 80%, 90%, 95%, or 99% sequence identity to a naturally occurring Cbl-b (e.g., SEQ ID NOs: 37-44 and 45-50). The term specifically includes human Cbl-b nucleic acid and amino acid sequences and the sequences presented in the Examples.

The Cbl-b-AP, POSH, intersects with and regulates a wide range of key cellular functions that may be manipulated by affecting the level of and/or activity of POSH polypeptides or POSH-AP polypeptides. Many features of POSH, and particularly human POSH, are described in PCT patent publications WO03/095971A2 (application no. WO2002US0036366) and WO03/078601A2 (application no. WO2003US0008194) the teachings of which are incorporated by reference herein.

As described in the above-referenced publications, native human POSH is a large polypeptide containing a RING domain and four SH3 domains. POSH is a ubiquitin ligase (also termed an “E3” enzyme); the RING domain mediates ubiquitination of, for example, the POSH polypeptide itself. POSH interacts with a large number of proteins and participates in a host of different biological processes. As demonstrated in this disclosure, POSH associates with a number of different proteins in the cell. POSH co-localizes with proteins that are known to be located in the trans-Golgi network, implying that POSH participates in the trafficking of proteins in the secretory system. The term “secretory system” should be understood as referring to the membrane compartments and associated proteins and other molecules that are involved in the movement of proteins from the site of translation to a location within a vacuole, a compartment in the secretory pathway itself, a lysosome or endosome or to a location at the plasma membrane or outside the cell. Commonly cited examples of compartments in the secretory system include the endoplasmic reticulum, the Golgi apparatus and the cis and trans Golgi networks. In addition, Applicants have demonstrated that POSH is necessary for proper secretion, localization or processing of a variety of proteins, including phospholipase D, HIV Gag, HIV Nef, Rapsyn and Src. Many of these proteins are myristoylated, indicating that POSH plays a general role in the processing and proper localization of myristoylated proteins. Accordingly, in certain aspects, Cbl-b may play a role in the processing and proper localization of myristolyated proteins. N-myristoylation is an acylation process, which results in covalent attachment of myristate, a 14-carbon saturated fatty acid to the N-terminal glycine of proteins (Farazi et al., J. Biol. Chem. 276: 39501-04 (2001)). N-myristoylation occurs co-translationaly and promotes weak and reversible protein-mernbrane interaction. Myristoylated proteins are found both in the cytoplasm and associated with membrane. Membrane association i s d ependent o n p rotein c onfiguration, i.e., s urface accessibility o f t he myristoyl group may be regulated by protein modifications, such as phosphorylation, ubiquitination etc. Modulation of intracellular transport of myristoylated proteins in the application includes effects on transport and localization of these modified proteins.

As described herein, POSH and Cbl-b are involved in viral maturation, including the production, post-translational processing, assembly and/or release of proteins in a viral particle. Accordingly, viral infections may be ameliorated by inhibiting an activity (e.g., ubiquitin ligase activity or target protein interaction) of POSH or Cbl-b (e.g., inhibition of ubiquitin ligase activity), and in preferred embodiments, the virus is a retroid virus, an RNA virus or an envelope virus, including HIV, Ebola, HBV, HCV, HTLV, West Nile Virus (WNV) or Moloney Murine Leukemia Virus (MMuLV). Additional viral species are described in greater detail below. In certain instances, a decrease of a POSH function is lethal to cells infected with a virus that employs POSH in release of viral particles.

POSH polypeptides have been shown to bind directly to the POSH-APs PACS-1, HLA-A, and HLA-B in a 2-hybrid assay. PACS-1 has been shown to bind to HIV Nef and is involved in the Nef-mediated process of HLA down-modulation from the surface of a cell infected with HIV. Accordingly, POSH may interact with Nef through its association with PACS-1. In certain aspects, POSH inhibition results in inhibition of PACS-1 activity, including inhibition of PACS-1 interaction with Nef and PACS-1 activity associated with Nef-mediated down-modulation of MHC class I molecules. In additional aspects, the application relates to inhibition of the down-regulation of HLA-A and HLA-B by Nef. In certain embodiments, POSH may interact with Nef through its interaction with HLA-A. In certain embodiments, POSH may interact with Nef through its interaction with HLA-B. In fther embodiments of the application, POSH inhibition results in inhibition of HLA-A and/or HLA-B interaction with Nef.

To the extent that Nef down-modulates CD4 and MHC class I molecules, in certain embodiments, by inhibiting POSH, CD4 and MHC class I molecule cell surface levels are accordingly increased. Additionally, in certain aspects, by inhibiting Cbl-b activity in a cell infected with HIV, Nef-mediated down-modulation of CD4 and MHC class I molecule cell surface levels may be inhibited.

Another Nef-mediated process inhibited by methods of the present application is T cell activation. Nef has been implicated in T cell activation, for instance, in the production of IL-2. Its expression has been linked to the up-regulation of genes whose products are known to activate the HIV long terminal repeat (LTR), which contains enhancer and promoter sequences (3 Virol (1999) 6094-6099; Immunity (2001) 14:763-777). Nef has been shown to form a complex with the cellular serine/threonine kinase p21-activated kinase 2 (Pak2) and to mediate Pak2 activation. Paks have been implicated in T cell activation. Accordingly, a Nef-mediated process includes Pak2 activation. (See, for example, Curr Biol (1999) 9:1407-1410; J Virol (2000) 74:11081-11087). In certain embodiments, inhibition of POSH (e.g., POSH polypeptide expression) results in inhibition of Pak2 activation. Nef has also been associated with nuclear factor of activated T cell (NFAT) transcriptional activity (J Virol (2001) 75:3034-3037). Additionally, Nef may associate with known activators of Paks, such as the Rho family GTPases, CDC42 and Racl, through its interaction with the guanine I nucleotide exchange factor, Vav (or Vav2) (Mol Cell (1999) 3:729-739) or Pix (3 Virol (1999) 73:9899-9907). In certain embodiments, POSH associates with the GTPase, Rac1. Accordingly, in certain aspects, POSH may interact with Nef through its association with Rac1.

Additionally, Vav is a Cbl-b-AP. Cbl-b has been shown to interact with Vav directly. Also, an increase in Cbl-b expression has been noted in peripheral blood mononuclear cells (PBMCs) from immune activated HIV-1 infected individuals in response to non-specific T-cell receptor stiumlation (Biochem Biophys Res Commun (2002) 298:464-7). Accordingly, in certain embodiments, Cbl-b may interact with HIV Nef through its association with Vav. Cbl-b polypeptides have been implicated in the negative regulation of T cell activation. Accordingly, in further embodiments, modulation of a complex comprising Cbl-b and a Cbl-b-AP, such as Vav or Nef, results in inhibition of the Nef-mediated process of Pak2 activation.

In certain aspects, the application describes an HPOSH interaction with Rac, a small GTPase and the POSH associated kinases MLK, MKK and JNK. Rho, Rac and Cdc42 operate together to regulate organization of the actin cytoskeleton and the MLK-MKK-JNK MAP kinase pathway (referred to herein as the “JNK pathway” or “Rac-JNK pathway” (Xu et al., 2003, EMBO J. 2: 252-61). Ectopic expression of mouse POSH (“mPOSH”) activates the JNK pathway and causes nuclear localization of NF-κB. Overexpression of mPOSH in fibroblasts stimulates apoptosis. (Tapon et al. (1998) EMBO J. 17:1395-404). In Drosophila, POSH may interact with, or otherwise influence the signaling of, another GTPase, Ras. (Schnorr et al. (2001) Genetics 159: 609-22). The JNK pathway and NF-κB regulate a variety of key genes involved in, for example, immune responses, inflammation, cell proliferation and apoptosis. For example, NF-κB regulates the production of interleukin 1, interleukin 8, tumor necrosis factor and many cell adhesion molecules. NF-κB has both pro-apoptotic and anti-apoptotic roles in the cell (e.g., in FAS-induced cell death and TNF-alpha signaling, respectively). NF-KB is negatively regulated, in part, by the inhibitor proteins IκBa and IκBβ (collectively termed “IκB”). Phosphorylation of IκB permits activation and nuclear localization of NF-κB. Phosphorylation of IκB triggers its degradation by the ubiquitin system. In an additional embodiment, a POSH polypeptide promotes nuclear localization of NF-κB. In further embodiments, manipulation of POSH levels and/or activities may be used to manipulate apoptosis. By upregulating POSH, apoptosis may be stimulated in certain cells, and this will generally be desirable in conditions characterized by excessive cell proliferation (e.g., in certain cancers). By downregulating POSH, apoptosis may be diminished in certain cells, and this will generally be desirable in conditions characterized by excessive cell death, such as myocardial infarction, stroke, degenerative diseases of muscle and nerve (particularly Alzheimer's disease), and for organ preservation prior to transplant. In a further embodiment, a POSH polypeptide associates with a vesicular trafficking complex, such as a clathrin- or coatomer- containing complex, and particularly a trafficking complex that localizes to the nucleus and/or Golgi apparatus.

As described in WO03/078601A2 (application no. WO2003US0008194), POSH is overexpressed in a variety of cancers, and downregulation of POSH is associated with a decrease in proliferation in at least one cancer cell line. Accordingly, agents that modulate POSH itself or a POSH-AP, such as Cbl-b, may be used to treat POSH associated cancers. POSH associated cancers include those cancers in which POSH is overexpressed and/or in which downregulation of POSH leads to a decrease in the proliferation or survival of cancer cells. POSH-associated cancers are described in more detail below. In addition, it is notable that many proteins shown herein to be affected by POSH downregulation are themselves involved in cancers. Phospholipase D and SRC are both aberrantly processed in a POSH-impaired cell, and therefore modulation of POSH and/or a POSH-AP, such as Cbl-b, may affect the wide range of cancers in which PLD and SRC play a significant role.

As described in WO03/095971A2 (application no. WO2002US0036366) and WO03/078601A2 (application no. WO2003US0008194), POSH polypeptides function as E3 enzymes in the ubiquitination system. Accordingly, downregulation or upregulation of POSH ubiquitin ligase activity can be used to manipulate biological processes that are affected by protein ubiquitination. Modulation of POSH ubiquitin ligase activity may be used to affect Cbl-b and related biological processes, and likewise, modulation of Cbl-b may be used to affect POSH ubiquitin ligase activity and related processes. Downregulation or upregulation may be achieved at any stage of POSH formation and regulation, including transcriptional, translational or post-translational regulation. For example, POSH transcript levels may be decreased by RNAi targeted at a POSH gene sequence. As another example, POSH ubiquitin ligase activity may be inhibited by contacting POSH with an antibody that binds to and interferes with a POSH RING domain or a domain of POSH that mediates interaction with a target protein (a protein that is ubiquitinated at least in part because of POSH activity). As a further example, small molecule inhibitors of POSH ubiquitin ligase activity are provided herein. As another example, POSH activity may be increased by causing increased expression of POSH or an active portion thereof. POSH, and POSH-APs that modulate POSH ubiquitin ligase activity may participate in biological processes including, for example, one or more of the various stages of a viral lifecycle, such as viral entry into a cell, production of viral proteins, assembly of viral proteins and release of viral particles from the cell. POSH may participate in diseases characterized by the accumulation of ubiquitinated proteins, such as dementias (e.g., Alzheimer's and Pick's), inclusion body myositis and myopathies, polyglucosan body myopathy, and certain forms of amyotrophic lateral sclerosis. POSH may participate in diseases characterized by excessive or inappropriate ubiquitination and/or protein degradation. 10

### 4. METHODS AND COMPOSITIONS FOR TREATING CBL-B AND CBL-B-AP-ASSOCIATED DISEASES

In certain aspects, the application provides methods and compositions for treatment of Cbl-b-associated diseases (disorders), including cancer and viral disorders, as well as disorders of the immune system, such as, for example, autoimmune disorders. In certain aspects, the application provides methods and compositions for treatment of Cbl-b-AP-associated diseases (disorders), such as POSH-associated disorders, including cancer and viral disorders, as well as neural disorders and disorders associated with unwanted apoptosis, including, for example a variety of neurodegenerative disorders, such as Alzheimer's disease.

In certain embodiments, the application relates to viral disorders (e.g., viral infections), and particularly disorders caused by retroid viruses, RNA viruses and/or envelope viruses. In view of the teachings herein, one of skill in the art will understand that the methods and compositions of the application are applicable to a wide range of viruses such as, for example, retroid viruses, RNA viruses, and envelope viruses. In a preferred embodiment, the present application is applicable to retroid viruses. In a more preferred embodiment, the present application is further applicable to retroviruses (retroviridae). In another more preferred embodiment, the present application is applicable to lentivirus, including primate lentivirus group. In a most preferred embodiment, the present application is applicable to Human Immunodeficiency virus (HIV), Human Immunodeficiency virus type-1 (HIV-1), Hepatitis B Virus (HBV) and Human T-cell Leulkemia Virus (HTLV).

While not intended to be limiting, relevant retroviruses include: C-type retrovirus which causes lymphosarcoma in Northern Pike, the C-type retrovirus which infects mink, the caprine lentivirus which infects sheep, the Equine Infectious Anemia Virus (EIAV), the C-type retrovirus which infects pigs, the Avian Leukosis Sarcoma Virus (ALSV), the Feline Leukemia Virus (FeLV), the Feline Aids Virus, the Bovine Leukemia Virus (BLV), Moloney Murine Leukemia Virus (MMuLV), the Simian Leukemia Virus (SLV), the Simian Immuno-deficiency Virus (SIV), the Human T-cell Leukemia Virus type-I (HTLV-I), the Human T-cell Leukemia Virus type-II (HTLV-U), Human Immunodeficiency virus type-2 (HIV-2) and Human Immunodeficiency virus type-1 (HIV-1).

The method and compositions of the present application are further applicable to RNA viruses, including ssRNA negative-strand viruses and ssRNA positive-strand viruses. The ssRNA positive-strand viruses include Hepatitis C Virus (HCV). In a preferred embodiment, the present application is applicable to mononegavirales, including filoviruses. Filoviruses further include Ebola viruses and Marburg viruses. In another preferred embodiment, the present invention is applicable to flaviviruses, including West Nile Virus (WNV).

Other RNA viruses include picomaviruses such as enterovirus, poliovirus, coxsackievirus and hepatitis A virus, the caliciviruses, including Norwalk-like viruses, the rhabdoviruses, including rabies virus, the togaviruses including alphaviruses, Semliki Forest virus, denguevirus, yellow fever virus and rubella virus, the orthomyxoviruses, including Type A, B, and C influenza viruses, the bunyaviruses, including the Rift Valley fever virus and the hantavirus, the filoviruses such as Ebola virus and Marburg virus, and the paramyxoviruses, including mumps virus and measles virus. Additional viruses that may be treated include herpes viruses.

The methods and compositions of the present application are farther applicable to hepatotrophic viruses, including HAV, HBV, HCV, HDV, and HEV. In certain aspects, the application relates to a method of inhibiting a hepatotrophic virus, comprising administering a Cbl-b-AP inhibitor, for example, a POSH inhibitor, to a subject in need thereof. In further aspects, the application relates to a method of treating a viral hepatitis infection, comprising administering a Cbl-b-AP inhibitor, such as a POSH inhibitor, to a subject in need thereof. A viral hepatitis infection may be caused by a hepatotrophic virus, such as HAV, HBV, HCV, HDV, or HEV. In certain embodiments, the application relates to a method of treating an HBV infection by administering a Cbl-b-AP inhibitor, such as a POSH inhibitor, to a subject in need thereof.

In other embodiments, the application relates to methods of treating or preventing cancer diseases. The terms “cancer,” “tumor,” and “neoplasia” are used interchangeably herein. As used herein, a cancer (tumor or neoplasia) is characterized by one or more of the following properties: cell growth is not regulated by the normal biochemical and physical influences in the environment; anaplasia (e.g., lack of normal coordinated cell differentiation); and in some instances, metastasis. Cancer diseases include, for example, anal carcinoma, bladder carcinoma, breast carcinoma, cervix carcinoma, chronic lymphocytic leukemia, chronic myelogenous leukemia, endometrial carcinoma, hairy cell leukemia, head and neck carcinoma, lung (small cell) carcinoma, multiple myeloma, non-Hodgkin's lymphoma, follicular lymphoma, ovarian carcinoma, brain tumors, colorectal carcinoma, hepatocellular carcinoma, Kaposi's sarcoma, lung (non-small cell carcinoma), melanoma, pancreatic carcinoma, prostate carcinoma, renal cell carcinoma, and soft tissue sarcoma. Additional cancer disorders can be found in, for example, Isselbacher et al. (1994) Harrison's Principles of Internal Medicine 1814-1877, herein incorporated by reference.

In a specific embodiment, anticancer therapeutics of the application are used in treating a Cbl-b-AP-associated cancer, particularly a POSH-associated cancer. As described herein, POSH-associated cancers include, but are not limited to, the thyroid carcinoma, liver cancer (hepatocellular cancer), lung cancer, cervical cancer, ovarian cancer, renal cell carcinoma, lymphoma, osteosacoma, liposarcoma, leukemia, breast carcinoma, and breast adeno-carcinoma.

Preferred antiviral and anticancer therapeutics of the application can function by disrupting the biological activity of a Cbl-b polypeptide or Cbl-b complex in viral maturation. Certain therapeutics of the application function by disrupting the activity of a Cbl-b-AP, such as POSH, in viral maturation. Certain therapeutics of the application function by disrupting the activity of Cbl-b by inhibiting the ubiquitin ligase activity of a Cbl-b polypeptide. Additionally, certain therapeutics of the application function by disrupting the activity of a Cbl-b-AP polypeptide (e.g., POSH) by inhibiting the ubiquitin ligase activity of a Cbl-b-AP (e.g., POSH) polypeptide.

In other embodiments, the application relates to methods of treating or preventing neurological disorders. In one aspect, the invention provides methods and compositions for the identification of compositions that interfere with the function of a Cbl-b or a Cbl-b-AP, such as POSH, which function may relate to aberrant protein processing associated with a neurodegenerative disorder, such as for example, the processing of amyloid beta precursor protein associated with Alzheimer's disease. Neurological disorders include disorders associated with increased levels of amyloid P production, such as for example, Alzheimer's disease. Neurological disorders also include Parkinson's disease, Huntington's disease, schizophrenia, Niemann-Pick's disease, and prion-associated diseases

Exemplary therapeutics of the application include nucleic acid therapies such as, for example, RNAi constructs (small inhibitory RNAs), antisense oligonucleotides, ribozyme, and DNA enzymes. Other therapeutics include polypeptides, peptidomimetics, antibodies and small molecules.

Antisense therapies of the application include methods of introducing antisense nucleic acids to disrupt the expression of Cbl-b polypeptides or proteins that are necessary for Cbl-b function. Antisense therapies of the application also include methods of introducing antisense nucleic acids to disrupt the expression of Cbl-b-AP polypeptides, such as POSH polypeptides, or proteins that are necessary for Cbl-b-AP (e.g., POSH) function.

RNAi therapies include methods of introducing RNAi constructs to downregulate the expression of Cbl-b polypeptides or POSH polypeptides. Exemplary RNAi therapeutics include any one of SEQ ID NOs: 59-64. Exemplary RNAi therapeutics also include any one of SEQ ID NOs: 15, 16, 18, 19, 21, 22, 24 and 25.

Therapeutic polypeptides may be generated by designing polypeptides to mimic certain protein domains important in the formation of Cbl-b: Cbl-b-AP complexes (e.g., Cbl-b:POSH complexes), such as, for example, SH3 or RING domains. For example, a polypeptide comprising a Cbl-b domain such as, for example, an SH2 domain of a Cbl-b polypeptide, will compete for binding to a Cbl-b SH2 domain and will therefore act to disrupt binding of a partner protein. Also, for example, a polypeptide comprising a POSH SH3 domain such as, for example, the SH3 domain as set forth in SEQ ID NO: 30 will compete for binding to a POSH SH3 domain and will therefore act to disrupt binding of a partner protein. In one embodiment, a binding partner may be a Gag polypeptide. In another embodiment, a binding partner may be Rac. In a further embodiment, a polypeptide that resembles an L domain may disrupt recruitment of Gag to the POSH complex.

In view of the specification, methods for generating antibodies directed to epitopes of Cbl-b and POSH are known in the art. Antibodies may be introduced into cells by a variety of methods. One exemplary method comprises generating a nucleic acid encoding a single chain antibody that is capable of disrupting a Cbl-b:POSH complex. Such a nucleic acid may be conjugated to antibody that binds to receptors on the surface of target cells. It is contemplated that in certain embodiments, the antibody may target viral proteins that are present on the surface of infected cells, and in this way deliver the nucleic acid only to infected cells. Once bound to the target cell surface, the antibody is taken up by endocytosis, and the conjugated nucleic acid is transcribed and translated to produce a single chain antibody that interacts with and disrupts the targeted Cbl-b:POSH complex. Nucleic acids expressing the desired single chain antibody may also be introduced into cells using a variety of more conventional techniques, such as viral transfection (e.g., using an adenoviral system) or liposome-mediated transfection.

Small molecules of the application may be identified for their ability to modulate the formation of Cbl-b:POSH complexes.

Certain embodiments of the disclosure relate to use of a small molecule as an inhibitor of Cbl-b. Examples of such small molecules include the following compounds:

| | MW | |
|---|---|---|
| CAS | (gr/mol) | Structure |
| 412945- 52-9 | 398.33 | 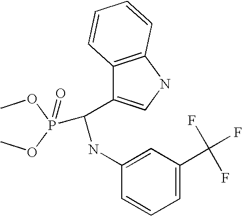 |
| 52686- 41-6 | 368.44 | 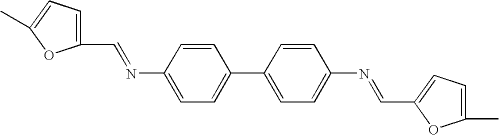 |
| 38536- 86-6 | 252.36 | 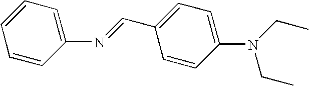 |
| 57182- 49-7 | 263.3 | 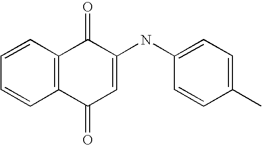 |
| 63245- 76-1 | 289.27 | 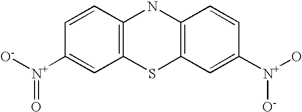 |
| 120999- 01-1 | 263.3 | 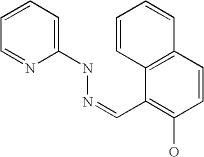 |
| 126324- 76-3 | 203.2 | 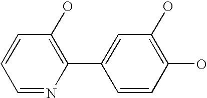 |
| 164399- 38-0 | 386.25 | 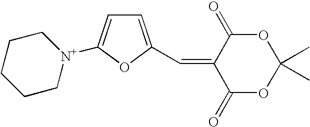 |
| 324526- 59-2 | 352.63 | 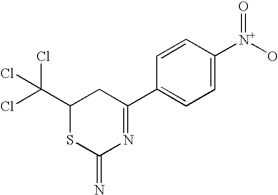 |
| 295345- 11-8 | 256.31 | 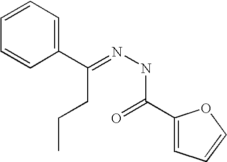 |
| no cas | 336.31 | 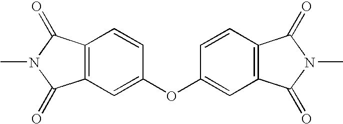 |
| 325958- 44-9 | 323.14 | 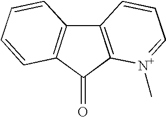 |
| 88680- 99-3 | 275.33 | 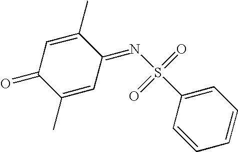 |

Certain embodiments of the disclosure relate to use of a small molecule as an inhibitor of the Cbl-b-AP, POSH. Examples of such small molecules include the following compounds:

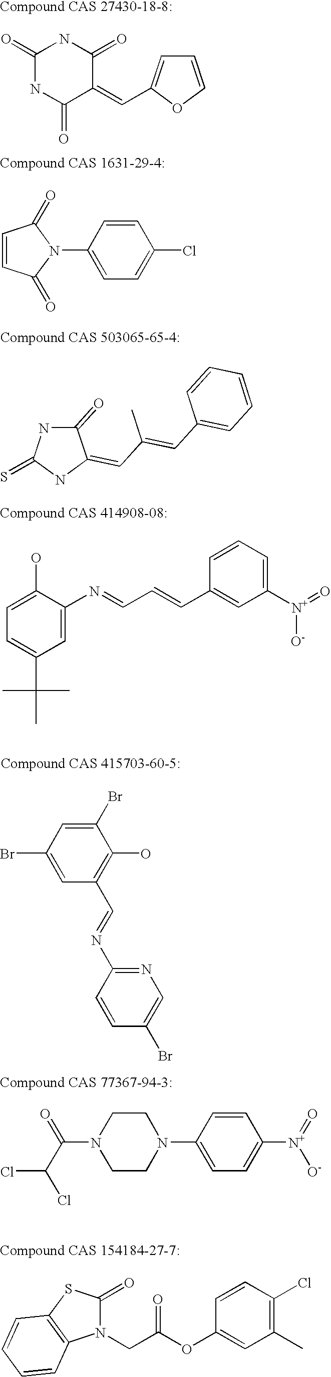

In certain embodiments, compounds useful in the instant compositions and methods include heteroarylmethylene-dihydro-2,4,6-pyrimidinetriones and their thione analogs. Preferred heteroaryl moieties include 5-membered rings such as thienyl, furyl, pyrrolyl, oxazolyl, thiazolyl, and imidazolyl moieties.

In certain embodiments, compounds useful in the instant compositions and methods include N-arylmaleimides, especially N-phenylmaleimides, in which the phenyl group may be substituted or unsubstituted.

In certain embodiments, compounds useful in the instant compositions and methods include arylallylidene-2,4-imidazolidinediones and their thione analogs. Preferred aryl groups are phenyl groups, and both the aryl and allylidene portions of the molecule may be substituted or unsubstituted.

In certain embodiments, compounds useful in the instant compositions and methods include substituted distyryl compounds and aza analogs thereof such as substituted 1,4-diphenylazabutadiene compounds.

In certain other embodiments, compounds useful in the instant compositions and methods include substituted styrenes and aza analogs thereof, such as 1,2-diphenylazaethylenes and 1-phenyl-2-pyridyl-azaethelenes.

In yet other embodiments, compounds useful in the instant compositions and methods include N-aryl-N′-acylpiperazines. In such compounds, the aryl ring, the acyl substituent, and/or the piperazine ring may be substituted or unsubstituted.

In additional embodiments, compounds useful in the instant compositions and methods include aryl esters of (2-oxo-benzooxazol-3-yl)-acetic acid, and analogs thereof in which one or more oxygen atoms are replaced by sulfur atoms.

The generation of nucleic acid based therapeutic agents directed to Cbl-b and Cbl-b-APs, such as POSH, is described below.

Methods for identifying and evaluating further modulators of Cbl-b and Cbl-b-APs, such as POSH, are also provided below.

### 5. RNA INTERFERENCE RIBOZYMES, ANTISENSE AND RELATED CONSTRUCTS

In certain aspects, the application relates to RNAi, ribozyme, antisense and other nucleic acid-related methods and compositions for manipulating (typically decreasing) a Cbl-b activity. Exemplary RNAi and ribozyme molecules may comprise a sequence as shown in any of SEQ ID NOs: 59-64. Additionally, specific instances of nucleic acids that may be used to design nucleic acids for RNAi, ribozyme, antisense are provided in the Examples. In certain aspects, the application relates to RNAi, ribozyme, antisense and other nucleic acid-related methods and compositions for manipulating (typically decreasing) a Cbl-b-AP (e.g., POSH) activity. Exemplary RNAi and ribozyme molecules may comprise a sequence as shown in any of SEQ ID NOs: 15, 16, 18, 19, 21, 22, 24 and 25.

Certain embodiments of the application make use of materials and methods for effecting knockdown of one or more Cbl-b or Cbl-b-AP (e.g., POSH) genes by means of RNA interference (RNAi). RNAi is a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Furthermore, Accordingly, RNAi may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the RNAi (sequence-specific) pathway, the initiating dsRNA is firstbroken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length. The nonspecific effects occur because dsRNA activates two enzymes: PKR, which in its active form phosphorylates the translation initiation factor eIF2 to shut down all protein synthesis, and 2′, 5′ oligoadenylate synthetase (2′, 5′-AS), which synthesizes a molecule that activates Rnase L, a nonspecific enzyme that targets all mRNAs. The nonspecific pathway may represent a host response to stress or viral infection, and, in general, the effects of the nonspecific pathway are preferably minimized under preferred methods of the present application. Significantly, longer dsRNAs appear to be required to induce the nonspecific pathway and, accordingly, dsRNAs shorter than about 30 bases pairs are preferred to effect gene repression by RNAi (see Hunter et al. (1975) J Biol Chem 250: 409-17; Manche et al. (1992) Mol Cell Biol 12: 5239-48; Minks et al. (1979) J Biol Chem 254: 10180-3; and Elbashir et al. (2001) Nature 411: 494-8).

RNAi has been shown to be effective in reducing or eliminating the expression of genes in a number of different organisms including Caenorhabditiis elegans (see e.g., Fire et al. (1998) Nature 391: 806-11), mouse eggs and embryos (Wianny et al. (2000) Nature Cell Biol 2: 70-5; Svoboda et al. (2000) Development 127: 4147-56), and cultured RAT-1 fibroblasts (Bahramina et al. (1999) Mol Cell Biol 19: 274-83), and appears to be an anciently evolved pathway available in eukaryotic plants and animals (Sharp (2001) Genes Dev. 15: 485-90). RNAi has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass (2001) Nature 411: 428-9). In mammalian cells, siRNAs are effective at concentrations that are several orders of magnitude below the concentrations typically used in antisense experiments (Elbashir et al. (2001) Nature 411: 494-8).

The double stranded oligonucleotides used to effect RNAi are preferably less than 30 base pairs in length and, more preferably, comprise about 25, 24, 23, 22,21, 20, 19, 18 or 17 base pairs of ribonucleic acid. Optionally the dsRNA oligonucleotides of the application may include 3 ′ overhang ends. Exemplary 2-nucleotide 3′ overhangs maybe composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashir et al. (2001) Nature 411: 494-8). Longer dsRNAs of 50, 75, 100 or even 500 base pairs or more may also be utilized in certain embodiments of the application. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable the skilled artisan. Exemplary dsRNAs may be synthesized chemically or produced in vitro or in vivo using appropriate expression vectors. Exemplary synthetic RNAs include 21 nucleotide RNAs chemically synthesized using methods known in the art (e.g., Expedite RNA phophoramidites and thymidine phosphoramidite (Proligo, Germany). Synthetic oligonucleotides are preferably deprotected and gel-purified using methods known in the art (see e.g., Elbashir et al. (2001) Genes Dev. 15: 188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species will be designed to include a portion of nucleic acid sequence represented in a Cbl-b or Cbl-b-AP, such as POSH, nucleic acid, such as, for example, a nucleic acid that hybridizes, under stringent and/or physiological conditions, to any of the Cbl-b sequences presented in the Examples, such as, for example, the sequences depicted in any of SEQ ID NOs: 37-44 and 51-54 and complements thereof or to any of the Cbl-b-AP, POSH, sequences depicted in SEQ ID NOs: 1, 3, 4, 6, 8 and 10 and complements thereof.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. Nos. 6,251,588, the contents of which are incorporated herein by reference. Messenger RNA (mRNA) is generally thought of as a linear molecule which contains the information for directing protein synthesis within the sequence of ribonucleotides, however studies have revealed a number of secondary and tertiary structures that exist in most mRNAs. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g., Jaeger et al. (1989) Proc. Natl. Acad. Sci. USA 86:7706 (1989); and Turner et al. (1988) Annu. Rev. Biophys. Biophys. Chem. 17:167) . The rules are useful in identification of RNA structural elements and, in particular, for identifying single stranded RNA regions which may represent preferred segments of the mRNA to target for silencing RNAi, ribozyme or antisense technologies. Accordingly, preferred segments of the mRNA target can be identified for design of the RNAi mediating dsRNA oligonucleotides as well as for design of appropriate ribozyme and hammerheadribozyme compositions of the application.

The dsRNA oligonucleotides may be introduced into the cell by transfection with an heterologous target gene using carrier compositions such as liposomes, which are known in the art—e.g., Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al. (1998) J Cell Biol 141: 863-74). The effectiveness of the RNAi may be assessed by any of a number of assays following introduction of the dsRNAs. These include Western blot analysis using antibodies which recognize the Cbl-b or Cbl-b-AP (e.g., POSH) gene product following sufficient time for turnover of the endogenous pool after new protein synthesis is repressed, reverse transcriptase polymerase chain reaction and Northern blot analysis to determine the level of existing Cbl-b or Cbl-b-AP (e.g., POSH) target mRNA.

Further compositions, methods and applications of RNAi technology are provided in U.S. patent application Nos. 6,278,039, 5,723,750 and 5,244,805, which are incorporated herein by reference.

Ribozyme molecules designed to catalytically cleave Cbl-b or Cbl-b-AP(e.g., POSH) mRNA transcripts can also be used to prevent translation of subject Cbl-b or Cbl-b-AP (e.g., POSH) mRNAs and/or expression of Cbl-b or Cbl-b-AP, such as POSH (see, e.g., PCT International Publication WO90/11364, published Oct. 4, 1990; Sarver et al. (1990) Science 247:1222-1225 and U.S. Pat. No. 5,093,246). Ribozymes are enzymatic RNA molecules capable of catalyzing the specific cleavage of RNA. (For a review, see Rossi (1994) Current Biology 4: 469-471). The mechanism of ribozyme action involves sequence specific hybridization of the ribozyme molecule to complementary target RNA, followed by an endonucleolytic cleavage event. The composition of ribozyme molecules preferably includes one or more sequences complementary to a Cbl-b or Cbl-b-AP (e.g., POSH) mRNA, and the well known catalytic sequence responsible for mRNA cleavage or a functionally equivalent sequence (see, e.g., U.S. Pat. No. 5,093,246, which is incorporated herein by reference in its entirety).

While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art and is described more fully in Haseloff and Gerlach ((1988) Nature 334:585-591; and see PCT Appln. No. WO89/05852, the contents of which are incorporated herein by reference). Hammerhead ribozyme sequences can be embedded in a stable RNA such as a transfer RNA (tRNA) to increase cleavage efficiency in vivo (Perriman et al. (1995) Proc. Natl. Acad. Sci. USA, 92: 6175-79; de Feyter, and Gaudron, Methods in Molecular Biology, Vol. 74, Chapter 43, “Expressing Ribozymes in Plants”, Edited by Turner, P. C, Humana Press Inc., Totowa, N.J). In particular, RNA polymerase III-mediated expression of tRNA fusion ribozymes are well known in the art (see Kawasaki et al. (1998) Nature 393: 284-9; Kuwabara et al. (1998) Nature Biotechnol. 16: 961-5; and Kuwabara et al. (1998) Mol. Cell 2: 617-27; Koseki et al. (1999) J Virol 73: 1868-77; Kuwabara et al. (1999) Proc Natl Acad Sci USA 96: 1886-91; Tanabe et al. (2000) Nature 406: 473-4). There are typically a number of potential hammerhead ribozyme cleavage sites within a given target cDNA sequence. Preferably the ribozyme is engineered so that the cleavage recognition site is located near the 5′ end of the target mRNA—to increase efficiency and minimize the intracellular accumulation of non-functional mRNA transcripts. Furthermore, the use of any cleavage recognition site located in the target sequence encoding different portions of the C-terminal amino acid domains of, for example, long and short forms of target would allow the selective targeting of one or the other form of the target, and thus, have a selective effect on one form of the target gene product.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a Cbl-b or Cbl-b-AP mRNA, such as an mRNA of a sequence represented in any of SEQ ID NOs: 37-44 and 51-54 or an mRNA of a sequence represented in any of SEQ ID NOs: 1, 3, 4, 6, 8 or 10. In addition, ribozymes possess highly specific endoribonuclease activity, which autocatalytically cleaves the target sense mRNA. The present application extends to ribozymes which hybridize to a sense mRNA encoding a Cbl-b or Cbl-b-AP (e.g., POSH) gene such as a therapeutic drug target candidate gene, thereby hybridizing to the sense mRNA and cleaving it, such that it is no longer capable of being translated to synthesize a functional polypeptide product.

The ribozymes of the present application also include RNA endoribonucleases (hereinafter “Cech-type ribozymes”) such as the one which occurs naturally in Tetrahymena thermnophila (known as the IVS, or L-19 IVS RNA) and which has been extensively described by Thomas Cech and collaborators (Zaug, et al. (1984) Science 224:574-578; Zaug, et al. (1986) Science 231:470-475; Zaug, et al. (1986) Nature 324:429-433; published International patent application No. WO88/04300 by University Patents Inc.; Been, et al. (1986) Cell 47:207-216). The Cech-type ribozymes have an eight base pair active site which hybridizes to a target RNA sequence whereafter cleavage of the target RNA takes place. The application encompasses those Cech-type ribozymes which target eight base-pair active site sequences that are present in a target gene or nucleic acid sequence.

Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target messages and inhibit translation. Because ribozymes, unlike antisense molecules, are catalytic, a lower intracellular concentration is required for efficiency.

In certain embodiments, a ribozyme may be designed by first identifying a sequence portion sufficient to cause effective knockdown by RNAi. The same sequence portion may then be incorporated into a ribozyme. In this aspect of the application, the gene-targeting portions of the ribozyme or RNAi are substantially the same sequence of at least 5 and preferably 6, 7, 8, 9, 10, 11, 12, 13, 14, 15, 16, 17, 18, 19 or 20 or more contiguous nucleotides of a Cbl-b nucleic acid, such as a nucleic acid of any of SEQ ID NOs: 37-44 and 51-54 or a Cbl-b-AP nucleic acid, such as a POSH nucleic acid of any of SEQ ID NOs: 1, 3, 4, 6, 8, or 10. In a long target RNA chain, significant numbers of target sites are not accessible to the ribozyme because they are hidden within secondary or tertiary structures (Birikh et al. (1997) Eur J Biochem 245: 1-16). To overcome the problem of target RNA accessibility, computer generated predictions of secondary structure are typically used to identify targets that are most likely to be single-stranded or have an “open” configuration (see Jaeger et al. (1989) Methods Enzymol 183: 281-306). Other approaches utilize a systematic approach to predicting secondary structure which involves assessing a huge number of candidate hybridizing oligonucleotides molecules (seeMilner et al. (1997) Nat Biotechnol 15: 537-41; and Patzel and Sczakiel (1998) Nat Biotechnol 16: 64-8). Additionally, U.S. Pat. No. 6,251,588, the contents of which are hereby incorporated herein, describes methods for evaluating oligonucleotide probe sequences so as to predict the potential for hybridization to a target nucleic acid sequence. The method of the application provides for the use of such methods to select preferred segments of a target mRNA sequence that are predicted to be single-stranded and, further, for the opportunistic utilization of the same or substantially identical target mRNA sequence, preferably comprising about 10-20 consecutive nucleotides of the target mRNA, in the design of both the RNAi oligonucleotides and ribozymes of the application.

A further aspect of the application relates to the use of the isolated “antisense” nucleic acids to inhibit expression, e.g., by inhibiting transcription and/or translation of a Cbl-b or Cbl-b-AP (e.g., POSH) nucleic acid. The antisense nucleic acids may bind to the potential drug target by conventional base pair complementarity, or, for example, in the case of binding to DNA duplexes, through specific interactions in the major groove of the double helix. In general, these methods refer to the range of techniques generally employed in the art, and include any methods that rely on specific binding to oligonucleotide sequences.

An antisense construct of the present application can be delivered, for example, as an expression plasmid which, when transcribed in the cell, produces RNA which is complementary to at least a unique portion of the cellular mRNA which encodes a Cbl-b or Cbl-b-AP, such as POSH, polypeptide. Alternatively, the antisense construct is an oligonucleotide probe, which is generated ex vivo and which, when introduced into the cell causes inhibition of expression by hybridizing with the mRNA and/or genomic sequences of a Cbl-b or Cbl-b-AP, such as POSH, nucleic acid. Such oligonucleotide probes are preferably modified oligonucleotides, which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659- 2668.

With respect to antisense DNA, oligodeoxyribonucleotides derived from the translation initiation site, e.g., between the −10 and +10 regions of the target gene, are preferred. Antisense approaches involve the design of oligonucleotides (either DNA or RNA) that are complementary to mRNA encoding a Cbl-b or Cbl-b-AP (e.g., POSH) polypeptide. The antisense oligonucleotides will bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R. 1994. Nature 372:333). Therefore, oligonucleotides complementary to either the 5′ or 3′ untranslated, non-coding regions of a gene could be used in an antisense approach to inhibit translation of that mRNA. Oligonucleotides complementary to the 5′ untranslated region of the mRNA should include the complement of the AUG start codon. Antisense oligonucleotides complementary to mRNA coding regions are less efficient inhibitors of translation but could also be used in accordance with the application. Whether designed to hybridize to the 5′, 3′ or coding region of mRNA, antisense nucleic acids should be at least six nucleotides in length, and are preferably less that about 100 and more preferably less than about 50, 25, 17 or 10 nucleotides in length.

It is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Results obtained using the antisense oligonucleotide may be compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The antisense oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or the blood- brain barrier (see, e.g., PCT Publication No. WO89/10134, published Apr. 25, 1988), hybridization-triggered cleavage agents. (See, e.g., Krol et al., 1988, BioTechniques 6:958- 976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an alpha-anomeric oligonucleotide. An alpha-anomeric oligonucleotide forms specific double-stranded hybrids with complementary RNA in which, contrary to the usual antiparallel orientation, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

While antisense nucleotides complementary to the coding region of a Cbl-b or Cbl-b-AP, such as POSH, mRNA sequence can be used, those complementary to the transcribed untranslated region may also be used.

In certain instances, it may be difficult to achieve intracellular concentrations of the antisense sufficient to suppress translation on endogenous mRNAs. Therefore a preferred approach utilizes a recombinant DNA construct in which the antisense oligonucleotide is placed under the control of a strong pol III or pol II promoter. The use of such a construct to transfect target cells will result in the transcription of sufficient amounts of single stranded RNAs that will form complementary base pairs with the endogenous potential drug target transcripts and thereby prevent translation. For example, a vector can be introduced such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1 981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct, which can be introduced directly into the tissue site.

Alternatively, Cbl-b or Cbl-b-AP (e.g., POSH) gene expression can be reduced by targeting deoxyribonucleotide sequences complementary to the regulatory region of the gene (i.e., the promoter and/or enhancers) to form triple helical structures that prevent transcription of the gene in target cells in the body. (See generally, Helene, C. 1991, Anticancer Drug Des., 6(6):569-84; Helene, C., et al., 1992, Ann. N.Y. Acad. Sci., 660:27-36; and Maher, L. J., 1992, Bioassays 14(12):807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, Cbl-b or Cbl-b-AP (e.g., POSH) sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

A further aspect of the application relates to the use of DNA enzymes to inhibit expression of a Cbl-b or Cbl-b-AP gene, such as a POSH gene. DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid.

There are currently two basic types of DNA enzymes, and both of these were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that will target the enzyme to the message is divided so that it comprises the two arms of the DNA enzyme, and the DNA enzyme loop is placed between the two specific arms.

Methods of making and administering DNA enzymes can be found, for example, in U.S. Pat. No. 6,110,462. Similarly, methods of delivery DNA ribozymes in vitro or in vivo include methods of delivery RNA ribozyme, as outlined in detail above. Additionally, one of skill in the art will recognize that, like antisense oligonucleotide, DNA enzymes can be optionally modified to improve stability and improve resistance to degradation.

Antisense RNA and DNA, ribozyme, RNAi and triple helix molecules of the application may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. A lternatively, antisense cDNA constructs that synthesize antisense RNA constitutively or inducibly, depending on the promoter used, can be introduced stably into cell lines. Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

### 6. DRUG SCREENING ASSAYS

In certain aspects, the present application provides assays for identifying therapeutic agents which either interfere with or promote Cbl-b or Cbl-b-AP function. In certain aspects, the present application also provides assays for identifying therapeutic agents which either interfere with or promote the complex formation between a Cbl-b polypeptide and a Cbl-b-AP polypeptide. In preferred embodiments of the application, the application provides assays for identifying therapeutic agents which either interfere with or promote Cbl-b or POSH function. In certain further preferred aspects, the present application also provides assays for identifying therapeutic agents which either interfere with or promote the complex formation between a Cbl-b polypeptide and a POSH polypeptide.

In certain embodiments, agents of the application are antiviral agents, optionally interfering with viral maturation, and preferably where the virus is an envelope virus, and optionally a retroid virus or an RNA virus. In other embodiments, agents of the application are anticancer agents. In further embodiments, agents of the application inhibit the progression of a neurological disorder. In certain embodiments, an antiviral or anticancer agent or an agent that inhibits the progression of a neurological disorder interferes with the ubiquitin ligase catalytic activity of Cbl-b (e.g., Cbl-b auto-ubiquitination or transfer to a target protein). In certain embodiments, an antiviral or anticancer agent or an agent that inhibits the progression of a neurological disorder interferes with the ubiquitin ligase activity of Cbl-b-AP (e.g., POSH auto-ubiquitination or transfer to a target protein). In other embodiments, agents disclosed herein inhibit or promote Cbl-b and Cbl-b-AP, such as POSH, mediated cellular processes such as apoptosis, protein processing in the secretory pathway, and negative regulation of T cell receptor-coupled signaling pathways.

In certain preferred embodiments, an antiviral agent interferes with the interaction between Cbl-b and a Cbl-b-AP polypeptide, for example an antiviral agent may disrupt or render irreversible interaction between a Cbl-b polypeptide and a POSH polypeptide. In further embodiments, agents of the application are anti-apoptotic agents, optionally interfering with INK and/or NF-κB signaling. In yet additional embodiments, agents of the application interfere with the signaling of a GTPase, such as Rac or Ras, optionally disrupting the interaction between a Cbl-b-AP polypeptide, such as POSH, and a Rac protein. In certain embodiments, agents of the application modulate the ubiquitin ligase activity of Cbl-b and may be used to treat certain diseases related to ubiquitin ligase activity. In certain embodiments, agents of the application modulate the ubiquitin ligase activity of the Cbl-b-AP, POSH, and may be used to treat certain diseases related to ubiquitin ligase activity. In certain embodiments, agents of the application interfere with the trafficking of a protein through the secretory pathway. In certain embodiments, agents of the application interfere with the negative regulation of T cell receptor-coupled signaling pathways

In certain embodiments, the application provides assays to identify, optimize or otherwise assess agents that increase or decrease a ubiquitin-related activity of a Cbl-b polypeptide. Ubiquitin-related activities of Cbl-b polypeptides may include the self-ubiquitination activity of a Cbl-b polypeptide, generally involving the transfer of ubiquitin from an E2 enzyme to the Cbl-b polypeptide, and the ubiquitination of a target protein, generally involving the transfer of a ubiquitin from a Cbl-b polypeptide to the target protein. In certain embodiments, a Cbl-b activity is mediated, at least in part, by a Cbl-b RING domain.

In certain embodiments, the application provides assays to identify, optimize or otherwise assess agents that increase or decrease a ubiquitin-related activity of a Cbl-b polypeptide. Ubiquitin-related activities of Cbl-b polypeptides may include the self-ubiquitination activity of a Cbl-b polypeptide, generally involving the transfer of ubiquitin from an E2 enzyme to the Cbl-b polypeptide, and the ubiquitination of a target protein, generally involving the transfer of a ubiquitin from a Cbl-b polypeptide to the target protein. In certain embodiments, a Cbl-b activity is mediated, at least in part, by a Cbl-b RING domain.

In certain embodiments, an assay comprises forming a mixture comprising a Cbl-b polypeptide, an E2 polypeptide and a source of ubiquitin (which may be the E2 polypeptide pre-complexed with ubiquitin). Optionally the mixture comprises an E1 polypeptide and optionally the mixture comprises a target polypeptide. Additional components of the mixture may be selected to provide conditions consistent with the ubiquitination of the Cbl-b polypeptide. One or more of a variety of parameters may be detected, such as Cbl-b-ubiquitin conjugates, E2-ubiquitin thioesters, free ubiquitin and target polypeptide-ubiquitin complexes. The term “detect” is used herein to include a determination of the presence or absence of the subject of detection (e.g., Cbl-b-ubiqutin, E2-ubiquitin, etc.), a quantitative measure of the amount of the subject of detection, or a mathematical calculation of the presence, absence or amount of the subject of detection, based on the detection of other parameters. The term “detect” includes the situation wherein the subject of detection is determined to be absent or below the level of sensitivity. Detection may comprise detection of a label (e.g., fluorescent label, radioisotope label, and other described below), resolution and identification by size (e.g., SDS-PAGE, mass spectroscopy), purification and detection, and other methods that, in view of this specification, will be available to one of skill in the art. For instance, radioisotope labeling may be measured by scintillation counting, or by densitometry after exposure to a photographic emulsion, or by using a device such as a Phosphorimager. Likewise, densitometry may be used to measure bound ubiquitin following a reaction with an enzyme label substrate that produces an opaque product when an enzyme label is used. In a preferred embodiment, an assay comprises detecting the Cbl-b-ubiquitin conjugate.

In certain embodiments, an assay comprises forming a mixture comprising a Cbl-b polypeptide, a target polypeptide and a source of ubiquitin (which may be the Cbl-b polypeptide pre-complexed with ubiquitin). Optionally the mixture comprises an E1 and/or E2 polypeptide and optionally the mixture comprises an E2-ubiquitin thioester. Additional components of the mixture may be selected to provide conditions consistent with the ubiquitination of the target polypeptide. One or more of a variety of parameters may be detected, such as Cbl-b-ubiquitin conjugates and target polypeptide-ubiquitin conjugates. In a preferred embodiment, an assay comprises detecting the target polypeptide-ubiquitin conjugate. In another preferred embodiment, an assay comprises detecting the Cbl-b-ubiquitin conjugate.

An assay described above may be used in a screening assay to identify agents that modulate a ubiquitin-related activity of a Cbl-b polypeptide. A screening assay will generally involve adding a test agent to one of the above assays, or any other assay designed to assess a ubiquitin-related activity of a Cbl-b polypeptide. The parameter(s) detected in a screening assay may be compared to a suitable reference. A suitable reference may be an assay run previously, in parallel or later that omits the test agent. A suitable reference may also be an average of previous measurements in the absence of the test agent. In general the components of a screening assay mixture may be added in any order consistent with the overall activity to be assessed, but certain variations may be preferred. For example, in certain embodiments, it may be desirable to pre-incubate the test agent and the E3 (e.g., the Cbl-b polypeptide), followed by removing the test agent and addition of other components to complete the assay. In this manner, the effects of the agent solely on the Cbl-b polypeptide may be assessed. In certain embodiments, a screening assay for an antiviral agent employs a target polypeptide comprising an L domain, and preferably an HIV L domain. In certain embodiments, a screening assay for an antiviral agent employs a target polypeptide comprising the p85 subunit of PI3K.

In certain embodiments, an assay is performed in a high-throughput format. For example, one of the components of a mixture may be affixed to a solid substrate and one or more of the other components is labeled. For example, the Cbl-b polypeptide may be affixed to a surface, such as a 96-well plate, and the ubiquitin is in solution and labeled. An E2 and E1 are also in solution, and the Cbl-b-ubiquitin conjugate formation may be measured by washing the solid surface to remove uncomplexed labeled ubiquitin and detecting the ubiquitin that remains bound. Other variations may be used. For example, the amount of ubiquitin in solution may be detected. In certain embodiments, the formation of ubiquitin complexes may be measured by an interactive technique, such as FRET, wherein a ubiquitin is labeled with a first label and the desired complex partner (e.g., Cbl-b polypeptide or target polypeptide) is labeled with a second label, wherein the first and second label interact when they come into close proximity to produce an altered signal. In FRET, the first and second labels are fluorophores. FRET is described in greater detail below. The formation of polyubiquitin complexes may be performed by mixing two or more pools of differentially labeled ubiquitin that interact upon formation of a polyubiqutin (see, e.g., US Patent Publication 20020042083). High-throughput may be achieved by performing an interactive assay, such as FRET, in solution as well. In addition, if a polypeptide in the mixture, such as the Cbl-b polypeptide or target polypeptide, is readily purifiable (e.g., with a specific antibody or via a tag such as biotin, FLAG, polyhistidine, etc.), the reaction may be performed in solution and the tagged polypeptide rapidly isolated, along with any polypeptides, such as ubiquitin, that are associated with the tagged polypeptide. Proteins may also be resolved by SDS-PAGE for detection.

In certain embodiments, the ubiquitin is labeled, either directly or indirectly. This typically allows for easy and rapid detection and measurement of ligated ubiquitin, making the assay useful for high-throughput screening applications. As descrived above, certain embodiments may employ one or more tagged or labeled proteins. A “tag” is meant to include moieties that facilitate rapid isolation of the tagged polypeptide. A tag may be used to facilitate attachment of a polypeptide to a surface. A “label” is meant to include moieties that facilitate rapid detection of the labeled polypeptide. Certain moieties may be used both as a label and a tag (e.g., epitope tags that are readily purified and detected with a well-characterized antibody). Biotinylation of polypeptides is well known, for example, a large number of biotinylation agents are known, including amine-reactive and thiol-reactive agents, for the biotinylation of proteins, nucleic acids, carbohydrates, carboxylic acids; see chapter 4, Molecular Probes Catalog, Haugland, 6th Ed. 1996, hereby incorporated by reference. A biotinylated substrate can be attached to a biotinylated component via avidin or streptavidin. Similarly, a large number of haptenylation reagents are also known.

An “E1” is a ubiquitin activating enzyme. In a preferred embodiment, E1 is capable of transferring ubiquitin to an E2. In a preferred embodiment, E1 forms a high energy thiolester bond with ubiquitin, thereby “activating” the ubiquitin. An “E2” is a ubiquitin carrier enzyme (also known as a ubiquitin conjugating enzyme). In a preferred embodiment, ubiquitin is transferred from E1 to E2. In a preferred embodiment, the transfer results in a thiolester bond formed between E2 and ubiquitin. In a preferred embodiment, E 2 is capable of transferring ubiquitin to a Cbl-b polypeptide.

In an alternative embodiment, a Cbl-b polypeptide, E2 or target polypeptide is bound to a bead, optionally with the assistance of a tag. Following ligation, the beads may be separated from the unbound ubiquitin and the bound ubiquitin measured. In a preferred embodiment, Cbl-b polypeptide is bound to beads and the composition used includes labeled ubiquitin. In this embodiment, the beads with bound ubiquitin may be separated using a fluorescence-activated cell sorting (FACS) machine. Methods for such use are described in U.S. patent application Ser. No. 09/047,119, which is hereby incorporated in its entirety. The amount of bound ubiquitin can then be measured.

In a screening assay, the effect of a test agent may be assessed by, for example, assessing the effect of the test agent on kinetics, steady-state and/or endpoint of the reaction.

The components of the various assay mixtures provided herein may be combined in varying amounts. In a preferred embodiment, ubiquitin (or E2 complexed ubiquitin) is combined at a final concentration of from 5 to 200 ng per 100 microliter reaction solution. Optionally E1 is used at a final concentration of from 1 to 50 ng per 100 microliter reaction solution. Optionally E2 is combined at a final concentration of 10 to 100 ng per 100 microliter reaction solution, more preferably 10-50 ng per 100 microliter reaction solution. In a preferred embodiment, Cbl-b polypeptide is combined at a final concentration of from 1 to 500 ng per 100 microliter reaction solution.

Generally, an assay mixture is prepared so as to favor ubiquitin ligase activity and/or ubiquitination activity. Generally, this will be physiological conditions, such as 50-200 mM salt (e.g., NaCl, KCl), pH of between 5 and 9, and preferably between 6 and 8. Such conditions may be optimized through trial and error. Incubations may be performed at any temperature which facilitates optimal activity, typically between 4 and 40° C. Incubation periods are selected for optimum activity, but may also be optimized to facilitate rapid high through put screening. Typically between 0.5 and 1.5 hours will be sufficient. A variety of other reagents may be included in the compositions. These include reagents like salts, solvents, buffers, neutral proteins, e.g., albumin, detergents, etc. which may be used to facilitate optimal ubiquitination enzyme activity and/or reduce non-specific or background interactions. Also reagents that otherwise improve the efficiency of the assay, such as protease inhibitors, nuclease inhibitors, anti-microbial agents, etc., may be used. The compositions will also preferably include adenosine tri-phosphate (ATP). The mixture of components may be added in any order that promotes ubiquitin ligase activity or optimizes identification of candidate modulator effects. In a preferred embodiment, ubiquitin is provided in a reaction buffer solution, followed by addition of the ubiquitination enzymes. In an alternate preferred embodiment, ubiquitin is provided in a reaction buffer solution, a candidate modulator is then added, followed by addition of the ubiquitination enzymes.

In general, a test agent that decreases a Cbl-b ubiquitin-related activity may be used to inhibit Cbl-b function in vivo, while a test agent that increases a Cbl-b ubiquitin-related activity may be used to stimulate Cbl-b function in vivo. Test agent may be modified for use in vivo, e.g., by addition of a hydrophobic moiety, such as an ester.

In certain embodiments, a ubiquitination assay as described above for Cbl-b can similarly be conducted for a POSH polypeptide. In certain embodiments, the application provides assays to identify, optimize or otherwise assess agents that increase or decrease a ubiquitin-related activity of a POSH polypeptide. Ubiquitin-related activities of POSH polypeptides may include the self-ubiquitination activity of a POSH polypeptide, generally involving the transfer of ubiquitin from an E2 enzyme to the POSH polypeptide, and the ubiquitination of a target protein, e.g., HERPUD1, e.g., PKA, generally involving the transfer of a ubiquitin from a Cbl-b polypeptide to the target protein, e.g, HERPUD1, e.g., PKA. In certain embodiments, a POSH activity is mediated, at least in part, by a RING domain of a POSH polypeptide.

An additional Cbl-b-AP may be added to a Cbl-b ubiquitination assay to assess the effect of the Cbl-b-AP (e.g., POSH) on Cbl-b-mediated ubiquitination and/or to assess whether the Cbl-b-AP is a target for Cbl-b-mediated ubiquitination.

Certain embodiments of the application relate to assays for identifying agents that bind to a Cbl-b or Cbl-b-AP, such as POSH, polypeptide, optionally a particular domain of Cbl-b such as a TKB domain, an SH2 domain, a proline rich domain, or a RING domain or a particular domain of a Cbl-b-AP, such as an SH3 or RING domain of a POSH polypeptide. A wide variety o f assays may be used for this purpose, including labeled in vitro protein-protein binding assays, electrophoretic mobility shift assays, immunoassays for protein binding, and the like. The purified protein may also be used for determination of three-dimensional crystal structure, which can be used for modeling intermolecular interactions and design of test agents. In one embodiment, an assay detects agents which inhibit interaction of one or more subject Cbl-b polypeptides with a Cbl-b-AP, such as POSH. In another embodiment, the assay detects agents which modulate the intrinsic biological activity of a Cbl-b polypeptide or Cbl-b complex, such as an enzymatic activity, binding to other cellular components, cellular compartmentalization, and the like.

In one aspect, the application provides methods and compositions for the identification of compositions that interfere with the function of Cbl-b or Cbl-b-AP polypeptides, such as POSH polypeptides. Given the role of Cbl-b polypeptides in viral production, compositions that perturb the formation or stability of the protein-protein interactions between Cbl-b polypeptides and the proteins that they interact with, such as POSH, and particularly Cbl-b complexes comprising a viral protein, are candidate pharmaceuticals for the treatment of viral infections.

While not wishing to be bound to mechanism, it is postulated that Cbl-b polypeptides promote the assembly of protein complexes that are important in release of virions and other biological processes. Complexes of the application may include a combination o f a Cbl-b polypeptide and a Cbl-b-AP, such as a POSH polypeptide.

A variety of assay formats will suffice and, in light of the present disclosure, those not expressly described herein will nevertheless be comprehended by one of ordinary skill in the art. Assay formats which approximate such conditions as formation of protein complexes, enzymatic activity, and even a Cbl-b polypeptide-mediated membrane reorganization or vesicle formation activity, may be generated in many different forms, and include assays based on cell-free systems, e.g., purified proteins or cell lysates, as well as cell-based assays which utilize intact cells. Simple binding assays can also be used to detect agents which bind to Cbl-b. Such binding assays may also identify agents that act by disrupting the interaction between a Cbl-b polypeptide and a Cbl-b interacting protein, such as a POSH protein, or the binding of a Cbl-b polypeptide or complex to a substrate. Agents to be tested can be produced, for example, by bacteria, yeast or other organisms (e.g., natural products), produced chemically (e.g., small molecules, including peptidomimetics), or produced recombinantly. In a preferred embodiment, the test agent is a small organic molecule, e.g., other than a peptide or oligonucleotide, having a molecular weight of less than about 2,000 daltons.

In many drug screening programs which test libraries of compounds and natural extracts, high throughput assays are desirable in order to maximize the number of compounds surveyed in a given period of time. Assays of the present application which are performed in cell-free systems, such as may be developed with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target.

In preferred in vitro embodiments of the present assay, a reconstituted Cbl-b complex comprises a reconstituted mixture o f at 1 east semi-purified proteins. B y semi-purified, it is meant that the proteins utilized in the reconstituted mixture have been previously separated from other cellular or viral proteins. For instance, in contrast to cell lysates, the proteins involved in Cbl-b complex formation are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins (such as of cellular or viral origin) which might interfere with or otherwise alter the ability to measure Cbl-b complex assembly and/or disassembly.

Assaying Cbl-b complexes, in the presence and absence of a candidate inhibitor, can be accomplished in any vessel suitable for containing the reactants. Examples include microtitre plates, test tubes, and micro-centrifuge tubes.

In one embodiment of the present application, drug screening assays can be generated which detect inhibitory agents on the basis of their ability to interfere with assembly or stability of the Cbl-b complex in an exemplary binding assay, the compound of interest is contacted with a mixture comprising a Cbl-b polypeptide and at least one interacting polypeptide. Detection and quantification of Cbl-b complexes provides a means for determining the compound's efficacy at inhibiting (or potentiating) interaction between the two polypeptides. The efficacy of the compound can be assessed by generating dose response curves from data obtained using various concentrations of the test compound. Moreover, a control assay can also be performed to provide a baseline for comparison. In the control assay, the formation of complexes is quantitated in the absence of the test compound.

Complex formation between the Cbl-b polypeptides and a substrate polypeptide may be detected by a variety of techniques, many of which are effectively described above. For instance, modulation in the formation of complexes can be quantitated using, for example, detectably labeled proteins (e.g., radiolabeled, fluorescently labeled, or enzymatically labeled), by immunoassay, or by chromatographic detection. Surface plasmon resonance systems, such as those available from Biacore International AB (Uppsala, Sweden), may also be used to detect protein-protein interaction

Often, it will be desirable to immobilize one of the polypeptides to facilitate separation of complexes from uncomplexed forms of one of the proteins, as well as to accommodate automation of the assay. In an illustrative embodiment, a fusion protein can be provided which adds a domain that permits the protein to be bound to an insoluble matrix. For example, GST-Cbl-b fusion proteins can be adsorbed onto glutathione sepharose beads (Sigma Chemical, St. Louis, Mo.) or glutathione derivatized microtitre plates, which are then combined with a potential interacting protein, e.g., an ³⁵S-labeled polypeptide, and the test compound and incubated under conditions conducive to complex formation. Following incubation, the beads are washed to remove any unbound interacting protein, and the matrix bead-bound radiolabel determined directly (e.g., beads placed in scintillant), or in the supernatant after the complexes are dissociated, e.g., when microtitre plate is used. Alternatively, after washing away unbound protein, the complexes can be dissociated from the matrix, separated by SDS-PAGE gel, and the level of interacting polypeptide found in the matrix-bound fraction quantitated from the gel using standard electrophoretic techniques.

In a further embodiment, agents that bind to a Cbl-b or Cbl-b-AP (e.g., POSH) may be identified by using an immobilized Cbl-b or Cbl-b-AP. In an illustrative embodiment, a fusion protein can be provided which adds a domain that permits the protein to be bound to an insoluble matrix. For example, GST-Cbl-b fusion proteins can be adsorbed onto glutathione sepharose beads (Sigma Chemical, St. Louis, Mo.) or glutathione derivatized microtitre plates, which are then combined with a potential labeled binding agent and incubated under conditions conducive to binding. Following incubation, the beads are washed to remove any unbound agent, and the matrix bead-bound label determined directly, or in the supernatant after the bound agent is dissociated.

In yet another embodiment, the Cbl-b polypeptide and potential interacting polypeptide can be used to generate an interaction trap assay (see also, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and iwabuchi et al. (1993) Oncogene 8:1693-1696), for subsequently detecting agents which disrupt binding of the proteins to one and other.

In particular, the method makes use of chimeric genes which express hybrid proteins. To illustrate, a first hybrid gene comprises the coding sequence for a DNA-binding domain of a transcriptional activator can be fused in frame to the coding sequence for a “bait” protein, e.g., a Cbl-b polypeptide of sufficient length to bind to a potential interacting protein. The second hybrid protein encodes a transcriptional activation domain fused in frame to a gene encoding a “fish” protein, e.g., a potential interacting protein of sufficient length to interact with the Cbl-b polypeptide portion of the bait fusion protein. If the bait and fish proteins are able to interact, e.g., form a Cbl-b complex, they bring into close proximity the two domains of the transcriptional activator. This proximity causes transcription of a reporter gene which is operably linked to a transcriptional regulatory site responsive to the transcriptional activator, and expression of the reporter gene can be detected and used to score for the interaction of the bait and fish proteins.

One aspect of the present application provides reconstituted protein preparations including a Cbl-b polypeptide and one or more interacting polypeptides.

In still further embodiments of the present assay, the Cbl-b complex is generated in whole cells, taking advantage of cell culture techniques to support the subject assay. For example, as described below, the Cbl-b complex can be constituted in a eukaryotic cell culture system, including mammalian and yeast cells. It may be desirable to express one or more viral proteins (e.g., Gag or Env) in such a cell along with a subject Cbl-b polypeptide. It may also be desirable to infect the cell with a virus of interest. Advantages to generating the subject assay in an intact cell include the ability to detect inhibitors which are functional in an environment more closely approximating that which therapeutic use of the inhibitor would require, including the ability of the agent to gain entry into the cell. Furthermore, certain of the in vivo embodiments of the assay, such as examples given below, are amenable to high through-put analysis of candidate agents.

The components of the Cbl-b complex can be endogenous to the cell selected to support the assay. Alternatively, some or all of the components can be derived from exogenous sources. For instance, fusion proteins can be introduced into the cell by recombinant techniques (such as through the use of an expression vector), as well as by microinjecting the fusion protein itself or mRNA encoding the fusion protein.

In many embodiments, a cell is manipulated after incubation with a candidate agent and assayed for a Cbl-b or Cbl-b-AP activity. In certain embodiments a Cbl-b or Cbl-b-AP activity, such as POSH activity, is represented by production of virus like particles. As demonstrated herein, an agent that disrupts Cbl-b or Cbl-b-AP (e.g., POSH) activity can cause a decrease in the production of virus like particles. Other bioassays for Cbl-b or Cbl-b-AP (e.g., POSH) activities may include apoptosis assays (e.g., cell survival assays, apoptosis reporter gene assays, etc.) and NF-kB nuclear localization assays (see e.g., Tapon et al. (1998) EMBO J. 17: 1395-1404).

In certain embodiments, Cbl-b or Cbl-b-AP activities, such as POSH activities, may include, without limitation, complex formation, ubiquitination and membrane fusion events (e.g., release of viral buds or fusion of vesicles). Cbl-b complex formation may be assessed by immunoprecipitation and analysis of co-immunoprecipiated proteins or affinity purification and analysis of co-purified proteins. Fluorescence Resonance Energy Transfer (FRET)-based assays or other energy transfer assays may also be used to determine complex formation.

Additional bioassays for assessing Cbl-b and Cbl-b-AP activities may include assays to detect the improper processing of a protein that is associated with a neurological disorder. One assay that may be used is an assay to detect the presence, including an increase or a decrease in the amount, of a protein associated with a neurological disorder. For example, the use of RNAi may be employed to knockdown the expression of a Cbl-b or Cbl-b-AP polypeptide, such as POSH, in cells (e.g., CHO cells or COS cells). The production of a secreted protein such as for example, amyloid beta, in the cell culture media, can then be assessed and compared to production of the secreted protein from control cells, which may be cells in which the Cbl-b or Cbl-b-AP activity (e.g., POSH activity) has not been inhibited. The production of secreted proteins may be assessed, such as amyloid beta protein, which is associated with Alzheimer's disease. In some instances, a label may be incorporated into a secreted protein and the presence of the labeled secreted protein detected in the cell culture media. Proteins secreted from any cell type may be assessed, including for example, neural cells.

The effect of an agent that modulates the activity of Cbl-b or a Cbl-b-AP, such as POSH, may be evaluated for effects on mouse models of various neurological disorders. For example, mouse models of Alzheimer's disease have been described. See, for example, U.S. Pat. No. 5,612,486 for “Transgenic Animals Harboring APP Allele Having Swedish Mutation,” U.S. Pat. No. 5,850,003 (the '003 patent) for “Transgenic Rodents Harboring APP Allele Having Swedish Mutation,” and U.S. Pat. No. 5,455,169 entitled “Nucleic Acids for Diagnosing and Modeling Alzheimer's Disease”. Mouse models of Alzheimer's disease tend to produce elevated levels of beta-amyloid protein in the brain, and the increase or decrease of such protein in response to treatment with a test agent may be detected. In some instances, it may also be desirable to assess the effects of a test agent on cognitive or behavioral characteristics of a mouse model for Alzheimer's disease, as well as mouse models for other neurological disorders.

In a further embodiment, transcript levels may be measured in cells having higher or lower levels of Cbl-b or Cbl-b-AP activity, such as POSH activity, in order to identify genes that are regulated by Cbl-b or Cbl-b-APs. Promoter regions for such genes (or larger portions of such genes) may be operatively linked to a reporter gene and used in a reporter gene-based assay to detect agents that enhance or diminish Cbl-b- or Cbl-b-AP-regulated gene expression. Transcript levels may be determined in any way known in the art, such as, for example, Northern blotting, RT-PCR, microarray, etc. Increased Cbl-b activity may be achieved, for example, by introducing a strong Cbl-b expression vector. Decreased Cbl-b activity may be achieved, for example, by RNAi, antisense, ribozyme, gene knockout, etc.

In general, where the screening assay is a binding assay (whether protein-protein binding, agent-protein binding, etc.), one or more of the molecules may be joined to a label, where the label can directly or indirectly provide a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

In further embodiments, the application provides methods for identifying targets for therapeutic intervention. A polypeptide that interacts with Cbl-b or participates in a Cbl-b-mediated process (such as viral maturation) may be used to identify candidate therapeutics. Such targets may be identified by identifying proteins that associated with Cbl-b (Cbl-b-APs) by, for example, immunoprecipitation with an anti-Cbl-b antibody, in silico analysis of high-throughput binding data, two-hybrid screens, and other protein-protein interaction assays described herein or otherwise known in the art in view of this disclosure. Agents that bind to such targets or disrupt protein-protein interactions thereof, or inhibit a biochemical activity thereof may be used in such an assay. Targets that have been identified by such approaches include POSH.

A variety of other reagents may be included in the screening assay. These include reagents like salts, neutral proteins, e.g., albumin, detergents, etc that are used to facilitate optimal protein-protein binding and/or reduce nonspecific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4° C. and 40° C. Incubation periods are selected for optimum activity, but may also be optimized to facilitate rapid high-throughput screening.

In certain embodiments, a test agent may be assessed for antiviral or anticancer activity by assessing effects on an activity (function) of a Cbl-b-AP, such as, for example, POSH. Activity (function) may be affected by an agent that acts at one or more of the transcriptional, translational or post-translational stages. For example, an siRNA directed to a Cbl-b-AP encoding gene will decrease activity, as will a small molecule that interferes with a catalytic activity of a Cbl-b-AP. In certain embodiments, the agent inhibits the activity of one or more POSH polypeptides.

### 7. EXEMPLARY NUCLEIC ACIDS AND EXPRESSION VECTORS

In certain aspects, the application relates to nucleic acids encoding Cbl-b polypeptides. For example, Cbl-b polypeptides of the disclosure are listed in the Examples. Nucleic acid sequences encoding these Cbl-b polypeptides are provided in the Examples. In certain embodiments, variants will also include nucleic acid sequences that will hybridize under highly stringent conditions to a nucleotide sequence of a coding sequence of a Cbl-b polypeptide. Preferred nucleic acids of the application are human Cbl-b sequences and variants thereof.

In certain aspects, the application relates to nucleic acids encoding Cbl-b polypeptides, such as, for example, SEQ ID NOs: 37-44 and 51-54. Nucleic acids of the application are further understood to include nucleic acids that comprise variants of SEQ ID NOs: 37-44 and 51-54. Variant nucleotide sequences include sequences that differ by one or more nucleotide substitutions, additions or deletions, such as allelic variants; and will, therefore, include coding sequences that differ from the nucleotide sequence of the coding sequence designated in SEQ ID NOs: 37-44 and 51-54, e.g., due to the degeneracy of the genetic code. In other embodiments, variants will also include sequences that will hybridize under highly stringent conditions to a nucleotide sequence of a coding sequence designated in any of SEQ ID NOs: 37-44 and 51-54 Preferred nucleic acids of the application are human Cbl-b sequences, including, for example, any of SEQ ID NOs: 37-44 and variants thereof and nucleic acids encoding an amino acid sequence selected from among SEQ ID NOs: 45-50. In certain embodiments, nucleic acids of the application are human Cbl-b sequences designated in any of SEQ ID NOS: 43-44.

In one aspect, the application provides an isolated nucleic acid comprising a nucleotide sequence which hybridizes under stringent conditions to a sequence of SEQ ID NOs: 43 and/or 44 or a sequence complementary thereto. In a related embodiment, the nucleic acid is at least about 80%, 90%, 95%, or 97-98%, or 100% identical to a sequence corresponding to at least about 12, at least about 15, at least about 25, at least about 40, at least about 100, at least about 300, at least about 500, at least about 1000, or at least about 2500 consecutive nucleotides up to the full length of SEQ ID NO: 43 and/or 44, or a sequence complementary thereto.

In one aspect, the application provides an isolated nucleic acid comprising a nucleotide sequence which hybridizes under stringent conditions to a sequence of SEQ ID NOs: 59-64 or a sequence complementary thereto. In a related embodiment, the nucleic acid is at least about 80%, 90%, 95%, or 97-98%, or 100% identical to a sequence corresponding to at least about 12, at least about 15, at least about 25, consecutive nucleotides up to the full length of SEQ ID NO: 59-64, or a sequence complementary thereto.

In other embodiments, the application provides a nucleic acid comprising a nucleotide sequence which hybridizes under stringent conditions to a sequence of SEQ ID NOs: 43 and/or 44, or a nucleotide sequence that is at least about 80%, 90%, 95%, or 97-98%, or 100% identical to a sequence corresponding to at least about 12, at least about 15, at least about 25, at least about 40, at least about 100, at least about 300, at least about 500, at least about 1000, or at least about 2500 consecutive nucleotides up to the full length of SEQ ID NO: 43 and/or 44, or a sequence complementary thereto, and a transcriptional regulatory sequence operably linked to the nucleotide sequence to render the nucleotide sequence suitable for use as an expression vector. In another embodiment, the nucleic acid may be included in an expression vector capable of replicating in a prokaryotic or eukaryotic cell. In a related embodiment, the application provides a host cell transfected with the expression vector.

In a further embodiment, the application provides a nucleic acid comprising a nucleic acid encoding an amino acid sequence as set forth in any of SEQ ID NOs: 45 and 46, or a nucleic acid complement thereof. In a related embodiment, the encoded amino acid sequence is at least about 80%, 90%, 95%, or 97-98%, or 100% identical to a sequence corresponding to at least about 12, at least about 15, at least about 25, or at least about 40 consecutive amino acids up to the full length of any of SEQ ID NOs: 45 or 46.

In another aspect, the application provides polypeptides. In one embodiment, the application pertains to a polypeptide including an amino acid sequence encoded by a nucleic acid comprising a nucleotide sequence which hybridizes under stringent conditions to a sequence of SEQ ID NOs: 43 and/or 44, or a sequence complementary thereto, or a fragment comprising at least about 25, or at least about 40 amino acids thereof.

In certain aspects, the application relates to nucleic acids encoding Cbl-b-AP polypeptides. In preferred embodiments, the application relates to nucleic acids encoding the Cbl-b-AP, POSH, polypeptides, such as, for example, SEQ ID NOs: 2, 5, 7, 9, 11, 26, 27, 28, 29 and 30. Nucleic acids of the application are further understood to include nucleic acids that comprise variants of SEQ ID Nos:1, 3, 4, 6, 8, 10, 31, 32, 33, 34, and 35. Variant nucleotide sequences include sequences that differ by one or more nucleotide substitutions, additions or deletions, such as allelic variants; and will, therefore, include coding sequences that differ from the nucleotide sequence of the coding sequence designated in SEQ ID Nos:1, 3, 4, 6, 8 10, 31, 32, 33, 34, and 35, e.g., due to the degeneracy of the genetic code. In other embodiments, variants will also include sequences that will hybridize under highly stringent conditions to a nucleotide sequence of a coding sequence designated in any of SEQ ID Nos:1, 3, 4, 6, 8 10, 31, 32, 33, 34, and 35. Preferred nucleic acids of the application are human POSH sequences, including, for example, any of SEQ ID Nos: 1, 3, 4, 6, 31, 32, 33, 34, 35 and variants thereof and nucleic acids encoding an amino acid sequence selected from among SEQ ID Nos: 2, 5, 7, 26, 27, 28, 29 and 30.

One of ordinary skill in the art will understand readily that appropriate stringency conditions which promote DNA hybridization can be varied. For example, one could perform the hybridization at 6.0×sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the application provides nucleic acids which hybridize under low stringency conditions of 6×SSC at room temperature followed by a wash at 2×SSC at room temperature.

Isolated nucleic acids which differ from the Cbl-b nucleic acid sequences or from the Cbl-b-AP nucleic acid sequences, such as the POSH nucleic acid sequences, due to degeneracy in the genetic code are also within the scope of the application. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this application.

Optionally, a Cbl-b or a Cbl-b-AP (e.g., POSH) nucleic acid of the application will genetically complement a partial or complete loss of function phenotype in a cell. For example, a Cbl-b nucleic acid of the application may be expressed in a cell in which endogenous Cbl-b has been reduced by RNAi, and the introduced Cbl-b nucleic acid will mitigate a phenotype resulting from the RNAi. An exemplary Cbl-b loss of function phenotype is a decrease in virus-like particle production in a cell transfected with a viral vector, optionally an HIV vector.

Another aspect of the application relates to Cbl-b and Cbl-b-AP nucleic acids, such as POSH nucleic acids, that are used for antisense, RNAi or ribozymes. As used herein, nucleic acid therapy refers to administration or in situ generation of a nucleic acid or a derivative thereof which specifically hybridizes (e.g., binds) under cellular conditions with the cellular mRNA and/or genomic DNA encoding one of the Cbl-b or Cbl-b-AP, such as POSH, polypeptides so as to inhibit production of that protein, e.g., by inhibiting transcription and/or translation. The binding may be by conventional base pair complementarity, or, for example, in the case of binding to DNA duplexes, through specific interactions in the major groove of the double helix.

A nucleic acid therapy construct of the present application can be delivered, for example, as an expression plasmid which, when transcribed in the cell, produces RNA which is complementary to at least a unique portion of the cellular mRNA which encodes a Cbl-b or Cbl-b-AP polypeptide, such as a POSH polypeptide. Alternatively, the construct is an oligonucleotide which is generated ex vivo and which, when introduced into the cell causes inhibition of expression by hybridizing with the mRNA and/or genomic sequences encoding a Cbl-b or Cbl-b-AP (e.g., POSH) polypeptide. Such oligonucleotide probes are optionally modified oligonucleotide which are resistant to endogenous nucleases, e.g., exonucleases and/or endonucleases, and is therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in nucleic acid therapy have been reviewed, for example, by van der Krol et al., (1988) Biotechniques 6:958-976; and Stein et al., (1988) Cancer Res 48:2659-2668.

Accordingly, the modified oligomers of the application are useful in therapeutic, diagnostic, and research contexts. In therapeutic applications, the oligomers are utilized in a manner appropriate for nucleic acid therapy in general.

In another aspect of the application, the subject nucleic acid is provided in an expression vector comprising a nucleotide sequence encoding a Cbl-b or Cbl-b-AP, such as POSH, polypeptide and operably linked to at least one regulatory sequence. Regulatory sequences are art-recognized and are selected to direct expression of the Cbl-b or Cbl-b-AP polypeptide. Accordingly, the term regulatory sequence includes promoters, enhancers and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding a Cbl-b or Cbl-b-AP polypeptide. Such useful expression control sequences, include, for example, the early and late promoters of SV40, tet promoter, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., PhoS, the promoters of the yeast α-mating factors, the polyhedron promoter of the baculovirus system and other sequences known to control the expression of genes of prokaryotic or eukaryotic cells or their viruses, and various combinations thereof It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

As will be apparent, the subject gene constructs can be used to cause expression of the Cbl-b or Cbl-b-AP polypeptides in cells propagated in culture, e.g., to produce proteins or polypeptides, including fusion proteins or polypeptides, for purification.

This application also pertains to a host cell transfected with a recombinant gene including a coding sequence for one or more of the Cbl-b or Cbl-b-AP (e.g., POSH) polypeptides. The host cell may be any prokaryotic or eukaryotic cell. For example, a polypeptide of the present application may be expressed in bacterial cells such as E. coli, insect cells (e.g., using a baculovirus expression system), yeast, or mammalian cells. Other suitable host cells are known to those skilled in the art. Accordingly, the present application further pertains to methods of producing the Cbl-b or Cbl-b-AP (e.g., POSH) polypeptides. For example, a host cell transfected with an expression vector encoding a Cbl-b polypeptide can be cultured under appropriate conditions to allow expression of the polypeptide to occur. The polypeptide may be secreted and isolated from a mixture of cells and medium containing the polypeptide. Alternatively, the polypeptide may be retained cytoplasmically and the cells harvested, lysed and the protein isolated. A cell culture includes host cells, media and other byproducts. Suitable media for cell culture are well known in the art. The polypeptide can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptide. In a preferred embodiment, the Cbl-b or Cbl-b-AP polypeptide is a fusion protein containing a domain which facilitates its purification, such as a Cbl-b-GST fusion protein, Cbl-b-intein fusion protein, Cbl-b-cellulose binding domain fusion protein, Cbl-b-polyhistidine fusion protein etc.

A recombinant Cbl-b or Cbl-b-AP, such as POSH, nucleic acid can be produced by ligating the cloned gene, or a portion thereof, into a vector suitable for expression in either prokaryotic cells, eukaryotic cells, or both. Expression vehicles for production of recombinant Cbl-b or Cbl-b-AP polypeptides include plasmids and other vectors. For instance, suitable vectors for the expression of a Cbl-b polypeptide include plasmids of the types: pBR322-derived plasmids, pEMBL-derived plasmids, pEX-derived plasmids, pBTac-derived plasmids and pUC-derived plasmids for expression in prokaryotic cells, such as E. coli.

The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd End., ed. by Sambrook, Fritsch and Maniatis (Cold Spring Harbor Laboratory Press, 1989) Chapters 16 and 17. In some instances, it may be desirable to express the recombinant Cbl-b or Cbl-b-AP polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

Alternatively, the coding sequences for the polypeptide can be incorporated as a part of a fusion gene including a nucleotide sequence encoding a different polypeptide. This type of expression system can be useful under conditions where it is desirable, e.g., to produce an immunogenic fragment of a Cbl-b or Cbl-b-AP (e.g., POSH) polypeptide. For example, the VP6 capsid protein of rotavirus can be used as an immunologic carrier protein for portions of polypeptide, either in the monomeric form or in the form of a viral particle. The nucleic acid sequences corresponding to the portion of the Cbl-b or Cbl-b-AP polypeptide to which antibodies are to be raised can be incorporated into a fusion gene construct which includes coding sequences for a late vaccinia virus structural protein to produce a set of recombinant viruses expressing fusion proteins comprising a portion of the protein as part of the virion. The Hepatitis B surface antigen can also be utilized in this role as well. Similarly, chimeric constructs coding for fusion proteins containing a portion of a Cbl-b polypeptide and the poliovirus capsid protein can be created to enhance immunogenicity (see, for example, EP Publication NO: 0259149; and Evans et al.,, (1989) Nature 339:385; Huang et al., (1988) J. Virol. 62:3855; and Schlienger et al., (1992) J. Virol. 66:2).

The Multiple Antigen Peptide system for peptide-based immunization can be utilized, wherein a desired portion of a Cbl-b or Cbl-b-AP polypeptide is obtained directly from organo-chemical synthesis of the peptide onto an oligomeric branching lysine core (see, for example, Posnett et al., (1988) JBC 263:1719 and Nardelli et al., (1992) J. Immunol. 148:914). Antigenic determinants of a Cbl-b or Cbl-b-AP polypeptide can also be expressed and presented by bacterial cells.

In another embodiment, a fusion gene coding for a purification leader sequence, such as a poly-(His)/enterokinase cleavage site sequence at the N-terminus of the desired portion of the recombinant protein, can allow purification of the expressed fusion protein by affinity chromatography using a Ni²⁺ metal resin. The purification leader sequence can then be subsequently removed by treatment with enterokinase to provide the purified Cbl-b or Cbl-b-AP polypeptide (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

TABLE 2
Exemplary Cbl-b nucleic acids and polypeptides and their related
Sequence Identification Numbers.

| | | Sequence |
|---|---|---|
| | | Identification |
| | Public gi | Number |
| Sequence Information | no. | (SEQ ID NO:) |
| Human CBL-B mRNA Sequence - var 1 | 4757919 | SEQ ID NO: 37 |
| Human CBL-B mRNA Sequence - var 2 | 23273908 | SEQ ID NO: 38 |
| Human CBL-B mRNA Sequence - var 3 | 862406 | SEQ ID NO: 39 |
| Human CBL-B mRNA Sequence - var 4 | 862408 | SEQ ID NO: 40 |
| Human CBL-B mRNA Sequence - var 5 | 862410 | SEQ ID NO: 41 |
| Human CBL-B mRNA Sequence - var 6 | 21753192 | SEQ ID NO: 42 |
| Human CBL-B mRNA Sequence - var 7 | — | SEQ ID NO: 43 |
| Human CBL-B Protein Sequence - var 7 | — | SEQ ID NO: 45 |
| Human CBL-B clone 3Gd114 | — | SEQ ID NO: 44 |
| Human CblB protein in 3Gd114 | — | SEQ ID NO: 46 |
| Translation of cbl-B clone 3Gd114 | | |
| starting at base pair 3 | | |
| Human CBL-B Protein Sequence - var 1 | 4757920 | SEQ ID NO: 47 |
| Human CBL-B Protein Sequence - var 2 | 23273909 | SEQ ID NO: 48 |
| Human CBL-B Protein Sequence - var 3 | 862407 | SEQ ID NO: 49 |
| Human CBL-B Protein Sequence - var 4 | 862409 | SEQ ID NO: 50 |
| Rat CBL-B mRNA Sequence | 21886623 | SEQ ID NO: 51 |
| Rat CBL-B Protein Sequence | 21886624 | SEQ ID NO: 55 |
| Mouse CBL-B mRNA Sequence | 2634665 | SEQ ID NO: 52 |
| Mouse CBL-B Protein Sequence | 26324666 | SEQ ID NO: 56 |
| Drosophila CBL-B mRNA Sequence | 1842452 | SEQ ID NO: 53 |
| Drosophila CBL-B Protein Sequence | 1842453 | SEQ ID NO: 57 |
| C. elegans CBL-B mRNA Sequence | 25150544 | SEQ ID NO: 54 |
| C. elegans CBL-B Protein Sequence | 25150545 | SEQ ID NO: 58 |

TABLE 3
Exemplary POSH nucleic acids

| | | Accession |
|---|---|---|
| Sequence Name | Organism | Number |
| cDNA FLJ11367 fis, clone | Homo sapiens | AK021429 |
| HEMBA1000303 | | |
| Plenty of SH3 domains | Mus musculus | NM_021506 |
| (POSH) mRNA | | |
| Plenty of SH3s (POSH) | Mus musculus | AF030131 |
| mRNA | | |
| Plenty of SH3s (POSH) | Drosophila melanogaster | NM_079052 |
| mRNA | | |
| Plenty of SH3s (POSH) | Drosophila melanogaster | AF220364 |
| mRNA | | |

TABLE 4
Exemplary POSH polypeptides

| Sequence Name | Organism | Accession Number |
|---|---|---|
| SH3 domains- | Mus musculus | T09071 |
| containing protein POSH | | |
| plenty of SH3 domains | Mus musculus | NP_067481 |
| Plenty of SH3s; POSH | Mus musculus | AAC40070 |
| Plenty of SH3s | Drosophila melanogaster | AAF37265 |
| LD45365p | Drosophila melanogaster | AAK93408 |
| POSH gene product | Drosophila melanogaster | AAF57833 |
| Plenty of SH3s | Drosophila melanogaster | NP_523776 |

In addition the following Tables provide the nucleic acid sequence and related SEQ ID NOs for domains of human POSH protein and a summary of POSH sequence identification numbers used in this application.

TABLE 5

Nucleic Acid Sequences and related SEQ ID NOs
for domains in human POSH

| | | SEQ |
|---|---|---|
| Name of the | | ID |
| sequence | Sequence | NO. |
| RING domain | TGTCCGGTGTGTCTAGAGCGCCTTGATGCTTCTGCGAAGGTCT | 31 |
| | TGCCTTGCCAGCATACGTTTTGCAAGCGATGTTTGCTGGGGAT | |
| | CGTAGGTTCTCGAAATGAACTCAGATGTCCCGAGT | |
| 1^{st} SH₃ | CCATGTGCCAAAGCGTTATACAACTATGAAGGAAAAGAGCCTG | 32 |
| domain | GAGACCTTAAATTCAGCAAAGGCGACATCATCATTTTGCGAAG | |
| | ACAAGTGGATGAAAATTGGTACCATGGGGAAGTCAATGGAATC | |
| | CATGGCTTTTTCCCCACCAACTTTGTGCAGATTATT | |
| 2^{nd} SH₃ | CCTCAGTGCAAAGCACTTTATGACTTTGAAGTGAAAGACAAGG | 33 |
| domain | AAGCAGACAAAGATTGCCTTCCATTTGCAAAGGATGATGTTCT | |
| | GACTGTGATCCGAAGAGTGGATGAAAACTGGGCTGAAGGAATG | |
| | CTGGCAGACAAAATAGGAATATTTCCAATTTCATATGTTGAGT | |
| | TTAAC | |
| 3^{rd} SH₃ | AGTGTGTATGTTGCTATATATCCATACACTCCTCGGAAAGAGG | 34 |
| domain | ATGAACTAGAGCTGAGAAAAGGGGAGATGTTTTTAGTGTTTGA | |
| | GCGCTGCCAGGATGGCTGGTTCAAAGGGACATCCATGCATACC | |
| | AGCAAGATAGGGGTTTTCCCTGGCAATTATGTGGCACCAGTC | |
| 4^{th} SH₃ | GAAAGGCACAGGGTGGTGGTTTCCTATCCTCCTCAGAGTGAGG | 35 |
| domain | CAGAACTTGAACTTAAAGAAGGAGATATTGTGTTTGTTCATAA | |
| | AAAACGAGAGGATGGCTGGTTCAAAGGCACATTACAACGTAAT | |
| | GGGAAAACTGGCCTTTTCCCAGGAAGCTTTGTGGAAAACA | |

TABLE 6
Summary of POSH sequence Identification Numbers

| | Sequence Identification |
|---|---|
| Sequence Information | Number (SEQ ID NO) |
| Human POSH Coding Sequence | SEQ ID No: 1 |
| Human POSH Amino Acid Sequence | SEQ ID No: 2 |
| Human POSH cDNA Sequence | SEQ ID No: 3 |
| 5′ cDNA Fragment of Human POSH | SEQ ID No: 4 |
| N-terminus Protein Fragment of | SEQ ID No: 5 |
| Human POSH | |
| 3′ mRNA Fragment of Human POSH | SEQ ID No: 6 |
| C-terminus Protein Fragment of | SEQ ID No: 7 |
| Human POSH | |
| Mouse POSH mRNA Sequence | SEQ ID No: 8 |
| Mouse POSH Protein Sequence | SEQ ID No: 9 |
| Drosophila melanogaster POSH | SEQ ID No: 10 |
| mRNA Sequence | |
| Drosophila melanogaster POSH | SEQ ID No: 11 |
| Protein Sequence | |
| Human POSH RING Domain Amino | SEQ ID No: 26 |
| Acid Sequence | |
| Human POSH 1^{st} SH₃ Domain Amino | SEQ ID No: 27 |
| Acid Sequence | |
| Human POSH 2^{nd} SH₃ Domain Amino | SEQ ID No: 28 |
| Acid Sequence | |
| Human POSH 3^{rd} SH₃ Domain Amino | SEQ ID No: 29 |
| Acid Sequence | |
| Human POSH 4^{th} SH₃ Domain Amino | SEQ ID No: 30 |
| Acid Sequence | |
| Human POSH RING Domain Nucleic | SEQ ID No: 31 |
| Acid Sequence | |
| Human POSH 1^{st} SH₃ Domain Nucleic | SEQ ID No: 32 |
| Acid Sequence | |
| Human POSH 2^{nd} SH₃ Domain Nucleic | SEQ ID No: 33 |
| Acid Sequence | |
| Human POSH 3^{rd} SH₃ Domain Nucleic | SEQ ID No: 34 |
| Acid Sequence | |
| Human POSH 4^{th} SH₃ Domain Nucleic | SEQ ID No: 35 |
| Acid Sequence | |

### 8. EXEMPLARY POLYPEPTIDES

In certain aspects, the present application relates to Cbl-b polypeptides, which are isolated from, or otherwise substantially free of, other intracellular proteins which might normally be associated with the protein or a particular complex including the protein. In certain embodiments, Cbl-b polypeptides have an, amino acid sequence that is at least 60% identical to an amino acid sequence as set forth in any of SEQ ID NOs: 45-50 and 55-58. In certain embodiments, Cbl-b polypeptides have an amino acid sequence that is at least 60% identical to an amino acid sequence as set for in any of SEQ ID NOs: 45-46. In other embodiments, the polypeptide has an amino acid sequence at least 65%, 70%, 75%, 80%, 85%, 90%, 95%, 97%, 98%, 99% or 100% identical to an amino acid sequence as set forth in any of SEQ ID NOs: 45-50 and 55-58. In certain embodiments, the polypeptide has an amino acid sequence at least 65%, 70%, 75%, 80%, 85%, 90%, 95%, 97%, 98%, 99% or 100% identical to an amino acid sequence as set forth in any of SEQ ID NOs: 45-46. Amino acid sequences of Cbl-b polypeptides are provided in the Examples.

In certain aspects, the application relates to Cbl-b-AP polypeptides. In preferred embodiments, the present application relates to the Cbl-b-AP, POSH, polypeptides, which are isolated from, or otherwise substantially free of, other intracellular proteins which might normally be associated with the protein or a particular complex including the protein. In certain embodiments, POSH polypeptides have an amino acid sequence that is at least 60% identical to an amino acid sequence as set forth in any of SEQ ID NOs: 2, 5, 7, 9, 11, 26, 27, 28, 29 and 30. In other embodiments, the polypeptide has an amino acid sequence at least 65%, 70%, 75%, 80%, 85%, 90%, 95%, 97%, 98%, 99% or 100% identical to an amino acid sequence as set forth in any of SEQ ID NOs: 2, 5, 7, 9, 11, 26, 27, 28, 29 and 30.

Optionally, a Cbl-b or Cbl-b-AP polypeptide of the application will function in place of an endogenous Cbl-b or Cbl-b-AP polypeptide, for example by mitigating a partial or complete loss of function phenotype in a cell. For example, a Cbl-b polypeptide of the application may be produced in a cell in which endogenous Cbl-b has been reduced by RNAi, and the introduced Cbl-b polypeptide will mitigate a phenotype resulting from the RNAi. An exemplary Cbl-b loss of flnction phenotype is a decrease in virus-like particle production in a cell transfected with a viral vector, optionally an HIV vector.

In another aspect, the application provides polypeptides that are agonists or antagonists of a Cbl-b or Cbl-b-AP polypeptide. In certain embodiments, the application provides antagonists of the Cbl-b-AP, POSH. Variants and fragments of a Cbl-b or Cbl-b-AP polypeptide may have a hyperactive or constitutive activity, or, alternatively, act to prevent Cbl-b or Cbl-b-AP polypeptides from performing one or more functions. For example, a mutant form of a Cbl-b or Cbl-b-AP protein domain may have a dominant negative effect, such as, for example, a Cbl-b polypeptide comprising a mutant RING domain as decribed in the Examples.

Another aspect of the application relates to polypeptides derived from a fall-length Cbl-b or Cbl-b-AP (e.g., POSH) polypeptide. Isolated peptidyl portions of the subject proteins can be obtained by screening polypeptides recombinantly produced from the corresponding fragment of the nucleic acid encoding such polypeptides. In addition, fragments can be chemically synthesized using techniques known in the art such as conventional Merrifield solid phase f-Moc or t-Boc chemistry. For example, any one of the subject proteins can be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of the formation of a specific protein complex, or more generally of a Cbl-b:Cbl-b-AP complex, such as by microinjection assays.

It is also possible to modify the structure of the Cbl-b or Cbl-b-AP polypeptides for such purposes as enhancing therapeutic or prophylactic efficacy, or stability (e.g., ex vivo shelf life and resistance to proteolytic degradation in vivo). Such modified polypeptides, when designed to retain at least one activity of the naturally-occurring form of the protein, are considered functional equivalents of the Cbl-b or Cbl-b-AP (e.g., POSH) polypeptides described in more detail herein. Such modified polypeptides can be produced, for instance, by amino acid substitution, deletion, or addition.

For instance, it is reasonable to expect, for example, that an isolated replacement of a leucine with an isoleucine or valine, an aspartate with a glutamate, a threonine with a serine, or a similar replacement of an amino acid with a structurally related amino acid (i.e,. conservative mutations) will not have a major effect on the biological activity of the resulting molecule. Conservative replacements are those that take place within a family of amino acids that are related in their side chains. Genetically encoded amino acids are can be divided into four families (see, for example, Biochemistry, 2nd ed., Ed. by L. Stryer, W.H. Freeman and Co., 1981). Whether a change in the amino acid sequence of a polypeptide results in a functional homolog can be readily determined by assessing the ability of the variant polypeptide to produce a response in cells in a fashion similar to the wild-type protein. For instance, such variant forms of a Cbl-b polypeptide can be assessed, e.g., for their ability to bind to another polypeptide, e.g., another Cbl-b polypeptide or another protein involved in viral maturation, such as the Cbl-b-AP, POSH. Polypeptides in which more than one replacement has taken place can readily be tested in the same manner.

This application further contemplates a method of generating sets of combinatorial mutants of the Cbl-b or Cbl-b-AP (e.g., POSH) polypeptides, as well as truncation mutants, and is especially useful for identifying potential variant sequences ( e.g., homologs) that are functional in binding to a Cbl-b or Cbl-b-AP polypeptide. The purpose of screening such combinatorial libraries is to generate, for example, Cbl-b homologs which can act as either agonists or antagonist, or alternatively, which possess novel activities all together. Combinatorially-derived homologs can be generated which have a selective potency relative to a naturally occurring Cbl-b or Cbl-b-AP polypeptide. Such proteins, when expressed from recombinant DNA constructs, can be used in gene therapy protocols.

Likewise, mutagenesis can give rise to homologs which have intracellular half-lives dramatically different than the corresponding wild-type protein. For example, the altered protein can be rendered either more stable or less stable to proteolytic degradation or other cellular process which result in destruction of, or otherwise inactivation of the Cbl-b or Cbl-b-AP polypeptide of interest. Such homologs, and the genes which encode them, can be utilized to alter Cbl-b or Cbl-b-AP levels by modulating the half-life of the protein. For instance, a short half-life can give rise to more transient biological effects and, when part of an inducible expression system, can allow tighter control of recombinant Cbl-b or Cbl-b-AP levels within the cell. As above, such proteins, and particularly their recombinant nucleic acid constructs, can be used in gene therapy protocols.

In similar fashion, Cbl-b or Cbl-b-AP homologs can be generated by the present combinatorial approach to act as antagonists, in that they are able to interfere with the ability of the corresponding wild-type protein to function.

In a representative embodiment of this method, the amino acid sequences for a population of Cbl-b or Cbl-b-AP homologs are aligned, preferably to promote the highest homology possible. Such a population of variants can include, for example, homologs from one or more species, or homologs from the same species but which differ due to mutation. Amino acids which appear at each position of the aligned sequences are selected to create a degenerate set of combinatorial sequences. In a preferred embodiment, the combinatorial library is produced by way of a degenerate library of genes encoding a library of polypeptides which each include at least a portion of potential Cbl-b or Cbl-b-AP sequences. For instance, a mixture of synthetic oligonucleotides can be enzymatically ligated into gene sequences such that the degenerate set of potential Cbl-b or Cbl-b-AP nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential Cbl-b or Cbl-b-AP sequences. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. A G Walton, Amsterdam: Elsevier pp273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: .404-406; Cwirla et al., (1990) PNAS USA 87: 6378-6382; as well as U.S. Pat. Nos. 5,223,409, 5,198,346, and 5,096,815).

Alternatively, other fQrms of mutagenesis can be utilized to generate a combinatorial library. For example, Cbl-b or Cbl-b-AP homologs (both agonist and antagonist forms) can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of Cbl-b or Cbl-b-AP polypeptides.

A wide range of techniques are known in the art for screening gene products of combinatorial libraries made by point mutations and truncations, and, for that matter, for screening cDNA libraries for gene products having a c ertain property. Such techniques will be generally adaptable for rapid screening of the gene libraries generated by the combinatorial mutagenesis of Cbl-b or Cbl-b-AP homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In an illustrative embodiment of a screening assay, candidate combinatorial gene products of one of the subject proteins are displayed on the surface of a cell or virus, and the ability of particular cells or viral particles to bind a Cbl-b or Cbl-b-AP polypeptide is detected in a “panning assay”. For instance, a library of Cbl-b variants can be cloned into the gene for a surface membrane protein of a bacterial cell (Ladner et al.,, WO 88/06630; Fuchs et al., (1991) Bio/Technology 9:1370-1371; and Goward et al., (1992) TIBS 18:136-140), and the resulting fusion protein detected by panning, e.g., using a fluorescently labeled molecule which b inds the Cbl-b polypeptide, to score for potentially finctional homologs. Cells can be visually inspected and separated under a fluorescence microscope, or, where the morphology of the cell permits, separated by a fluorescence-activated cell sorter.

In similar fashion, the gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at very high concentrations, a large number of phage can be screened at one time. Second, since each infectious phage displays the combinatorial gene product on its surface, if a particular phage is recovered from an affinity matrix in low yield, the phage can be amplified by another round of infection. The group of almost identical E. coli filamentous phages M13, fd, and fl are most often used in phage display libraries, as either of the phage gill or gVIII coat proteins can be used to generate fusion proteins without disrupting the ultimate packaging of the viral particle (Ladner et al., PCT publication WO 90/02909; Garrard et al., PCT publication WO 92/09690; Marks et al., (1992) J. Biol. Chem. 267:16007-16010; Griffiths et al., (1993) EMBO J. 12:725-734; Clackson et al., (1991) Nature 352:624-628; and Barbas et al., (1992) PNAS USA 89:4457-4461).

The application also provides for reduction of the Cbl-b or Cbl-b-AP polypeptides to generate mimetics, e.g., peptide or non-peptide agents, which are able to mimic binding of the authentic protein to another cellular partner. Such mutagenic techniques as described above, as well as the thioredoxin system, are also particularly useful for mapping the determinants of a Cbl-b or Cbl-b-AP polypeptide which participate in protein-protein interactions involved in, for example, binding of proteins involved in viral maturation to each other. To illustrate, the critical residues of a Cbl-b or Cbl-b-AP polypeptide which are involved in molecular recognition of a substrate protein can be determined and used to generate its derivative peptidomimetics which bind to the substrate protein, and by inhibiting Cbl-b or Cbl-b-AP binding, act to inhibit its biological activity. By employing, for example, scanning mutagenesis to map the amino acid residues of a Cbl-b polypeptide which are involved in binding to another polypeptide, peptidomimetic compounds can be generated which mimic those residues involved in binding. For instance, non-hydrolyzable peptide analogs of such residues can be generated using benzodiazepine (e.g., see Freidinger et al., in Peptides: Chemistry and Biology, G.R. Marshall ed., ESCOM Publisher: Leiden, Netherlands, 1988), azepine (e.g., see Huffman et al., in Peptides: Chemistry and Biology, G. R. Marshall ed., ESCOM Publisher: Leiden, Netherlands, 1988), substituted gamma lactam rings (Garvey et al., in Peptides: Chemistry and Biology, G. R. Marshall ed., ESCOM Publisher: Leiden, Netherlands, 1988), keto-methylene pseudopeptides (Ewenson et al., (1986) J. Med. Chem. 29:295; and Ewenson et al., in Peptides: Structure and Function (Proceedings of the 9th American Peptide Symposium) Pierce Chemical Co. Rockland, Ill., 1985), b-turn dipeptide cores (Nagai et al., (1985) Tetrahedron Lett 26:647; and Sato et al., (1986) J Chem Soc Perkin Trans 1:1231), and b-aminoalcohols (Gordon et al., (1985) Biochem Biophys Res Commun 126:419; and Dann et al., (1986) Biochem Biophys Res Commun 134:71).

The following table provides the sequences of the RING domain and the various SH3 domains of POSH.

TABLE 7

Amino Acid Sequences and related SEQ ID NOs for domains in
human POSH

| | | SEQ |
|---|---|---|
| Name of the | | ID |
| sequence | Sequence | NO. |
| RING domain | CPVCLERLDASAKVLPCQHTFCKRCLLGIVGSRNELRCPEC | 26 |
| 1^{st} SH₃ | PCAKALYNYEGKEPGDLKFSKGDIIILRRQVDENWYHGEVNGIHGF | 27 |
| domain | FPTNFVQIIK | |
| 2^{nd} SH₃ | PQCKALYDFEVKDKEADKDCLPFAKDDVLTVIRRVDENWAEGMLAD | 28 |
| domain | KIGIFPISYVEFNS | |
| 3^{rd} SH₃ | SVYVAIYPYTPRKEDELELRKGEMFLVFERCQDGWFKGTSMHTSKI | 29 |
| domain | GVFPGNYVAPVT | |
| 4^{th} SH₃ | ERHRVVVSYPPQSEAELELKEGDIVFVHKICREDGWFKGTLQRNGKT | 30 |
| domain | GLFPGSFVENI | |

### 10. ANTIBODIES AND USES THEREOF

Another aspect of the invention pertains to an antibody specifically reactive with a Cbl-b protein. For example, by using immunogens derived from a Cbl-b protein, e.g., based on the cDNA sequences, anti-protein/anti-peptide antisera or monoclonal antibodies can be made by standard protocols (See, for example, Antibodies: A L aboratory M anual ed. by H arlow and L ane (Cold Spring H arbor Press: 1988)). A mammal, such as a mouse, a hamster or rabbit can be immunized with an immunogenic form of the peptide (e.g., a Cbl-b polypeptide or an antigenic fragment which is capable of eliciting an antibody response, or a fusion protein as described above). Techniques for conferring immunogenicity on a protein or peptide include conjugation to carriers or other techniques well known in the art. An immunogenic portion of a Cbl-b protein can be administered in the presence of adjuvant. The progress of immunization can be monitored by detection of antibody titers in plasma or serum. Standard ELISA or other immunoassays can be used with the immunogen as antigen to assess the levels of antibodies. In a preferred embodiment, the subject antibodies are immunospecific for antigenic determinants of a Cbl-b protein of a mammal, e.g., antigenic determinants of a protein set forth in SEQ ID NO: 45 or SEQ ID NO: 46.

In one embodiment, antibodies are specific for a RING domain, a TKl3 domain, a proline rich domain, or an SH2 domain, and preferably the domain is part of a Cbl-b protein. In a certain embodiment, the domain is part of an amino acid sequence set forth in SEQ ID NO: 45 or SEQ ID NO: 46. In another embodiment, the antibodies are immunoreactive with one or more proteins having an amino acid sequence that is at least 80% identical to an amino acid sequence as set forth in SEQ ID NO: 45 or SEQ ID NO: 46. In other embodiments, an antibody is immunoreactive with one or more proteins having an amino acid sequence that is 85%, 90%, 95,O, 98%, 99% or identical to an amino acid sequence as set forth in any one of SEQ ID NOS: 45-46.

Following immunization of an animal with an antigenic preparation of a Cbl-b protein, anti-Cbl-b protein antisera can be obtained and, if desired, polyclonal anti-Cbl-b protein antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a mammalian Cbl-b protein of the present invention and monoclonal antibodies isolated from a culture comprising such hybridoma cells. In one embodiment anti-human Cbl-b antibodies specifically react with the protein encoded by a nucleic acid having any one of SEQ ID NOS: 45-46.

The term antibody as used herein is intended to include fragments thereof which are also specifically reactive with one of the subject Cbl-b proteins. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for a Cbl-b protein conferred by at least one CDR region of the antibody. In preferred embodiments, the antibodies, the antibody further comprises a label attached thereto and able to be detected, (e.g., the label can be a radioisotope, fluorescent compound, enzyme or enzyme co-factor).

Anti-Cbl-b protein antibodies can be used, e.g., to monitor Cbl-b protein levels, respectively, in an individual, particularly the presence of Cbl-b protein at the plasma membrane for determining whether or not said patient is infected with a virus such as an RNA virus, a retroid virus, and an envelop virus, or allowing determination of the efficacy of a given treatment regimen for an individual afflicted with such a disorder. In addition, Cbl-b protein polypeptides are expected to localize, occasionally, to the released viral particle. Viral particles may be collected and assayed for the presence of a Cbl-b protein. The level of Cbl-b protein may be measured in a variety of sample types such as, for example, cells and/or in bodily fluid, such as in blood samples.

Another application of anti-Cbl-b protein antibodies of the present invention is in the immunological screening of cDNA libraries constructed in expression vectors such as gt11, gt18-23, ZAP, and ORF8. Messenger libraries of this type, having coding sequences inserted in the correct reading frame and orientation, can produce fusion proteins. For instance, gt11 will produce fusion proteins whose amino termini consist of β-galactosidase amino acid sequences and whose carboxy termini consist of a foreign polypeptide. Antigenic epitopes of a Cbl-b protein, e.g., other orthologs of a particular protein or other paralogs from the same species, can then be detected with antibodies, as, for example, reacting nitrocellulose filters lifted from infected plates with the appropriate anti-Cbl-b protein antibodies. Positive phage detected by this assay can then be isolated from the infected plate. Thus, the presence of Cbl-b protein homologs can be detected and cloned from other animals, as can alternate isoforms (including splice variants) from humans.

### 10. EFFECTIVE DOSE

Toxicity and therapeutic efficacy of such compounds can be determined by standard pharmaceutical procedures in cell cultures or experimental animals, e.g., for determining The LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the application, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### 11. FORMULATION AND USE

Pharmaceutical compositions for use in accordance with the present application may be formulated in conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration.

An exemplary composition of the application comprises an RNAi mixed with a delivery system, such as a liposome system, and optionally including an acceptable excipient. In a preferred embodiment, the composition is formulated for topical administration for, e.g., herpes virus infections.

For such therapy, the compounds of the application can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the application can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present application are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives in addition, detergents may be used to facilitate permeation. Transmucosal administration may be through nasal sprays or using suppositories. For topical administration, the oligomers of the application are formulated into ointments, salves, gels, or creams as generally known in the art. A wash solution can be used locally to treat an injury or inflammation to accelerate healing.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

For therapies involving the administration of nucleic acids, the oligomers of the application can be formulated for a variety of modes of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, intranodal, and subcutaneous for injection, the oligomers of the application can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the oligomers are formulated into conventional oral administration forms such as capsules, tablets, and tonics. For topical administration, the oligomers of the application are formulated into ointments, salves, gels, or creams as generally known in the art.

The application now being generally described, it will be more readily understood by reference to the following examples, which are included merely for purposes of illustration of certain aspects and embodiments of the present application, and are not intended to limit the application.

### EXAMPLES

### Example 1

### Role of POSH in Virus-Like Particle (VLP) Budding

1. Objective:

Use RNAi to inhibit POSH gene expression and compare the efficiency of viral budding and GAG expression and processing in treated and untreated cells.

2. Study Plan:

HeLa SS-6 cells are transfected with mRNA-specific RNAi in order to knockdown the target proteins. Since maximal reduction of target protein by RNAi is achieved after 48 hours, cells are transfected twice—first to reduce target mRNAs, and subsequently to express the viral Gag protein. The second transfection is performed with pNLenv (plasmid that encodes HIV) and with low amounts of RNAi to maintain the knockdown of target protein during the time of gag expression and budding of VLPs. Reduction in mRNA levels due to RNAi effect is verified by RT-PCR amplification of target mRNA.

3. Methods, Materials, Solutions

a. Methods
i. Transfections according to manufacturer's protocol and as described in procedure.
ii. Protein determined by Bradford assay.
iii. SDS-PAGE in Hoeffer miniVE electrophoresis system. Transfer in Bio-Rad mini-protean II wet transfer system. Blots visualized using Typhoon system, and ImageQuant software (ABbiotech)

b. Materials

| Material | Manufacturer | Catalog # | Batch # |
|---|---|---|---|
| Lipofectamine 2000 | Life Technologies | 11668-019 | 1112496 |
| (LF2000) | | | |
| OptiMEM | Life Technologies | 31985-047 | 3063119 |
| RNAi Lamin A/C | Self | 13 | |
| RNAi TSG101 688 | Self | 65 | |
| RNAi Posh 524 | Self | 81 | |
| plenvl1 PTAP | Self | 148 | |
| plenvl1 ATAP | Self | 149 | |
| Anti-p24 polyclonal | Seramun | | A-0236/5- |
| antibody | | | 10-01 |
| Anti-Rabbit Cy5 | Jackson | 144-175-115 | 48715 |
| conjugated antibody | | | |
| 10% acrylamide Tris- | Life Technologies | NP0321 | 1081371 |
| Glycine SDS-PAGE gel | | | |
| Nitrocellulose | Schleicher & | 401353 | BA-83 |
| membrane | Schuell | | |
| NuPAGE 20X transfer | Life Technologies | NP0006-1 | 224365 |
| buffer | | | |
| 0.45 μm filter | Schleicher & | 10462100 | CS1018-1 |
| | Schuell | | |

c. Solutions

| | Compound | Concentration |
|---|---|---|
| Lysis Buffer | Tris-HCl pH 7.6 | 50 mM |
| | MgCl₂ | 15 mM |
| | NaCl | 150 mM |
| | Glycerol | 10% |
| | EDTA | 1 mM |
| | EGTA | 1 mM |
| | ASB-14 (add immediately | 1% |
| | before use) | |
| 6X Sample | Tris-HCl, pH = 6.8 | 1M |
| Buffer | Glycerol | 30% |
| | SDS | 10% |
| | DTT | 9.3% |
| | Bromophenol Blue | 0.012% |
| TBS-T | Tris pH = 7.6 | 20 mM |
| | NaCl | 137 mM |
| | Tween-20 | 0.1% |

4. Procedure

a. Schedule

Day

| 1 | 2 | 3 | 4 | 5 |
|---|---|---|---|---|
| Plate | Transfection I | Passage | Transfection II | Extract RNA |
| cells | (RNAi only) | cells | (RNAi and | for RT-PCR |
| | | (1:3) | pNlenv) | (post |
| | | | (12:00, PM) | transfection) |
| | | | Extract RNA for | Harvest VLPs |
| | | | RT-PCR | and cells |
| | | | (pre-transfection) | |

b. Day I

Plate HeLa SS-6 cells in 6-well plates (35mm wells) at concentration of 5 X105 cells/well.

c. Day 2

2 hours before transfection replace growth medium with 2 ml growth medium without antibiotics.

Transfection I:

| | | | | | RNAi | A | B |
|---|---|---|---|---|---|---|---|
| | | | | | [20 μM] | OPtiMEM | LF2000 mix |
| Reaction | RNAi name | TAGDA# | Reactions | RNAi [nM] | μl | (μl) | (μl) |
| 1 | Lamin A/C | 13 | 2 | 50 | 12.5 | 500 | 500 |
| 2 | Lamin A/C | 13 | 1 | 50 | 6.25 | 250 | 250 |
| 3 | TSG101 688 | 65 | 2 | 20 | 5 | 500 | 500 |
| 5 | Posh 524 | 81 | 2 | 50 | 12.5 | 500 | 500 |

Transfections:

Prepare LF2000 mix: 250 μl OptiMEM+5 μl LF2000 for each reaction. Mix by inversion, 5 times. Incubate 5 minutes at room temperature.

Prepare RNA dilution in OptiMEM (Table 1, column A). Add LF2000 mix dropwise to diluted RNA (Table 1, column B). Mix by gentle, vortex. Incubate at room temperature 25 minutes, covered with aluminum foil.

Add 500 μl transfection mixture to cells dropwise and mix by rocking side to side.

Incubate overnight.

d. Day 3

Split 1:3 after 24 hours. (Plate 4 wells for each reaction, except reaction 2 which is plated into 3 wells.)

e. Day 4

2 hours pre-transfection replace medium with DMEM growth medium without antibiotics.

Transfection II

| | | | | | B | | |
|---|---|---|---|---|---|---|---|
| | | | | A | RNAi | | |
| | | | | Plasmid | [20 μM] for | C | D |
| RNAi | | | | for 2.4 μg | 10 nM | OPtiMEM | LF2000 mix |
| name | TAGDA# | Plasmid | Reaction # | (μl) | (μl) | (μl) | (μl) |
| Lamin | 13 | PTAP | 3 | 3.4 | 3.75 | 750 | 750 |
| A/C | | | | | | | |
| Lamin | 13 | ATAP | 3 | 2.5 | 3.75 | 750 | 750 |
| A/C | | | | | | | |
| TSG101 | 65 | PTAP | 3 | 3.4 | 3.75 | 750 | 750 |
| 688 | | | | | | | |
| Posh 524 | 81 | PTAP | 3 | 3.4 | 3.75 | 750 | 750 |

Prepare LF2000 mix: 250 μl OptiMEM+5 μl LF2000 for each reaction. Mix by inversion, 5 times. Incubate 5 minutes at room temperature.

Prepare RNA+DNA diluted in OptiMEM (Transfection II, A+B+C) Add LF2000 mix (Transfection II, D) to diluted RNA+DNA dropwise, mix by gentle vortex, and incubate 1 h while protected from light with aluminum foil.

Add LF2000 and DNA+RNA to cells, 500 μl/well, mix by gentle rocking and incubate overnight.

f. Day 5

Collect samples for VLP assay (approximately 24 hours post-transfection) by the following procedure (cells from one well from each sample is taken for RNA assay, by RT-PCR).

g. Cell Extracts
i. Pellet floating cells by centrifugation (5 min, 3000 rpm at 4° C.), save supernatant (continue with supernatant immediately to step h), scrape remaining cells in the medium which remains in the well, add to the corresponding floating cell pellet and centrifuge for 5 minutes, 1800rpm at 4° C.
ii. Wash cell pellet twice with ice-cold PBS.
iii. Resuspend cell pellet in 100 μl lysis buffer and incubate 20 minutes on ice.
iv. Centrifuge at 14,000 rpm for 15 min. Transfer supernatant to a clean tube. This is the cell extract.
v. Prepare 10 μl of cell extract samples for SDS-PAGE by adding SDS-PAGE sample buffer to 1×, and boiling for 10 minutes. Remove an aliquot of the remaining sample for protein determination to verify total initial starting material. Save remaining cell extract at −80° C.

h. Purification of VLPs from Cell Media
i. Filter the supernatant from step g through a 0.45 m filter.
ii. Centrifuge supernatant at 14,000 rpm at 4° C. for at least 2 h.
iii. Aspirate supernatant carefully.
iv. Re-suspend VLP pellet in hot (100° C. warmed for 10 min at least) 1× sample buffer.
v. Boil samples for 10 minutes, 100° C.

i. Western Blot analysis
i. Run all samples from stages A and B on Tris-Glycine SDS-PAGE 10% (120V for 1.5 h).
ii. Transfer samples to nitrocellulose membrane (65V for 1.5 h).
iii. Stain membrane with ponceau S solution.
iv. Block with 10% low fat milk in TBS-T for 1 h.
v. Incubate with anti p24 rabbit 1:500 in TBS-T o/n.
vi. Wash 3 times with TBS-T for 7 min each wash.
vii. Incubate with secondary antibody anti rabbit cy5 1:500 for 30 min.
viii. Wash five times for 10 min in TBS-T.
ix. View in Typhoon gel imaging system (Molecular Dynamics/APBiotech) for fluorescence signal.

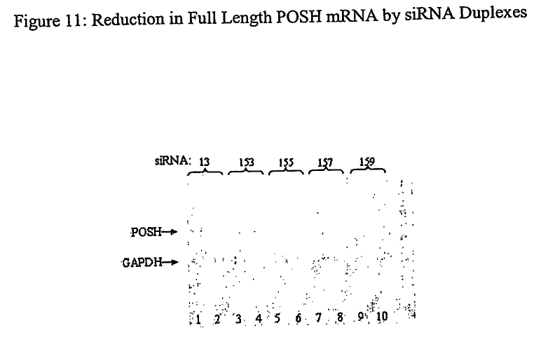

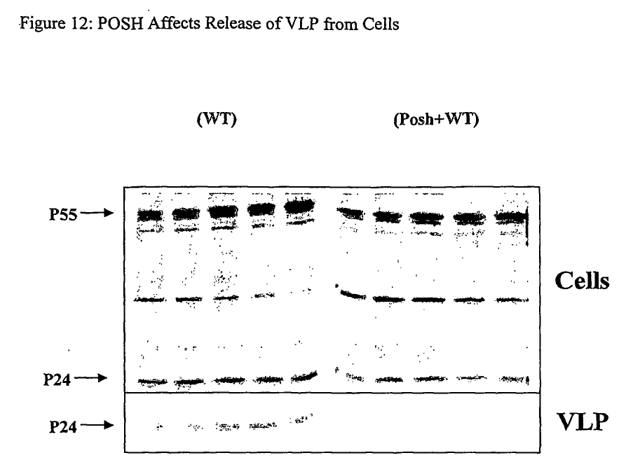

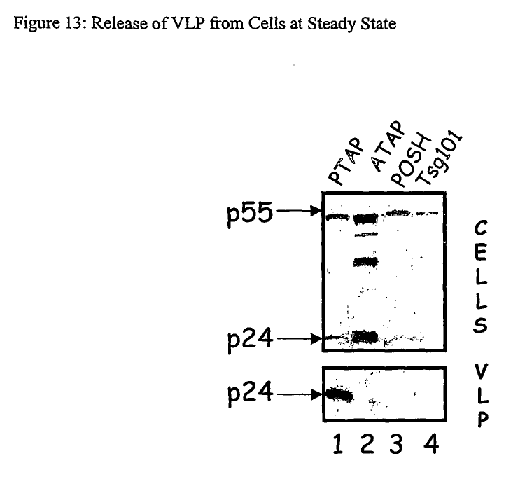

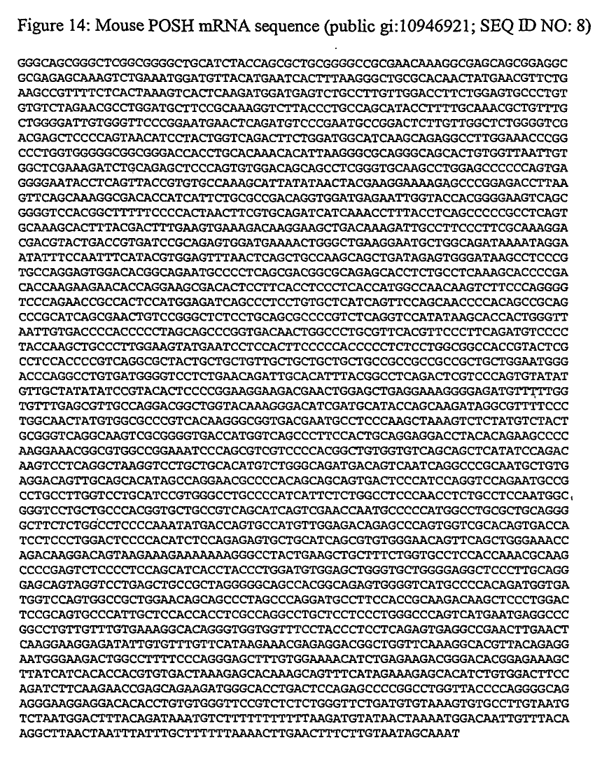

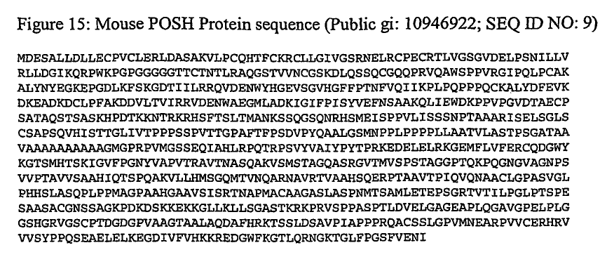

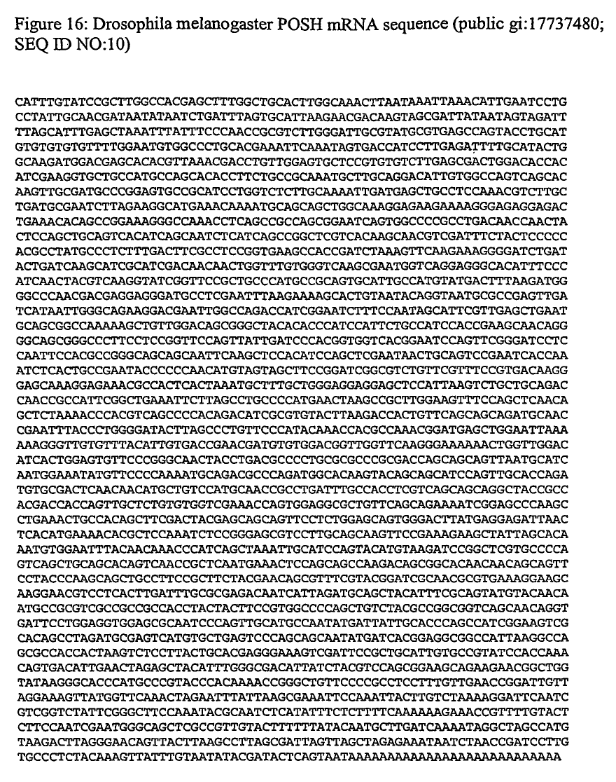

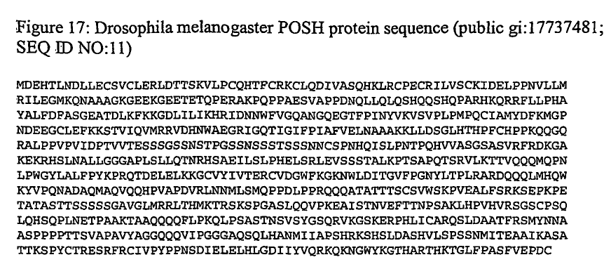

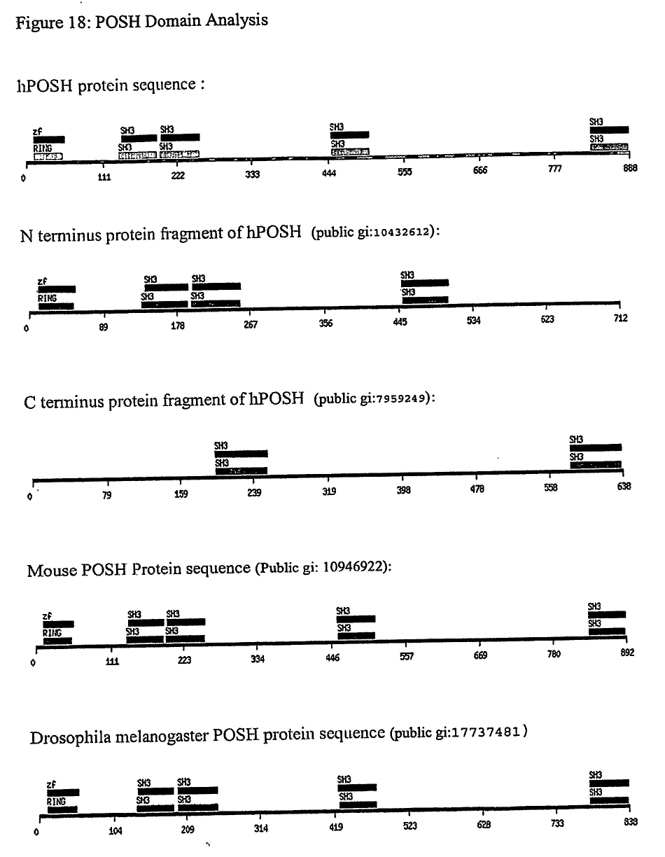

Results are shown in FIGS. 11-13.

### Example 2

### Exemplary POSH RT-PCR Primers and siRNA Duplexes

RT-PCR primers

| | | |
|---|---|---|
| Name | Position | Sequence |

| | | | |
|---|---|---|---|
| Sense primer | POSH = 271 | 271 | 5′ CTTGCCTTGCCAGCATAC 3′(SEQ ID NO: 12) |
| Anti-sense | POSH = 926c | 926C | 5′ CTGCCAGCATTCCTTCAG 3′(SEQ ID NO: 13) |
| primer | | | |

siRNA Duplexes:

| | | |
|---|---|---|
| siRNA No: | 153 | |
| siRNA Name: | POSH-230 | |
| Position in mRNA | 426-446 | |
| Target sequence: | 5′ AACAGAGGCCTTGGAAACCTG 3′ | SEQ ID NO: 1 |
| siRNA sense strand: | 5′ dTdTCAGAGGCCUUGGAAACCUG 3′ | SEQ ID NO: 1 |
| siRNA anti-sense strand: | 5′ dTdTCAGGUUUCGAAGGCCUCUG 3′ | SEQ ID NO: 1 |
| siRNA No: | 155 | |
| siRNA Name: | POSH-442 | |
| Position in mRNA | 638-658 | |
| Target sequence: | 5′ AAAGAGCCTGGAGACCTTAAA 3′ | SEQ ID NO: 1 |
| siRNA sense strand: | 5′ ddTdTAGAGCCUGGAGACCUUAAA 3′ | SEQ ID NO: 1 |
| siRNA anti-sense strand: | 5′ ddTdTUUUAAGGUCUCCAGGCUCU 3′ | SEQ ID NO: 1 |
| siRNA No: | 157 | |
| siRNA Name: | POSH-U111 | |
| Position in mRNA | 2973-2993 | |
| Target sequence: | 5′ AAGGATTGGTATGTGACTCTG 3′ | SEQ ID NO: 2 |
| siRNA sense strand: | 5′ dTdTGGAUUGGUAUGUGACUCUG 3′ | SEQ ID NO: 2 |
| siRNA anti-sense strand: | 5′ dTdTCAGAGUCACAUACCAAUCC 3′ | SEQ ID NO: 2 |
| siRNA No: | 159 | |
| siRNA Name: | POSH-U410 | |
| Position in mRNA | 3272-3292 | |
| Target sequence: | 5′ AAGCTGGATTATCTCCTGTTG 3′ | SEQ ID NO: 2 |
| siRNA sense strand: | 5′ ddTdTGCUGGAUUAUCUCCUGUUG 3′ | SEQ ID NO: 2 |
| siRNA anti-sense strand: | 5′ ddTdTCAACAGGAGAUAAUCCAGC 3′ | SEQ ID NO: 2 |
| siRNA No.: | 187 | |
| siRNA Name: | POSH-control | |
| Position in mRNA: | None. Reverse to #153 | |
| Target sequence: | 5′ AAGTCCAAAGGTTCCGGAGAC 3′ | SEQ ID NO: 36 |

### Example 3

### Knock-Down of HPOSH Entraps HIV Virus Particles in Intracellular Vesicles

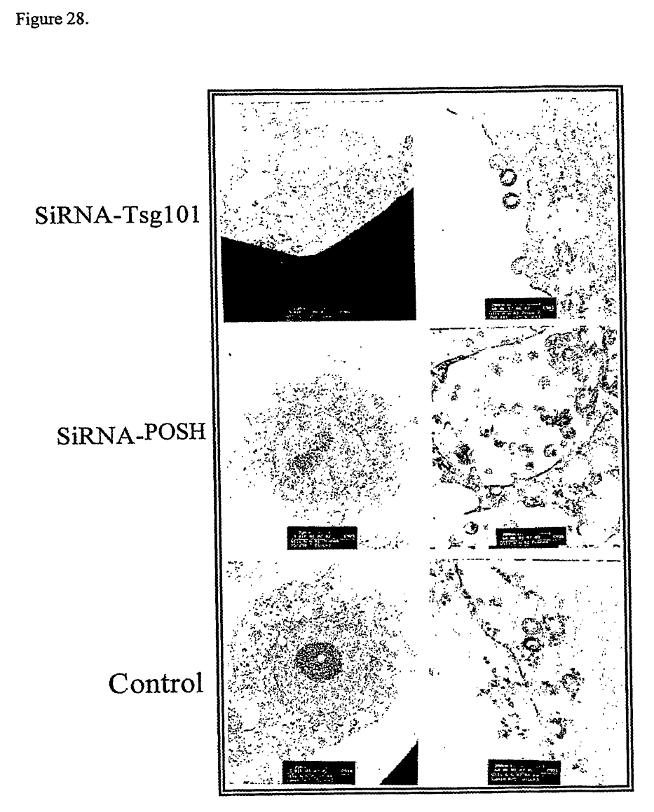

HIV virus release was analyzed by electron microscopy following siRNA and full-length HIV plasmid (missing the envelope coding region) transfection. Mature viruses were secreted by cells transfected with HIV plasmid and non-relevant siRNA (control, lower panel). Knockdown of Tsg101 protein resulted in a budding defect, the viruses that were released had an immature phenotype (upper panel). Knockdown of hPOSH levels resulted in accumulation of viruses inside the cell in intracellular vesicles (middle panel). Results, shown in FIG. 28, indicate that inhibiting HPOSH entraps HIV virus particles in intracellular vesicles. As accumulation of HIV virus particles in the cells accelerate cell death, inhibition of HPOSH therefore destroys HIV reservoir by killing cells infected with HWV.

### Example 4

### In-Vitro Assay of Human POSH Self-Ubiquitination

Recombinant hPOSH was incubated with ATP in the presence of E1, E2 and ubiquitin as indicated in each lane. Following incubation at 37° C. for 30 minutes, reactions were terminated by addition of SDS-PAGE sample buffer. The samples were subsequently resolved on a 10% polyacrylamide gel. The separated samples were then transferred to nitrocellulose and subjected to immunoblot analysis with an anti ubiquitin polyclonal antibody. The position of migration of molecular weight markers is indicated on the right.

Poly-Ub: Ub-hPOSHconjugates, detected as high molecular weight adducts only in reactions containing E1, E2 and ubiquitin. hPOSH-176 and hPOSH-178 are a short and a longer derivatives (respectively) of bacterially expressed hPOSH; C, control E3.

Preliminary Steps in H high-Throughput Screen

Materials

1. E1 recombinant from bacculovirus
2. E2 Ubch5c from bacteria
3. Ubiquitin
4. POSH #178 (1-361) GST fusion-purified but degraded
5. POSH # 176 (1-269) GST fusion-purified but degraded
6. hsHRD1 soluble ring containing region
5. Bufferx12 (Tris 7.6 40 mM, DTT 1 mM, MgCk₂ 5 mM, ATP 2 uM)

6. Dilution buffer (Tris 7.6 40 mM, DTT 1 mM, ovalbumin 1 ug/ul) protocol

| | 0.1 ug/ul | 0.5 ug/ul | 5 ug/ul | 0.4 ug/ul | 2.5 ug/u/ | 0.8 ug/ul | |
|---|---|---|---|---|---|---|---|
| | E1 | E2 | Ub | 176 | 178 | Hrd1 | Bx12 |
| −E1 (E2 + 176) | — | 0.5 | 0.5 | 1 | — | — | 10 |
| −E2 (E1 + 176) | 1 | — | 0.5 | 1 | — | — | 9.5 |
| −ub (E1 + E2 + 176) | 1 | 0.5 | — | 1 | — | — | 9.5 |
| E1 + E2 + 176 + Ub | 1 | 0.5 | 0.5 | 1 | | — | 9 |
| −E1 (E2 + 178) | — | 0.5 | 0.5 | — | 1 | — | 10 |
| −E2 (E1 + 178) | 1 | — | 0.5 | — | 1 | — | 9.5 |
| −ub (E1 + E2 + 178) | 1 | 0.5 | — | — | 1 | — | 9.5 |
| E1 + E2 + 178 + Ub | 1 | 0.5 | 0.5 | — | 1 | —1 | 9 |
| Hrd1, E1 + E2 + Ub | 1 | 0.5 | 0.5 | — | — | 1 | 8.5 |

*

1. Incubate for 30 minutes at 37° C.
2. Run 12% SDS PAGE gel and transfer to nitrocellulose membrane
3. Incubate with anti-Ubiquitin antibody.

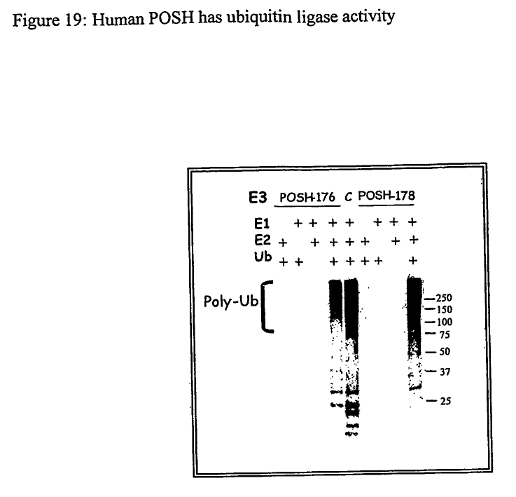

Results, shown in FIG. 19, demonstrate that human POSH has ubiquitin ligase activity.

### Example 5

### POSH Reduction Results in Decreased Secretion of Phospholipase D (PLD)

Hela SS6 cells (two wells of 6-well plate) were transfected with POSH siRNA or control siRNA (100 nM). 24 hours later each well was split into 5 wells of a 24-well plate. The next day cells were transfected again with 100 nM of either POSH siRNA or control siRNA. The next day cells were washed three times with 1×PBS and than 0.5 ml of PLD incubation buffer (118 mM NaCl, 6 mM KCl, 1 mM CaCl₂, 1.2 mM MgSO4, 12.4 mM HEPES, pH7.5 and 1% fatty acid free bovine serum albumin) were added.

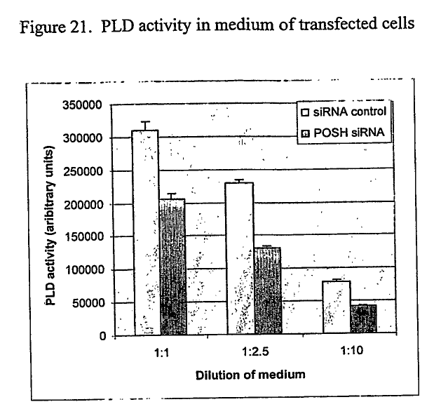

48 hours later medium was collected and centrifuged at 800×g for 15 minutes. The medium was diluted with 5×PLD reaction buffer (Amplex red PLD kit) and assayed for PLD by using the Amplex Red PLD kit (Molecular probes, A-12219). The assay results were quantified and presented below in as a bar graph. The cells were collected and lysed in 1% Triton X-100 lysis buffer (20 mM HEPES-NaOH, pH 7.4, 150 mM NaCl, 1.5 mM MgCl₂, 1 mM EDTA, 1% Triton X-100 and 1× protease inhibitors) for 15 minutes on ice. Lysates were cleared by centrifugation and protein concentration was determined. There were equal protein concentrations between the two transfectants. Equal amount of extracts were immunoprecipitated with anti-POSH antibodies, separated by SDS-PAGE and immunoblotted with anti-POSH antibodies to assess the reduction of POSH levels. There was approximately 40% reduction in POSH levels (FIG. 21).

### Example 6

### Effect of hPOSH on Gag-EGFP Intracellular Distribution

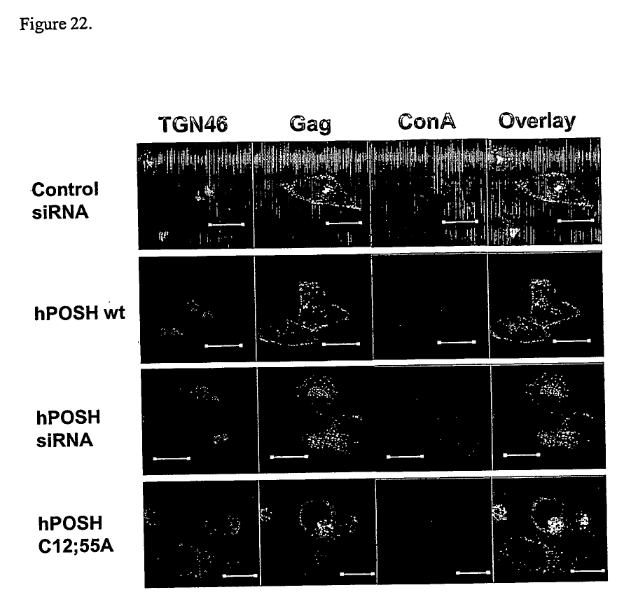

HeLa SS6 were transfected with Gag-EGFP, 24 hours after an initial transfection with either hPOSH-specific or scrambled siRNA (control) (100 nM) or with plasmids encoding either wild type hPOSH or hPOSH C(12,55)A. Fixation and staining was preformed 5 hours after Gag-EGFP transfection. Cells were fixed, stained with Alexa fluor 647-conjugated Concanavalin A (ConA) (Molecular Probes), permeabilized and then stained with sheep anti-human TGN46. After the primary antibody incubation cells were incubated with Rhodamin-conjugated goat anti-sheep. Laser scanning confocal microscopy was performed on LSM510 confocal microscope (Zeiss) equipped with Axiovert 100M inverted microscope using ×40 magnification and 1.3-numerical-aperture oil-immersion lens for imaging. For co-localization experiments, 10 optical horizontal sections with intervals of 1 μm were taken through each preparation (Z-stack). A single median section of each preparation is shown. See FIG. 22.

### Example 7

### POSH-Regulated Intracellular Transport of Myristoyated Proteins

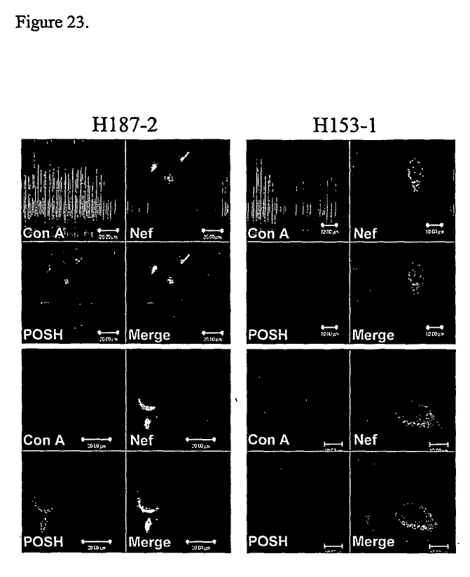

The localization of myristoylated proteins, Gag (see FIG. 22), HIV-1 Nef, Src and Rapsyn, in cells depleted of hPOSH were analyzed by immunofluorescence. In control cells, HIV-1 Nef was found in a perinuclear region co-localized with hPOSH, indicative of a TGN localization (FIG. 23). When hPOSH expression was reduced by siRNA treatment, Nef expression was weaker relative to control and nef lost its TGN, perinuclear localization. Instead it accumulated in punctated intracellular loci segregated from the TGN.

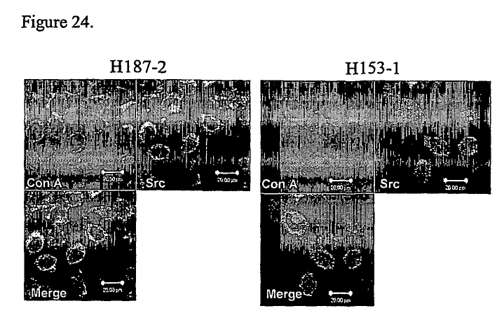

Src is expressed at the plasma membrane and in intracellular vesicles, which are found close to the plasma membrane (FIG. 24, H187 cells). However, when hPOSH levels were reduced, Src was dispersed in the cytoplasm and loses its plasma membrane proximal localization detected in control (H187) cells (FIG. 24, compare H153-1 and H187-2 panels).

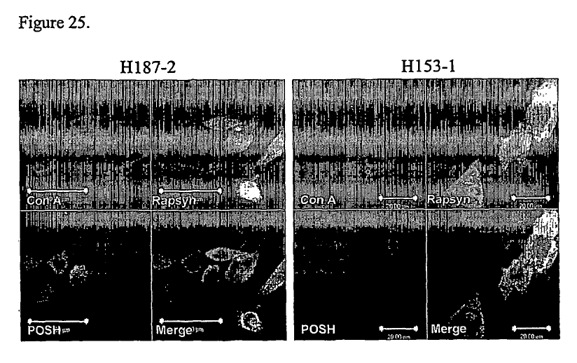

Rapsyn, a peripheral membrane protein expressed in skeletal muscle, plays a critical role in organizing the structure of the nicotinic postsynaptic membrane (Sanes and Lichtrnan, Annu. Rev. Neurosci. 22: 389-442 (1999)). Newly synthesized Rapsyn associates with the TGN and than transported to the plasma membrane (archand et al., J. Neurosci. 22: 8891-01 (2002)). In hPOSH-depleted cells (H153-1) Rapsyn was dispersed in the cytoplasm, while in control cells it had a punctuated pattern and plasma membrane localization, indicating that hPOSH influences its intracellular transport (FIG. 25).

Materials and Methods Used:

Antibodies:

Src antibody was purchased from Oncogene research products(Darmstadt, Germany). Nef antibodies were pusrchased from ABI (Columbia, Mass.) and Fitzgerald Industries Interantional (Concord, Mass.). Alexa Fluor conjugated antibodies were pusrchased from Molecular Probes Inc. (Eugene, Oreg.).

hPOSH antibody: Glutathione S-transferase (GST) fusion plasmids were constructed by PCR amplification of hPOSH codons 285-430. The amplified PCR products was cloned into pGEX-6P-2 (Amersham Pharmacia Biotech, Buckinghamshire, UK). The truncated HPOSH protein was generated in E. coli BL21. Bacterial cultures were grown in LB media with carbenicillin (100 μg/ml) and recombinant protein production was induced with 1 mM IPTG for 4 hours at 30° C. Cells were lysed by sonication and the recombinant protein was then isolated from the cleared bacterial lysate by affinity chromatography on a glutathione-sepharose resin (Amersham Pharmacia Biotech, Buckinghamshire, UK). The HPOSH portion of the fusion protein was then released by incubation with PreScission protease (Amersham Pharmacia Biotech, Buckinghamshire, UK) according to the manufacturer's instructions and the GST portion was then removed by a second glutathione-sepharose affinity chromatography. The purified partial hPOSH polypeptide was used to immunize New Zealand white rabbits to generate antibody 15B (Washington Biotechnology, Baltimore, Md.).

Construction of siRNA Retroviral Vectors:

HPOSH scrambled oligonucleotide (5′-CACACACTGCCG TCAACT GTTCAAGAGAC AGTTGACGGCAGTGTGTGTTTTTT-3′; and 5′-AATTAAAAAACACA CACTGCCGTCAACTGTC TCTTGAACAGTTGA CGGCAGTGTGTGGGCC-3′) were annealed and cloned into the ApaI-EcoRI digested pSilencer 1.0-US (Ambion) to generate pSIL-scrambled. Subsequently, the U6-promoter and RNAi sequences were digested with BamHI, the ends filled in and the insert cloned into the Olil site in the retroviral vector, pMSVhyg (Clontech), generating pMSCVhyg-U6-scrambled. HPOSH oligonucleotide encoding RNAi against hPOSH (5′-AACAGAGGCCTTGGAAA CCTGGAAGC TTGCAGGTTT CCAAGGCCTCTGTT-3′; and 5′-GATCAACAGAG GCCTTGGAAACCTGC AAGCTTCCAGGTTTCCAA GGCCTCTGTT-3′) were annealed and cloned into the BamHI-EcoRI site of pLIT-U6, generating pLIT-U6 hPOSH-230. pLIT-U6 is an shRNA vector containing the human U6 promoter (amplified by PCR from human genomic DNA with the primers, 5′-GGCCCACTAGTCA AGGTCG GGCA GGAAGA-3′ and 5′-GCCGAATT CAAAAAGGATC CGGCGATATCCGG TGTTTCGTCCTTTCCA-3′) cloned into pLITMUS38 (New England Biolabs) digested with SpeI-EcoRI. Subsequently, the U6 promoter-hPOSH shRNA (pLIT-U6 hPOSH-230 digested with SnaBI and PvuI) was cloned into the Olil site of pMSVhyg (Clontech), generating pMSCVhyg U6-hPOSH-230.

Generation of Stable Clones:

HEK 293T cells were transfected with retroviral RNAi plasmids (pMSCVhyg-U6-POSH-230 and pMSCVhyg-U6-scrambled and with plasmids encoding VSV-G and moloney gag-pol. Two days post transfection, medium containing retroviruses was collected and filtered and polybrene was added to a final concentration of 8 μg/ml. This was used to infect HeLa SS6 cells grown in 60 mm dishes. Forty-eight hours post-infection cells were selected for RNAi expression by the addition of hygromycin to a final concentration of 300 μg/ml. Clones expressing RNAi against HPOSH were named H153, clones expressing scrambled RNAi were named H187.

Transfection and Immunofluorescent Analysis:

Gag-EGFP experiments are described in Example 6 and FIG. 22.

H153 or H187 cells were transfected with Src or Rapsyn-GFP (Image clone image: 3530551 or pNLenv-1). Eighteen hours post transfection cells were washed with PBS and incubated on ice with Alexa Fluor 647 conjugated C on A to label plasma membrane glycoproteins. Subsequently cells were fixed in 3% paraformaldehyde, blocked with PBS containing 4% bovine serum albumin and 1% gelatin. Staining with rabbit anti-Src, rabbit anti-hPOSH (15B) or mouse anti-nef was followed with secondary antibodies as indicated.

Laser scanning confocal microscopy was performed on LSM510 confocal microscope (Zeiss) equipped with Axiovert 100M inverted microscope using x40 magnification and 1.3-numerical-aperture oil-immersion lens for imaging. For co-localization experiments, 10 optical horizontal sections with intervals of 1 μm were taken through each preparation (Z-stack). A single median section of each preparation is shown.

### Example 8

### POSH Reduction by siRNA Abrogates West Nile Virus (“WNV”) Infectivity

HeLa SS6 cells were transfected with either control or POSH-specific siRNA. Cells were subsequently infected with WNV (4×10⁴ PFU/well). Viruses were harvested 24 hours and 48 hours post-infection, serially diluted, and used to infect Vero cells. As a control WNV (4×10⁴ PFU/well), that was not passed through HeLa SS6 cells, was used to infect Vero cells. Virus titer was determined by plaque assay in Vero cells.

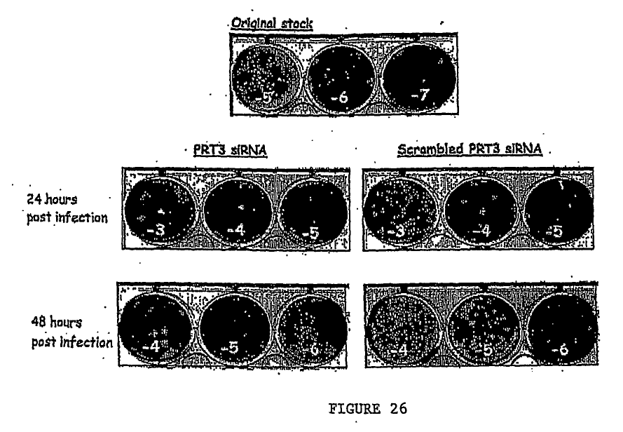

Virus titer was reduced by 2.5-log in cells treated with POSH-specific siRNA relative to cells transfected with control siRNA, thereby indicating that WNV requires POSH for virus secretion. See FIG. 26.

Experimental Procedure:

Cell Culture, Transfections and Infection:

Hela SS6 cells were grown in Dulbecco's modified Eagle's medium (DMEM) supplemented with 10% heat-inactivated fetal calf serum and 100 units/ml penicillin and 100 μg/ml streptomycin. For transfections, HeLa SS6 cells were grown to 50% confluency in DMEM containing 10% FCS without antibiotics. Cells were then transfected with the relevant double-stranded siRNA (100 nM) using lipofectamin 2000 (Invitrogen, Paisley, UK). On the day following the initial transfection, cells were split 1:3 in complete medium and transfected with a second portion of double-stranded siRNA (50 nM). Six hours post-transfection medium was replaced and cells infected with WNV (4×10⁴ PFU/well). Medium was collected from infected HeLa SS6 cells twenty-four and forty-eight post-infection (200 μl), serially diluted, and used to infect Vero cells. Virus titer was determined by plaque assay (Ben-Nathan D, Lachmi B, Lustig S, Feuerstien G (1991) Protection of dehydroepiandrosterone (DHEA) in mice ifected with viral encephalitis. Arch Viro; 120, 263-271).

### EXAMPLE 9

### Analysis of the Effects of POSH Knockdown on M-MuLV Expression and Budding

Experimental Protocol:

Transfections:

A day before transfection, Hela SS6 cells were plated in two 6 wells plates at 5×10⁵ cells per well. 24 hours later the following transfections were performed:
4 wells were transfected with control siRNA and a plasmid encoding MMuLV.
4 wells were transfected with POSH siRNA and a plasmid encoding MMuLV.
1 well was a control without any siRNA or DNA transfected.
1 well was transfected with a plasmid encoding MMuLV.

For each well to be transfected 100 nM (12.5 μl) POSH siRNA or 100 nM (12.5 μl) control siRNA were diluted in 250 μl Opti-MEM (Invitrogen). Lipofectamin 2000 (5 μl) (Invitrogen, Cat. 11668-019) was mixed with 250 μl of OptiMEM per transfected well. The diluted siRNA was mixed with the lipofectamin 2000 mix and the solution incubated at room temperature for 30 min. The mixture was added directly to each well containing 2 ml DMEM +10% FBS (w/o antibiotics).

24 hours later, four wells of the same siRNA treatment were split to eight wells, and two wells without siRNA were split to four wells.

24 hours later all wells were transfected with 100 nM control siRNA or 100 nM POSH siRNA with or without a plasmid encoding MMuLV (see table below). 48 hours later virions and cells were harvested.

| | | Amount | | | |
|---|---|---|---|---|---|
| | | of RNAi | | The volume of | |
| No of | | (μl) per | Amount of DNA | DNA (μl) per | |
| wells | RNAi | well | (μg) per well | well | Application |
| 5 | POSH | 12.5 | MMuLV (2 μg) | 10 | 4 wells for |
| | 100 nM(1^{st} | | | | VLPs assay |
| | and 2^{nd} | | | | and 1 well for |
| | transfection) | | | | RT |
| 5 | Control | 12.5 | MMuLV (2 μg) | 10 | 4 wells for |
| | 100 nM (1^{st} | | | | VLPs assay |
| | and 2^{nd} | | | | and 1 well for RT |
| | transfection) | | | | |
| 1 | — | — | — | 10 μl H₂O | VLPs assay |
| 1 | — | — | MMuLV (2 μg) | 10 | VLPs assay |

Steady State VLP Assay

Cell Extracts:
1. Pellet floating cells by centrifugation (10 min, 500×g at 4° C.), save supernatant (continued at step 7), wash cells once, scrape cells in ice-cold 1×PBS, add to the corresponding cell pellet and centrifuge for 5 min 1800 rpm at 4° C.
2. Wash cell pellet once with ice-cold 1×PBS.
3. Resuspend cell pellet in 150 μt 1% Triton X-100 lysis buffer (20 mM HEPES-NaOH, pH 7.4, 150 mM NaCl, 1.5 mM MgCl₂, 1 mM EDTA, 1% Triton X-100 and 1× protease inhibitors) and incubate 20 minutes on ice.
4. Centrifuge at 14,000rpm for 15 min. Transfer supernatant to a clean tube.
5. Determine protein concentration by BCA.
6. Prepare samples for SDS-PAGE by adding 2 μl of 6×SB to 20 μg extract (add lysis buffer to a final volume of 12 μl), heat to 80° C. for 10 min.

Purification of Virions from Cell Media
7. Filtrate the supernatant through a 0.45 μm filter.
8. Transfer 1500 μl of virions fraction to an ultracentrifuge tube (swinging rotor).
9. Add 300 μl of fresh sucrose cushion (20% sucrose in TNE) to the bottom of the tube.
10. Centrifuge supernatant at 35000 rpm at 4° C. for 2 hr.
11. Resuspend virion pellet in 50 μl hot 1× sample buffer each (samples 153-1, 2, 3, 187-1, 2, 3). Resuspend VLPs pellet (153-4, 5 and 187 4, 5) in 25 μl hot 1× sample buffer. Vortex shortly, transfer to an eppendorf tube, unite VLPs from wells 153-4+5 and 187-4+5. Heat to 80° C. for 10 min.
12. Load equal amounts of VLPs relatively to cells extracts amounts.

Western Blot Analysis
1. Separate all samples on 12% SDS-PAGE.
2. Transfer samples to nitrocellulose membrane (100V for 1.15 hr).
3. Dye membrane with ponceau solution.
4. Block with 10% low fat milk in TBS-T for 1 hour.
5. Incubate membranes with Goat anti p30 (81S-263) (1:5000) in 10% low fat milk in TBS-T over night at 4° C. Incubate with secondary antibody rabbit anti goat-HRP 1:8000 for 60 min at room temperature.
6. Detect signal by ECL reaction.
7. Following the ECL detection incubate memebranes with Donkey anti rabbit Cy3 (Jackson Laboratories, Cat 711-165-152) 1:500 and detect signal by Typhoon scanning and quantitate.

Results:

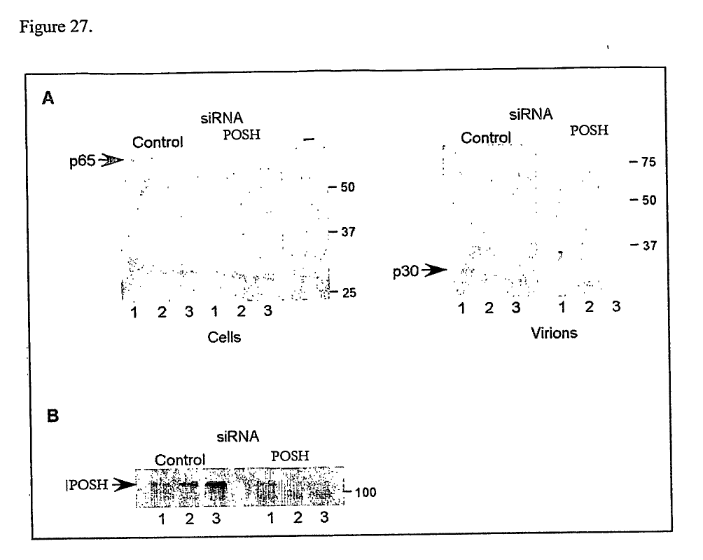

As shown in FIG. 27, POSH knockdown decreases the release of extracellular MMuLV particles.

### Example 10

### POSH Protein-Protein Interactions by Yeast Two Hybrid Assay

POSH-associated proteins were identified by using a yeast two-hybrid assay.

Procedure:

Bait plasmid (GAL4-BD) was transformed into yeast strain AH109 (Clontech) and transformants were selected on defined media lacking tryptophan. Yeast strain Y187 containing pre-transformed Hela cDNA prey (GAL4-AD) library (Clontech) was mated according to the Clontech protocol with bait containing yeast and plated on defined media lacking tryptophan, leucine, histidine and containing 2 mM 3 amino triazol. Colonies that grew on the selective media were tested for beta-galactosidase activity and positive clones were further characterized. Prey clones were identified by amplifying cDNA insert and sequencing using vector derived primers.

Bait:

Plasmid vector: pGBK-T7 (Clontech)

Plasmid name: pPL269- pGBK-T7 GAL4 POSHdR

Protein sequence: Corresponds to aa 53-888 of POSH (RING domain deleted)

RTLVGSGVEELPSNILLVRLLDGIKQRPWKPGPGGGSGTNCTNALRSQSS
TVANCSSKDLQSSQGGQQPRVPSWSPPVRGIPQLPCAKALYNYEGKEPGD
LKFSKGDIIILRRQVDENWYHGEVNGIHGFFPTNFVQIIKPLPQPPPQCK
ALYDFEVKDKEADKDCLPFAKDDVLTVIRRVDENWAEGMLADKIGIFPIS
YVEFNSAAKQLIEWDKPPVPGVDAGECSSAAAQSSTAPKHSDTKKNTKKR
HSFTSLTMANKSSQASQNRHSMEISPPVLISSSNPTAAARISELSGLSCS
APSQVHISTTGLIVTPPPSSPVTTGPSFTFPSDVPYQAALGTLNPPLPPP
PLLAATVLASTPPGATAAAAAAGMGPRPMAGSTDQIAHLRPQTRPSVYVA
IYPYTPRKEDELELRKGEMFLVFERCQDGWFKGTSMHTSKIGVFPGNYVA
PVTRAVTNASQAKVPMSTAGQTSRGVTMVSPSTAGGPAQKLQGNGVAGSP
SVVPAAVVSAAHIQTSPQAKVLLHMTGQMTVNQARNAVRTVAAHNQERPT
AAVTPIQVQNAAGLSPASVGLSHHSLASPQPAPLMPGSATHTAAISISRA
SAPLACAAAAPLTSPSITSASLEAEPSGRIVTVLPGLPTSPDSASSACGN
SSATKPDKDSKKEKKGLLKLLSGASTKRKPRVSPPASPTLEVELGSAELP
LQGAVGPELPPGGGHGRAGSCPVDGDGPVTTAVAGAALAQDAFHRKASSL
DSAVPIAPPPRQACSSLGPVLNESRPVVCERHRVVVSYPPQSEAELELKE
GDIVFVHKKREDGWFKGTLQRNGKTGLFPGSFVENI

Library screened: Hela pretransformed library (Clontech).

The POSH-AP, Cbl-b, was identified by yeast two-hybrid assay. Examples of nucleic acid and amino acid sequences of Cbl-b are provided below, including examples of sequences (SEQ ID NOS: 43-46) for additional Cbl-b polypeptides identified by yeast two-hybrid screen.

| Clone | BLAST hit | UniGene | Name | Remarks |
|---|---|---|---|---|
| 3Gd114 | AK094184 | Hs.381921 | Homo sapiens cDNA | 1 seq file |
| | | | FLJ36865 fis, clone | in |
| | | | ASTRO2016148, | Unigene |
| | | | highly similar to Signal | |
| | | | transduction protein | |
| | | | CBL-B | |
| | BC032851 | Hs.3144 | CBLB Cas-Br-M | aa631- |
| | | | (murine) ecotropic | COOH |
| | | | retroviral transforming | |
| | | | sequence b | |

Human CBL-B mRNA sequence - var1
(public gi: 4757919)

(SEQ ID NO: 37)

CTGGGTCCTGTGTGTGCCACAGGGGTGGGGTGTCCAGCGAGCGGTCTCCT
CCTCCTGCTAGTGCTGCTGCGGCGTCCCGCGGCCTCCCCGAGTCGGGCGG
GAGGGGAGAGCGGGTGTGGATTTGTCTTGACGGTAATTGTTGCGTTTCCA
CGTCTCGGAGGCCTGCGCGCTGGGTTGCTCCTTCTTCGGGAGCGAGCTGT
TCTCAGCGATCCCACTCCCAGCCGGGGCTCCCCACACACACTGGGCTGCG
TGCGTGTGGAGTGGGACCCGCGCACACGCGTGTCTCTGGACAGCTACGGC
GCCGAAAGAACTAAAATTCCAGATGGCAAACTCAATGAATGGCAGAAACC
CTGGTGGTCGAGGAGGAAATCCCCGAAAAGGTCGAATTTTGGGTATTATT
GATGCTATTCAGGATGCAGTTGGACCCCCTAAGCAAGCTGCCGCAGATCG
CAGGACCGTGGAGAAGACTTGGAAGCTCATGGACAAAGTGGTAAGACTGT
GCCAAAATCCCAAACTTCAGTTGAAAAATAGCCCACCATATATACTTGAT
ATTTTGCCTGATACATATCAGCATTTACGACTTATATTGAGTAAATATGA
TGACAACCAGAAACTTGCCCAACTCAGTGAGAATGAGTACTTTAAAATCT
ACATTGATAGCCTTATGAAAAAGTCAAAACGGGCAATAAGACTCTTTAAA
GAAGGCAAGGAGAGAATGTATGAAGAACAGTCACAGGACAGACGAAATCT
CACAAAACTGTCCCTTATCTTCAGTCACATGCTGGCAGAAATCAAAGCAA
TCTTTCCCAATGGTCAATTCCAGGGAGATAACTTTCGTATCACAAAAGCA
GATGCTGCTGAATTCTGGAGAAAGTTTTTTGGAGACAAAACTATCGTACC
ATGGAAAGTATTCAGACAGTGCCTTCATGAGGTCCACCAGATTAGCTCTA
GCCTGGAAGCAATGGCTCTAAAATCAACAATTGATTTAACTTGCAATGAT
TACATTTCAGTTTTTGAATTTGATATTTTTACCAGGCTGTTTCAGCCTTG
GGGCTCTATTTTGCGGAATTGGAATTTCTTAGCTGTGACACATCCAGGTT
ACATGGCATTTCTCACATATGATGAAGTTAAAGCACGACTACAGAAATAT
AGCACCAAACCCGGAAGCTATATTTTCCGGTTAAGTTGCACTCGATTGGG
ACAGTGGGCCATTGGCTATGTGACTGGGGATGGGAATATCTTACAGACCA
TACCTCATAACAAGCCCTTATTTCAAGCCCTGATTGATGGCAGCAGGGAA
GGATTTTATCTTTATCCTGATGGGAGGAGTTATAATCCTGATTTAACTGG
ATTATGTGAACCTACACCTCATGACCATATAAAAGTTACACAGGAACAAT
ATGAATTATATTGTGAAATGGGCTCCACTTTTCAGCTCTGTAAGATTTGT
GCAGAGAATGACAAAGATGTCAAGATTGAGCCTTGTGGGCATTTGATGTG
CACCTCTTGCCTTACGGCATGGCAGGAGTCGGATGGTCAGGGCTGCCCTT
TCTGTCGTTGTGAAATAAAAGGAACTGAGCCCATAATCGTGGACCCCTTT
GATCCAAGAGATGAAGGCTCCAGGTGTTGCAGCATCATTGACCCCTTTGG
CATGCCGATGCTAGACTTGGACGACGATGATGATCGTGAGGAGTCCTTGA
TGATGAATCGGTTGGCAAACGTCCGAAAGTGCACTGACAGGCAGAACTCA
CCAGTCACATCACCAGGATCCTCTCCCCTTGCCCAGAGAAGAAAGCCACA
GCCTGACCCACTCCAGATCCCACATCTAAGCCTGCCACCCGTGCCTCCTC
GCCTGGATCTAATTCAGAAAGGCATAGTTAGATCTCCCTGTGGCAGCCCA
ACAGGTTCACCAAAGTCTTCTCCTTGCATGGTGAGAAAACAAGATAAACC
ACTCCCAGCACCACCTCCTCCCTTAAGAGATCCTCCTCCACCGCCACCTG
AAAGACCTCCACCAATCCCACCAGACAATAGACTGAGTAGACACATCCAT
CATGTGGAAAGCGTGCCTTCCAGAGACCCGCCAATGCCTCTTGAAGCATG
GTGCCCTCGGGATGTGTTTGGGACTAATCAGCTTGTGGGATGTCGACTCC
TAGGGGAGGGCTCTCCAAAACCTGGAATCACAGCGAGTTCAAATGTCAAT
GGAAGGCACAGTAGAGTGGGCTCTGACCCAGTGCTTATGCGGAAACACAG
ACGCCATGATTTGCCTTTAGAAGGAGCTAAGGTCTTTTCCAATGGTCACC
TTGGAAGTGAAGAATATGATGTTCCTCCCCGGCTTTCTCCTCCTCCTCCA
GTTACCACCCTCCTCCCTAGCATAAAGTGTACTGGTCCGTTAGCAAATTC
TCTTTCAGAGAAAACAAGAGACCCAGTAGAGGAAGATGATGATGAATACA
AGATTCCTTCATCCCACCCTGTTTCCCTGAATTCACAACCATCTCATTGT
CATAATGTAAAACCTCCTGTTCGGTCCTGTGATAATGGTCACTGTATGCT
GAATGGAACACATGGTCCATCTTCAGAGAAGAAATCAAACATCCCTGACT
TAAGCATATATTTAAAGGGTACGTATAGAATATAATTTCCTTTGTGATGT
ACATCTTAATGGTCAGAATTTAAAGGCAAAATTTCATGCCATTGTACTGA
AAATACATTAAGGTTTTGTGTTATCCTCTAGGAGATGTTTTTGATTCAGC
CTCTGATCCCGTGCCATTACCACCTGCCAGGCCTCCAACTCGGGACAATC
CAAAGCATGGTTCTTCACTCAACAGGACGCCCTCTGATTATGATCTTCTC
ATCCCTCCATTAGGTTGAAACCTTTAAAAAAGTTTTGAACAACCCACCCC
TCCTTCTTTTAATTTCAGAATTTTCAGAATTCAGAGTTCAGTATAACACA
GACTCACTGGGTTGTGAATTTGCCTGAAATTTGAATGGGTTCTCCAGGTG
CCGGTGACTCCCAAGTTCACGAGACCATTACTCCATGTAGATGATTAAGG
TAGTAGTGTAGTAGTTGGGCATCAGTCAGGTTTTAAGCAAGTTGTTTTGT
CCATACTAAATGTAGTCTAAAAACACATGAGAGCTTTGTGCTCTAGTAGT
TTTGAAGTGATGACTTGAAGTGTTGAGATTTTCTTTAAGTATAATAATTC
TTAATAAATATGAACTTGCTTTTCTTGCAGCATGAGCACCAGTTCCACTT
ACGCTAATTAAATTATGCAAAATTAAATAGTTGTATGTAGAGAACTGATA
ATAAATTCTGTTTTATTCTAATCATTACAACTGTAACACATTCAAAAAAA
AAAA
Human CBL-B mRNA sequence - var2
(public gi: 23273908)

(SEQ ID NO: 38)

AGCGGAGTGCTGCTGCGGCGTCCCGCGGCCTCCCCGAGTCGGGCGGGAGG
GGAGAGCGGGTGTGGATTTGTCTTGACGGTAATTGTTGCGTTTCCACGTC
TCGGAGGCCTGCGCGCTGGGTTGCTCCTTCTTCGGGAGCGAGCTGTTCTC
AGCGATCCCACTCCCAGCCGGGGCTCCCCACACACACTGGGGTGCGTGCG
TGTGGAGTGGGACCCGCGCACACGCGTGTCTCTGGACAGCTACGGCGCCG
AAAGAACTAAAATTCCAGATGGCAAACTCAATGAATGGCAGAAACCCTGG
TGGTCGAGGAGGAAATCCCCGAAAAGGTCGAATTTTGGGTATTATTGATG
CTATTCAGGATGCAGTTGGACCCCCTAAGCAAGCTGCCGCAGATCGCAGG
ACCGTGGAGAAGACTTGGAAGCTCATGGACAAAGTGGTAAGACTGTGCCA
AAATCCCAAACTTCAGTTGAAAAATAGCCCACCATATATACTTGATATTT
TGCCTGATACATATCAGCATTTACGACTTATATTGAGTAAATATGATGAC
AACCAGAAACTTGCCCAACTCAGTGAGAATGAGTACTTTAAAATCTACAT
TGATAGCCTTATGAAAAAGTCAAAACGGGCAATAAGACTCTTTAAAGAAG
GCAAGGAGAGAATGTATGAAGAACAGTCACAGGACAGACGAAATCTCACA
AAACTGTCCCTTATCTTCAGTCACATGCTGGCAGAAATCAAAGCAATCTT
TCCCAATGGTCAATTCCAGGGAGATAACTTTCGTATCACAAAAGCAGATG
CTGCTGAATTCTGGAGAAAGTTTTTTGGAGACAAAACTATCGTACCATGG
AAAGTATTCAGACAGTGCCTTCATGAGGTCCACCAGATTAGCTCTGGCCT
GGAAGCAATGGCTCTAAAATCAACAATTGATTTAACTTGCAATGATTACA
TTTCAGTTTTTGAATTTGATATTTTTACCAGGCTGTTTCAGCCTTGGGGC
TCTATTTTGCGGAATTGGAATTTCTTAGCTGTGACACATCCAGGTTACAT
GGCATTTCTCACATATGATGAAGTTAAAGCACGACTACAGAAATATAGCA
CCAAACCCGGAAGCTATATTTTCCGGTTAAGTTGCACTCGATTGGGACAG
TGGGCCATTGGCTATGTGACTGGGGATGGGAATATCTTACAGACCATACC
TCATACAAGCCCTTATTTCAAGCCCTGATTGATGGCAGCAGGGAAGGATT
TTAATCTTTATCCTGATGGGAGGAGTIATAATCCTGATTTAACTGGATTA
TGTGAACCTACACCTCATGACCATATAAAAGTTACACAGGAACAATATGA
ATTATATTGTGAAATGGGCTCCACTTTTCAGCTCTGTAAGATTTGTGCAG
AGAATGACAAAGATGTCAAGATTGAGCCTTGTGGGCATTTGATGTGCACC
TCTTGCCTTACGGCATGGCAGGAGTCGGATGGTCAGGGCTGCCCTTTCTG
TCGTTGTGAAATAAAAGGAACTGAGCCCATAATCGTGGATCCCTTTGATC
CAAGAGATGAAGGCTCCAGGTGTTGCAGCATCATTGACCCCTTTGGCATG
CCGATGCTCGACTTGGACGACGATGATGATCGTGAGGAGTCCTTGATGAT
GAATCGGTTGGCAAACGTCCGAAAGTGCACTGACAGGCAGAACTCACCAG
TCACATCACCAGGATCCTCTCCCCTTGCCCAGAGAAGAAAGCCACAGCCT
GACCCACTCCAGATCCCACATCTAAGCCTGCCACCCGTGCCTCCTCGCCT
GGATCTAATTCAGAAAGGCATAGTTAGATCTCCCTGTGGCAGCCCAACGG
GTTCACCAAAGTCTTCTCCTTGCATGGTGAGAAAACAAGATAAACCACTC
CCAGCACCACCTCCTCCCTTAAGAGATCCTCCTCCACCGCCACCTGAAAG
ACCTCCACCAATCCCACCAGACAATAGACTGAGTAGACACATCCATCATG
TGGAAAGCGTGCCTTCCAAAGACCCGCCAATGCCTCTTGAAGCATGGTGC
CCTCGGGATGTGTTTGGGACTAATCAGCTTGTGGGATGTCGACTCCTAGG
GGAGGGCTCTCCAAAACCTGGAATCACAGCGAGTTCAAATGTCAATGGAA
GGCACAGTAGAGTGGGCTCTGACCCAGTGCTTATGCGGAAACACAGACGC
CATGATTTGCCTTTAGAAGGAGCTAAGGTCTTTTCCAATGGTCACCTTGG
AAGTGAAGAATATGATGTTCCTCCCCGGCTTTCTCCTCCTCCTCCAGTTA
CCACCCTCCTCCCTAGCATAAAGTGTACTGGTCCGTTAGCAAATTCTCTT
TCAGAGAAAACAAGAGACCCAGTAGAGGAAGATGATGATGAATACAAGAT
TCCTTCATCCCACCCTGTTTCCCTGAATTCACAACCATCTCATTGTCATA
ATGTAAAACCTCCTGTTCGGTCTTGTGATAATGGTCACTGTATGCTGAAT
GGAACACATGGTCCATCTTCAGAGAAGAAATCAAACATCCCTGACTTAAG
CATATATTTAAAGGGAGATGTTTTTGATTCAGCCTCTGATCCCGTGCCAT
TACCACCTGCCAGGCCTCCAACTCGGGACAATCCAAAGCATGGTTCTTCA
CTCAACAGGACGCCCTCTGATTATGATCTTCTCATCCCTCCATTAGGTGA
AGATGCTTTTGATGCCCTCCCTCCATCTCTCCCACCTCCCCCACCTCCTG
CAAGGCATAGTCTCATTGAACATTCAAAACCTCCTGGCTCCAGTAGCCGG
CCATCCTCAGGACAGGATCTTTTTCTTCTTCCTTCAGATCCCTTTGTTGA
TCTAGCAAGTGGCCAAGTTCCTTTGCCTCCCGCTAGAAGGTTACCAGGTG
AAAATGTCAAAACTAACAGAACATCACAGGACTATGATCAGCTTCCTTCA
TGTTCAGATGGTTCACAGGCACCAGCCAGACCCCCTAAACCACGACCGCG
CAGGACTGCACCAGAAATTCACCACAGAAAACCCCATGGGCCTGAGGCGG
CATTGGAAAATGTCGATGCAAAAATTGCAAAACTCATGGGAGAGGGTTAT
GCCTTTGAAGAGGTGAAGAGAGCCTTAGAGATAGCCCAGAATAATGTCGA
AGTTGCCCGGAGCATCCTCCGAGAATTTGCCTTCCCTCCTCCAGTATCCC
CACGTCTAAATCTATAGCAGCCAGAACTGTAGACACCAAAATGGAAAGCA
ATCGATGTATTCCAAGAGTGTGGAAATAAAGAGAACTGAGATGGAATTCA
AGAGAGAAGTGTCTCCTCCTCGTGTAGCAGCTTGAGAAGAGGCTTGGGAG
TGCAGCTTCTCAAAGGAGACCGATGCTTGCTCAGGATGTCGACAGCTGTG
GCTTCCTTGTTTTTGCTAGCCATATTTTTAAATCAGGGTTGAACTGACAA
AAATAATTTAAAGACGTTTACTTCCCTTGAACTTTGAACCTGTGAAATGC
TTTACCTTGTTTACAATTTGGCAAAGTTGCAGTTTGTTCTTGTTTTTAGT
TTAGTTTTGTTTTGGTGTTTTGATACCTGTACTGTGTTCTTCACAGACCC
TTTGTAGCGTGGTCAGGTCTGCTGTAACATTTCCCACCAACTCTCTTGCT
GTCCACATCAACAGCTAAATCATTTATTCATATGGATCTCTACCATCCCC
ATGCCTTGCCCAGGTCCAGTTCCATTTCTCTCATTCACAAGATGCTTTGA
AGGTTCTGATTTTCAACTGATCAAACTAATGCAAAAAAAAAAGTATGTAT
TCTTCACTACTGAGTTTCTTCTTTGGAAACCATCACTATTGAGAGATGGG
AAAAACCTGAATGTATAAAGCATTTATTTGTCAATAAACTGCCTTTTGTA
AGGGGTTTTCACAAAAAAAAAAAAAAAA
Human CBL-B mRNA sequence - var3
(public gi: 862406)

(SEQ ID NO: 39)

CTGGGTCCTGTGTGTGCCACAGGGGTGGGGTGTCCAGCGAGCGGTCTCCT
CCTCCTGCTAGTGCTGCTGCGGCGTCCCGCGGCCTCCCCGAGTCGGGCGG
GAGGGGAGAGCGGGTGTGGATTTGTCTTGACGGTAATTGTTGCGTTTCCA
CGTCTCGGAGGCCTGCGCGCTGGGTTGCTCCTTCTTCGGGAGCGAGCTGT
TCTCAGCGATCCCACTCCCAGCCGGGGCTCCCCACACACACTGGGCTGCG
TGCGTGTGGAGTGGGACCCGCGCACACGCGTGTCTCTGGACAGCTACGGC
GCCGAAAGAACTAAAATTCCAGATGGCAAACTCAATGAATGGCAGAAACC
CTGGTGGTCGAGGAGGAAATCCCCGAAAAGGTCGAATTTTGGGTATTATT
GATGCTATTCAGGATGCAGTTGGACCCCCTAAGCAAGCTGCCGCAGATCG
CAGGACCGTGGAGAAGACTTGGAAGCTCATGGACAAAGTGGTAAGACTGT
GCCAAAATCCCAAACTTCAGTTGAAAAATAGCCCACCATATATACTTGAT
ATTTTGCCTGATACATATCAGCATTTACGACTTATATTGAGTAAATATGA
TGACAACCAGAAACTTGCCCAACTCAGTGAGAATGAGTACTTTAAAATCT
ACATTGATAGCCTTATGAAAAAGTCAAAACGGGCAATAAGACTCTTTAAA
GAAGGCAAGGAGAGAATGTATGAAGAACAGTCACAGGACAGACGAAATCT
CACAAAACTGTCCCTTATCTTCAGTCACATGCTGGCAGAAATCAAAGCAA
TCTTTCCCAATGGTCAATTCCAGGGAGATAACTTTCGTATCACAAAAGCA
GATGCTGCTGAATTCTGGAGAAAGTTTTTTGGAGACAAAACTATCGTACC
ATGGAAAGTATTCAGACAGTGCCTTCATGAGGTCCACCAGATTAGCTCTA
GCCTGGAAGCAATGGCTCTAAAATCAACAATTGATTTAACTTGCAATGAT
TACATTTCAGTTTTTGAATTTGATATTTTTACCAGGCTGTTTCAGCCTTG
GGGCTCTATTTTGCGGAATTGGAATTTCTTAGCTGTGACACATCCAGGTT
ACATGGCTTTTCTCACATATGATGAAGTTAAAGCACGACTACAGAAATAT
AGCACCAAACCCGGAAGCTATATTTTCCGGTTAAGTTGCACTCGATTGGG
ACAGTGGGCCATTGGCTATGTGACTGGGGATGGGAATATCTTACAGACCA
TACCTCATAACAAGCCCTTATTTCAAGCCCTGATTGATGGCAGCAGGGAA
GGATTTTATCTTTATCCTGATGGGAGGAGTTATAATCCTGATTTAACTGG
ATTATGTGAACCTACACCTCATGACCATATAAAAGTTACACAGGAACAAT
ATGAATTATATTGTGAAATGGGCTCCACTTTTCAGCTCTGTAAGATTTGT
GCAGAGAATGACAAAGATGTCAAGATTGAGCCTTGTGGGCATTTGATGTG
CACCTCTTGCCTTACGGCATGGCAGGAGTCGGATGGTCAGGGCTGCCCTT
TCTGTCGTTGTGAAATAAAAGGAACTGAGCCCATAATCGTGGACCCCTTT
GATCCAAGAGATGAAGGCTCCAGGTGTTGCAGCATCATTGACCCCTTTGG
CATGCCGATGCTAGACTTGGACGACGATGATGATCGTGAGGAGTCCTTGA
TGATGAATCGGTTGGCAAACGTCCGAAAGTGCACTGACAGGCAGAACTCA
CCAGTCACATCACCAGGATCCTCTCCCCTTGCCCAGAGAAGAAAGCCACA
GCCTGACCCACTCCAGATCCCACATCTAAGCCTGCCACCCGTGCCTCCTC
GCCTGGATCTAATTCAGAAAGGCATAGTTAGATCTCCCTGTGGCAGCCCA
ACAGGTTCACCAAAGTCTTCTCCTTGCATGGTGAGAAAACAAGATAAACC
ACTCCCAGCACCACCTCCTCCCTTAAGAGATCCTCCTCCACCGCCACCTG
AAAGACCTCCACCAATCCCACCAGACAATAGACTGAGTAGACACATCCAT
CATGTGGAAAGCGTGCCTTCCAGAGACCCGCCAATGCCTCTTGAAGCATG
GTGCCCTCGGGATGTGTTTGGGACTAATCAGCTTGTGGGATGTCGACTCC
TAGGGGAGGGCTCTCCAAAACCTGGAATCACAGCGAGTTCAAATGTCAAT
GGAAGGCACAGTAGAGTGGGCTCTGACCCAGTGCTTATGCGGAAACACAG
ACGCCATGATTTGCCTTTAGAAGGAGCTAAGGTCTTTTCCAATGGTCACC
TTGGAAGTGAAGAATATGATGTTCCTCCCCGGCTTTCTCCTCCTCCTCCA
GTTACCACCCTCCTCCCTAGCATAAAGTGTACTGGTCCGTTAGCAAATTC
TCTTTCAGAGAAAACAAGAGACCCAGTAGAGGAAGATGATGATGAATACA
AGATTCCTTCATCCCACCCTGTTTCCCTGAATTCACAACCATCTCATTGT
CATAATGTAAAACCTCCTGTTCGGTCCTGTGATAATGGTCACTGTATGCT
GAATGGAACACATGGTCCATCTTCAGAGAAGAAATCAAACATCCCTGACT
TAAGCATATATTTAAAGGGAGATGTTTTTGATTCAGCCTCTGATCCCGTG
CCATTACCACCTGCCAGGCCTCCAACTCGGGACAATCCAAAGCATGGTTC
TTCACTCAACAGGACGCCCTCTGATTATGATCTTCTCATCCCTCCATTAG
GTGAAGATGCTTTTGATGCCCTCCCTCCATCTCTCCCACCTCCCCCACCT
CCTGCAAGGCATAGTCTCATTGAACATTCAAAACCTCCTGGCTCCAGTAG
CCGGCCATCCTCAGGACAGGATCTTTTTCTTCTTCCTTCAGATCCCTTTG
TTGATCTAGCAAGTGGCCAAGTTCCTTTGCCTCCTGCTAGAAGGTTACCA
GGTGAAAATGTCAAAACTAACAGAACATCACAGGACTATGATCAGCTTCC
TTCATGTTCAGATGGTTCACAGGCACCAGCCAGACCCCCTAAACCACGAC
CGCGCAGGACTGCACCAGAAATTCACCACAGAAAACCCCATGGGCCTGAG
GCGGCATTGGAAAATGTCGATGCAAAAATTGCAAAACTCATGGGAGAGGG
TTATGCCTTTGAAGAGGTGAAGAGAGCCTTAGAGATAGCCCAGAATAATG
TCGAAGTTGCCCGGAGCATCCTCCGAGAATTTGCCTTCCCTCCTCCAGTA
TCCCCACGTCTAAATCTATAGCAGCCAGAACTGTAGACACCAAAATGGAA
AGCAATCGATGTATTCCAAGAGTGTGGAAATAAAGAGAACTGAGATGGAA
TTCAAGAGAGAAGTGTCTCCTCCTCGTGTAGCAGCTTGAGAAGAGGCTTG
GGAGTGCAGCTTCTCAAAGGAGACCGATGCTTGCTCAGGATGTCGACAGC
TGTGGCTTCCTTGTTTTTGCTAGCCATATTTTTAAATCAGGGTTGAACTG
ACAAAAATAATTTAAAGACGTTTACTTCCCTTGAACTTTGAACCTGTGAA
ATGCTTTACCTTGTTTACAATTTGGCAAAGTTGCAGTTTGTTCTTGTTTT
TAGTTTAGTTTTGTTTTGGTGTTTTGATACCTGTACTGTGTTCTTCACAG
ACCCTTTGTAGCGTGGTCAGGTCTGCTGTAACATTTCCCACCAACTCTCT
TGCTGTCCACATCAACAGCTAAATCATTTATTCATATGGATCTCTACCAT
CCCCATGCCTTGCCCAGGTCCAGTTCCATTTCTCTCATTCACAAGATGCT
TTGAAGGTTCTGATTTTCAACTGATCAAACTAATGCAAAAAAAAAAAGTA
TGTATTCTTCACTACTGAGTTTCTTCTTTGGAAACCATCACTATTGAGAG
ATGGGAAAAACCTGAATGTATAAAGCATTTATTTGTCAATAAACTGCCTT
TTGTAAGGGGTTTTCACATAAAAAAAAAAAAA
Human CBL-B mRNA sequence - var4
(public gi: 862408)

(SEQ ID NO: 40)

CTGGGTCCTGTGTGTGCCACAGGGGTGGGGTGTCCAGCGAGCGGTCTCCT
CCTCCTGCTAGTGCTGCTGCGGCGTCCCGCGGCCTCCCCGAGTCGGGCGG
GAGGGGAGAGCGGGTGTGGATTTGTCTTGACGGTAATTGTTGCGTTTCCA
CGTCTCGGAGGCCTGCGCGCTGGGTTGCTCCTTCTTCGGGAGCGAGCTGT
TCTCAGCGATCCCACTCCCAGCCGGGGCTCCCCACACACACTGGGCTGCG
TGCGTGTGGAGTGGGACCCGCGCACACGCGTGTCTCTGGACAGCTACGGC
GCCGAAAGAACTAAAATTCCAGATGGCAAACTCAATGAATGGCAGAAACC
CTGGTGGTCGAGGAGGAAATCCCCGAAAAGGTCGAATTTTGGGTATTATT
GATGCTATTCAGGATGCAGTTGGACCCCCTAAGCAAGCTGCCGCAGATCG
CAGGACCGTGGAGAAGACTTGGAAGCTCATGGACAAAGTGGTAAGACTGT
GCCAAAATCCCAAACTTCAGTTGAAAAATAGCCCACCATATATACTTGAT
ATTTTGCCTGATACATATCAGCATTTACGACTTATATTGAGTAAATATGA
TGACAACCAGAAACTTGCCCAACTCAGTGAGAATGAGTACTTTAAAATCT
ACATTGATAGCCTTATGAAAAAGTCAAAACGGGCAATAAGACTCTTTAAA
GAAGGCAAGGAGAGAATGTATGAAGAACAGTCACAGGACAGACGAAATCT
CACAAAACTGTCCCTTATCTTCAGTCACATGCTGGCAGAAATCAAAGCAA
TCTTTCCCAATGGTCAATTCCAGGGAGATAACTTTCGTATCACAAAAGCA
GATGCTGCTGAATTCTGGAGAAAGTTTTTTGGAGACAAAACTATCGTACC
ATGGAAAGTATTCAGACAGTGCCTTCATGAGGTCCACCAGATTAGCTCTA
GCCTGGAAGCAATGGCTCTAAAATCAACAATTGATTTAACTTGCAATGAT
TACATTTCAGTTTTTGAATTTGATATTTTTACCAGGCTGTTTCAGCCTTG
GGGCTCTATTTTGCGGAATTGGAATTTCTTAGCTGTGACACATCCAGGTT
ACATGGCATTTCTCACATATGATGAAGTTAAAGCACGACTACAGAAATAT
AGCACCAAACCCGGAAGCTATATTTTCCGGTTAAGTTGCACTCGATTGGG
ACAGTGGGCCATTGGCTATGTGACTGGGGATGGGAATATCTTACAGACCA
TACCTCATAACAAGCCCTTATTTCAAGCCCTGATTGATGGCAGCAGGGAA
GGATTTTATCTTTATCCTGATGGGAGGAGTTATAATCCTGATTTAACTGG
ATTATGTGAACCTACACCTCATGACCATATAAAAGTTACACAGGAACAAT
ATGAATTATATTGTGAAATGGGCTCCACTTTTCAGCTCTGTAAGATTTGT
GCAGAGAATGACAAAGATGTCAAGATTGAGCCTTGTGGGCATTTGATGTG
CACCTCTTGCCTTACGGCATGGCAGGAGTCGGATGGTCAGGGCTGCCCTT
TCTGTCGTTGTGAAATAAAAGGAACTGAGCCCATAATCGTGGACCCCTTT
GATCCAAGAGATGAAGGCTCCAGGTGTTGCAGCATCATTGACCCCTTTGG
CATGCCGATGCTAGACTTGGACGACGATGATGATCGTGAGGAGTCCTTGA
TGATGAATCGGTTGGCAAACGTCCGAAAGTGCACTGACAGGCAGAACTCA
CCAGTCACATCACCAGGATCCTCTCCCCTTGCCCAGAGAAGAAAGCCACA
GCCTGACCCACTCCAGATCCCACATCTAAGCCTGCCACCCGTGCCTCCTC
GCCTGGATCTAATTCAGAAAGGCATAGTTAGATCTCCCTGTGGCAGCCCA
ACAGGTTCACCAAAGTCTTCTCCTTGCATGGTGAGAAAACAAGATAAACC
ACTCCCAGCACCACCTCCTCCCTTAAGAGATCCTCCTCCACCGCCACCTG
AAAGACCTCCACCAATCCCACCAGACAATAGACTGAGTAGACACATCCAT
CATGTGGAAAGCGTGCCTTCCAGAGACCCGCCAATGCCTCTTGAAGCATG
GTGCCCTCGGGATGTGTTTGGGACTAATCAGCTTGTGGGATGTCGACTCC
TAGGGGAGGGCTCTCCAAAACCTGGAATCACAGCGAGTTCAAATGTCAAT
GGAAGGCACAGTAGAGTGGGCTCTGACCCAGTGCTTATGCGGAAACACAG
ACGCCATGATTTGCCTTTAGAAGGAGCTAAGGTCTTTTCCAATGGTCACC
TTGGAAGTGAAGAATATGATGTTCCTCCCCGGCTTTCTCCTCCTCCTCCA
GTTACCACCCTCCTCCCTAGCATAAAGTGTACTGGTCCGTTAGCAAATTC
TCTTTCAGAGAAAACAAGAGACCCAGTAGAGGAAGATGATGATGAATACA
AGATTCCTTCATCCCACCCTGTTTCCCTGAATTCACAACCATCTCATTGT
CATAATGTAAAACCTCCTGTTCGGTCCTGTGATAATGGTCACTGTATGCT
GAATGGAACACATGGTCCATCTTCAGAGAAGAAATCAAACATCCCTGACT
TAAGCATATATTTAAAGGGAGATGTTTTTGATTCAGCCTCTGATCCCGTG
CCATTACCACCTGCCAGGCCTCCAACTCGGGACAATCCAAAGCATGGTTC
TTCACTCAACAGGACGCCCTCTGATTATGATCTTCTCATCCCTCCATTAG
GTTGAAACCTTTAAAAAAGTTTTGAACAACCCACCCCTCCTTCTTTTAAT
TTCAGAATTTTCAGAATTCAGAGTTCAGTATAACACAGACTCACTGGGTT
GTGAATTTGCCTGAAATTTGAATGGGTTCTCCAGGTGCCGGTGACTCCCA
AGTTCACGAGACCATTACTCCATGTAGATGATTAAGGTAGTAGTGTAGTA
GTTGGGCATCAGTCAGGTTTTAAGCAAGTTGTTTTGTCCATACTAAATGT
AGTCTAAAAACACATGAGAGCTTTGTGCTCTAGTAGTTTTGAAGTGATGA
CTTGAAGTGTTGAGATTTTCTTTAAGTATAATAATTCTTAATAAATATGA
ACTTGCTTTTCTTGCAGCATGAGCACCAGTTCCACTTACGCTAATTAAAT
TATGCAAAATTAAATAGTTGTATGTAGAGAACTGATAATAAATTCTGTTT
TATTCTAATCATTACAACTGTAACACATTAAAAAAAAAAA
Human CBL-B mRNA sequence - var5
(public gi: 862410)

(SEQ ID NO: 41)

CTGGGTCCTGTGTGTGCCACAGGGGTGGGGTGTCCAGCGAGCGGTCTCCT
CCTCCTGCTAGTGCTGCTGCGGCGTCCCGCGGCCTCCCCGAGTCGGGCGG
GAGGGGAGAGCGGGTGTGGATTTGTCTTGACGGTAATTGTTGCGTTTCCA
CGTCTCGGAGGCCTGCGCGCTGGGTTGCTCCTTCTTCGGGAGCGAGCTGT
TCTCAGCGATCCCACTCCCAGCCGGGGCTCCCCACACACACTGGGCTGCG
TGCGTGTGGAGTGGGACCCGCGCACACGCGTGTCTCTGGACAGCTACGGC
GCCGAAGAACTAAAATTCCAGATGGCAAACTCAATTGAATGGCAGAAACC
CTGGTGGTCGAGGAGGAAATCCCCGAAAAGGTCGAATTTTGGGTATTATT
GATGCTATTCAGGATGCAGTTGGACCCCCTAAGCAAGCTGCCGCAGATCG
CAGGACCGTGGAGAAGACTTGGAAGCTCATGGACAAAGTGGTAAGACTGT
GCCAAAATCCCAAACTTCAGTTGAAAAATAGCCCACCATATATACTTGAT
ATTTTGCCTGATACATATCAGCATTTACGACTTATATTGAGTAAATATGA
TGACAACCAGAAACTTGCCCAACTCAGTGAGAATGAGTACTTTAAAATCT
ACATTGATAGCCTTATGAAAAAGTCAAAACGGGCAATAAGACTCTTTAAA
GAAGGCAAGGAGAGAATGTATGAAGAACAGTCACAGGACAGACGAAATCT
CACAAAACTGTCCCTTATCTTCAGTCACATGCTGGCAGAAATCAAAGCAA
TCTTTCCCAATGGTCAATTCCAGGGAGATAACTTTCGTATCACAAAAGCA
GATGCTGCTGAATTCTGGAGAAAGTTTTTTGGAGACAAAACTATCGTACC
ATGGAAAGTATTCAGACAGTGCCTTCATGAGGTCCACCAGATTAGCTCTA
GCCTGGAAGCAATGGCTCTAAAATCAACAATTGATTTAACTTGCAATGAT
TACATTTCAGTTTTTGAATTTGATATTTTTACCAGGCTGTTTCAGCCTTG
GGGCTCTATTTTGCGGAATTGGAATTTCTTAGCTGTGACACATCCAGGTT
ACATGGCATTTCTCACATATGATGAAGTTAAAGCACGACTACAGAAATAT
AGCACCAAACCCGGAAGCTATATTTTCCGGTTAAGTTGCACTCGATTGGG
ACAGTGGGCCATTGGCTATGTGACTGGGGATGGGAATATCTTACAGACCA
TACCTCATAACAAGCCCTTATTTCAAGCCCTGATTGATGGCAGCAGGGAA
GGATTTTATCTTTATCCTGATGGGAGGAGTTATAATCCTGATTTAACTGG
ATTATGTGAACCTACACCTCATGACCATATAAAAGTTACACAGGAACAAT
ATGAATTATATTGTGAAATGGGCTCCACTTTTCAGCTCTGTAAGATTTGT
GCAGAGAATGACAAAGATGTCAAGATTGAGCCTTGTGGGCATTTGATGTG
CACCTCTTGCCTTACGGCATGGCAGGAGTCGGATGGTCAGGGCTGCCCTT
TCTGTCGTTGTGAAATAAAAGGAACTGAGCCCATAATCGTGGACCCCTTT
GATCCAAGAGATGAAGGCTCCAGGTGTTGCAGCATCATTGACCCCTTTGG
CATGCCGATGCTAGACTTGGACGACGATGATGATCGTGAGGAGTCCTTGA
TGATGAATCGGTTGGCAAACGTCCGAAAGTGCACTGACAGGCAGAACTCA
CCAGTCACATCACCAGGATCCTCTCCCCTTGCCCAGAGAAGAAAGCCACA
GCCTGACCCACTCCAGATCCCACATCTAAGCCTGCCACCCGTGCCTCCTC
GCCTGGATCTAATTCAGAAAGGCATAGTTAGATCTCCCTGTGGCAGCCCA
ACAGGTTCACCAAAGTCTTCTCCTTGCATGGTGAGAAAACAAGATAAACC
ACTCCCAGCACCACCTCCTCCCTTAAGAGATCCTCCTCCACCGCCACCTG
AAAGACCTCCACCAATCCCACCAGACAATAGACTGAGTAGACACATCCAT
CTTGTGGAAAGCGTGCCTTCCAGAGACCCGCCAATGCCTCTTGAAGCATG
GTGCCCTCGGGATGTGTTTGGGACTAATCAGCTTGTGGGATGTCGACTCC
TAGGGGAGGGCTCTCCAAAACCTGGAATCACAGCGAGTTCAAATGTCAAT
GGAAGGCACAGTAGAGTGGGCTCTGACCCAGTGCTTATGCGGAAACACAG
ACGCCATGATTTGCCTTTAGAAGGAGCTAAGGTCTTTTCCAATGGTCACC
TTGGAAGTGAAGAATATGATGTTCCTCCCCGGCTTTCTCCTCCTCCTCCA
GTTACCACCCTCCTCCCTAGCATAAAGTGTACTGGTCCGTTAGCAAATTC
TCTTTCAGAGAAAACAAGAGACCCAGTAGAGGAAGATGATGATGAATACA
AGATTCCTTCATCCCACCCTGTTTCCCTGAATTCACAACCATCTCATTGT
CATAATGTAAAACCTCCTGTTCGGTCCTGTGATAATGGTCACTGTATGCT
GAATGGAACACATGGTCCATCTTCAGAGAAGAAATCAAACATCCCTGACT
TAAGCTATATTTAAGGGTACGTATAGAATATAATTTCCTTTGTGATGTAC
ATCTTATAATGGTCAGAATTTAAAGGCAAAATTTCATGCCATTGTACTGA
AAATACATTAAGGTTTTGTGTTATCCTCTAGGAGATGTTTTTGATTCAGC
CTCTGATCCCGTGCCATTACCACCTGCCAGGCCTCCAACTCGGGACAATC
CAAAGCATGGTTCTTCACTCAACAGGACGCCCTCTGATTATGATCTTCTC
ATCCCTCCATTAGGTTGAAACCTTTAAAAAAGTTTTGAACAACCCACCCC
TCCTTCTTTTAATTTCAGAATTTTCAGAATTCAGAGTTCAGTATAACACA
GACTCACTGGGTTGTGAATTTGCCTGAAATTTGAATGGGTTCTCCAGGTG
CCGGTGACTCCCAAGTTCACGAGACCATTACTCCATGTAGATGATTAAGG
TAGTAGTGTAGTAGTTGGGCATCAGTCAGGTTTTAAGCAAGTTGTTTTGT
CCATACTAAATGTAGTCTAAAAACACATGAGAGCTTTGTGCTCTAGTAGT
TTTGAAGTGATGACTTGAAGTGTTGAGATTTTCTTTAAGTATAATAATTC
TTAATAAATATGAACTTGCTTTTCTTGCAGCATGAGCACCAGTTCCACTT
ACGCTAATTAATTTATGCAATATTAAATAGTTGTATGTAGAGAACTGATA
ATAAATTCTGTTTTATTCTAATCATTACAACTGTAACACATTAAAAAAAA
AAA
Human CBL-B mRNA sequence - var6
(public gi: 21753192)

(SEQ ID NO: 42)

AGTGCTGCTGCGGCGTCCCGCGGCCTCCCCGAGTCGGGCGGGAGGGGAGA
GCGGGTGTGGATTTGTCTTGACGGTAATTGTTGCGTTTCCACGTCTCGGA
GGCCTGCGCGCTGGGTTGCTCCTTCTTCGGGAGCGAGCTGTTCTCAGCGA
TCCCACTCCCAGCCGGGGCTCCCCACACACACTGGGCTGCGTGCGTGTGG
AGTGGGACCCGCGCACACGCGTGTCTCTGGACAGCTACGGCGCCGAAAGA
ACTAAAATTCCAGATGGCAAACTCAATGAATGGCAGAAACCCTGGTGGTC
GAGGAGGAAATCCCCGAAAAGGTCGAATTTTGGGTATTATTGATGCTATT
CAGGATGCAGTTGGACCCCCTAAGCAAGCTGCCGCAGATCGCAAAACCTG
GAATCACAGCGAGTTCAAATGTCAATGGAAGGCACAGTAGAGTGGGCTCT
GACCCAGTGCTTATGCGGAAACACAGACGCCATGATTTGCCTTTAGAAGG
AGCTAAGGTCTTTTCCAATGGTCACCTTGGAAGTGAAGAATATGATGTTC
CTCCCCGGCTTTCTCCTCCTCCTCCAGTTACCACCCTCCTCCCTAGCATA
AAGTGTACTGGTCCGTTAGCAAATTCTCTTTCAGAGAAAACAAGAGACCC
AGTAGAGGAAGATGATGATGAATACAAGATTCCTTCATCCCACCCTGTTT
CCCTGAATTCACAACCATCTCATTGTCATAATGTAAAACCTCCTGTTCGG
TCTTGTGATAATGGTCACTGTATGCTGAATGGAACACATGGTCCATCTTC
AGAGAAGAAATCAAACATCCCTGACTTAAGCATATATTTAAAGGGAGATG
TTTTTGATTCAGCCTCTGATCCCGTGCCATTACCACCTGCCAGGCCTCCA
ACTCGGGACAATCCAAAGCATGGTTCTTCACTCAACAGGACGCCCTCTGA
TTATGATCTTCTCATCCCTCCATTAGGTGAAGATGCTTTTGATGCCCTCC
CTCCATCTCTCCCACCTCCCCCACCTCCTGCAAGGCATAGTCTCATTGAA
CATTCAAAACCTCCTGGCTCCAGTAGCCGGCCATCCTCAGGACAGGATCT
TTTTCTTCTTCCTTCAGATCCCTTTGTTGATCTAGCAAGTGGCCAAGTTC
CTTTGCCTCCTGCTAGAAGGTTACCAGGTGAAAATGTCAAAACTAACAGA
ACATCACAGGACTATGATCAGCTTCCTTCATGTTCAGATGGTTCACAGGC
ATCAGCCAGACCCCCTAAACCACGACCGCGCAGGACTGCACCAGAAATTC
ACCACAGAAAACCCCATGGGCCTGAGGCGGCATTGGAAAATGTCGATGCA
AAAATTGCAAAACTCATGGGAGAGGGTTATGCCTTTGAAGAGGTGAAGAG
AGCCTTAGAGATAGCCCAGAATAATGTCGAAGTTGCCCGGAGCATCCTCC
GAGAATTTGCCTTCCCTCCTCCAGTATCCCCACGTCTAAATCTATAGCAG
CCAGAACTGTAGACACCAAAATGGAAAGCAATCGATGTATTCCAAGAGTG
TGGAAATAAAGAGAACTGAGATGGAATTCAAGAGAGAAGTGTCTCCTCCT
CGTGTAGCAGCTTGAGAAGAGGCTTGGGAGTGCAGCTTCTCAAAGGAGAC
CGATGCTTGCTCAGGATGTCGACAGCTGTGGCTTCCTTGTTTTTGCTAGC
CATATTTTTAAATCAGGGTTGAACTGACAAAAATAATTTAAAGACGTTTA
CTTCCCTTGAACTTTGAACCTGTGAAATGCTTTACCTTGTTTACAGTTTG
GCAAAGTTGCAGTTTGTTCTTGTTTTTAGTTTAGTTTTGTTTTGGTGTTT
TGTACCTGTACTGTGTTCTTCACAGACCCTTTGTAGCGTGGTCAGGTCTG
CTGTAACATTTCCCACCAACTCTCTTGCTGTCCACATCAACAGCTAAATC
ATTTATTCATATGGATCTCTACCATCCCCATGCCTTGCCCAGGTCCAGTT
CCATTTCTCTCATTCACAAGATGCTTTGAAGGTTCTGATTTTCAACTGAT
CAAACTAATGCAAAAAAAAAAAAAAAAAAAAAAAAAAG
Human Cbl-b mRNA sequence - var 7

(SEQ ID NO: 43)

CGTNTTTGGNANNCACTACAGGGGATGTTTAATACACACTCACAATGCGC
ATGATGTNTATAACTATCTATTGNATGATG
TAAGATACCCCACTCAAACCCATAAAAAAGAGCATCTTTAATACGACTCA
CTATANGGCGAGCGCACGCCATGGCAGGTA
CCCATACGACGTACCAGATTACGCTCATATGGCCATGGAGGCCAGNGAAT
TCCACCCAAGCNGTGGTATCAACGCANAGT
GGACTCTGACCCANTGCTTATGCGGAAACACAGACGCCATGATTTGCCTT
TAGAAGGAGCTAAGGTCTCTTCCAATGGTC
ACCTTGGAAGTGAAGAATATGATGTTCCTCCCCGGGTTTCTCCTCCTCCT
CCAGTTACCACCCTNCTCCCTAGCATAAAG
TGTACTGGTCCGTTAGCAAATTCTCTTTCAGAGAAAACAAGAGACCCAGT
AGAGGAAGATGATGATGAATACAAGATTCC
TTCATCCCACCCTGTTTCCCTGAATTCACAACCATCTCATTGTCATAATG
TAAAACCTCCTGTTCGGTCTTGTGATAATG
GTCACTGTATGCTGAATGGAACACATGGTCCATCTTCAGAGAAGAAATCA
AACATCCCTGACTTAAGCATATATTTAAAG
GGTGAAGATGCTTTTGATGCCCTCCCTCCATCTCTCCCACCTCCCCCACC
TCCTGCAAGGCATAGTCTCATTGAACAATC
AAAACCTCCTGGCTCCAGTAGCCGGCCATCCTCAGGACAGGATCTTTTTC
TTCTTCCTTCAGATCCCTTTGTTGATCTAG
CAAGTGGCCAAGTTCCTTTGCCTCCCGCTAGAAGGTTACCAGGTGAAAAT
GTCAAAACTAACAGGACATCACAGGACTAT
GATCAGCTTCCTTCATGTTCAGATGGTTCACAGGCACCAGCCAGACCCCC
TAAACCACGACCGCGCAGGACTGCACCAGA
AATTCACCACAGAAAACCCCATGGGCCTGAGGCGGCATTGGAAAATGTCG
ATGCAAAAATTGCAAAACTCATGGGAGAGG
GTTATGCCTTTGAAGAGGTGAAGAGAGCCTTAGAGATAGCCCAGAATAAT
GTCGAAGTTGCCCGGAGCATCCTCCGAGAA
TTTGCCTTCCCTCCTCCAGTATCCCCACGTCTAAATCTATAGCAGCCAGA
ACTGTAGACACCAAAATGGAAAGCAATCGA
TGTATTCCAAGAGTGTGGAAATAAAGAGAACTGAGATGGAATTCAAGAGA
GAAGTGTCTCCTCCTCGTGTAGCAGCTTGA
GAAGAGGCTTGGGAGTGCAGCTTCTCAAAGAAAACCGATGCTTGCTCAGG
ATGTCNACAGCTGNGGNCTNCCTTGTTTTT
GCTAGCCATTTTTTTAAATNAGGGTTGAACTNGANAAAANTATTTAAAAA
CGTTTACCTCCCTTGAACTTTGAACCTGGG
AAAGNC
Human Cbl-b Protein sequence - var 7

(SEQ ID NO: 45)

MRKHRRHDLPLEGAKVSSNGHLGSEEYDVPPRLSPPPPVTTLLPSIKCTG
PLANSLSEKTRDPVEEDDDEYKIPSSHPVS
LNSQPSHCHNVKPPVRSCDNGHCMLNGTHGPSSEKKSNIPDLSIYLKGED
AFDALPPSLPPPPPPARHSLIEHSKPPGSS
SRPSSGQDLFLLPSDPFVDLASGQVPLPPARRLPGENVKTNRTSQDYDQL
PSCSDGSQAPARPPKPRPRRTAPEIHHRKP
HGPEAALENVDAKIAKLMGEGYAFEEVKRALEIAQNNVEVARSILREFAF
PPPVSPRLNL
Human cbl-B clone3Gd114 (partial sequence)

(SEQ ID NO: 44)

ACTCTGACCCAGTGCTTATGCGGAAACACAGACGCCATGATTTGCCTTTA
GAAGGAGCTAAGGTCTCTTCCAATGGTCACCTTGGAAGTGAAGAATATGA
TGTTCCTCCCCGGCTTTCTCCTCCTCCTCCAGTTACCACCCTCCTCCCTA
GCATAAAGTGTACTGGTCCGTTAGCAAATTCTCTTTCAGAGAAAACAAGA
GACCCAGTAGAGGAAGATGATGATGAATACAAGATTCCTTCATCCCACCC
TGTTTCCCTGAATTCACAACCATCTCATTGTCATAATGTAAAACCTCCTG
TTCGGTCTTGTGATAATGGTCACTGTATGCTGAATGGAACACATGGTCCA
TCTTCAGAGAAGAAATCAAACATCCCTGACTTAAGCATATATTTAAAGGG
TGAAGATGCTTTTGATGCCCTCCCTCCATCTCTCCCACCTCCCCCACCTC
CTGCAAGGCATAGTCTCATTGAACATTCAAAACCTCCTGGCTCCAGTAGC
CGGCCATCCTCAGGACAGGATCTTTTTCTTCTTCCTTCAGATCCCTTTGT
TGATCTAGCAAGTGGCCAAGTTCCTTTGCCTCCCGCTAGAAGGTTACCAG
GTGAAAATGTCAAAACTAACAGGACATCACAGGACTATGATCAGCTTCCT
TCATGTTCAGATGGTTCACAGGCACCAGCCAGACCCCCTAAACCACGACC
GCGCAGGACTGCACCAGAAATTCACCACAGAAAACCCCATGGGCCTGAGG
CGGCATTGGAAAATGTCGATGCAAAAATTGCAAAACTCATGGGAGAGGGT
TATGCCTTTGAAGAGGTGAAGAGAGCCTTAGAGATAGCCCAGAATAATGT
CGAAGTTGCCCGGAGCATCCTCCGAGAATTTGCCTTCCCTCCTCCAGTAT
CCCCACGTCTAAATCTATAGCAGCCAGAACTGTAGACACCAAAATGGAAA
GCAATCGATGTATTCCAAGAGTGTGGAAATAAAGAGAACTGAGATGGAAT
TCAAGAGAGAAGTGTCTCCTCCTCGTGTAGCAGCTTGAGAAGAGGCTTGG
GAGTGCAGCTTCTCAAAGAAAACCGATGCTTGCTCAGGATGTCGACAGCT
GTGGCTTCCTTGTTTTTGCTAGCCATTTTTTTAAATCAGGGTTGAACTGG
AAAAAATTATTTAAAAACGTTTACCTCCCTTGAACTTTGAACCTGGGAAA
GGC
Human CblB protein in 3Gd114 Translation of cbl-B
clone3Gd114 starting at base pair 3

(SEQ ID NO: 46)

SDPVLMRKHRRHDLPLEGAKVSSNGHLGSEEYDVPPRLSPPPPVTTLLPS
IKCTGPLANSLSEKTRDPVEEDDDEYKIPSSHPVSLNSQPSHCHNVKPPV
RSCDNGHCMLNGTHGPSSEKKSNIPDLSIYLKGEDAFDALPPSLPPPPPP
ARHSLIEHSKPPGSSSRPSSGQDLFLLPSDPFVDLASGQVPLPPARRLPG
ENVKTNRTSQDYDQLPSCSDGSQAPARPPKPRPRRTAPEIHHRKPHGPFA
Human CBL-B Protein sequence - var1
(public gi: 4757920)

(SEQ ID NO: 47)

MANSMNGRNPGGRGGNPRKGRILGIIDAIQDAVGPPKQAAADRRTVEKTW
KLMDKVVRLCQNPKLQLKNSPPYILDILPDTYQHLRLILSKYDDNQKLAQ
LSENEYFKIYIDSLMKKSKRAIRLFKEGKERMYEEQSQDRRNLTKLSLIF
SHMLAEIKAIFPNGQFQGDNFRITKADAAEFWRKFFGDKTIVPWKVFRQC
LHEVHQISSSLEAMALKSTIDLTCNDYISVFEFDIFTRLFQPWGSILRNW
NFLAVTHPGYMAFLTYDEVKARLQKYSTKPGSYIFRLSCTRLGQWAIGYV
TGDGNILQTIPHNKPLFQALIDGSREGFYLYPDGRSYNPDLTGLCEPTPH
DHIKVTQEQYELYCEMGSTFQLCKICAENDKDVKIEPCGHLMCTSCLTAW
QESDGQGCPFCRCEIKGTEPIIVDPFDPRDEGSRCCSIIDPFGMPMLDLD
DDDDREESLMNNRLANVRKCTDRQNSPVTSPGSSPLAQRRKPQPDPLQIP
HLSLPPVPPRLDLIQKGIVRSPCGSPTGSPKSSPCMVRKQDKPLPAPPPP
LRDPPPPPPERPPPIPPDNRLSRHIHHVESVPSRDPPMPLFAWCPRDVFG
TNQLVGCRLLGEGSPKPGITASSNVNGRHSRVGSDPVLMRKHRRHDLPLE
GAKVFSNGHLGSEEYDVPPRLSPPPPVTTLLPSIKCTGPLANSLSEKTRD
PVEEDDDEYKIPSSHPVSLNSQPSHCHNVKPPVRSCDNGHCNLNGTHGPS
SEKKSNIPDLSIYLKGTYRI
Human CBL-B Protein sequence - var2
(public gi: 23273909)

(SEQ ID NO: 48)

MANSMNGRNPGGRGGNPRKGRILGIIDAIQDAVGPPKQAAADRRTVEKTW
KLMDKVVRLCQNPKLQLKNSPPYILDILPDTYQHLRLILSKYDDNQKLAQ
LSENEYFKIYIDSLMKKSKRAIRLFKEGKERMYEEQSQDRRNLTKLSLIF
SHMLAEIKAIPPNGQPQGDNFRITKADAABFWRKFFGDKTIVPWKVFRQC
LHEVHQISSGLEAMALKSTIDLTCNDYISVFEFDIFTRLFQPWGSILRNW
NFLAVTHPGYMAFLTYDEVKARLQKYSTKPGSYIFRLSCTRLGQWAIGYV
TGDGNILQTIPHNKPLFQALIDGSREGFYLYPDGRSYNPDLTGLCEPTPH
DHIKVTQEQYELYCEMGSTFQLCKICAENDKDVKIEPCGHLMCTSCLTAW
QESDGQGCPFCRCEIKGTEPIIVDPFDPRDEGSRCCSIIDPFGMPMLDLD
DDDDREESLMMNRLANVRKCTDRQNSPVTSPGSSPLAQRRKPQPDPLQIP
HLSLPPVPPRLDLIQKGIVRSPCGSPTGSPKSSPCMVRKQDKPLPAPPPP
LRDPPPPPPERPPPIPPDNRLSRHIHHVESVPSKDPPMPLEAWCPRDVFG
TNQLVGCRLLGEGSPKPGITASSNVNGRHSRVGSDPVLMRKHRRHDLPLE
GAKVFSNGHLGSEEYDVPPRLSPPPPVTTLLPSIKCTGPLANSLSEKTRD
PVEEDDDEYKIPSSHPVSLNSQPSHCHNVKPPVRSCDNGHCMLNGTHGPS
SEKKSNIPDLSIYLKGDVFDSASDPVPLPPARPPTRDNPKHGSSLNRTPS
DYDLLIPPLGEDAFDALPPSLPPPPPPARHSLIEHSKPPGSSSRPSSGQD
LFLLPSDPFVDLASGQVPLPPARRLPGENVKTNRTSQDYDQLPSCSDGSQ
APARPPKPRPRRTAPEIHHRKPHGPEAALENVDAKIAKLMGEGYAFEEVK
RALEIAQNNVEVARSILREFAFPPPVSPRL
Human CBL-B Protein sequence - var3
(public gi: 862407)

(SEQ ID NO: 49)

MANSMNGRNPGGRGGNPRKGRILGIIDAIQDAVGPPKQAAADRRTVEKTW
KLMDKVVRLCQNPKLQLKNSPPYILDILPDTYQHLRLILSKYDDNQKLAQ
LSENEYFKIYIDSLMKKSKRAIRLFKEGKERMYEEQSQDRRNLTKLSLIF
SHMLAEIKAIFPNGQFQGDNFRITKADAAEFWRKFFGDKTIVPWKVFRQC
LHEVHQISSSLEAMALKSTIDLTCNDYISVFEFDIFTRLFQPWGSILRNW
NFLAVTHPGYMAFLTYDEVKARLQKYSTKPGSYIFRLSCTRLGQWAIGYV
TGDGNILQTIPHNKPLFQALIDGSREGFYLYPDGRSYNPDLTGLCEPTPH
DHIKVTQEQYELYCEMGSTFQLCKICAENDKDVKIEPCGHLMCTSCLTAW
QESDGQGCPFCRCEIKGTEPIIVDPFDPRDEGSRCCSIIDPFGMPMLDLD
DDDDREESLMMNRLANVRKCTDRQNSPVTSPGSSPLAQRRKPQPDPLQIP
HLSLPPVPPRLDLIQKGIVRSPCGSPTGSPKSSPCMVRXQDKPLPAPPPP
LRDPPPPPPERPPPIPPDNRLSRHIHHVESVPSRDPPMPLEAWCPRDVFG
TNQLVGCRLLGEGSPKPGITASSNVNGRHSRVGSDPVLMRKHRRHDLPLE
GAKVFSNGHLGSEEYDVPPRLSPPPPVTTLLPSIKCTGPLANSLSEKTRD
PVEEDDDEYKIPSSHPVSLNSQPSHCHNVKPPVRSCDNGHCMLNGTHGPS
SEKKSNIPDLSIYLKGDVFDSASDPVPLPPARPPTRDNPKHGSSLNRTPS
DYDLLIPPLGEDAFDALPPSLPPPPPPARHSLIEHSKPPGSSSRPSSGQD
LFLLPSDPFVDLASGQVPLPPARRLPGENVKTNRTSQDYDQLPSCSDGSQ
APARPPKPRPRRTAPEIHHRKPHGPEAALENVDAKIAKLMGEGYAFEEVK
RALEIAQNNVEVARSILREFAFPPPVSPRLNL
Human CBL-B Protein sequence - var4
(public gi: 862409)

(SEQ ID NO: 50)

MANSMNGRNPGGRGGNPRKGRILGIIDAIQDAVGPPKQAAADRRTVEKTW
KLMDKVVRLCQNPKLQLKNSPPYILDILPDTYQHLRLILSKYDDNQKLAQ
LSENEYFKIYIDSLMKKSKRAIRLFKEGKERMYEEQSQDRRNLTKLSLIF
SHMLAEIKAIFPNGQFQGDNFRITKADAAEFWRKFFGDKTIVPWKVFRQC
LHEVHQISSSLEAMALKSTIDLTCNDYISVFEFDIFTRLFQPWGSILRNW
NFLAVTHPGYMAFLTYDEVKARLQKYSTKPGSYIFRLSCTRLGQWAIGYV
TGDGNILQTIPHNKPLFQALIDGSREGFYLYPDGRSYNPDLTGLCEPTPH
DHIKVTQEQYELYCEMGSTFQLCKICABNDKDVKIEPCGHLMCTSCLTAW
QESDGQGCPFCRCEIKGTEPIIVDPFDPRDEGSRCCSIIDPFGMPMLDLD
DDDDREESLMMNRLANVRKCTDRQNSPVTSPGSSPLAQRRKPQPDPLQIP
HLSLPPVPPRLDLIQKGIVRSPCGSPTGSPKSSPCMVRKQDKPLPAPPPP
LRDPPPPPPERPPPIPPDNRLSRHIHHVESVPSRDPPMPLEAWCPRDVFG
TNQLVGCRLLGEGSPKPGITASSNVNGRHSRVGSDPVLMRKHRPHDLPLE
GAKVFSNGHLGSEEYDVPPRLSPPPPVTTLLPSIKCTGPLANSLSEKTRD
PVEEDDDEYKIPSSHPVSLNSQPSHCHNVKPPVRSCDNGHCMLNGTHGPS
SEKKSNIPDLSIYLKGDVFDSASDPVPLPPARPPTRDNPKHGSSLNRTPS
DYDLLIPPLG
Rat CBL-B mRNA sequence
(public gi: 21886623)

(SEQ ID NO: 51)

CGGGCGGGCGTGGAGCTGTCTGCACGAAAGGACTAAGATTCCAGATGGCA
AATTCTATGAATGGCAGAAATCCTGGTGGTCGAGGAGGAAACCCCCGCAA
AGGTCGAATTTTGGGGATTATTGATGCCATTCAGGATGCAGTTGGACCCC
CAAAGCAAGCTGCAGCTGACCGCAGGACAGTGGAGAAGACTTGGAAACTC
ATGGACAAAGTGGTAAGACTGTGCCAAAATCCGAAACTTCAGTTGAAAAA
CAGCCCACCATATATCCTCGACATTTTACCTGATACGTATCAGCATTTGC
GGCTTATATTGAGTAAGTATGACGACAACCAGAAGCTGGCTCAACTGAGC
GAGAATGAGTACTTTAAAATCTACATCGACAGTCTCATGAAGAAGTCAAA
GCGAGCGATCCGGCTCTTCAAAGAAGGCAAGGAGAGGATGTACGAGGAGC
AGTCGCAGGACAGACGGAATCTCACAAAGCTGTCCCTTATCTTCAGTCAC
ATGCTGGCAGAAATCAAGGCGATCTTTCCCAATGGCCAGTTCCAGGGAGA
TAACTTCCGGATCACCAAAGCAGATGCTGCCGAATTCTGGAGGAAGTTTT
TTGGAGACAAAACTATCGTACCATGGAAAGTCTTCAGACAGTGCCTGCAT
GAGGTCCATCAGATCAGCTCTGGCCTGGAGGCCATGGCTCTGAAGTCAAC
CATTGACTTAACTTGTAATGATTACATCTCCGTGTTTGAATTTGATATTT
TTACCAGGCTATTTCAGCCCTGGGGCTCTATTTTACGGAATTGGAACTTC
TTAGCTGTGACACACCCGGGGTACATGGCATTTCTCACATATGATGAAGT
TAAAGCTCGACTACAGAAATACAGCACCAAGCCTGGAAGCTACATTTTCC
GGTTAAGCTGCACTCGGCTGGGACAATGGGCCATTGGCTATGTGACTGGG
GACGGCAATATCCTACAGACCATACCTCATAACAAGCCCCTGTTCCAAGC
CCTGATTGATGGTAGCAGGGAAGGCTTTTACCTTTATCCAGATGGACGAA
GCTATAACCCTGATTTAACCGGATTATGTGAACCTACACCTCATGATCAT
ATAAAAGTTACACAGGAGCAATATGAACTGTATTGTGAAATGGGCTCCAC
TTTTCAGCTGTGCAAGATCTGTGCAGAGAATGACAAAGATGTCAAGATCG
AGCCTTGTGGGCATCTCATGTGCACTTCGTGCCTTACCGCGTGGCAGGAG
TCTGATGGCCAAGGCTGCCCCTTCTGTCGCTGTGAGATAAAAGGAACCGA
ACCTATCATCGTGGATCCCTTTGACCCCAGAGACGAAGGCTCCAGGTGCT
GCAGCATCATCGACCCTTTCAGCATCCCCATGCTCGACTTGGATGATGAC
GATGATCGAGAGGAGTCTCTGATGATGAACCGGCTGGCGAGTGTTCGCAA
GTGCACAGACAGGCAGAACTCGCCAGTCACATCGCCAGGATCCTCACCCC
TTGCCCAGAGAAGAAAGCCTCAGCCAGACCCTCTCCAGATCCCCCACCTC
AGCCTGCCACCAGTGCCTCCCCGCCTGGACCTCATTCAGAAAGGCATCGT
GCGCTCTCCCTGTGGCAGCCCCACGGGCTCCCCGAAGTCTTCTCCATGCA
TGGTTAGAAAACAAGACAAACCACTCCCAGCACCCCCTCCTCCCTTGCGA
GATCCTCCGCCTCCACCAGAGCGGCCTCCGCCAATCCCGCCTGACAGTAG
ACTGAGCAGACACTTCCACCACGGAGAGAGTGTGCCTTCCAGGGACCAGC
CAATGCCTCTTGAAGCCTGGTGCCCTCGGGATGCCTTCGGGACTAATCAG
GTGATGGGATGTCGCATCCTAGGGGATGGCTCTCCAAAGCCTGGCGTCAC
AGCAAACTCCAACTTAAATGGACGTCACAGTCGAATGGGCTCTGACCAGG
TTCTTATGAGGAAACACAGACGCCACGATTTGCCTTCAGAAGGCGCCAAG
GTCTTTTCCAATGGACACCTTGCCCCTGAAGAATACGACGTTCCTCCTCG
GCTTTCCCCTCCTCCTCCAGTCACTGCCCTTCTCCCTAGCATAAAGTGTA
CTGGTCCAATAGCAAATTGTCTCTCCGAGAAAACAAGAGACACAGTAGAA
GAAGATGATGATGAATACAAGATTCCTTCATCCCATCCTGTTTCCCTGAA
TTCACAACCATCTCATTGTCATAATGTCAAACCTCCTGTTCGGTCTTGTG
ATAATGGTCACTGTATACTGAATGGAACTCATGGTACGCCTTCAGAGATG
AAGAAATCAAACATCCCAGATTTAGGCATCTATTTGAAGGGTGAAGATGC
TTTTGATGCCCTCCCCCCATCCCTTCCTCCTCCCCCACCTCCTGCAAGAC
ATAGTCTCATCGAGCATTCAAAACCTCCAGGCTCCAGTAGCCGGCCTTCC
TCAGGACAGGACCTTTTCCTTCTTCCTTCAGATCCCTTTTTTGACCCAGC
AAGTGGCCAAGTTCCATTGCCTCCGGCCAGGAGAGCACCAGGAGATGGTG
TCAAATCCAACAGAGCCTCCCAGGACTATGACCAGCTCCCTTCATCTTCC
GATGGTTCGCAAGCACCAGCTAGACCCCCCAAACCACGACCCCGAAGGAC
TGCACCAGAAATTCATCACAGAAAGCCCCATGGGCCCGAGGCGGCACTGG
AAAATGTGGATGCGAAAATTGCAAAACTCATGGGAGAGGGGTATGCCTTT
GAAGAGGTGAAGAGAGCCTTAGAGATCGCCCAGAATAACCTGGAAGTGGC
CAGGAGCATACTTCGAGAATTCGCCTTCCCTCCTCCCGTCTCGCCACGTC
TCAATCTATAGCAGCCCAGACTGCAAACACCAAAGGGTAAAACAGTTAAC
AAATATTCCAGGAGTATGGGACAGAAGGACTGAGAGGGAATGCAGGAGCC
ATGGTGTCTTTTCATGTGGCGTCTCCAGAAGGCAGCCTTGAGTCCAGCTT
CTCTGGTACCACAGCTCCCTGAGGATGCCCACGCTGCAGCTTCTGTGTTT
GTGCTAGCCATACTTTTAAATCAGGGTTGAACTGAGAAAATAATTTAAAG
ACGTTTACTCCCCCTTGAACTTTGAATCTGTGAAATGCTTTCCTTGTTTA
CACGTTGGCAGAATTGCAGTTTGTCTCTGTTTTTGATTCCTGTACTGTGT
TCCTGACAGGCCCTTGGCAGAGTTGGTCAGGTCTGCTGTAAGTTTGTCCA
TGCCCACCCTGCTGCCCACATTGGCAGCTAAAGCATCTCTTCGTGTTGCT
GTCTATCCGGGCCCCACCTCATGTGTCCACGTCCAGTTCATTTCTCTCAT
TCACACAGCATGCTAGTCTGAGG
Rat CBL-B Protein sequence
(public gi: 21886624)

(SEQ ID NO: 55)

MANSMNGRNPGGRGGNPRKGRILGIIDAIQDAVGPPKQAAADRRTVEKTW
KLMDKVVRLCQNPKLQLKNSPPYILDILPDTYQHLRLILSKYDDNQKLAQ
LSENEYFKIYIDSLMKKSKRAIRLFKEGKERMYEEQSQDRRNLTKLSLIF
SHMLAEIKAIFPNGQFQGDNFRITKADAAEFWRKFFGDKTIVPWKVPRQC
LHEVHQISSGLEAMALKSTIDLTCNDYISVFEFDIFTRLFQPWGSILRNW
NFLAVTHPGYMAFLTYDEVKARLQKYSTKPGSYIFRLSCTRLGQWAIGYV
TGDGNILQTIPHNKPLFQMALDGSREGFYLYPDGRSYNPDLTGLCEPTPR
DHIKVTQEQYELYCEMGSTFQLCKICAENDKDVKIEPCGHLMCTSCLTAW
QESDGQGCPFCRCEIKGTEPIIVDPFDPRDEGSRCCSIIDPFSIPMLDLD
DDDDREESLMHNRLASVRKCTDRQNSPVTSPGSSPLAQRRKPQPDPLQIP
HLSLPPVPPRLDLIQKGIVRSPCGSPTGSPKSSPCMVRKQDKPLPAPPPP
LRDPPPPPERPPPIPPDSRLSRHFHHGESVPSRDQPMPLEAWCPRDAFGT
NQVMGCRILGDGSPKPGVTANSNLNGRHSRMGSDQVLMRKHRRHDLPSEG
AKVFSNGHLAPEEYDVPPRLSPPPPVTALLPSIKCTGPIANCLSEKTRDT
VEEDDDEYKIPSSHPVSLNSQPSHCHNVKPPVRSCDNGHCIIMGTHGTPS
EMKKSNIPDLGIYLKGEDAFDALPPSLPPPPPPARHSLIEHSKPPGSSSR
PSSGQDLFLLPSDPFFDPASGQVPLPPARRAPGDGVKSNRASQDYDQLPS
SSDGSQAPARPPKPRPRRTAPEIRHRKPHGPEAALENVDAKIAKLMGEGY
AFEEVKRALEIAQNNLEVARSILREFAFPPPVSPRLNL
Mousc CBL-B mRNA sequence
(public gi: 26324665)

(SEQ ID NO: 52)

GACTCCCTGGGCTGCGAGCGCCGGCGGTGGTTGCCGGAGAGGCCCCTCCT
TCTCGCCCGGCTCCATTCCCTCGCTCGCGGCCGAGCGGGCTCCCGACCCT
CCGCTGGCCATGGCCGGCAACGTGAAGAAGAGCTCGGGCGCCGGCGGCGG
CGGCTCTGGGGGCTCGGGAGCGGGCGGCCTGATCGGGCTCATGAAGGACG
CCTTCCAGCCGCACCACCACCACCACCACCTCAGCCCGCACCCTCCCTGC
ACGGTGGACAAGAAGATGGTGGAGAAGTGCTGGAAGCTCATGGACAAGGT
GGTGCGGTTGTGTCAAAACCCAAAGCTGGCGCTCAAGAACAGCCCGCCTT
ATATCTTAGACCTGCTGCCTGACACCTACCAGCACCTCCGCACTGTCTTG
TCAAGATATGAGGGGAAGATGGAGACGCTTGGAGAAAATGAGTATTTCAG
GGTGTTCATGGAAAATTTGATGAAGAAAACTAAGCAGACTATCAGCCTCT
TCAAGGAGGGAAAAGAAAGGATGTATGAGGAGAATTCCCAGCCTAGGCGA
AACCTGACCAAATTATCCCTGATCTTCAGCCACATGCTGGCAGAACTGAA
AGGCATCTTTCCGAGCGGACTCTTCCAAGGAGACACTTTCCGGATTACTA
AAGCTGATGCTGCCGAATTTTGGAGAAAAGCTTTTGGTGAAAAGACGATA
GTCCCGTGGAAGAGCTTTCGACAGGCCCTGCATGAAGTGCATCCCATCAG
TTCTGGGCTGGACGCCATGGCTCTGAAGTCCACTATTGATCTGACCTGCA
ATGATTATATTTCTGTCTTTGAATTTGATATTTTTACACGGCTGTTTCAG
CCCTGGTCCTCTTTGCTCAGAAATTGGAACAGCCTTGCTGTAACTCACCC
TGGTTACATGGCTTTCCTGACATACGATGAAGTGAAAGCGCGCCTGCAGA
AGTTCATCCACAAACCTGGCAGTTACATCTTTCGGCTGAGCTGTACTCGT
TTGGGTCAGTGGGCTATTGGGTATGTTACTGCCGATGGGAACATTCTGCA
GACAATCCCACACAATAAACCGCTCTTCCAAGCACTGATTGATGGCTTCA
GGGAAGGCTTCTATTTGTTTCCTGATGGACGAAATCAAAATCCTGACCTG
ACAGGTTTATGTGAACCAACTCCTCAAGATCATATCAAAGTAACCCAGGA
ACAATATGAATTATACTGTGAAATGGGCTCCACATTTCAACTGTGTAAGA
TATGTGCTGAGAATGATAAGGATGTGAAGATTGAGCCCTGTGGACACCTC
ATGTGCACATCCTGCCTCACGTCGTGGCAGGAATCAGAAGGTCAGGGCTG
TCCTTTTTGCCGATGTGAAATCAAAGGTACTGAGCCCATCGTGGTGGATC
CGTTTGACCCCAGAGGCAGTGGCAGCCTATTAAGGCAAGGAGCAGAAGGT
GCTCCTTCCCCAAATTACGACGATGATGATGATGAACGAGCTGATGATTC
TCTCTTCATGATGAAGGAGTTGGCAGGTGCCAAGGTGGAAAGGCCTTCCT
CTCCATTCTCCATGGCCCCACAAGCTTCCCTTCCTCCAGTGCCACCAAGA
CTTGACCTTCTACAGCAGCGAGCACCTGTTCCTGCCAGCACTTCAGTTCT
GGGGACTGCTTCCAAGGCTGCTTCTGGCTCCCTTCATAAAGACAAACCAT
TGCCAATACCTCCCACACTTCGAGATCTTCCACCACCACCCCCTCCAGAC
CGGCCTTACTCTGTTGGAGCAGAAACAAGGCCTCAGAGACGCCCTCTGCC
TTGTACACCAGGCGATTGTCCATCTAGAGACAAACTGCCCCCTGTCCCTT
CTAGCCGCCCAGGGGACTCGTGGTTGTCTCGGCCAATCCCTAAAGTACCA
GTAGCTACTCCAAACCCTGGTGATCCTTGGAATGGGAGAGAATTGACCAA
TCGGCACTCGCTTCCATTCTCATTGCCCTCACAAATGGAACCCAGAGCAG
ATGTCCCTAGGCTTGGAAGCACATTTAGTCTGGATACCTCTATGACTATG
AATAGCAGCCCAGTAGCAGGTCCAGAGAGTGAGCACCCAAAGATCAAGCC
TTCCTCGTCTGCCAACGCCATTTACTCTCTGGCTGCCAGGCCTCTTCCTA
TGCCAAAACTGCCACCTGGGGAGCAAGGGGAAAGTGAAGAGGACACAGAA
TATATGACTCCCACATCTAGGCCTGTAGGGGTTCAGAAGCCAGAGCCCAA
ACGGCCGTTAGAGGCAACCCAGAGTTCACGAGCATGTGACTGTGACCAGC
AGATCGACAGCTGTACCTATGAAGCGATGTATAACATCCAGTCCCAAGCA
CTGTCTGTAGCAGAAAACAGCGCCTCTGGGGAAGGGAATCTGGCCACAGC
TCACACGAGTACTGGCCCTGAGGAATCCGAAAACGAGGATGATGGCTATG
ATGTGCCTAAGCCACCCGTGCCAGCTGTACTGGCCCGCCGGACCCTGTCT
GACATCTCCAATGCCAGCTCCTCCTTTGGCTGGTTGTCTTTGGATGGTGA
CCCTACAAACTTCAATGAGGGTTCCCAAGTTCCTGAGCGGCCCCCCAAAC
CATTCCCTCGGAGAATCAACTCAGAACGAAAAGCCAGTAGCTATCAACAA
GGCGGAGGTGCCACTGCTAACCCTGTGGCCACAGCACCCTCACCGCAGCT
CTCAAGTGAGATTGAACGCCTCATGAGTCAGGGCTATTCCTACCAGGACA
TTCAGAAAGCTTTGGTCATTGCCCACAACAACATTGAGATGGCTAAAAAC
ATCCTCCGGGAATTTGTTTCTATTTCTTCTCCTGCTCACGTAGCCACCTA
GCACATCTCTCCCTGCCACGGCTTCAGAGGACCCATGAGCCAGGCTCTTA
CTCAAGGACCACCTAGGAAAGCAGTGGCTTCTTTTGGGACGTCACAGTAA
GGTCCTGCCTTTCCTGTGGGGATCGACACATATGGTTCCAAGATTTCAAA
GCAGTGGAATGAAAATGGAGCAGCTGATGTGTTTCATTGTTGTATTGGTC
TTAAGAGTGTTTTTGTAGTCCTGCAGTCTCCAGTAGGAGAGAGTGGGTTT
TTATTAAATGGTAACCTACCCCAGAAACAGC
Mouse CBL-B Protein sequence
(public gi: 26324666)

(SEQ ID NO: 56)

MAGNVKKSSGAGGGGSGGSGAGGLIGLMKDAFQPHHHHHHLSPHPPCTVD
KKMVEKCWKLMDKVVRLCQNPKLALKNSPPYILDLLPDTYQHLRTVLSRY
EGKMETLGENEYFRVFMENLMKKTKQTISLFKEGKERMYEENSQPRRNLT
KLSLIFSHMLAELKGIFPSGLFQGDTFRITKADAAEFWRKAFGEKTIVPW
KSFRQALHEVHPISSGLDAMALKSTIDLTCNDYISVFEFDIFTRLFQPWS
SLLRNWNSLAVTHPGYMAFLTYDEVKARLQKFIHKPGSYIFRLSCTRLGQ
WAIGYVTADGNILQTIPHNKPLFQALIDGFREGFYLFPDGRNQNPDLTGL
CEPTPQDHIKVTQEQYELYCEMGSTFQLCKICAENDKDVKIEPCGHLMCT
SCLTSWQESEGQGCPFCRCEIKGTEPIVVDPFDPRGSGSLLRQGAEGAPS
PNYDDDDDERADDSLFMMKELAGAKVERPSSPFSMAPQASLPPVPPRLDL
LQQRAPVPASTSVLGTASKAASGSLHKDKPLPIPPTLRDLPPPPPPDRPY
SVGAETRPQRRPLPCTPGDCPSRDKLPPVPSSRPGDSWLSRPIPKVPVAT
PNPGDPWNGRELTNRHSLPFSLPSQMEPRADVPRLGSTFSLDTSMTMNSS
PVAGPESEHPKIKPSSSANAIYSLAARPLPMPKLPPGEQGESEEDTEYMT
PTSRPVGVQKPEPKRPLEATQSSRACDCDQQIDSCTYEAMYNIQSQALSV
AENSASGEGNLATAHTSTGPEESENEDDGYDVPKPPVPAVLARRTLSDIS
NASSSFGWLSLDGDPTNFNEGSQVPERPPKPFPRRINSERKASSYQQGGG
ATANPVATAPSPQLSSEIERLMSQGYSYQDIQKALVIAHNNIEMAKNILR
EFVSISSPAHVAT
Drosophila CBL-B mRNA sequence
(public gi: 1842452)

(SEQ ID NO: 53)

CATCTCGAAAATATTGTGTGGGTTTAAAAAACGTTAACGTCGCCGAAACG
CGTAGCCCCAAATGCACACGCCAGGTGCAAGGATAAAGCCGTGAGGATCG
GGCACCCAATCGGATAGATCGCGTTTGGTTAGCTTGTGGGGGAAAATCGT
ACTTAAGTCACCACTACTACTACACACGGGCACCACCAGCAACACCAACA
ACAACAACAACGAGAACAGCACCAGCAACAACAACAACAGCAGCAAGAAG
GAGAAGAGCTGAGAAGAGGAAGCAGAGGCAGCGCAGTCGGCAGCGCAGCG
GCAGAGAGAAAAGATGGCGACGAGAGGCAGTGGAACCCGTGTGCAATCGC
AGCCAAAGATTTTCCCATCGCTGCTTTCCAAGCTGCACGGCGCTATCTCG
GAAGCCTGCGTCTCGCAGCGTCTGTCCACCGACAAGAAGACGCTGGAGAA
GACCTGGAAGTTGATGGACAAGGTGGTCAAACTGTGCCAGCAGCCGAAGA
TGAATCTTAAGAATAGTCCACCGTTTATTTTGGACATCCTGCCGGATACG
TACCAGCGCCTGAGATTGATCTACTCAAAGAAGGAGGACCAGATGCACCT
GCTCCATGCCAACGAGCACTTCAACGTGTTCATCAACAACCTGATGCGAA
AGTGCAAGCGGGCCATCAAGTTGTTCAAGGAGGGCAAGGAGAAGATGTTC
GACGAGAACTCCCACTACCGCCGCAATCTCACCAAGCTCAGCCTGGTCTT
CTCCCACATGCTCAGCGAACTGAAGGCCATATTCCCCAACGGTGTCTTTG
CCGGGGATCAATTTCGGATCACCAAAGCGGATGCGGCTGACTTTTGGAAG
AGCAACTTCGGTAACAGCACATTGGTTCCCTGGAAAATCTTCCGGCAGGA
GCTTAGCAATGTACATCCCATAATCTCCGGCCTGGAGGCCATGGCCCTAA
AGACCACTATCGATCTTACCTGCAACGACTTCATTTCCAACTTCGAGTTC
GACGTCTTCACACGCCTCTTCCAGCCTTGGGTGACACTGCTACGCAACTG
GCAGATTCTGGCCGTCACACATCCGGGCTACGTGGCGTTTCTCACATACG
ACGAGGTGAAGGCTCGCCTACAGCGCTACATCCTCAAGGCGGGCAGCTAC
GTTTTCCGGCTCTCCTGCACGCGATTGGGCCAATGGGCCATCGGCTACGT
AACTGCCGAGGGAGAGATTCTGCAGACAATCCCTCAGAACAAGTCGCTGT
GCCAGGCGCTGCTCGATGGCCATCGAGAGGGCTTCTACTTGTACCCAGAT
GGCCAAGCGTACAATCCGGATCTGTCGTCTGCCGTTCAAAGTCCCACAGA
GGACCACATAACCGTTACCCAAGAGCAATACGAACTATACTGTGAAATGG
GCAGCACCTTTCAGCTGTGCAAAATTTGTGCGGAGAACGACAAAGATATC
CGCATCGAGCCCTGTGGCCACTTGTTGTGCACTCCCTGCCTTACCTCCTG
GCAAGTGGATTCCGAGGGACAGGGCTGCCCCTTCTGTCGGGCCGAAATCA
AGGGCACCGAACAAATCGTTGTGGACGCTTTCGATCCGCGCAAGCAACAC
AACCGGAACGTCACCAATGGGCGACAGCAGCAGCAGGAAGAAGACGACAC
TGAGGTATAGTTTTGTTCACAGCCTGATCAGCCTGATCCGCCTGCTCCGC
TGCCGCGTGTGCTGCTATTTATATACATATTACTCTTATGATTACCTTTG
GTTCGTTTATACAGTTATATATGCCTATATATACATTATATATTTTAGAT
TTTACAACTGCTATTGTTTATATAAGTTTAATGTTTAGCCTGCAGTTCGC
AGTGGCAGTTTCGAGTTTAATTTTGTTTGTTTAGCTGTAACATATTTAAA
TTATTAGCCAAACTCATGCAACTAACATCCACAGACCCACGCACACACGC
CCAATCACAAGCACAAGTACAACCATAACCATTGTCCATCCATCGAGCAC
ATGCATAACGTAGTTAAAGTTCTTTGACCGGAAGTCGCTCATCAACCATC
GTTTGCTATCGCTTCCTCTGTTTTCTCTCCGCCGGTTTGGTTTGGTTTGG
TTTGTGTGCGTTCGTTTAGTTGTTCGTTCTTCCACTCTCACGCTCTCTCT
ATCTATTGATCACGTTCGCCTCTGTTTATGAATCATATTTTAATCGATTC
GATTCGCCCTCGATTGCACTTTTGTACATAGGCACTATGGAATTTATAAT
TGGTAACCTTGTTCTTGTATTATTCGGGTGAATTTTCTCCTTTCACATCC
AGCTTGATTATCCCCTTGATTATGTATGCCCGCCAGTAATTTTTGTATCT
ATCCCCTACTCTAGAATCATTCTCTTAATCATTGTACTCCGTTATGTGTT
TATTTCATTTTAGTTTATTGTTTAATACTTCCAAAGATACATTTAGTTTG
TAGTAGCGTGCGTTTACTTCCCCCCCCATATCAATTCAATTTTATTTGTA
AGCAGCCAAYGCGTGCCCTAAGACTGTAATTTATTATTAACAMAAAAAAR
AAAATCGAAAAAGTTTAAGAAATCAGGCTAAACATAGGAGGCCTCGAATC
GATCGATAATTTAGTTAGATTGYATGTAAATTAATTATTGATTTCCTGTG
TCACAAGGCCA
Drosophila CBL-B Protein sequence
(public gi: 1842453)

(SEQ ID NO: 57)

MATRGSGTRVQSQPKIFPSLLSKLHGAISEACVSQRLSTDKKTLEKTWKL
MDKVVKLCQQPKMNLKNSPPPILDILPDTYQRLRLIYSKKEDQMHLLHAN
EHFNVFINNLMRXCKRAIKLFKEGKEKMFDENSHYRRNLTKLSLVFSHML
SELKAIFPNGVFAGDQFRITKADAADFWKSNFGNSTLVPWKIFRQELSKV
HPIISGLEAMALKTTIDLTCNDFISNFEFDVFTRLFQPWVTLLRNWQILA
VTHPGYVAFLTYDEVKARLQRYILKAGSYVFRLSCTRLGQWAIGYVTAEG
EILQTIPQNKSLCQALLDGHREGFYLYPDGQAYNPDLSSAVQSPTEDHIT
VTQEQYELYCEMGSTFQLCKICAENDKDIRIEPCGHLLCTPCLTSWQVDS
EGQGCPFCRAEIKGTEQIVVDAFDPRKQHNRNVTNGRQQQQEEDDTEV
C. elegans CBL-B mRNA sequence
(public gi: 25150544)

(SEQ ID NO: 54)

CTATGATCATTACATCCTAATTAATTGCCACTGGACTTCACATCATATCA
CCGTTTCACCGGGAATGGGTTCAATAAACACAATTTTTCACCGGATACAT
CGGTTTGTCAATGGCACAGGCAATAATGCGCGATTTGTTCCCAGCACAAA
CAACTCGACGGAAGCGTTGACACTCAGTCCGAGAGCTGTTCCCAGCACAG
TTTCACTATTCGAAATCCCATCAGCTTCGGAGATGCCCGGTTTCTGCAGT
GAAGAGGATCGTCGATTTTTGCTCAAAGCATGCAAGTTTATGGATCAAGT
AGTGAAGAGTTGTCATAGCCCAAGACTGAATTTGAAAAATTCGCCGCCTT
TCATTTTGGACATTCTACCTGATACTTATACGCATTTAATGCTGATATTC
ACACAAAACAATGACATACTCCAAGACAACGACTACTTGAAAATCTTTCT
GGAGAGTATGATCAACAAGTGCAAAGAGATCATCAAACTGTTCAAGACGT
CAGCTATCTACAATGACCAGTCTGAAGAACGACGGAAGCTTACGAAAATG
TCACTAACATTTTCACATATGCTTTTCGAGATTAAAGCATTATTTCCGGA
AGGTATCTATATTGAAGACCGGTTTCGGATGACAAAGAAGGAAGCCGAAA
GCTTTTGGAGTCATCATTTTACAAAAAAAAACATTGTACCCTGGTCAACA
TTTTTTACTGCATTAGAAAAGCACCATGGATCAACGATAGGAAAAATGGA
AGCAGCCGAATTAAAAGCTACGATAGACTTGAGCGGAGATGATTTTATTT
CGAATTTTGAGTTTGATGTGTTTACAAGGTTATTCTACCCTTTCAAAACA
CTGATCAAAAATTGGCAAACACTCACCACCGCCCATCCCGGATACTGTGC
ATTTCTCACATACGATGAGGTCAAAAAACGGTTAGAAAAATTAACGAAAA
AACCTGGAAGCTACATCTTCCGGTTATCATGCACACGTCCTGGACAATGG
GCAATAGGATACGTAGCTCCGGATGGAAAGATTTATCAGACAATACCACA
GAATAAAAGTTTGATTCAAGCACTACATGAAGGCCATAAAGAAGGATTTT
ATATTTACCCGAACGGTAGAGATCAAGATATTAACTTATCCAAATTGATG
GATGTGCCACAAGCGGACAGAGTGCAAGTGACCAGTGAACAATACGAGTT
GTATTGTGAGATGGGCACAACATTCGAGTTGTGCAAAATTTGTGACGATA
ACGAGAAGAACATCAAAATTGAGCCATGTGGACATTTGCTCTGCGCAAAA
TGTTTGGCTAACTGGCAGGATTCGGATGGTGGTGGCAACACATGTCCATT
CTGCCGCTACGAAATCAAAGGAACAAATCGTGTGATTATTGACAGGTTCA
AGCCCACTCCGGTAGAAATTGAAAAAGCGAAAAATGTAGCTGCTGCGGAG
AAGAAGCTGATCTCATTAGTTCCCGACGTGCCTCCCAGAACGTATGTGTC
CCAATGTTCTCAAAGTTTGCTGCATGACGCGTCAAACTCAATTCCGTCGG
TCGACGAGTTGCCGTTGGTGCCGCCACCGTTGCCACCGAAAGCATTGGGT
ACCCTGGACACTTTGAATTCGTCACAAACATCCTCTTCATACGTGAACAT
CAAAGAGCTGGAAAATGTTGAAACAAGCGGAGAAGCATTGGCACAAGTGG
TAAACCGGCAACGGGCGCCTTCAATCCAAGCTCCACCACTACCGCCAAGG
TTATCAGCGAGCGAGCACCAACCACACCACCCATACACAAATACGAACAG
TGAGCGGGAGTAGACTTGTGTAAATGTTCATCTTACCGCTTTATACTGCA
ATTTTCATTCCCCCACTTATCATAGAACTATTCTTCCACAACAACATATT
GCCGTGACTAGAACTGGTAACACTACATCATTCTTTGTTAAAACGTTATT
ATATCTCTATTTCTTTTTCGCCTACTCCTTTCCGTTTTTTTTTCAAATTT
TGTCAATTTTCCTACAGCGTTCTGACTCCTATTGGTAAGCAATCATGTCA
TATCTTGTTAAATTTTCATGTTAATTTCTTACTCTCGCTGTCCCAGATTT
TACGGAGTTTTCAGGAAACGTTTGATTTTGTTCTATTCTACAATTTCCAT
CGCCCCCAACCTGTCGTGTATTTTCTATGTGTCACTCTGAAGAAAACAAG
TTTAGACTTTTTAAAAATCGTTTTATTACTCTAAAACTTAAAAGCTGAAA
TGTCAGCTATAGTAAAAATACATA
C. elegans CBL-B Protein sequence
(public gi: 25150545)

(SEQ ID NO: 58)

MGSINTIFHRIHRFVNGTGNNARFVPSTNNSTEALTLSPRAVPSTVSLFE
IPSASEMPGFCSEEDRRFLLKACKFMDQVVKSCHSPRLNLKNSPPFILDI
LPDTYTHLMLIFTQNNDILQDNDYLKIFLESMINKCKEIIKLFKTSAIYN
DQSEERRKLTKMSLTFSHMLFEIKALFPEGIYIEDRFRMTKKEAESFWSH
HFTKKNIVPWSTFFTALEKHMGSTIGKMEAAELKATIDLSGDDFISNFEF
DVFTRLFYPFKTLIKNWQTLTTAHPGYCAFLTYDEVKKRLEKLTKKPGSY
IFRLSCTRPGQWAIGYVAPDGKIYQTIPQNKSLIQALHEGHKEGFYIYPN
GRDQDINLSKLMDVPQADRVQVTSEQYELYCEMGTTFELCKICDDNEKNI
KIEPCGHLLCAKCLANWQDSDGGGNTCPPCRYEIKGTNRVIIDRPKPTPV
EIEKAKNVAAAEKKLISLVPDVPPRTYVSQCSQSLLHDASNSIPSVDELP
LVPPPLPPKALGTLDTLNSSQTSSSYVNIKELENVETSGEALAQVVNRQR
APSIQAPPLPPRLSASEHQPHHPYTNTNSERE

### Example 11

### Cbl-b Affects VLP Production

Pulse-Chase Kinetics

A. Transfections
1. One day before transfection plate cells at a concentration of 5*10⁶ cell/plate in four 15 cm plates.
2. Two hours before transfection, replace cell media to 16 ml complete DMEM without antibiotics.
3. siRNA dilution: for each transfection dilute 100 μl siRNA in 2 ml OptiMEM (2 plates with scrambled siRNA (187) and 2 plates with Cbl-b siRNA (275).
4. LF 2000 dilution: for each transfection dilute 50 μl lipofectamine reagent in 2 ml OptiMEM.
5. Incubate diluted siRNA and LF 2000 for 5 minutes at RT.
6. Mix the diluted siRNA with diluted LF2000 and incubated for 25 minutes at RT.
7. Add the mixture to the cells (drop wise) and incubate for 24 hours at 37° C. in CO₂ incubator.

8. Next day, perform HIV trasfection (pNLenv-1 # 111), 11 μg/plate with the appropriate siRNA at a concentration of 100 nM.

| | Day 2 | Day 3 | Day 4 |
|---|---|---|---|
| | SiRNA | Exchange | SiRNA as in day 2 + |
| Plate | 100 μl/plate | medium | 11 μg #111/plate |
| 1 | 187 | | 187 + 111 |
| 2 | 187 | | 187 + 111 |
| 3 | 275 | | 275 + 111 |
| 4 | 275 | | 275 + 111 |

B. Pulse-Chase
1. Discard medium and wash with PBS. Scrape cells in 12 ml PBS. Wash plate again with 10 ml PBS. Tansfer gently cells into 50 ml conical tube. Centrifuge to pellet cells at 1800 rpm for 5-10 minutes at RT.
2. Remove supernatant and resuspend cells in 20 ml of starvation medium. Incubate in the incubator for 1 hour. Invert the tube every 15 minutes. Take 1 plate for checking Cbl-b expression by IP/IB, (30% and 70% respectively) pellet cells and freeze (protocol at section D). Count cells during incubation!
   Starvation medium
   RPMI without methionine and no FCS.
   5 mM HEPES pH 7.5
   Glutamine (1:100)
   Pen/Strep (1:100)
3. At the end of incubation pellet cells at 1800 rpm for 5-10 minutes at RT (as in step 1), remove supernatant and resuspend cells gently in 120 μl starvation medium (˜1.5 10⁷ cells in 150 μl RPIM without Met). Transfer cells to an eppendorf tube with an O-ring caps and place in the thermo mixer. If necessary add another 50 μl to splash the rest of the cells out (all specimens should have the same volume of labeling reaction!). First break cell pellet by gentle tapping and vortex and then use cut tips!
4. Pulse: Add 50 μl of ³⁵S-methionine (specific activity 14.2 μCi/μl), tightly cap tubes and place in thermomixer. Set the mixing speed to the lowest possible (700-750 rpm), 37° C. and incubate for 25 minutes.
5. Stop the pulse by adding 1 ml ice-cold chase/stop medium. Shake tube very gently three times and pellet cells at 14,000 rpm for 6 sec. Remove supernatant by tip to a 50 ml tube (high radioactivity). Add gently 0.9 ml ice-cold chase/stop medium to the pelleted cells and invert gently. Transfer 200 μl sample (time 0) to a tube containing 1 ml ice-cold stop/chase medium (marked as cell). Place the rest of the samples in the thermomixer and start chase incubation. Pellet the cells immediately (14,000 rpm, 1 min) and transfer sup to a fresh tube (marked as VLP) and freeze the cell pellet at 80° C. Spin the sup (VLPs) for 2 hours, 14,000 at 4° C. and in the end remove the sup carefully by vacuum (leave ˜20 μl).
6. Chase: the chase is done at 0, 1, 3 and 6 hours as described in step 5 for the first chase time (time 0).

Chase/Stop Medium
Complete RPMI
10% FCS
10 mM cold methionine
5 mM HEPES pH7.5
Glutamine (1:100)
Pen/Strep (1:100)
Prepare 50 ml aliquots and freeze at −20° C.
Prior to use, thaw, shake intensively and place on ice.

C. IP with anti-p24
1. Wash protein G beads (calculated below—for preclearing and conjugation of Ab) 3 times with lysis buffer (1 ml). Put the beads for preclearing at 4° C. Centrifuge at 8000 rpm, 1 minute.
2. Conjugate anti-p24 rabbit antibody with protein G beads. Anti-p24 protein G beads conjugation (for 20 samples): Use 40 μl ProG beads (Sigma) and 6 μl anti-p24r (Seramon) per sample.
   a. Add to an ependorff tube: prewashed ProG beads, p24-rabbit antibody and lysis buffer.
   b. Incubate in thermomixer at 25° C. for 2 hours, 1400 rpm.
   c. Wash three times with lysis buffer and resuspend to initial volume of lysis buffer (conjugated beads can be kept up to a week at 4° C.). Centrifuge at 8000 rpm, 1 minute.
3. Lyse cell/VLP pellet by adding 500 μl of lysis buffer (listed below), resuspend well (cells by pipettation and VLP by 10 sec vortex) and incubate on ice for 20 minutes. Spin at 14,000rpm, at 4° C. for 15 minutes. Remove supernatant to a fresh tube (already contains protein G beads as described in the next step).

Lysis Buffer
5 mM Tris-HCl pH 7.6
1.5 mM MgCl₂
150 mM NaCl
10% Glycerol
0.5% NP40
0.5% DOC
1 mM EDTA
1 mM EGTA
Prior to use add 1:200 PI₃C.
4. Pre-clear by addition of 10 μl protein G beads (re-washed three times with lysis buffer). Incubate at 4° C. for 1 hour at the orbital shaker. It's possible to freeze the samples after preclearing.
5. Spin samples 1 min at 14000 rpm and transfer supernatant to a fresh tube.
6. Add to all samples 40 μl of anti-p24-protein G conjugated beads and incubate in the orbital shaker for 4 hours at 4° C.

7. At the end of incubation, transfer sup+beads to fresh tubes, spin down beads and wash twice with 1 ml high salt buffer, once with medium salt buffer and twice with low salt buffer (listed below).

| High salt buffer | Medium salt buffer | Low salt buffer |
|---|---|---|
| 50 mM Tris-HCl, pH | 50 mM Tris-HCl, pH | 50 mM Tris-HCl, pH |
| 8.0 | 8.0 | 8.0 |
| 500 mM NaCl | 150 mM NaCl | — |
| 0.1% SDS | 0.1% SDS | — |
| 0.1% Triton X-100 | 0.1% Triton X-100 | 0.1% Triton X-100 |
| 5 mM EGTA | — | — |
| 5 mM EDTA | 5 mM EDTA | 5 mM EDTA |

12. Add to each tube 30 μl 2× SDS sample buffer. Heat to 70° C. for 10 minutes.
13. Separate all samples on 1 mm, 12.5% SDS-PAGE. 40 mA/gel
14. Fix gel in 25% ethanol and 10% acetic acid for 15 minutes (minimum).
15. Pour off the fixation solution and soak gels in water until they reach their original size (˜20 min).
16. Dry gels on warm plate (80° C.) under vacuum for 2-4 hours.
17. Expose gels to screen for at least 4 hours and scan by typhoon.

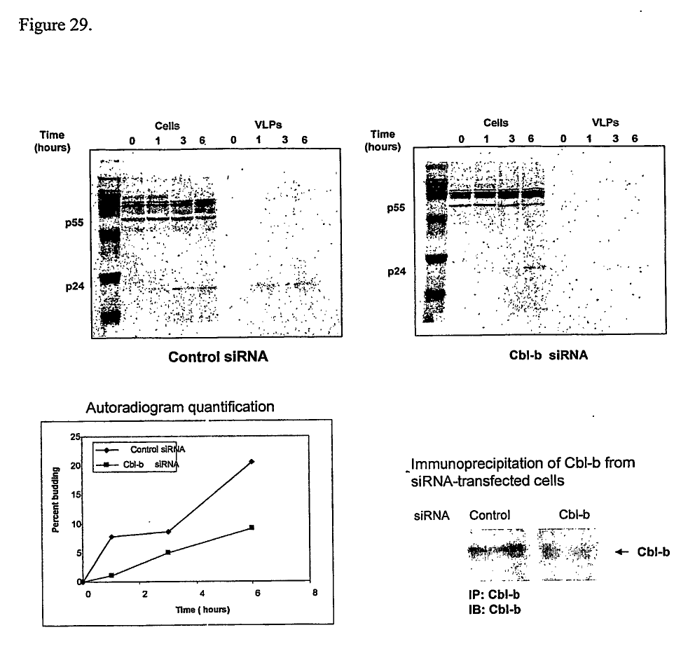

Results are presented in FIG. 29.

D. Check Cbl-b levels by IP/IB.
1. Resuspend cell pellets from step B2 in 0.5 ml lysis buffer (described in C-7)
2. Incubate on ice for 10 min.
3. Spin in 4° C. for 15 min at 14,000 rpm and transfer the sup into clean tubes.
4. Perform IP Cbl-b:—
   a. Add 4 μg (20 μl) of anti Cbl-b.
   b. Incubate by rotation, in cold, 2.5 hours.
   c. Wash 160 μl of recombinant anti mouse beads three times with 1 ml cold lysis buffer.
   d. Resuspend beads in 160 μl of lysis buffer and add 20 μl (10 μl sepharose) to each IP reaction (mix well between samples and use cut tips).
   e. Rotate IP tubes another 45 minutes.
   f. Pellet in cold centrifuge (30 seconds is sufficient) and wash IP beads 3 times with 1 ml cold HNTG buffer, removing as much as possible between washes.
   g. Add 25 μl 2× Sample buffer, boil 5 minutes, and store −20° C.
   h. Thaw and boil samples additional 3 minute before loading on gel.
   i. Separate on 7.5% gel.
   j. Western Blot: 1 hour blocking TBS-T+skim milk 10%.
   k. 1 hour 1^{st} Ab 1:100, in block solution overnight.
   l. Wash X3, -7 minutes each wash in TBS-T.
   m. Anti-IgG mouse 1:10,000 in TBS-T 31 1 hour, RT.
   n. Wash X3, ˜7 minutes each wash in TBS-T and perform ECL.

Results are presented in FIG. 29.

### Example 12

### Cbl-b Affects the Release of VLP at Steady State

1. Day 1: plate two 6-wells plates with HeLaSS6 cells at 4×10⁵ cells/well (50% confluence on the next day).
2. Day 2: transfect as indicate in the table. (0.25 ml OptiMBM+5 μl Lipofectamine2000)+0.25 ml OptiMEM+DNA as indicated in the table).

Plasmid no. 111: pNlenv-1.

Transfections:

| | Day 2 | Day 3 |
|---|---|---|
| | Transfection with | Transfection with 100 nM |
| | 100 nM siRNA | siRNA + 0.75 ug #111 |
| A1 | 187 (Control) | 187 (Control) + 0.75 ug #111 |
| A2 | 275 (Cbl-b) | 275 (Cbl-b) + 0.75 ug #111 |

Steady State VLP Assay

A. Cell Extracts
1. Collect 2 ml medium and pellet floating cells by centrifugation (1 min, 1400 rpm at 4° C.), save sup (continue with sup immediately to step B), scrape cells in ice-cold PBS, add to the corresponding floated cell pellet and centrifuge for 5 min 1800 rpm at 4° C.
2. Wash cell pellet once with ice-cold PBS.
3. Resuspend cell pellet (from 6 well) in 100 μl NP40-DOC lysis buffer and incubate 10 minutes on ice.
4. Centrifuge at 14,000rpm for 15 min. Transfer supernatant to a clean eppendorf.
5. Prepare samples for SDS-PAGE by adding them sample buffer and boil for 10 min—take the same volume for each reaction (15 μl).

B. Purification of VLP from Cell Media
1. Filtrate the supernatant through a 0.45μ filter.
2. Centrifuge supernatant at 14,000 rpm at 4° C. for at least 2 h.
3. Resuspend VLP pellet of A1-A7 in 50 μl 1× sample buffer and boil for 10 min. Load 25 μl of each sample.

C. Western Blot analysis
1. Run all samples from stages A and B on Tris-Gly SDS-PAGE 12.5%.
2. Transfer samples to nitrocellulose membrane (100V for 1.15 h.).
3. Dye membrane with ponceau solution.
4. Block with 10% low fat milk in TBS-t for 1 h.
5. Incubate with anti p24 rabbit 1:500 in TBS-t 2 hour (room temperature)—o/n (4° C).
6. Wash 3 times with TBS-t for 7min each wash.
7. Incubate with secondary antibody anti rabbit cy5 1:500 for 30 min.
8. Wash five times for 10 min in TBS-t.
9. View in Typhoon for fluorescence signal (650).

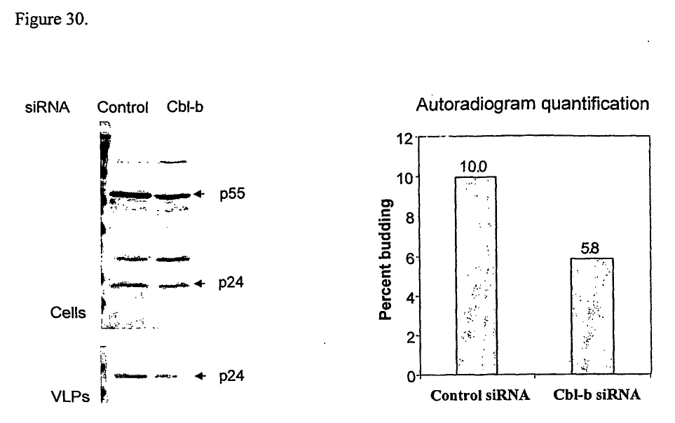

Results are presented in FIG. 30.

### Example 13

### Cbl-b Associates with POSH in-vivo

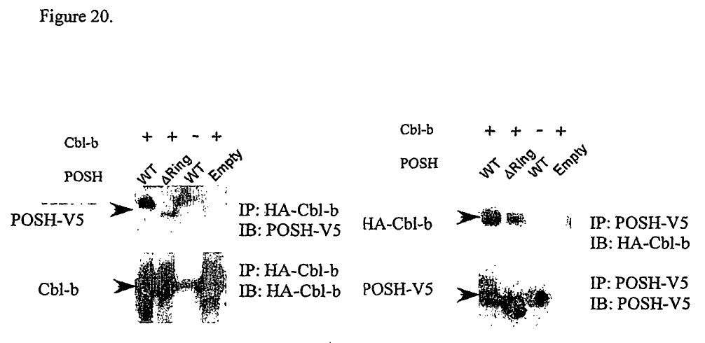

293T cells in 10 cm plates were transfected with HA-Cbl-b (1.5 μg) and POSH-V5 (5 μg) or POSH-deIRING-V5 (1.5 μg) or empty vector to a final plasmid amount of 6.5 μg, using calcium phosphate transfection. Cells were harvested after 24 hours and lysed in cold buffer containing: 0.5% NP-40, 0.5% Sodium Deoxycholate, 20 mM HEPES pH=7.9, 100 mM KCl, 250 mM NaCl, 0.5 mM DTT, and phosphatase/protease inhibitors. Lysate was cleared by centrifugation. Cleared supernatants were immunoprecipitated with anti-V5 antibody (Invitrogen) or anti-HA antibody (Roche) for 2.5 hours, and immune-complexes were precipitated on Protein A or G sepharose (Pharmacia) for 1 hour. Beads were washed 5 times with HNTG buffer and then boiled in 2× SDS sample buffer for 10 minutes. Samples were separated on 7.5% SDS-PAGE and electrotransferred to nitrocellulose membranes for western blot analysis with the indicated antibodies. See FIG. 20.

### Example 14

### In vitro Cbl-b Self Ubiguitination Assays

Cbl-b self-ubiquitination was determined by homogenous time-resolved fluorescence resonance energy transfer assay (TR-FRET). The conjugation of ubiquitin cryptate to GST tagged cbl-b and the binding of anti-GST tagged XL665 bring the two fluorophores into close proximity, which allows the FRET reaction to occur. To measure cbl-b ubiquitination activity, GST tagged cbl-b (60 nM) was incubated in reaction buffer (40 mM Hepes-NaOH, pH 7.5, 1 mM DTT, 2 mM ATP, 5 mM MgCl2, (with recombinant E1 (8 nM), UbcH5c (500 nM), and ubiquitin-cryptate (15 nM) (CIS bio International) for 30 minutes at 37° C. Reactions were stopped with 0.5M EDTA. Anti-GST-XL665 (CIS bio International) (50 nM) was then added to the reaction mixture for a further 45 minutes incubation at room temperature. -Emission at 620 nm and 665 nm was obtained after excitation at 380 run in a fluorescence reader (RUBYstar, BMG Labtechnologies). The generation of cbl-b-ubiquitin-cryptate adducts was then determined by calculating the fluorescence resonance energy transfer (FRET=(F) using the following formula:
)F=[(S665/S620−B665/B620)/(C665/C620−B665/B620)] where: S=actual fluorescence, B=Fluorescence obtained in parallel incubation without cbl-b, C=Fluorescence obtained in reaction without added compounds

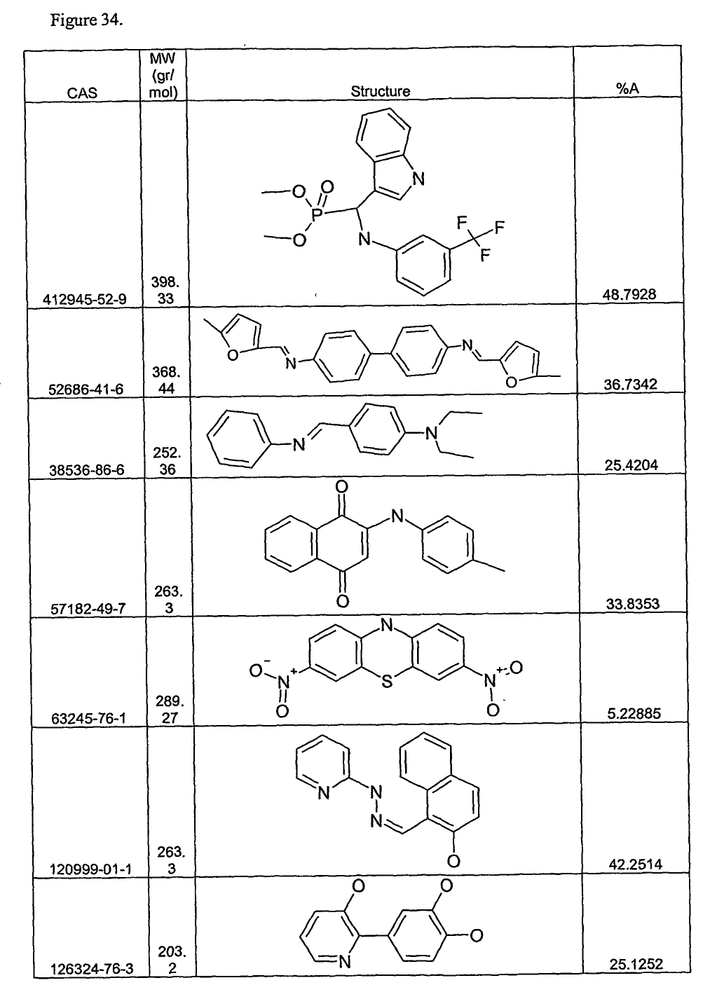

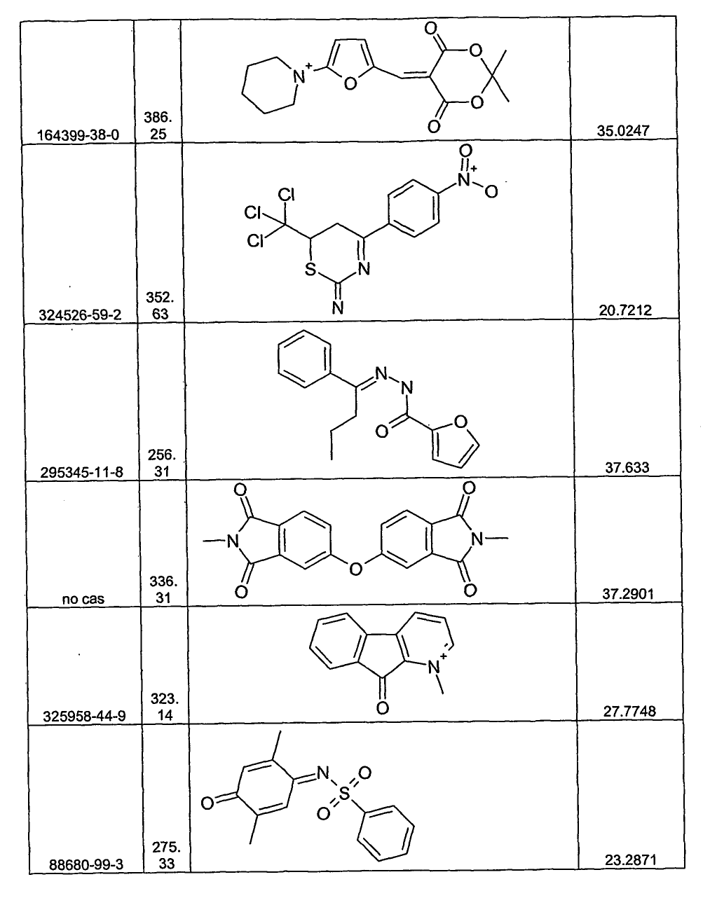

Inhibitors of Cbl-b activity are presented in FIG. 34. The compounds were all tested in a single concentration of 50 μM. The function F as described above is the basis on which the A % (activity) is calculated. 100% activity is F of the control (no compound). When an inhibitor is added, the A % will be the proportion between the F (control) and the “inhibitor F”.
A%=(inhibitorF)/(controlF)

Materials and solutions

ATP SigmaA-8937

DMSO Riedel-de Ha?n 34943, lot 2309C

DTT Sigma D-5545

E1 (in house preparation)—protein SOP preparation is in process

E2 (in house preparation)—protein SOP preparation is in process

EDTA

GST-hPOSH (in house preparation)—protein SOP preparation is in process

GST XL 665 Cis Bio

KH2PO4 Sigma -P0662

KF Riedel 1133

MgCl SigmaM-1028

Na2HPO4 Merek 6579

Ovalbumin Sigma A-5503

Ubiquitin U-6253

Ub-K Cis-Bio 61UbIKLA

Tris (pH=7.2) Sigma T2069

ddH20 JT Baker 4218

1. Assay Procedure

Microplates containing 10 μl compounds at 10 mM (from column 2-11).

a. Microplates Labeling
Prepare labels for microplate:
put labels to clear PS U-bottom clear microplate:
Put labels to 3 black microplates (triplicats).

b. Compounds Microplates Preparation
Put 90 μl DMSO in wells of origin plates (to 1 mM final).
Mix the microplates 30 sec at 800 RPM.
Transfer 5 μl of compounds from diluted plates to “inc. labeled” plates (including column 12 containing DMSO).

c. Incubation of cbl-b with Compounds
Set biocontrol to medium speed. Add 100 μl E3 solution in wells of the “inc.” labeled microplates, exept wells A12-D12.
Negative control: add 100 μl H₂O in wells A12-D12.
(positive control: E12-H12).
Mix the microplate 30 sec at 800 RPM.
Incubation 30 min at RT.

d. Distribution of Enzyme Solution×4
Add ATP to 2.9 ml of enzyme solution×4.
Set biocontrol to fast speed and (from 11×230 μl) put 8 μl enzymes solution×4 to 3 black microplates.

e. Enzymatic (Ubiguitination Step)
Distribution of (triplicat) 3×23 μl e3-compounds into 3 black microplates containing enzymatic solution (on splitting apparatus).
Incubation 30 minutes at 37° C.
Addition of 8 μl EDTA 0.5 M (from study 2536).
Incubation 12 minutes at RT.
Addition of 30 μl GST XL 665 in reconstitution buffer.
Incubation 45 min at RT.
Reading fluorescence.
Record results in computer.
   2. Solutions Preparation for Assay

Cbl-b

| | | | | | Amount |
|---|---|---|---|---|---|
| Thawing | | | | Final | for |
| cycles | Material | Lot | Stock conc. | conc. | 60.0 ml |
| 1 + 2 | cbl-b | NB110/p.12 | 2.2 mg/ml | 60 nM | 0.276 ml |
| — | Hepes | | 1 M | 40 mM | 2.40 ml |
| | pH = 7.2 | | | | |

| | | | | |
|---|---|---|---|---|
| — | H₂O | J. T. Baker 4218, | — | 57.3 ml |
| | | lot 0323010014 | | |

GST XL 665 in KF buffer

| | | | | | Amount |
|---|---|---|---|---|---|
| Thawing | | | Stock | | for |
| cycles | Material | Lot | conc. | Final conc. | 65 ml |
| 3.2.04 | GST XL 665 | 15 | 1 mg/ml | 50 nM | 0.488 ml |
| 3 | KF buffer | St. 2546 | — | — | 64.5 ml |

Enzyme solution×4—no ATP

| | | | | | | | Amount | |
|---|---|---|---|---|---|---|---|---|
| Thawing | | | | | | | for | |
| cycles | Material | Lot | Stock conc. | | Final conc. | | 21 ml | |
| — | Tris pH = 7.2 | T2069, 61K8942 | 1 | M | 40 | mM | 0.840 | ml |
| No | ATP | Study 2510 | 0.1 | M | 0.4 | mM | 0.084 | ml |
| — | MgCl₂ | M1028, 61K8927 | 1 | M | 20 | mM | 0.420 | ml |
| 1 | DTT | Study 2673 | 1 | M | 0.4 | mM | 0.0084 | ml |
| 6 + 1 | Ovalbumin | St. 2538 | 10% | | 0.2% | | 0.420 | ml |
| 2 | E1 | St. 2533 | 1.3 | mg/ml | 32 | nM | 0.057 | ml |
| 1 + 2 | E2 | NB98p82 | 0.15 | mg/ml | 2000 | nM | 5.60 | ml |
| 4 | Ubiquitin | 17.2.03 | 1 | mg/ml | 140 | nM | 0.025 | ml |
| 0 | Ub-K CisBio | 6TUBIKLA, lot | 10.6 | mg/L | 60 | nM | 1.01 | ml |
| | | 06 | | | | | | |

| | | | | | |
|---|---|---|---|---|---|
| — | H₂O | J. T. Baker 4218, lot 0323010014 | — | 12.5 | ml |

ATP adding: !

| | | | | | Amount |
|---|---|---|---|---|---|
| Thawing | | | Stock | Final | for |
| cycles | Material | Lot | conc. | conc. | 2.9 ml |
| 1 | ATP | Study 2510 | 0.1 M | 0.4 mM | 0.012 ml |

| | | | | |
|---|---|---|---|---|
| — | Enz. sol. × 4 | — | — | 2.89 ml |

KF buffer

| | | | | | | Amount |
|---|---|---|---|---|---|---|
| | | | | | | for |
| Material | Cat | Lot | Stock conc. | Final conc. | | 3 L |
| Na₂HPO₄.12H₂O | Merck 6579 | A122379 | 358.1 g/mol | 31.2 | mM | 33.5 g |
| KH₂PO₄ | Sigma P0662 | 39H0087 | 136.1 g/mol | 18.7 | mM | 7.64 g |
| KF, Riedel 1133 | Riedel 1133 | 1080B | 58.1 g/mol | 0.8 | M | 139.4 g |

| | | | | | |
|---|---|---|---|---|---|
| Ovalbumine | Sigma A5503 | 71K7028 | (100%) | 0.1% | 3.00 g |

Check pH=7
EDTA

pH=8

| | | | | Amount |
|---|---|---|---|---|
| | | | | for |
| Material | Lot | Stock conc. | Final conc. | 1000 ml |
| EDTA, E-5134 | 91K0133 | 372.2 g/mol | 0.5 M | 186.1 gr |
| NaOH | Iris 17.9.03 | 10 N | to pH = 8 | 65 ml |
| ddH₂O | — | — | — | to 1000 ml |

pH=8.3 (checked with pH meter)

Titrated ATP 0.1 M

| | | | | Amount |
|---|---|---|---|---|
| | | | | for |
| Material | Lot | Stock conc. | Final conc. | 34 ml |
| ATP, A8937 | 101K70005 | 583.4 g/mol | 0.1 M | 1.98 gr |
| NaOH | — | 10 N | To neutrality | 0.60 ml |
| ddH₂O | — | — | — | to 34 ml |

Checked with pH stick paper.

DTT 1 M

| | | | | Amount |
|---|---|---|---|---|
| | | | | for |
| Material | Lot | Stock conc. | Final conc. | 8.65 ml |
| DTT, D-5545 | 072K10411 | 154.3 g/mol | 1 M | 1335 mg |
| ddH₂O | — | — | — | to 9 ml |

Ovalbumine 10%

| | | | | Amount |
|---|---|---|---|---|
| | | | | for |
| Material | Lot | Stock conc. | Final conc. | 5 ml |
| Ovalbumine, A-5503 | | 100% | 10% | 500 mg |
| ddH₂O | — | — | — | to 5 ml |

### Example 15

### Cbl-b Reduction Inhibits Viral Release and Infectivity

Cbl-b Reduction Reduces Reverse Transcriptase (RT) Activity in Release Virus-Like-Particles (VLP)

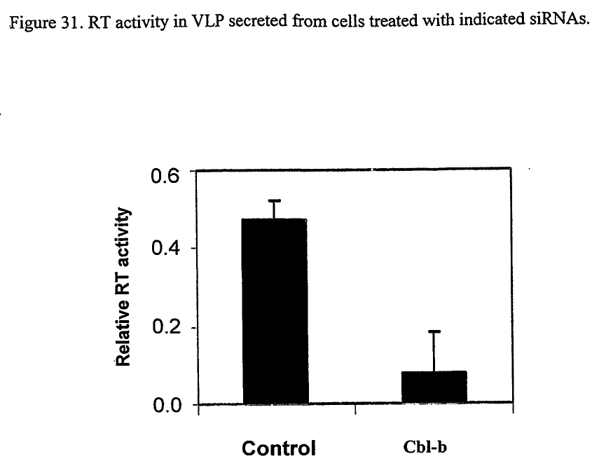

HeLa SS6 cell cultures (in triplicates) were transfected with siRNA targeting Cbl-b or with a control siRNA. Following gene silencing by siRNA, cells were transfected with pNLenvl, encoding an envelope-deficient subviral Gag-Pol expression system (Schubert et al., 1995) and RT activity in VLP released into the culture medium was determined (FIG. 31). Cells treated with Cbl-b-specific siRNA reduced RT activity by 80 percent.

Cbl-b Reduction Reduces HIV-1 Infectivity.

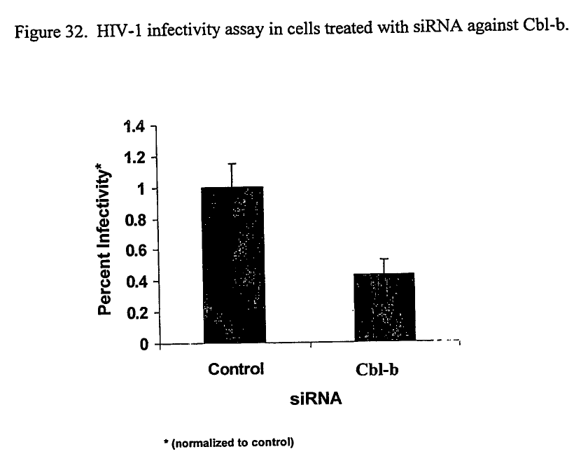

Applicants compared the production of infectious virus over a single cycle of HIV-1 replication in the presence of normal or reduced levels of Cbl-b. To this end, cells were initially transfected with either a control or Cbl-b specific siRNA and then co-transfected with three plasmids encoding HIV-1 gag-pol, HIV-LTR-GFP and VSV-G. Hence, the virus-producing cells release pseudotyped virions that contain VSV-G but do not by themselves encode an envelope protein and therefore, can infect target cells only once. Viruses were collected twenty-four hours post-trasnfection and used to infect HEK-293T cells. Infected target cells are detected by FACS analysis of GFP-positive cells. Cbl-b reduction resulted in 60% reduction of virus infectivity, indicting that Cbl-b is important for HIV-1 release. See FIG. 32.

Cell Culture and Transfections

Hela SS6 cells were grown in Dulbecco's modified Eagle's medium (DMEM) supplemented with 10% heat-inactivated fetal calf serum and 100 units/ml penicillin and 100 μg/ml streptomycin. For transfections, HeLa SS6 cells were grown to 50% confluency in DMEM containing 10% FCS without antibiotics. Cells were then transfected with the relevant double-stranded siRNA (50-100 nM) using lipofectamin 2000 (Invitrogen, Paisley, UK). On the day following the initial transfection, cells were split 1:3 in complete medium and co-transfected 24 hours later with HIV-1NLenvl (2 μg per 6-well) (Schubert et al., 1995) and a second portion of double-stranded siRNA.

Assays for Virus Release by RT Activity

Virus and virus-like particle (VLP) release was determined one day after transfection with the pro-viral DNA as previously described (Adachi et al., 1986; Fukumori et al., 2000; Lenardo et al., 2002). The culture medium of virus-expressing cells was collected and centrifuged at 500×g for 10 minutes. The resulting supernatant was passed through a 0.45 □m-pore filter and the filtrate was centrifuged at 14,000×g for 2 hours at 4° C. The resulting supernatant was removed and the viral-pellet was re-suspended in cell solubilization buffer (50 mM Tris-HCl, pH7.8, 80 mM potassium chloride, 0.75 mM EDTA and 0.5% Triton X-100, 2.5 mM DTT and protease inhibitors). The corresponding cells were washed three times with phosphate-buffered saline (PBS) and then solubilized by incubation on ice for 15 minutes in cell solubilization buffer. The cell detergent extract was then centrifuged for 15 minutes at 14,000×g at 4oC. The sample of the cleared extract (normally 1:10 of the initial sample) were resolved on a 12.5% SDS-polyacrylarnide gel, then transferred onto nitrocellulose paper and subjected to immunoblot analysis with rabbit anti-CA antibodies. The CA was detected after incubation with a secondary anti-rabbit antibody conjugated to Cy5 (Jackson Laboratories, West Grove, Pa.) and detected by fluorescence imaging (Typhoon instrument, Molecular Dynamics, Sunnyvale, Calif.). The Pr55 and CA were then quantified by densitometry. A colorimetric reverse transcriptase assay (Roche Diagnostics GmbH, Mannenheim, Germany) was used to measure reverse transcriptase activity in VLP extracts. RT activity was normalized to amount of Pr55 and CA produced in the cells.

Infectivity Assay

HeLa SS6 cells were grown to 50% confluency in DMEM containing 10% FCS without antibiotics. Cells were then transfected (in duplicates) with the relevant double-stranded siRNA (50-100 nM) using lipofectamin 2000 (Invitrogen, Paisley, UK). On the day following the initial transfection, cells were split 1:3 in complete medium and co-transfected 24 hours later with pCMVΔR8.2 (Naldini et al., 1996a), encoding HIV-1 gag-pol (5 μg), pHR′-CMV-GFP (4 μg) (Naldini et al., 1996b), pMD.G (Naldini et al., 1996a), encoding VSV-G (1.5 μg) and a second portion of double-stranded siRNA. Infection was performed twenty-four hours post-transfection, as follows: medium was collected from HeLa SS6 cells, polybrene was added to a final concentration of 8 μg/ml and the medium was palced on HEK-293T cells. Seventy-two hours post-infection cells were collected by trypsinization. Cells were fixed with 4% paraformaldehyde and analyzed for GFP-expression by FACS analysis.

### REFERENCES

Adachi, A., Gendelman, H. E., Koenig, S., Folks, T., Willey, R., Rabson, A., and Martin, M. A. (1986). Production of acquired immunodeficiency syndrome-associated retrovirus in human and nonhuman cells transfected with an infectious molecular clone. J Virol 59, 284-291.
Fukumori, T., Akari, H., Yoshida, A., Fujita, M., Koyama, A. H., Kagawa, S., and Adachi, A. (2000). Regulation of cell cycle and apoptosis by human immunodeficiency virus type 1 Vpr. Microbes Infect 2, 1011-1017.
Lenardo, M. J., Angleman, S. B., Bounkeua, V., Dimas, J., Duvall, M. G., Graubard, M. B., Hornung, F., SeLkirk, M. C., Speirs, C. K., Trageser, C., et al. (2002). Cytopathic killing of peripheral blood CD4(+) T lymphocytes by human immunodeficiency virus type 1 appears necrotic rather than apoptotic and does not require env. J Virol 76, 5082-5093.
Naldini, L., Blomer, U., Gage, F. H., Trono, D., and Verma, I. M. (1996a). Efficient transfer, integration, and sustained long-term expression of the transgene in adult rat brains injected with a lentiviral vector. Proc Natl Acad Sci U S A 93, 11382-11388.
Naldini, L., Blomer, U., Gallay, P., Ory, D., Mulligan, R., Gage, F. H., Verma, I. M., and Trono, D. (1996b). In vivo gene delivery and stable transduction of nondividing cells by a lentiviral vector. Science 272, 263-267.
Schubert, U., Clouse, K. A., and Strebel, K. (1995). Augmentation of virus secretion by the human immunodeficiency virus type 1 Vpu protein is cell type independent and occurs in cultured human primary macrophages and lymphocytes. J Virol 69, 7699-7711.

### Example 16

### Cbl-b RING Mutant Inhibits Viral Release and Infectivity

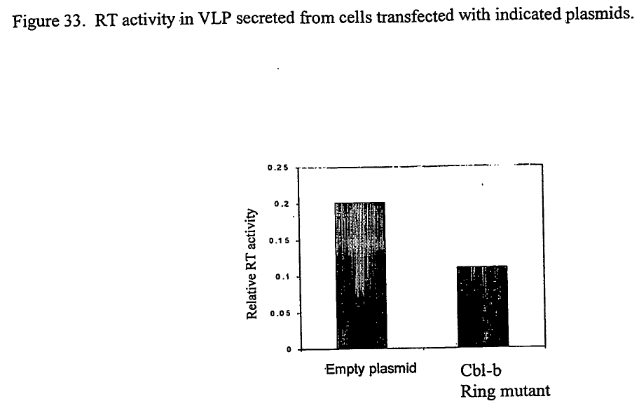

HeLa SS6 cell cultures (in triplicates) were co-transfected with vector encoding Cbl-b RING mutant (C373A) or with a control empty v ector and w aith with pNLenv1, encoding an envelope-deficient subviral Gag-Pol expression system (Schubert et al., 1995) and reverse transcriptase (RT) activity in VLP released into the culture medium was determined (FIG. 33). Cells transfected with Cbl-b-RING mutant reduced RT activity by 50 percent.

Cell Culture and Transfections

Hela SS6 cells were grown in Dulbecco's modified Eagle's medium (DMEM) supplemented with 10% heat-inactivated fetal calf serum and 100 units/ml penicillin and 100 μg/ml streptomycin. For transfections, HeLa SS6 cells were grown to 100% confluency in DMEM containing 10% FCS without antibiotics in 10 cm dishes. Cells were then transfected with control empty vector (pEF) or a vector expressing a Ring-mutant version of Cbl-b (C373A) (provided by Dr. Stanley Lipkowitz of the NIH/NC/CCR/LCMB B ethesda USA) and HIV-1NLenvl (5 μg per 10 cm dish) (Schubert et al., 1995) using lipofectamin 2000 (Invitrogen, Paisley, UK).

Assays for Virus Release by RT Activity

Virus and virus-like particle (VLP) release was determined one day after transfection with the pro-viral DNA as previously described (Adachi et al., 1986; Fukumori et al., 2000; Lenardo et al., 2002). The culture medium of virus-expressing cells was collected and centrifuged at 500×g for 10 minutes. The resulting supernatant was passed through a 0.45 μm-pore filter and the filtrate was centrifuged at 14,000×g for 2 hours at 4° C. The resulting supernatant was removed and the viral-pellet was re-suspended in cell solubilization buffer (50 mM Tris-HCl, pH7.8, 80 mM potassium chloride, 0.75 mM EDTA and 0.5% Triton X-100, 2.5 mM DTT and protease inhibitors). The corresponding cells were washed three times with phosphate-buffered saline (PBS) and then solubilized by incubation on ice for 15 minutes in cell solubilization buffer. The cell detergent extract was then centrifuged for 15 minutes at 14,000×g at 4° C. The sample of the cleared extract (normally 1:10 of the initial sample) were resolved on a 12.5% SDS-polyacrylamide gel, then transferred onto nitrocellulose paper and subjected to immunoblot analysis with rabbit anti-CA antibodies. The CA was detected after incubation with a secondary anti-rabbit antibody conjugated to Cy5 (Jackson Laboratories, West Grove, Pa.) and detected by fluorescence imaging (Typhoon instrument, Molecular Dynamics, Sunnyvale, Calif.). The Pr55 and CA were then quantified by densitometry. A colorimetric reverse transcriptase assay (Roche Diagnostics GmbH, Mannenheim, Germany) was used to measure reverse transcriptase activity in VLP extracts. RT activity was normalized to amount of Pr55 and CA produced in the cells.

Infectivity Assay

HeLa SS6 cells were grown to 50% confluency in DMEM containing 10% FCS without antibiotics. Cells were then transfected (in duplicates) with the relevant double-stranded siRNA (50-100 nM) using lipofectamin 2000 (Invitrogen, Paisley, UK). On the day following the initial transfection, cells were split 1:3 in complete medium and co-transfected 24 hours later with pCMVAR8.2 (Naldini et al., 1996a), encoding HIV-1 gag-pol (5 μg), pHR′-CMV-GFP (4 μg) (Naldini et al., 1996b), pMD.G (Naldini et al., 1996a), encoding VSV-G (1.5 μg) and a second portion of double-stranded siRNA. Infection was performed twenty-four hours post-transfection, as follows: medium was collected from HeLa SS6 cells, polybrene was added to a final concentration of 8 μg/ml and the medium was palced on HEK-293T cells. Seventy-two hours post-infection cells were collected by trypsinization. Cells were fixed with 4% paraformaldehyde and analyzed for GFP-expression by FACS analysis.

### REFERENCES

Adachi, A., Gendelman, H. E., Koenig, S., Folks, T., Willey, R., Rabson, A., and Martin, M. A. (1986). Production of acquired immunodeficiency syndrome-associated retrovirus in human and nonhuman cells transfected with an infectious molecular clone. J Virol 59, 284-291.
Fukumori, T., Akari, H., Yoshida, A., Fujita, M., Koyamna, A. H., Kagawa, S., and Adachi, A. (2000). Regulation of cell cycle and apoptosis by human immunodeficiency virus type 1 Vpr. Microbes Infect 2, 1011-1017.
Lenardo, M. J., Angleman, S. B., Bounkeua, V., Dimas, J., Duvall, M. G., Graubard, M. B., Homung, F., Selkirk, M. C., Speirs, C. K., Trageser, C., et al. (2002). Cytopathic killing of peripheral blood CD4(+) T lymphocytes by human immunodeficiency virus type 1 appears necrotic rather than apoptotic and does not require env. J Virol 76, 5082-5093.
Naldini, L., Blomer, U., Gage, F. H., Trono, D., and Verma, I. M. (1996a). Efficient transfer, integration, and sustained long-term expression of the transgene in adult rat brains injected with a lentiviral vector. Proc Natl Acad Sci U S A 93, 11382-11388.
Naldini, L., Blomer, U., Gallay, P., Ory, D., Mulligan, R., Gage, F. H., Verma, I. M., and Trono, D. (1996b). In vivo gene delivery and stable transduction of nondividing cells by a lentiviral vector. Science 272, 263-267.
Schubert, U., Clouse, K. A., and Strebel, K. (1995). Augmentation of virus secretion by the human irnmunodeficiency virus type 1 Vpu protein is cell type independent and occurs in cultured human primary macrophages and lymphocytes. J Virol 69, 7699-7711.

### Example 17

### Exemplary Cbl-b siRNA Duplexes

| | |
|---|---|
| CB-1876 Target: | |
| AATGGAAGGCACAGTAGAGTG | |
| sIRNA duplex: | |
| UGG AAG GCA CAG UAG AGU GdTdT | (SEQ ID NO: 59) |
| and | |
| CAC UCU ACU GUG CCU UCC AdTdT | (SEQ ID NO: 60) |
| B-U203 Target: | |
| GATTATGATCTTCTCATCCCT | |
| siRNA duplex: | |
| UUA UGA UCU UCU CAU CCC UdTdT | (SEQ ID NO: 61) |
| and | |
| AGG GAU GAG AAG AUC AUA AdTdT | (SEQ ID NO: 62) |
| CB-U170 | |
| Cb1B AA GCATGGTTCTTCACTCAAC | |
| siRNA duplex: | |
| GCA UGG UUC UUC ACU CAA CdTdT | (SEQ ID NO: 63) |
| and | |
| GUU GAG UGA AGA ACC AUG CdTdT | (SEQ ID NO: 64) |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject applications have been discussed, the above specification is illustrative and not restrictive. Many variations of the applications will become apparent to those skilled in the art upon review of this specification and the claims below. The full scope of the applications should be determined by reference to the claims, along with their full scope of equivalents, and the specification, along with such variations.

1. An isolated, purified or recombinant complex, comprising a Cbl-b polypeptide and a POSH polypeptide.

2. (canceled)

3. A method of identifying an antiviral agent, comprising identifying a test agent that disrupts a complex of claim 1.

4. The complex of claim 1, wherein the Cbl-b polypeptide is a human Cbl-b polypeptide.

5. The complex of claim 1, wherein the POSH polypeptide is a human POSH polypeptide.

6. A method of identifying an agent that modulates an activity of a Cbl-b polypeptide and a POSH polypeptide, comprising identifying an agent that disrupts a complex of claim 1, wherein an agent that disrupts a complex of claim 1 is an agent that modulates an activity of the Cbl-b polypeptide or the POSH polypeptide.

7. A method of identifying an antiviral agent, comprising:
a) identifying a test agent that disrupts a complex comprising a Cbl-b polypeptide and a Cbl-b-AP polypeptide; and
b) evaluating the effect of the test agent on a function of a virus, wherein an agent that inhibits a pro-infective or pro-replicative function of a virus is an antiviral agent.

8. The method of claim 7, wherein the Cbl-b-AP is POSH.

9. The method of claim 7, wherein the virus is an envelope virus.

10. The method of claim 9, wherein the virus is a Human Immunodeficiency Virus.

11. The method of claim 7, wherein evaluating the effect of the test agent on a function of the virus comprises evaluating the effect of the test agent on the budding, release, infectivity, or reverse transcriptase activity of the virus or a virus-like particle.

12. (canceled)

13. The method of claim 7, wherein said agent is selected from among: an siRNA construct, an antisense construct, an antibody, a polypeptide, and a small molecule.

14-24. (canceled)

25. A method of identifying an antiviral agent, comprising:
a) identifying a test agent that inhibits an activity of or expression of a Cbl-b polypeptide; and
b) evaluating an effect of the test agent on a function of a virus.

26. (canceled)

27. The method of claim 25, wherein the virus is an envelope virus.

28. The method of claim 25, wherein the virus is a Human Immunodeficiency Virus.

29. The method of claim 25, wherein evaluating the effect of the test agent on a function of the virus comprises evaluating the effect of the test agent on the budding, release, infectivity, or reverse transcriptase activity of the virus or a virus-like particle.

30. The method of claim 25, wherein the test agent is selected from among: an siRNA construct, an antisense construct, an antibody, a polypeptide, and a small molecule.

31. The method of claim 30, wherein the test agent is an siRNA construct that inhibits the expression of Cbl-b and is selected from among SEQ ID NOS: 59-64.

32-46. (canceled)

47. The method of claim 25, wherein the agent inhibits the ubiquitin ligase activity of the Cbl-b polypeptide.

48-49. (canceled)

50. The method of claim 25, wherein the An isolated Cbl-b polypeptide comprises the amino acid sequence depicted in SEQ ID NO: 45.

51-52. (canceled)

53. The method of claim 25, wherein the Cbl-b polypeptide, comprises the amino acid sequence depicted in SEQ ID NO: 46.

54-63. (canceled)


SEQUENCE LISTING (64 sequences)

SEQ ID NO: 1 - DNA, 2667 bp, Homo sapiens
atggatgaatcagccttgttggatcttttggagtgtccggtgtgtctagagcgccttgatgcttctgcgaaggtcttgccttgccagcatacgttttgcaagcgatgtttgctggggatcgtaggttctcgaaatgaactcagatgtcccgagtgcaggactcttgttggctcgggtgtcgaggagcttcccagtaacatcttgctggtcagacttctggatggcatcaaacagaggccttggaaacctggtcctggtgggggaagtgggaccaactgcacaaatgcattaaggtctcagagcagcactgtggctaattgtagctcaaaagatctgcagagctcccagggcggacagcagcctcgggtgcaatcctggagccccccagtgaggggtatacctcagttaccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattattaaaccgttacctcagcccccacctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgccgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagcaaaaaagaaaaaaagggtttgttgaagttgctttctggcgcctccactaaacggaagccccgcgtgtctcctccagcatcgcccaccctagaagtggagctgggcagtgcagagcttcctctccagggagcggtggggcccgaactgccaccaggaggtggccatggcagggcaggctcctgccctgtggacggggacggaccggtcacgactgcagtggcaggagcagccctggcccaggatgcttttcataggaaggcaagttccctggactccgcagttcccatcgctccacctcctcgccaggcctgttcctccctgggtcctgtcttgaatgagtctagacctgtcgtttgtgaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaacatatga

SEQ ID NO: 2 - protein, 888 aa, Homo sapiens
MDESALLDLLECPVCLERLDASAKVLPCQHTFCKRCLLGIVGSRNELRCPECRTLVGSGVEELPSNILLVRLLDGIKQRPWKPGPGGGSGTNCTNALRSQSSTVANCSSKDLQSSQGGQQPRVQSWSPPVRGIPQLPCAKALYNYEGKEPGDLKFSKGDIIILRRQVDENWYHGEVNGIHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDSKKEKKGLLKLLSGASTKRKPRVSPPASPTLEVELGSAELPLQGAVGPELPPGGGHGRAGSCPVDGDGPVTTAVAGAALAQDAFHRKASSLDSAVPIAPPPRQACSSLGPVLNESRPVVCERHRVVVSYPPQSEAELELKEGDIVFVHKKREDGWFKGTLQRNGKTGLFPGSFVENI

SEQ ID NO: 3 - DNA, 5128 bp, Homo sapiens
ctgagagacactgcgagcggcgagcgcggtggggccgcatctgcatcagccgccgcagccgctgcggggccgcgaacaaagaggaggagccgaggcgcgagagcaaagtctgaaatggatgttacatgagtcattttaagggatgcacacaactatgaacatttctgaagattttttctcagtaaagtagataaagatggatgaatcagccttgttggatcttttggagtgtccggtgtgtctagagcgccttgatgcttctgcgaaggtcttgccttgccagcatacgttttgcaagcgatgtttgctggggatcgtaggttctcgaaatgaactcagatgtcccgagtgcaggactcttgttggctcgggtgtcgaggagcttcccagtaacatcttgctggtcagacttctggatggcatcaaacagaggccttggaaacctggtcctggtgggggaagtgggaccaactgcacaaatgcattaaggtctcagagcagcactgtggctaattgtagctcaaaagatctgcagagctcccagggcggacagcagcctcgggtgcaatcctggagccccccagtgaggggtatacctcagttaccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattattaaaccgttacctcagcccccacctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgccgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagcaaaaaagaaaaaaagggtttgttgaagttgctttctggcgcctccactaaacggaagccccgcgtgtctcctccagcatcgcccaccctagaagtggagctgggcagtgcagagcttcctctccagggagcggtggggcccgaactgccaccaggaggtggccatggcagggcaggctcctgccctgtggacggggacggaccggtcacgactgcagtggcaggagcagccctggcccaggatgcttttcataggaaggcaagttccctggactccgcagttcccatcgctccacctcctcgccaggcctgttcctccctgggtcctgtcttgaatgagtctagacctgtcgtttgtgaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaacatatgaggagactgacactgaagaagcttaaaatcacttcacacaacaaagtagcacaaagcagtttaacagaaagagcacatttgtggacttccagatggtcaggagatgagcaaaggattggtatgtgactctgatgccccagcacagttaccccagcgagcagagtgaagaagatgtttgtgtgggttttgttagtctggattcggatgtataaggtgtgccttgtactgtctgatttactacacagagaaacttttttttttttttaagatatatgactaaaatggacaattgtttacaaggcttaactaatttatttgcttttttaaacttgaacttttcgtataatagatacgttctttggattatgattttaagaaattattaatttatgaaatgataggtaaggagaagctggattatctcctgttgagagcaagagattcgttttgacatagagtgaatgcattttcccctctcctcctccctgctaccattatattttggggttatgttttgcttctttaagatagaaatcccagttctctaatttggttttcttctttgggaaaccaaacatacaaatgaatcagtatcaattagggcctggggtagagagacagaaacttgagagaagagaagttagtgattccctctctttctagtttggtaggaatcaccctgaagacctagtcctcaatttaattgtgtgggtttttaattttcctagaatgaagtgactgaaacaatgagaaagaatacagcacaacccttgaacaaaatgtatttagaaatatatttagttttatagcagaagcagctcaattgtttggttggaaagtaggggaaattgaagttgtagtcactgtctgagaatggctatgaagcgtcatttcacattttaccccaactgacctgcatgcccaggacacaagtaaaacatttgtgagatagtggtggtaagtgatgcactcgtgttaagtcaaaggctataagaaacactgtgaaaagttcatattcatccattgtgattctttccccacgtcttgcatgtattactggattcccacagtaatatagactgtgcatggtgtgtatatttcattgcgatttcctgttaagatgagtttgtactcagaattgaccaattcaggaggtgtaaaaataaacagtgttctcttctctaccccaaagccactactgaccaaggtctcttcagtgcactcgctccctctctggctaaggcatgcattagccactacacaagtcattagtgaaagtggtcttttatgtcctcccagcagacagacatcaaggatgagttaaccaggagactactcctgtgactgtggagctctggaaggcttggtgggagtgaatttgcccacaccttacaattgtggcaggatccagaagagcctgtctttttatatccattccttgatgtcattggcctctcccaccgatttcattacggtgccacgcagtcatggatctgggtagtccggaaaacaaaaggagggaagacagcctggtaatgaataagatccttaccacagttttctcatgggaaatacataataaaccctttcatctttttttttttcctttaagaattaaaactgggaaatagaaacatgaactgaaaagtcttgcaatgacaagaggtttcatggtcttaaaaagatactttatatggttgaagatgaaatcattcctaaattaaccttttttttaaaaaaaaacaatgtatattatgttcctgtgtgttgaatttaaaaaaaaaaaatactttacttggatattcatgtaatatataaaggtttggtgaaatgaactttagttaggaaaaagctggcatcagctttcatctgtgtaagttgacaccaatgtgtcataatattctttattttgggaaattagtgtattttataaaaattttaaaaagaaaaaagactactacaggttaagataatttttttacctgtcttttctccatattttaagctatgtgattgaagtacctctgttcatagtttcctggtataaagttggttaaaatttcatctgttaatagatcattaggtaatataatgtatgggttttctattggttttttgcagacagtagagggagattttgtaacaagggcttgttacacagtgatatggtaatgataaaattgcaatttatcactccttttcatgttaataatttgaggactggataaaaggtttcaagattaaaatttgatgttcaaacctttgt

SEQ ID NO: 4 - DNA, 2331 bp, Homo sapiens
ctgagagacactgcgagcggcgagcgcggtggggccgcatctgcatcagccgccgcagccgctgcggggccgcgaacaaagaggaggagccgaggcgcgagagcaaagtctgaaatggatgttacatgagtcattttaaggatgcacacaactatgaacatttctgaagattttttctcagtaaagtagataaagatggatgaatcagccttgttggatcttttggagtgtccggtgtgtctagagcgccttgatgcttctgcgaaggtcttgccttgccagcatacgttttgcaagcgatgtttgctggggatcgtaggttctcgaaatgaactcagatgtcccgagtgcaggactcttgttggctcgggtgtcgaggagcttcccagtaacatcttgctggtcagacttctggatggcatcaaacagaggccttggaaacctggtcctggtgggggaagtgggaccaactgcacaaatgcattaaggtctcagagcagcactgtggctaattgtagctcaaaagatctgcagagctcccagggcggacagcagcctcgggtgcaatcctggagccccccagtgaggggtatacctcagttaccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattattaaaccgttacctcagcccccacctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgccgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagc

SEQ ID NO: 5 - protein, 712 aa, Homo sapiens
MDESALLDLLECPVCLERLDASAKVLPCQHTFCKRCLLGIVGSRNELRCPECRTLVGSGVEELPSNILLVRLLDGIKQRPWKPGPGGGSGTNCTNALRSQSSTVANCSSKDLQSSQGGQQPRVQSWSPPVRGIPQLPCAKALYNYEGKEPGDLKFSKGDIIILRRQVDENWYHGEVNGIHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDS

SEQ ID NO: 6 - DNA, 4182 bp, Homo sapiens
atttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgctgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagcaaaaaagaaaaaaagggtttgttgaagttgctttctggcgcctccactaaacggaagccccgcgtgtctcctccagcatcgcccaccctagaagtggagctgggcagtgcagagcttcctctccagggagcggtggggcccgaactgccaccaggaggtggccatggcagggcaggctcctgccctgtggacggggacggaccggtcacgactgcagtggcaggagcagccctggcccaggatgcttttcataggaaggcaagttccctggactccgcagttcccatcgctccacctcctcgccaggcctgttcctccctgggtcctgtcttgaatgagtctagacctgtcgtttgtgaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaacatatgaggagactgacactgaagaagcttaaaatcacttcacacaacaaagtagcacaaagcagtttaacagaaagagcacatttgtggacttccagatggtcaggagatgagcaaaggattggtatgtgactctgatgccccagcacagttaccccagcgagcagagtgaagaagatgtttgtgtgggttttgttagtctggattcggatgtataaggtgtgccttgtactgtctgatttactacacagagaaacttttttttttttttaagatatatgactaaaatggacaattgtttacaaggcttaactaatttatttgcttttttaaacttgaacttttcgtataatagatacgttctttggattatgattttaagaaattattaatttatgaaatgataggtaaggagaagctggattatctcctgttgagagcaagagattcgttttgacatagagtgaatgcattttcccctctcctcctccctgctaccattatattttggggttatgttttgcttctttaagatagaaatcccagttctctaatttggttttcttctttgggaaaccaaacatacaaatgaatcagtatcaattagggcctggggtagagagacagaaacttgagagaagagaagttagtgattccctctctttctagtttggtaggaatcaccctgaagacctagtcctcaatttaattgtgtgggtttttaattttcctagaatgaagtgactgaaacaatgagaaagaatacagcacaacccttgaacaaaatgtatttagaaatatatttagttttatagcagaagcagctcaattgtttggttggaaagtaggggaaattgaagttgtagtcactgtctgagaatggctatgaagcgtcatttcacattttaccccaactgacctgcatgcccaggacacaagtaaaacatttgtgagatagtggtggtaagtgatgcactcgtgttaagtcaaaggctataagaaacactgtgaaaagttcatattcatccattgtgattctttccccacgtcttgcatgtattactggattcccacagtaatatagactgtgcatggtgtgtatatttcattgcgatttcctgttaagatgagtttgtactcagaattgaccaattcaggaggtgtaaaaataaacagtgttctcttctctaccccaaagccactactgaccaaggtctcttcagtgcactcgctccctctctggctaaggcatgcattagccactacacaagtcattagtgaaagtggtcttttatgtcctcccagcagacagacatcaaggatgagttaaccaggagactactcctgtgactgtggagctctggaaggcttggtgggagtgaatttgcccacaccttacaattgtggcaggatccagaagagcctgtctttttatatccattccttgatgtcattggcctctcccaccgatttcattacggtgccacgcagtcatggatctgggtagtccggaaaacaaaaggagggaagacagcctggtaatgaataagatccttaccacagttttctcatgggaaatacataataaaccctttcatctttttttttttcctttaagaattaaaactgggaaatagaaacatgaactgaaaagtcttgcaatgacaagaggtttcatggtcttaaaaagatactttatatggttgaagatgaaatcattcctaaattaaccttttttttaaaaaaaaacaatgtatattatgttcctgtgtgttgaatttaaaaaaaaaaaatactttacttggatattcatgtaatatataaaggtttggtgaaatgaactttagttaggaaaaagctggcatcagctttcatctgtgtaagttgacaccaatgtgtcataatattctttattttgggaaattagtgtattttataaaaattttaaaaagaaaaaagactactacaggttaagataatttttttacctgtcttttctccatattttaagctatgtgattgaagtacctctgttcatagtttcctggtataaagttggttaaaatttcatctgttaatagatcattaggtaatataatgtatgggttttctattggttttttgcagacagtagagggagattttgtaacaagggcttgttacacagtgatatggtaatgataaaattgcaatttatcactccttttcatgttaataatttgaggactggataaaaggtttcaagattaaaatttgatgttcaaacctttgt

SEQ ID NO: 7 - protein, 638 aa, Homo sapiens
ISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDSKKEKKGLLKLLSGASTKRKPRVSPPASPTLEVELGSAELPLQGAVGPELPPGGGHGRAGSCPVDGDGPVTTAVAGAALAQDAFHRKASSLDSAVPIAPPPRQACSSLGPVLNESRPVVCERHRVVVSYPPQSEAELELKEGDIVFVHKKREDGWFKGTLQRNGKTGLFPGSFVENI

SEQ ID NO: 8 - DNA, 3206 bp, Homo sapiens
gggcagcgggctcggcggggctgcatctaccagcgctgcggggccgcgaacaaaggcgagcagcggaggcgcgagagcaaagtctgaaatggatgttacatgaatcactttaagggctgcgcacaactatgaacgttctgaagccgttttctcactaaagtcactcaagatggatgagtctgccttgttggaccttctggagtgccctgtgtgtctagaacgcctggatgcttccgcaaaggtcttaccctgccagcataccttttgcaaacgctgtttgctggggattgtgggttcccggaatgaactcagatgtcccgaatgccggactcttgttggctctggggtcgacgagctccccagtaacatcctactggtcagacttctggatggcatcaagcagaggccttggaaacccggccctggtgggggcggcgggaccacctgcacaaacacattaagggcgcagggcagcactgtggttaattgtggctcgaaagatctgcagagctcccagtgtggacagcagcctcgggtgcaagcctggagccccccagtgaggggaatacctcagttaccgtgtgccaaagcattatataactacgaaggaaaagagcccggagaccttaagttcagcaaaggcgacaccatcattctgcgccgacaggtggatgagaattggtaccacggggaagtcagcggggtccacggctttttccccactaacttcgtgcagatcatcaaacctttacctcagcccccgcctcagtgcaaagcactttacgactttgaagtgaaagacaaggaagctgacaaagattgccttcccttcgcaaaggacgacgtactgaccgtgatccgcagagtggatgaaaactgggctgaaggaatgctggcagataaaataggaatatttccaatttcatacgtggagtttaactcagctgccaagcagctgatagagtgggataagcctcccgtgccaggagtggacacggcagaatgcccctcagcgacggcgcagagcacctctgcctcaaagcaccccgacaccaagaagaacaccaggaagcgacactccttcacctccctcaccatggccaacaagtcttcccaggggtcccagaaccgccactccatggagatcagccctcctgtgctcatcagttccagcaaccccacagccgcagcccgcatcagcgaactgtccgggctctcctgcagcgccccgtctcaggtccatataagcaccactgggttaattgtgaccccaccccctagcagcccggtgacaactggccctgcgttcacgttcccttcagatgtcccctaccaagctgcccttggaagtatgaatcctccacttcccccaccccctctcctggcggccaccgtactcgcctccaccccgtcaggcgctactgctgctgttgctgctgctgctgccgccgccgccgctgctggaatgggacccaggcctgtgatggggtcctctgaacagattgcacatttacggcctcagactcgtcccagtgtatatgttgctatatatccgtacactccccggaaggaagacgaactggagctgaggaaaggggagatgtttttggtgtttgagcgttgccaggacggctggtacaaagggacatcgatgcataccagcaagataggcgttttccctggcaactatgtggcgcccgtcacaagggcggtgacgaatgcctcccaagctaaagtctctatgtctactgcgggtcaggcaagtcgcggggtgaccatggtcagcccttccactgcaggaggacctacacagaagccccaaggaaacggcgtggccggaaatcccagcgtcgtccccacggctgtggtgtcagcagctcatatccagacaagtcctcaggctaaggtcctgctgcacatgtctgggcagatgacagtcaatcaggcccgcaatgctgtgaggacagttgcagcacatagccaggaacgccccacagcagcagtgactcccatccaggtccagaatgccgcctgccttggtcctgcatccgtgggcctgccccatcattctctggcctcccaacctctgcctccaatggcgggtcctgctgcccacggtgctgccgtcagcatcagtcgaaccaatgcccccatggcctgcgctgcaggggcttctctggcctccccaaatatgaccagtgccatgttggagacagagcccagtggtcgcacagtgaccatcctccctggactccccacatctccagagagtgctgcatcagcgtgtgggaacagttcagctgggaaaccagacaaggacagtaagaaagaaaaaaagggcctactgaagctgctttctggtgcctccaccaaacgcaagccccgagtctcccctccagcatcacctaccctggatgtggagctgggtgctggggaggctcccttgcagggagcagtaggtcctgagctgccgctagggggcagccacggcagagtggggtcatgccccacagatggtgatggtccagtggccgctggaacagcagccctagcccaggatgccttccaccgcaagacaagctccctggactccgcagtgcccattgctccaccacctcgccaggcctgctcctccctgggcccagtcatgaatgaggcccggcctgttgtttgtgaaaggcacagggtggtggtttcctaccctcctcagagtgaggccgaacttgaactcaaggaaggagatattgtgtttgttcataagaaacgagaggacggctggttcaaaggcacgttacagaggaatgggaagactggccttttcccagggagctttgtggaaaacatctgagaagacgggacacggagaaagcttatcatcacaccacgtgtgactaaagagcacaaagcagtttcatagaaagagcacatctgtggacttccagatcttcaagaaccgagcagaagatgggcacctgactccagagccccggcctggttaccccaggggcagagggaaggaggacacacctgtgtgggttccgtctctctgggttctgatgtgtaaagtgtgccttgtaatgtctaatggactttacagataaatgtctttttttttttaagatgtataactaaaatggacaattgtttacaaggcttaactaatttatttgcttttttaaaacttgaactttcttgtaatagcaaat

SEQ ID NO: 9 - protein, 892 aa, Mouse
MDESALLDLLECPVCLERLDASAKVLPCQHTFCKRCLLGIVGSRNELRCPECRTLVGSGVDELPSNILLVRLLDGIKQRPWKPGPGGGGGTTCTNTLRAQGSTVVNCGSKDLQSSQCGQQPRVQAWSPPVRGIPQLPCAKALYNYEGKEPGDLKFSKGDTIILRRQVDENWYHGEVSGVHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDTAECPSATAQSTSASKHPDTKKNTRKRHSFTSLTMANKSSQGSQNRHSMEISPPVLISSSNPTAAARISELSGLSCSAPSQVHISTTGLIVTPPPSSPVTTGPAFTFPSDVPYQAALGSMNPPLPPPPLLAATVLASTPSGATAAVAAAAAAAAAAGMGPRPVMGSSEQIAHLRPQTRPSVYVAIYPYTPRKEDELELRKGEMFLVFERCQDGWYKGTSMHTSKIGVFPGNYVAPVTRAVTNASQAKVSMSTAGQASRGVTMVSPSTAGGPTQKPQGNGVAGNPSVVPTAVVSAAHIQTSPQAKVLLHMSGQMTVNQARNAVRTVAAHSQERPTAAVTPIQVQNAACLGPASVGLPHHSLASQPLPPMAGPAAHGAAVSISRTNAPMACAAGASLASPNMTSAMLETEPSGRTVTILPGLPTSPESAASACGNSSAGKPDKDSKKEKKGLLKLLSGASTKRKPRVSPPASPTLDVELGAGEAPLQGAVGPELPLGGSHGRVGSCPTDGDGPVAAGTAALAQDAFHRKTSSLDSAVPIAPPPRQACSSLGPVMNEARPVVCERHRVVVSYPPQSEAELELKEGDIVFVHKKREDGWFKGTLQRNGKTGLFPGSFVENI

SEQ ID NO: 10 - DNA, 3149 bp, Drosophila melanogaster
catttgtatccgcttggccacgagctttggctgcacttggcaaacttaataaattaaacattgaatcctgcctattgcaacgataatataatctgatttagtgcattaagaacgacaagtagcgattataatagtagattttagcatttgagctaaatttatttcccaaccgcgtcttgggattgcgtatgcgtgagccagtacctgcatgtgtgtgtgttttggaatgtggccctgcacgaaattcaaatagtgaccatccttgagattttgcatactggcaagatggacgagcacacgttaaacgacctgttggagtgctccgtgtgtcttgagcgactggacaccacatcgaaggtgctgccatgccagcacaccttctgccgcaaatgcttgcaggacattgtggccagtcagcacaagttgcgatgcccggagtgccgcatcctggtctcttgcaaaattgatgagctgcctccaaacgtcttgctgatgcgaatcttagaaggcatgaaacaaaatgcagcagctggcaaaggagaagaaaagggagaggagactgaaacacagccggaaagggccaaacctcagccgccagcggaatcagtggccccgcctgacaaccaactactccagctgcagtcacatcagcaatctcatcagccggctcgtcacaagcaacgtcgatttctactcccccacgcctatgccctctttgacttcgcctccggtgaagccaccgatctaaagttcaagaaaggggatctgatactgatcaagcatcgcatcgacaacaactggtttgtgggtcaagcgaatggtcaggagggcacatttcccatcaactacgtcaaggtatcggttccgctgcccatgccgcagtgcattgccatgtatgactttaagatggggcccaacgacgaggagggatgcctcgaatttaagaaaagcactgtaatacaggtaatgcgccgagttgatcataattgggcagaaggacgaattggccagaccatcggaatctttccaatagcattcgttgagctgaatgcagcggccaaaaagctgttggacagcgggctacacacccatccattctgccatccaccgaagcaacaggggcagcgggcccttcctccggttccagttattgatcccacggtggtcacggaatccagttcgggatcctccaattccacgccgggcagcagcaattcaagctccacatccagctcgaataactgcagtccgaatcaccaaatctcactgccgaataccccccaacatgtagtagcttccggatcggcgtctgttcgtttccgtgacaagggagcaaaggagaaacgccactcactaaatgctttgctgggaggaggagctccattaagtctgctgcagaccaaccgccattcggctgaaattcttagcctgccccatgaactaagccgcttggaagtttccagctcaacagctctaaaacccacgtcagccccacagacatcgcgtgtacttaagaccactgttcagcagcagatgcaaccgaatttaccctggggatacttagccctgttcccatacaaaccacgccaaacggatgagctggaattaaaaaagggttgtgtttacattgtgaccgaacgatgtgtggacggttggttcaagggaaaaaactggttggacatcactggagtgttcccgggcaactacctgacgcccctgcgcgcccgcgaccagcagcagttaatgcatcaatggaaatatgttccccaaaatgcagacgcccagatggcacaagtacagcagcatccagttgcaccagatgtgcgactcaacaacatgctgtccatgcaaccgcctgatttgccacctcgtcagcagcaggctaccgccacgaccaccagttgctctgtgtggtcgaaaccagtggaggcgctgttcagcagaaaatcggagcccaagcctgaaactgccacagcttcgactacgagcagcagttcctctggagcagtgggacttatgaggagattaactcacatgaaaacacgctccaaatctccgggagcgtccttgcagcaagttccgaaagaagctattagcacaaatgtggaatttacaacaaacccatcagctaaattgcatccagtacatgtaagatccggctcgtgccccagtcagctgcagcacagtcaaccgctcaatgaaactccagcagccaagacagcggcacaacaacagcagttcctacccaagcagctgccttccgcttctacgaacagcgtttcgtacggatcgcaacgcgtgaaaggaagcaaggaacgtcctcacttgatttgcgcgagacaatcattagatgcagctacatttcgcagtatgtacaacaatgccgcgtcgccgccgccacctactacttccgtggccccagctgtctacgccggcggtcagcaacaggtgattcctggaggtggagcgcaatcccagttgcatgccaatatgattattgcacccagccatcggaagtcgcacagcctagatgcgagtcatgtgctgagtcccagcagcaatatgatcacggaggcggccattaaggccagcgccaccactaagtctccttactgcacgagggaaagtcgattccgctgcattgtgccgtatccaccaaacagtgacattgaactagagctacatttgggcgacattatctacgtccagcggaagcagaagaacggctggtataagggcacccatgcccgtacccacaaaaccgggctgttccccgcctcctttgttgaaccggattgttaggaaagttatggttcaaactagaatttattaagcgaaattccaaattacttgtctaaaaggattcaatcgtcggtctattcgggcttccaaatacgcaatctcatatttctcttttcaaaaaagaaaccgttttgtactcttccaatcgaatgggcagctcgccgttgtacttttttatacaatgcttgatcaaaataggctagccatgtaagacttagggaacagttacttaagccttagcgattagttagctagagaaataatctaaccgatccttgtgccctctacaaagttatttgtaatatacgatactcagtaataaaaaaaaaaaaaaaaaaaaaaaaaaa

SEQ ID NO: 11 - protein, 838 aa, Drosophila melanogaster
MDEHTLNDLLECSVCLERLDTTSKVLPCQHTFCRKCLQDIVASQHKLRCPECRILVSCKIDELPPNVLLMRILEGMKQNAAAGKGEEKGEETETQPERAKPQPPAESVAPPDNQLLQLQSHQQSHQPARHKQRRFLLPHAYALFDFASGEATDLKFKKGDLILIKHRIDNNWFVGQANGQEGTFPINYVKVSVPLPMPQCIAMYDFKMGPNDEEGCLEFKKSTVIQVMRRVDHNWAEGRIGQTIGIFPIAFVELNAAAKKLLDSGLHTHPFCHPPKQQGQRALPPVPVIDPTVVTESSSGSSNSTPGSSNSSSTSSSNNCSPNHQISLPNTPQHVVASGSASVRFRDKGAKEKRHSLNALLGGGAPLSLLQTNRHSAEILSLPHELSRLEVSSSTALKPTSAPQTSRVLKTTVQQQMQPNLPWGYLALFPYKPRQTDELELKKGCVYIVTERCVDGWFKGKNWLDITGVFPGNYLTPLRARDQQQLMHQWKYVPQNADAQMAQVQQHPVAPDVRLNNMLSMQPPDLPPRQQQATATTTSCSVWSKPVEALFSRKSEPKPETATASTTSSSSSGAVGLMRRLTHMKTRSKSPGASLQQVPKEAISTNVEFTTNPSAKLHPVHVRSGSCPSQLQHSQPLNETPAAKTAAQQQQFLPKQLPSASTNSVSYGSQRVKGSKERPHLICARQSLDAATFRSMYNNAASPPPPTTSVAPAVYAGGQQQVIPGGGAQSQLHANMIIAPSHRKSHSLDASHVLSPSSNMITEAAIKASATTKSPYCTRESRFRCIVPYPPNSDIELELHLGDIIYVQRKQKNGWYKGTHARTHKTGLFPASFVEPDC

SEQ ID NO: 12 - DNA, 18 bp, Homo sapiens
cttgccttgccagcatac

SEQ ID NO: 13 - DNA, 18 bp, Homo sapiens
ctgccagcattccttcag

SEQ ID NO: 14 - DNA, 21 bp, Homo sapiens
aacagaggccttggaaacctg

SEQ ID NO: 15 - DNA, 21 bp, Homo sapiens
ttcagaggccuuggaaaccug

SEQ ID NO: 16 - DNA, 21 bp, Homo sapiens
ttcagguuuccaaggccucug

SEQ ID NO: 17 - DNA, 21 bp, Homo sapiens
aaagagcctggagaccttaaa

SEQ ID NO: 18 - DNA, 21 bp, Homo sapiens
ttagagccuggagaccuuaaa

SEQ ID NO: 19 - DNA, 21 bp, Homo sapiens
ttuuuaaggucuccaggcucu

SEQ ID NO: 20 - DNA, 21 bp, Homo sapiens
aaggattggtatgtgactctg

SEQ ID NO: 21 - DNA, 21 bp, Homo sapiens
ttggauugguaugugacucug

SEQ ID NO: 22 - DNA, 21 bp, Homo sapiens
ttcagagucacauaccaaucc

SEQ ID NO: 23 - DNA, 21 bp, Homo sapiens
aagctggattatctcctgttg

SEQ ID NO: 24 - DNA, 21 bp, Homo sapiens
ttgcuggauuaucuccuguug

SEQ ID NO: 25 - DNA, 21 bp, Homo sapiens
ttcaacaggagauaauccagc

SEQ ID NO: 26 - protein, 41 aa, Homo sapiens
CPVCLERLDASAKVLPCQHTFCKRCLLGIVGSRNELRCPEC

SEQ ID NO: 27 - protein, 56 aa, Homo sapiens
PCAKALYNYEGKEPGDLKFSKGDIIILRRQVDENWYHGEVNGIHGFFPTNFVQIIK

SEQ ID NO: 28 - protein, 60 aa, Homo sapiens
PQCKALYDFEVKDKEADKDCLPFAKDDVLTVIRRVDENWAEGMLADKIGIFPISYVEFNS

SEQ ID NO: 29 - protein, 58 aa, Homo sapiens
SVYVAIYPYTPRKEDELELRKGEMFLVFERCQDGWFKGTSMHTSKIGVFPGNYVAPVT

SEQ ID NO: 30 - protein, 57 aa, Homo sapiens
ERHRVVVSYPPQSEAELELKEGDIVFVHKKREDGWFKGTLQRNGKTGLFPGSFVENI

SEQ ID NO: 31 - DNA, 121 bp, Homo sapiens
tgtccggtgtgtctagagcgccttgatgcttctgcgaaggtcttgccttgccagcatacgttttgcaagcgatgtttgctggggatcgtaggttctcgaaatgaactcagatgtcccgagt

SEQ ID NO: 32 - DNA, 165 bp, Homo sapiens
ccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattatt

SEQ ID NO: 33 - DNA, 177 bp, Homo sapiens
cctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaac

SEQ ID NO: 34 - DNA, 171 bp, Homo sapiens
agtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtc

SEQ ID NO: 35 - DNA, 169 bp, Homo sapiens
gaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaaca

SEQ ID NO: 36 - DNA, 21 bp, Artificial Sequence
  target sequence
aagtccaaaggttccggagac

SEQ ID NO: 37 - DNA, 3354 bp, Homo sapiens
ctgggtcctgtgtgtgccacaggggtggggtgtccagcgagcggtctcctcctcctgctagtgctgctgcggcgtcccgcggcctccccgagtcgggcgggaggggagagcgggtgtggatttgtcttgacggtaattgttgcgtttccacgtctcggaggcctgcgcgctgggttgctccttcttcgggagcgagctgttctcagcgatcccactcccagccggggctccccacacacactgggctgcgtgcgtgtggagtgggacccgcgcacacgcgtgtctctggacagctacggcgccgaaagaactaaaattccagatggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaaaactatcgtaccatggaaagtattcagacagtgccttcatgaggtccaccagattagctctagcctggaagcaatggctctaaaatcaacaattgatttaacttgcaatgattacatttcagtttttgaatttgatatttttaccaggctgtttcagccttggggctctattttgcggaattggaatttcttagctgtgacacatccaggttacatggcatttctcacatatgatgaagttaaagcacgactacagaaatatagcaccaaacccggaagctatattttccggttaagttgcactcgattgggacagtgggccattggctatgtgactggggatgggaatatcttacagaccatacctcataacaagcccttatttcaagccctgattgatggcagcagggaaggattttatctttatcctgatgggaggagttataatcctgatttaactggattatgtgaacctacacctcatgaccatataaaagttacacaggaacaatatgaattatattgtgaaatgggctccacttttcagctctgtaagatttgtgcagagaatgacaaagatgtcaagattgagccttgtgggcatttgatgtgcacctcttgccttacggcatggcaggagtcggatggtcagggctgccctttctgtcgttgtgaaataaaaggaactgagcccataatcgtggacccctttgatccaagagatgaaggctccaggtgttgcagcatcattgacccctttggcatgccgatgctagacttggacgacgatgatgatcgtgaggagtccttgatgatgaatcggttggcaaacgtccgaaagtgcactgacaggcagaactcaccagtcacatcaccaggatcctctccccttgcccagagaagaaagccacagcctgacccactccagatcccacatctaagcctgccacccgtgcctcctcgcctggatctaattcagaaaggcatagttagatctccctgtggcagcccaacaggttcaccaaagtcttctccttgcatggtgagaaaacaagataaaccactcccagcaccacctcctcccttaagagatcctcctccaccgccacctgaaagacctccaccaatcccaccagacaatagactgagtagacacatccatcatgtggaaagcgtgccttccagagacccgccaatgcctcttgaagcatggtgccctcgggatgtgtttgggactaatcagcttgtgggatgtcgactcctaggggagggctctccaaaacctggaatcacagcgagttcaaatgtcaatggaaggcacagtagagtgggctctgacccagtgcttatgcggaaacacagacgccatgatttgcctttagaaggagctaaggtcttttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcctgtgataatggtcactgtatgctgaatggaacacatggtccatcttcagagaagaaatcaaacatccctgacttaagcatatatttaaagggtacgtatagaatataatttcctttgtgatgtacatcttaatggtcagaatttaaaggcaaaatttcatgccattgtactgaaaatacattaaggttttgtgttatcctctaggagatgtttttgattcagcctctgatcccgtgccattaccacctgccaggcctccaactcgggacaatccaaagcatggttcttcactcaacaggacgccctctgattatgatcttctcatccctccattaggttgaaacctttaaaaaagttttgaacaacccacccctccttcttttaatttcagaattttcagaattcagagttcagtataacacagactcactgggttgtgaatttgcctgaaatttgaatgggttctccaggtgccggtgactcccaagttcacgagaccattactccatgtagatgattaaggtagtagtgtagtagttgggcatcagtcaggttttaagcaagttgttttgtccatactaaatgtagtctaaaaacacatgagagctttgtgctctagtagttttgaagtgatgacttgaagtgttgagattttctttaagtataataattcttaataaatatgaacttgcttttcttgcagcatgagcaccagttccacttacgctaattaaattatgcaaaattaaatagttgtatgtagagaactgataataaattctgttttattctaatcattacaactgtaacacattcaaaaaaaaaaa

SEQ ID NO: 38 - DNA, 3928 bp, Homo sapiens
agcggagtgctgctgcggcgtcccgcggcctccccgagtcgggcgggaggggagagcgggtgtggatttgtcttgacggtaattgttgcgtttccacgtctcggaggcctgcgcgctgggttgctccttcttcgggagcgagctgttctcagcgatcccactcccagccggggctccccacacacactgggctgcgtgcgtgtggagtgggacccgcgcacacgcgtgtctctggacagctacggcgccgaaagaactaaaattccagatggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaaaactatcgtaccatggaaagtattcagacagtgccttcatgaggtccaccagattagctctggcctggaagcaatggctctaaaatcaacaattgatttaacttgcaatgattacatttcagtttttgaatttgatatttttaccaggctgtttcagccttggggctctattttgcggaattggaatttcttagctgtgacacatccaggttacatggcatttctcacatatgatgaagttaaagcacgactacagaaatatagcaccaaacccggaagctatattttccggttaagttgcactcgattgggacagtgggccattggctatgtgactggggatgggaatatcttacagaccatacctcataacaagcccttatttcaagccctgattgatggcagcagggaaggattttatctttatcctgatgggaggagttataatcctgatttaactggattatgtgaacctacacctcatgaccatataaaagttacacaggaacaatatgaattatattgtgaaatgggctccacttttcagctctgtaagatttgtgcagagaatgacaaagatgtcaagattgagccttgtgggcatttgatgtgcacctcttgccttacggcatggcaggagtcggatggtcagggctgccctttctgtcgttgtgaaataaaaggaactgagcccataatcgtggatccctttgatccaagagatgaaggctccaggtgttgcagcatcattgacccctttggcatgccgatgctcgacttggacgacgatgatgatcgtgaggagtccttgatgatgaatcggttggcaaacgtccgaaagtgcactgacaggcagaactcaccagtcacatcaccaggatcctctccccttgcccagagaagaaagccacagcctgacccactccagatcccacatctaagcctgccacccgtgcctcctcgcctggatctaattcagaaaggcatagttagatctccctgtggcagcccaacgggttcaccaaagtcttctccttgcatggtgagaaaacaagataaaccactcccagcaccacctcctcccttaagagatcctcctccaccgccacctgaaagacctccaccaatcccaccagacaatagactgagtagacacatccatcatgtggaaagcgtgccttccaaagacccgccaatgcctcttgaagcatggtgccctcgggatgtgtttgggactaatcagcttgtgggatgtcgactcctaggggagggctctccaaaacctggaatcacagcgagttcaaatgtcaatggaaggcacagtagagtgggctctgacccagtgcttatgcggaaacacagacgccatgatttgcctttagaaggagctaaggtcttttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcttgtgataatggtcactgtatgctgaatggaacacatggtccatcttcagagaagaaatcaaacatccctgacttaagcatatatttaaagggagatgtttttgattcagcctctgatcccgtgccattaccacctgccaggcctccaactcgggacaatccaaagcatggttcttcactcaacaggacgccctctgattatgatcttctcatccctccattaggtgaagatgcttttgatgccctccctccatctctcccacctcccccacctcctgcaaggcatagtctcattgaacattcaaaacctcctggctccagtagccggccatcctcaggacaggatctttttcttcttccttcagatccctttgttgatctagcaagtggccaagttcctttgcctcccgctagaaggttaccaggtgaaaatgtcaaaactaacagaacatcacaggactatgatcagcttccttcatgttcagatggttcacaggcaccagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatagcagccagaactgtagacaccaaaatggaaagcaatcgatgtattccaagagtgtggaaataaagagaactgagatggaattcaagagagaagtgtctcctcctcgtgtagcagcttgagaagaggcttgggagtgcagcttctcaaaggagaccgatgcttgctcaggatgtcgacagctgtggcttccttgtttttgctagccatatttttaaatcagggttgaactgacaaaaataatttaaagacgtttacttcccttgaactttgaacctgtgaaatgctttaccttgtttacaatttggcaaagttgcagtttgttcttgtttttagtttagttttgttttggtgttttgatacctgtactgtgttcttcacagaccctttgtagcgtggtcaggtctgctgtaacatttcccaccaactctcttgctgtccacatcaacagctaaatcatttattcatatggatctctaccatccccatgccttgcccaggtccagttccatttctctcattcacaagatgctttgaaggttctgattttcaactgatcaaactaatgcaaaaaaaaaagtatgtattcttcactactgagtttcttctttggaaaccatcactattgagagatgggaaaaacctgaatgtataaagcatttatttgtcaataaactgccttttgtaaggggttttcacaaaaaaaaaaaaaaaa

SEQ ID NO: 39 - DNA, 3982 bp, Homo sapiens
ctgggtcctgtgtgtgccacaggggtggggtgtccagcgagcggtctcctcctcctgctagtgctgctgcggcgtcccgcggcctccccgagtcgggcgggaggggagagcgggtgtggatttgtcttgacggtaattgttgcgtttccacgtctcggaggcctgcgcgctgggttgctccttcttcgggagcgagctgttctcagcgatcccactcccagccggggctccccacacacactgggctgcgtgcgtgtggagtgggacccgcgcacacgcgtgtctctggacagctacggcgccgaaagaactaaaattccagatggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaaaactatcgtaccatggaaagtattcagacagtgccttcatgaggtccaccagattagctctagcctggaagcaatggctctaaaatcaacaattgatttaacttgcaatgattacatttcagtttttgaatttgatatttttaccaggctgtttcagccttggggctctattttgcggaattggaatttcttagctgtgacacatccaggttacatggcatttctcacatatgatgaagttaaagcacgactacagaaatatagcaccaaacccggaagctatattttccggttaagttgcactcgattgggacagtgggccattggctatgtgactggggatgggaatatcttacagaccatacctcataacaagcccttatttcaagccctgattgatggcagcagggaaggattttatctttatcctgatgggaggagttataatcctgatttaactggattatgtgaacctacacctcatgaccatataaaagttacacaggaacaatatgaattatattgtgaaatgggctccacttttcagctctgtaagatttgtgcagagaatgacaaagatgtcaagattgagccttgtgggcatttgatgtgcacctcttgccttacggcatggcaggagtcggatggtcagggctgccctttctgtcgttgtgaaataaaaggaactgagcccataatcgtggacccctttgatccaagagatgaaggctccaggtgttgcagcatcattgacccctttggcatgccgatgctagacttggacgacgatgatgatcgtgaggagtccttgatgatgaatcggttggcaaacgtccgaaagtgcactgacaggcagaactcaccagtcacatcaccaggatcctctccccttgcccagagaagaaagccacagcctgacccactccagatcccacatctaagcctgccacccgtgcctcctcgcctggatctaattcagaaaggcatagttagatctccctgtggcagcccaacaggttcaccaaagtcttctccttgcatggtgagaaaacaagataaaccactcccagcaccacctcctcccttaagagatcctcctccaccgccacctgaaagacctccaccaatcccaccagacaatagactgagtagacacatccatcatgtggaaagcgtgccttccagagacccgccaatgcctcttgaagcatggtgccctcgggatgtgtttgggactaatcagcttgtgggatgtcgactcctaggggagggctctccaaaacctggaatcacagcgagttcaaatgtcaatggaaggcacagtagagtgggctctgacccagtgcttatgcggaaacacagacgccatgatttgcctttagaaggagctaaggtcttttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcctgtgataatggtcactgtatgctgaatggaacacatggtccatcttcagagaagaaatcaaacatccctgacttaagcatatatttaaagggagatgtttttgattcagcctctgatcccgtgccattaccacctgccaggcctccaactcgggacaatccaaagcatggttcttcactcaacaggacgccctctgattatgatcttctcatccctccattaggtgaagatgcttttgatgccctccctccatctctcccacctcccccacctcctgcaaggcatagtctcattgaacattcaaaacctcctggctccagtagccggccatcctcaggacaggatctttttcttcttccttcagatccctttgttgatctagcaagtggccaagttcctttgcctcctgctagaaggttaccaggtgaaaatgtcaaaactaacagaacatcacaggactatgatcagcttccttcatgttcagatggttcacaggcaccagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatagcagccagaactgtagacaccaaaatggaaagcaatcgatgtattccaagagtgtggaaataaagagaactgagatggaattcaagagagaagtgtctcctcctcgtgtagcagcttgagaagaggcttgggagtgcagcttctcaaaggagaccgatgcttgctcaggatgtcgacagctgtggcttccttgtttttgctagccatatttttaaatcagggttgaactgacaaaaataatttaaagacgtttacttcccttgaactttgaacctgtgaaatgctttaccttgtttacaatttggcaaagttgcagtttgttcttgtttttagtttagttttgttttggtgttttgatacctgtactgtgttcttcacagaccctttgtagcgtggtcaggtctgctgtaacatttcccaccaactctcttgctgtccacatcaacagctaaatcatttattcatatggatctctaccatccccatgccttgcccaggtccagttccatttctctcattcacaagatgctttgaaggttctgattttcaactgatcaaactaatgcaaaaaaaaaaagtatgtattcttcactactgagtttcttctttggaaaccatcactattgagagatgggaaaaacctgaatgtataaagcatttatttgtcaataaactgccttttgtaaggggttttcacataaaaaaaaaaaaa

SEQ ID NO: 40 - DNA, 3241 bp, Homo sapiens
ctgggtcctgtgtgtgccacaggggtggggtgtccagcgagcggtctcctcctcctgctagtgctgctgcggcgtcccgcggcctccccgagtcgggcgggaggggagagcgggtgtggatttgtcttgacggtaattgttgcgtttccacgtctcggaggcctgcgcgctgggttgctccttcttcgggagcgagctgttctcagcgatcccactcccagccggggctccccacacacactgggctgcgtgcgtgtggagtgggacccgcgcacacgcgtgtctctggacagctacggcgccgaaagaactaaaattccagatggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaaaactatcgtaccatggaaagtattcagacagtgccttcatgaggtccaccagattagctctagcctggaagcaatggctctaaaatcaacaattgatttaacttgcaatgattacatttcagtttttgaatttgatatttttaccaggctgtttcagccttggggctctattttgcggaattggaatttcttagctgtgacacatccaggttacatggcatttctcacatatgatgaagttaaagcacgactacagaaatatagcaccaaacccggaagctatattttccggttaagttgcactcgattgggacagtgggccattggctatgtgactggggatgggaatatcttacagaccatacctcataacaagcccttatttcaagccctgattgatggcagcagggaaggattttatctttatcctgatgggaggagttataatcctgatttaactggattatgtgaacctacacctcatgaccatataaaagttacacaggaacaatatgaattatattgtgaaatgggctccacttttcagctctgtaagatttgtgcagagaatgacaaagatgtcaagattgagccttgtgggcatttgatgtgcacctcttgccttacggcatggcaggagtcggatggtcagggctgccctttctgtcgttgtgaaataaaaggaactgagcccataatcgtggacccctttgatccaagagatgaaggctccaggtgttgcagcatcattgacccctttggcatgccgatgctagacttggacgacgatgatgatcgtgaggagtccttgatgatgaatcggttggcaaacgtccgaaagtgcactgacaggcagaactcaccagtcacatcaccaggatcctctccccttgcccagagaagaaagccacagcctgacccactccagatcccacatctaagcctgccacccgtgcctcctcgcctggatctaattcagaaaggcatagttagatctccctgtggcagcccaacaggttcaccaaagtcttctccttgcatggtgagaaaacaagataaaccactcccagcaccacctcctcccttaagagatcctcctccaccgccacctgaaagacctccaccaatcccaccagacaatagactgagtagacacatccatcatgtggaaagcgtgccttccagagacccgccaatgcctcttgaagcatggtgccctcgggatgtgtttgggactaatcagcttgtgggatgtcgactcctaggggagggctctccaaaacctggaatcacagcgagttcaaatgtcaatggaaggcacagtagagtgggctctgacccagtgcttatgcggaaacacagacgccatgatttgcctttagaaggagctaaggtcttttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcctgtgataatggtcactgtatgctgaatggaacacatggtccatcttcagagaagaaatcaaacatccctgacttaagcatatatttaaagggagatgtttttgattcagcctctgatcccgtgccattaccacctgccaggcctccaactcgggacaatccaaagcatggttcttcactcaacaggacgccctctgattatgatcttctcatccctccattaggttgaaacctttaaaaaagttttgaacaacccacccctccttcttttaatttcagaattttcagaattcagagttcagtataacacagactcactgggttgtgaatttgcctgaaatttgaatgggttctccaggtgccggtgactcccaagttcacgagaccattactccatgtagatgattaaggtagtagtgtagtagttgggcatcagtcaggttttaagcaagttgttttgtccatactaaatgtagtctaaaaacacatgagagctttgtgctctagtagttttgaagtgatgacttgaagtgttgagattttctttaagtataataattcttaataaatatgaacttgcttttcttgcagcatgagcaccagttccacttacgctaattaaattatgcaaaattaaatagttgtatgtagagaactgataataaattctgttttattctaatcattacaactgtaacacattcaaaaaaaaaaa

SEQ ID NO: 41 - DNA, 3354 bp, Homo sapiens; identical to SEQ ID NO: 37

SEQ ID NO: 42 - DNA, 2088 bp, Homo sapiens
agtgctgctgcggcgtcccgcggcctccccgagtcgggcgggaggggagagcgggtgtggatttgtcttgacggtaattgttgcgtttccacgtctcggaggcctgcgcgctgggttgctccttcttcgggagcgagctgttctcagcgatcccactcccagccggggctccccacacacactgggctgcgtgcgtgtggagtgggacccgcgcacacgcgtgtctctggacagctacggcgccgaaagaactaaaattccagatggcaaactcaatgaatggcagaaaccctggtggtcgaggaggaaatccccgaaaaggtcgaattttgggtattattgatgctattcaggatgcagttggaccccctaagcaagctgccgcagatcgcaaaacctggaatcacagcgagttcaaatgtcaatggaaggcacagtagagtgggctctgacccagtgcttatgcggaaacacagacgccatgatttgcctttagaaggagctaaggtcttttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcttgtgataatggtcactgtatgctgaatggaacacatggtccatcttcagagaagaaatcaaacatccctgacttaagcatatatttaaagggagatgtttttgattcagcctctgatcccgtgccattaccacctgccaggcctccaactcgggacaatccaaagcatggttcttcactcaacaggacgccctctgattatgatcttctcatccctccattaggtgaagatgcttttgatgccctccctccatctctcccacctcccccacctcctgcaaggcatagtctcattgaacattcaaaacctcctggctccagtagccggccatcctcaggacaggatctttttcttcttccttcagatccctttgttgatctagcaagtggccaagttcctttgcctcctgctagaaggttaccaggtgaaaatgtcaaaactaacagaacatcacaggactatgatcagcttccttcatgttcagatggttcacaggcatcagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatagcagccagaactgtagacaccaaaatggaaagcaatcgatgtattccaagagtgtggaaataaagagaactgagatggaattcaagagagaagtgtctcctcctcgtgtagcagcttgagaagaggcttgggagtgcagcttctcaaaggagaccgatgcttgctcaggatgtcgacagctgtggcttccttgtttttgctagccatatttttaaatcagggttgaactgacaaaaataatttaaagacgtttacttcccttgaactttgaacctgtgaaatgctttaccttgtttacagtttggcaaagttgcagtttgttcttgtttttagtttagttttgttttggtgttttgtacctgtactgtgttcttcacagaccctttgtagcgtggtcaggtctgctgtaacatttcccaccaactctcttgctgtccacatcaacagctaaatcatttattcatatggatctctaccatccccatgccttgcccaggtccagttccatttctctcattcacaagatgctttgaaggttctgattttcaactgatcaaactaatgcaaaaaaaaaaaaaaaaaaaaaaaaaag

SEQ ID NO: 43 - DNA, 1446 bp, Homo sapiens
  misc_feature 4, 10, 12, 13, 58, 74, 136, 206, 222, 237, 254, 385,1336, 1344, 1347, 1350, 1380, 1392, 1395, 1400, 1445: n = A,T,C or G
cgtntttggnanncactacaggggatgtttaatacacactcacaatgcgcatgatgtntataactatctattcnatgatgtaagataccccactcaaacccataaaaaagagcatctttaatacgactcactatanggcgagcgcacgccatggcaggtacccatacgacgtaccagattacgctcatatggccatggaggccagngaattccacccaagcngtggtatcaacgcanagtggactctgacccantgcttatgcggaaacacagacgccatgatttgcctttagaaggagctaaggtctcttccaatggtcaccttggaagtgaagaatatgatgttcctccccggctttctcctcctcctccagttaccaccctnctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcttgtgataatggtcactgtatgctgaatggaacacatggtccatcttcagagaagaaatcaaacatccctgacttaagcatatatttaaagggtgaagatgcttttgatgccctccctccatctctcccacctcccccacctcctgcaaggcatagtctcattgaacattcaaaacctcctggctccagtagccggccatcctcaggacaggatctttttcttcttccttcagatccctttgttgatctagcaagtggccaagttcctttgcctcccgctagaaggttaccaggtgaaaatgtcaaaactaacaggacatcacaggactatgatcagcttccttcatgttcagatggttcacaggcaccagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatagcagccagaactgtagacaccaaaatggaaagcaatcgatgtattccaagagtgtggaaataaagagaactgagatggaattcaagagagaagtgtctcctcctcgtgtagcagcttgagaagaggcttgggagtgcagcttctcaaagaaaaccgatgcttgctcaggatgtcnacagctgnggnctnccttgtttttgctagccatttttttaaatnagggttgaactnganaaaantatttaaaaacgtttacctcccttgaactttgaacctgggaaagnc

SEQ ID NO: 44 - DNA, 1203 bp, Homo sapiens
actctgacccagtgcttatgcggaaacacagacgccatgatttgcctttagaaggagctaaggtctcttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcttgtgataatggtcactgtatgctgaatggaacacatggtccatcttcagagaagaaatcaaacatccctgacttaagcatatatttaaagggtgaagatgcttttgatgccctccctccatctctcccacctcccccacctcctgcaaggcatagtctcattgaacattcaaaacctcctggctccagtagccggccatcctcaggacaggatctttttcttcttccttcagatccctttgttgatctagcaagtggccaagttcctttgcctcccgctagaaggttaccaggtgaaaatgtcaaaactaacaggacatcacaggactatgatcagcttccttcatgttcagatggttcacaggcaccagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatagcagccagaactgtagacaccaaaatggaaagcaatcgatgtattccaagagtgtggaaataaagagaactgagatggaattcaagagagaagtgtctcctcctcgtgtagcagcttgagaagaggcttgggagtgcagcttctcaaagaaaaccgatgcttgctcaggatgtcgacagctgtggcttccttgtttttgctagccatttttttaaatcagggttgaactggaaaaaattatttaaaaacgtttacctcccttgaactttgaacctgggaaaggc

SEQ ID NO: 45 - protein, 300 aa, Homo sapiens
MRKHRRHDLPLEGAKVSSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 46 - protein, 250 aa, Homo sapiens
SDPVLMRKHRRHDLPLEGAKVSSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEA

SEQ ID NO: 47 - protein, 770 aa, Homo sapiens
MANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSSLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGTYRI

SEQ ID NO: 48 - protein, 982 aa, Homo sapiens
MANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSKDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGDVFDSASDPVPLPPARPPTRDNPKHGSSLNRTPSDYDLLIPPLGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 49 - protein, 982 aa, Homo sapiens [sequence omitted]

SEQ ID NO: 50 - protein, 810 aa, Homo sapiens [sequence omitted]

SEQ ID NO: 51 - DNA, 3373 bp, Rattus norvegicus
cgggcgggcgtggagctgtctgcacgaaaggactaagattccagatggcaaattctatgaatggcagaaatcctggtggtcgaggaggaaacccccgcaaaggtcgaattttggggattattgatgccattcaggatgcagttggacccccaaagcaagctgcagctgaccgcaggacagtggagaagacttggaaactcatggacaaagtggtaagactgtgccaaaatccgaaacttcagttgaaaaacagcccaccatatatcctcgacattttacctgatacgtatcagcatttgcggcttatattgagtaagtatgacgacaaccagaagctggctcaactgagcgagaatgagtactttaaaatctacatcgacagtctcatgaagaagtcaaagcgagcgatccggctcttcaaagaaggcaaggagaggatgtacgaggagcagtcgcaggacagacggaatctcacaaagctgtcccttatcttcagtcacatgctggcagaaatcaaggcgatctttcccaatggccagttccagggagataacttccggatcaccaaagcagatgctgccgaattctggaggaagttttttggagacaaaactatcgtaccatggaaagtcttcagacagtgcctgcatgaggtccatcagatcagctctggcctggaggccatggctctgaagtcaaccattgacttaacttgtaatgattacatctccgtgtttgaatttgatatttttaccaggctatttcagccctggggctctattttacggaattggaacttcttagctgtgacacacccggggtacatggcatttctcacatatgatgaagttaaagctcgactacagaaatacagcaccaagcctggaagctacattttccggttaagctgcactcggctgggacaatgggccattggctatgtgactggggacggcaatatcctacagaccatacctcataacaagcccctgttccaagccctgattgatggtagcagggaaggcttttacctttatccagatggacgaagctataaccctgatttaaccggattatgtgaacctacacctcatgatcatataaaagttacacaggagcaatatgaactgtattgtgaaatgggctccacttttcagctgtgcaagatctgtgcagagaatgacaaagatgtcaagatcgagccttgtgggcatctcatgtgcacttcgtgccttaccgcgtggcaggagtctgatggccaaggctgccccttctgtcgctgtgagataaaaggaaccgaacctatcatcgtggatccctttgaccccagagacgaaggctccaggtgctgcagcatcatcgaccctttcagcatccccatgctcgacttggatgatgacgatgatcgagaggagtctctgatgatgaaccggctggcgagtgttcgcaagtgcacagacaggcagaactcgccagtcacatcgccaggatcctcaccccttgcccagagaagaaagcctcagccagaccctctccagatcccccacctcagcctgccaccagtgcctccccgcctggacctcattcagaaaggcatcgtgcgctctccctgtggcagccccacgggctccccgaagtcttctccatgcatggttagaaaacaagacaaaccactcccagcaccccctcctcccttgcgagatcctccgcctccaccagagcggcctccgccaatcccgcctgacagtagactgagcagacacttccaccacggagagagtgtgccttccagggaccagccaatgcctcttgaagcctggtgccctcgggatgccttcgggactaatcaggtgatgggatgtcgcatcctaggggatggctctccaaagcctggcgtcacagcaaactccaacttaaatggacgtcacagtcgaatgggctctgaccaggttcttatgaggaaacacagacgccacgatttgccttcagaaggcgccaaggtcttttccaatggacaccttgcccctgaagaatacgacgttcctcctcggctttcccctcctcctccagtcactgcccttctccctagcataaagtgtactggtccaatagcaaattgtctctccgagaaaacaagagacacagtagaagaagatgatgatgaatacaagattccttcatcccatcctgtttccctgaattcacaaccatctcattgtcataatgtcaaacctcctgttcggtcttgtgataatggtcactgtatactgaatggaactcatggtacgccttcagagatgaagaaatcaaacatcccagatttaggcatctatttgaagggtgaagatgcttttgatgccctccccccatcccttcctcctcccccacctcctgcaagacatagtctcatcgagcattcaaaacctccaggctccagtagccggccttcctcaggacaggaccttttccttcttccttcagatcccttttttgacccagcaagtggccaagttccattgcctccggccaggagagcaccaggagatggtgtcaaatccaacagagcctcccaggactatgaccagctcccttcatcttccgatggttcgcaagcaccagctagaccccccaaaccacgaccccgaaggactgcaccagaaattcatcacagaaagccccatgggcccgaggcggcactggaaaatgtggatgcgaaaattgcaaaactcatgggagaggggtatgcctttgaagaggtgaagagagccttagagatcgcccagaataacctggaagtggccaggagcatacttcgagaattcgccttccctcctcccgtctcgccacgtctcaatctatagcagcccagactgcaaacaccaaagggtaaaacagttaacaaatattccaggagtatgggacagaaggactgagagggaatgcaggagccatggtgtcttttcatgtggcgtctccagaaggcagccttgagtccagcttctctggtaccacagctccctgaggatgcccacgctgcagcttctgtgtttgtgctagccatacttttaaatcagggttgaactgagaaaataatttaaagacgtttactcccccttgaactttgaatctgtgaaatgctttccttgtttacacgttggcagaattgcagtttgtctctgtttttgattcctgtactgtgttcctgacaggcccttggcagagttggtcaggtctgctgtaagtttgtccatgcccaccctgctgcccacattggcagctaaagcatctcttcgtgttgctgtctatccgggccccacctcatgtgtccacgtccagttcatttctctcattcacacagcatgctagtctgagg

SEQ ID NO: 52 - DNA, 3131 bp, Mus musculus
gactccctgggctgcgagcgccggcggtggttgccggagaggcccctccttctcgcccggctccattccctcgctcgcggccgagcgggctcccgaccctccgctggccatggccggcaacgtgaagaagagctcgggcgccggcggcggcggctctgggggctcgggagcgggcggcctgatcgggctcatgaaggacgccttccagccgcaccaccaccaccaccacctcagcccgcaccctccctgcacggtggacaagaagatggtggagaagtgctggaagctcatggacaaggtggtgcggttgtgtcaaaacccaaagctggcgctcaagaacagcccgccttatatcttagacctgctgcctgacacctaccagcacctccgcactgtcttgtcaagatatgaggggaagatggagacgcttggagaaaatgagtatttcagggtgttcatggaaaatttgatgaagaaaactaagcagactatcagcctcttcaaggagggaaaagaaaggatgtatgaggagaattcccagcctaggcgaaacctgaccaaattatccctgatcttcagccacatgctggcagaactgaaaggcatctttccgagcggactcttccaaggagacactttccggattactaaagctgatgctgccgaattttggagaaaagcttttggtgaaaagacgatagtcccgtggaagagctttcgacaggccctgcatgaagtgcatcccatcagttctgggctggacgccatggctctgaagtccactattgatctgacctgcaatgattatatttctgtctttgaatttgatatttttacacggctgtttcagccctggtcctctttgctcagaaattggaacagccttgctgtaactcaccctggttacatggctttcctgacatacgatgaagtgaaagcgcgcctgcagaagttcatccacaaacctggcagttacatctttcggctgagctgtactcgtttgggtcagtgggctattgggtatgttactgccgatgggaacattctgcagacaatcccacacaataaaccgctcttccaagcactgattgatggcttcagggaaggcttctatttgtttcctgatggacgaaatcaaaatcctgacctgacaggtttatgtgaaccaactcctcaagatcatatcaaagtaacccaggaacaatatgaattatactgtgaaatgggctccacatttcaactgtgtaagatatgtgctgagaatgataaggatgtgaagattgagccctgtggacacctcatgtgcacatcctgcctcacgtcgtggcaggaatcagaaggtcagggctgtcctttttgccgatgtgaaatcaaaggtactgagcccatcgtggtggatccgtttgaccccagaggcagtggcagcctattaaggcaaggagcagaaggtgctccttccccaaattacgacgatgatgatgatgaacgagctgatgattctctcttcatgatgaaggagttggcaggtgccaaggtggaaaggccttcctctccattctccatggccccacaagcttcccttcctccagtgccaccaagacttgaccttctacagcagcgagcacctgttcctgccagcacttcagttctggggactgcttccaaggctgcttctggctcccttcataaagacaaaccattgccaatacctcccacacttcgagatcttccaccaccaccccctccagaccggccttactctgttggagcagaaacaaggcctcagagacgccctctgccttgtacaccaggcgattgtccatctagagacaaactgccccctgtcccttctagccgcccaggggactcgtggttgtctcggccaatccctaaagtaccagtagctactccaaaccctggtgatccttggaatgggagagaattgaccaatcggcactcgcttccattctcattgccctcacaaatggaacccagagcagatgtccctaggcttggaagcacatttagtctggatacctctatgactatgaatagcagcccagtagcaggtccagagagtgagcacccaaagatcaagccttcctcgtctgccaacgccatttactctctggctgccaggcctcttcctatgccaaaactgccacctggggagcaaggggaaagtgaagaggacacagaatatatgactcccacatctaggcctgtaggggttcagaagccagagcccaaacggccgttagaggcaacccagagttcacgagcatgtgactgtgaccagcagatcgacagctgtacctatgaagcgatgtataacatccagtcccaagcactgtctgtagcagaaaacagcgcctctggggaagggaatctggccacagctcacacgagtactggccctgaggaatccgaaaacgaggatgatggctatgatgtgcctaagccacccgtgccagctgtactggcccgccggaccctgtctgacatctccaatgccagctcctcctttggctggttgtctttggatggtgaccctacaaacttcaatgagggttcccaagttcctgagcggccccccaaaccattccctcggagaatcaactcagaacgaaaagccagtagctatcaacaaggcggaggtgccactgctaaccctgtggccacagcaccctcaccgcagctctcaagtgagattgaacgcctcatgagtcagggctattcctaccaggacattcagaaagctttggtcattgcccacaacaacattgagatggctaaaaacatcctccgggaatttgtttctatttcttctcctgctcacgtagccacctagcacatctctccctgccacggcttcagaggacccatgagccaggctcttactcaaggaccacctaggaaagcagtggcttcttttgggacgtcacagtaaggtcctgcctttcctgtggggatcgacacatatggttccaagatttcaaagcagtggaatgaaaatggagcagctgatgtgtttcattgttgtattggtcttaagagtgtttttgtagtcctgcagtctccagtaggagagagtgggtttttattaaatggtaacctaccccagaaacagc

SEQ ID NO: 53 - DNA, 2661 bp, Drosophila
catctcgaaaatattgtgtgggtttaaaaaacgttaacgtcgccgaaacgcgtagccccaaatgcacacgccaggtgcaaggataaagccgtgaggatcgggcacccaatcggatagatcgcgtttggttagcttgtgggggaaaatcgtacttaagtcaccactactactacacacgggcaccaccagcaacaccaacaacaacaacaacgagaacagcaccagcaacaacaacaacagcagcaagaaggagaagagctgagaagaggaagcagaggcagcgcagtcggcagcgcagcggcagagagaaaagatggcgacgagaggcagtggaacccgtgtgcaatcgcagccaaagattttcccatcgctgctttccaagctgcacggcgctatctcggaagcctgcgtctcgcagcgtctgtccaccgacaagaagacgctggagaagacctggaagttgatggacaaggtggtcaaactgtgccagcagccgaagatgaatcttaagaatagtccaccgtttattttggacatcctgccggatacgtaccagcgcctgagattgatctactcaaagaaggaggaccagatgcacctgctccatgccaacgagcacttcaacgtgttcatcaacaacctgatgcgaaagtgcaagcgggccatcaagttgttcaaggagggcaaggagaagatgttcgacgagaactcccactaccgccgcaatctcaccaagctcagcctggtcttctcccacatgctcagcgaactgaaggccatattccccaacggtgtctttgccggggatcaatttcggatcaccaaagcggatgcggctgacttttggaagagcaacttcggtaacagcacattggttccctggaaaatcttccggcaggagcttagcaaagtacatcccataatctccggcctggaggccatggccctaaagaccactatcgatcttacctgcaacgacttcatttccaacttcgagttcgacgtcttcacacgcctcttccagccttgggtgacactgctacgcaactggcagattctggccgtcacacatccgggctacgtggcgtttctcacatacgacgaggtgaaggctcgcctacagcgctacatcctcaaggcgggcagctacgttttccggctctcctgcacgcgattgggccaatgggccatcggctacgtaactgccgagggagagattctgcagacaatccctcagaacaagtcgctgtgccaggcgctgctcgatggccatcgagagggcttctacttgtacccagatggccaagcgtacaatccggatctgtcgtctgccgttcaaagtcccacagaggaccacataaccgttacccaagagcaatacgaactatactgtgaaatgggcagcacctttcagctgtgcaaaatttgtgcggagaacgacaaagatatccgcatcgagccctgtggccacttgttgtgcactccctgccttacctcctggcaagtggattccgagggacagggctgccccttctgtcgggccgaaatcaagggcaccgaacaaatcgttgtggacgctttcgatccgcgcaagcaacacaaccggaacgtcaccaatgggcgacagcagcagcaggaagaagacgacactgaggtatagttttgttcacagcctgatcagcctgatccgcctgctccgctgccgcctgtgctgctatttatatacatattactcttatgattacctttggttcgtttatacagttatatatgcctatatatacattatatattttagattttacaactgctattgtttatataagtttaatgtttagcctgcagttcgcagtggcagtttcgagtttaattttgtttgtttagctgtaacatatttaaattattagccaaactcatgcaactaacatccacagacccacgcacacacgcccaatcacaagcacaagtacaaccataaccattgtccatccatcgagcacatgcataacgtagttaaagttctttgaccggaagtcgctcatcaaccatcgtttgctatcgcttcctctgttttctctccgccggtttggtttggtttggtttgtgtgcgttcgtttagttgttcgttcttccactctcacgctctctctatctattgatcacgttcgcctctgtttatgaatcatattttaatcgattcgattcgccctcgattgcacttttgtacataggcactatggaatttataattggtaaccttgttcttgtattattcgggtgaattttctcctttcacatccagcttgattatccccttgattatgtatgcccgccagtaatttttgtatctatcccctactctagaatcattctcttaatcattgtactccgttatgtgtttatttcattttagtttattgtttaatacttccaaagatacatttagtttgtagtagcgtgcgtttacttcccccccatatcaattcaattttatttgtaagcagccaaygcgctgccctaagactgtaatttattattaacamaaaaaaraaaatcgaaaaagtttaagaaatcaggctaaacataggaggcctcgaatcgatcgataatttagttagattgyatgtaaattaattattgatttcctgtgtcacaaggcca

SEQ ID NO: 54 - DNA, 2274 bp, C. elegans
ctatgatcattacatcctaattaattgccactggacttcacatcatatcaccgtttcaccgggaatgggttcaataaacacaatttttcaccggatacatcggtttgtcaatggcacaggcaataatgcgcgatttgttcccagcacaaacaactcgacggaagcgttgacactcagtccgagagctgttcccagcacagtttcactattcgaaatcccatcagcttcggagatgcccggtttctgcagtgaagaggatcgtcgatttttgctcaaagcatgcaagtttatggatcaagtagtgaagagttgtcatagcccaagactgaatttgaaaaattcgccgcctttcattttggacattctacctgatacttatacgcatttaatgctgatattcacacaaaacaatgacatactccaagacaacgactacttgaaaatctttctggagagtatgatcaacaagtgcaaagagatcatcaaactgttcaagacgtcagctatctacaatgaccagtctgaagaacgacggaagcttacgaaaatgtcactaacattttcacatatgcttttcgagattaaagcattatttccggaaggtatctatattgaagaccggtttcggatgacaaagaaggaagccgaaagcttttggagtcatcattttacaaaaaaaaacattgtaccctggtcaacattttttactgcattagaaaagcaccatggatcaacgataggaaaaatggaagcagccgaattaaaagctacgatagacttgagcggagatgattttatttcgaattttgagtttgatgtgtttacaaggttattctaccctttcaaaacactgatcaaaaattggcaaacactcaccaccgcccatcccggatactgtgcatttctcacatacgatgaggtcaaaaaacggttagaaaaattaacgaaaaaacctggaagctacatcttccggttatcatgcacacgtcctggacaatgggcaataggatacgtagctccggatggaaagatttatcagacaataccacagaataaaagtttgattcaagcactacatgaaggccataaagaaggattttatatttacccgaacggtagagatcaagatattaacttatccaaattgatggatgtgccacaagcggacagagtgcaagtgaccagtgaacaatacgagttgtattgtgagatgggcacaacattcgagttgtgcaaaatttgtgacgataacgagaagaacatcaaaattgagccatgtggacatttgctctgcgcaaaatgtttggctaactggcaggattcggatggtggtggcaacacatgtccattctgccgctacgaaatcaaaggaacaaatcgtgtgattattgacaggttcaagcccactccggtagaaattgaaaaagcgaaaaatgtagctgctgcggagaagaagctgatctcattagttcccgacgtgcctcccagaacgtatgtgtcccaatgttctcaaagtttgctgcatgacgcgtcaaactcaattccgtcggtcgacgagttgccgttggtgccgccaccgttgccaccgaaagcattgggtaccctggacactttgaattcgtcacaaacatcctcttcatacgtgaacatcaaagagctggaaaatgttgaaacaagcggagaagcattggcacaagtggtaaaccggcaacgggcgccttcaatccaagctccaccactaccgccaaggttatcagcgagcgagcaccaaccacaccacccatacacaaatacgaacagtgagcgggagtagacttgtgtaaatgttcatcttaccgctttatactgcaattttcattcccccacttatcatagaactattcttccacaacaacatattgccgtgactagaactggtaacactacatcattctttgttaaaacgttattatatctctatttctttttcgcctactcctttccgtttttttttcaaattttgtcaattttcctacagcgttctgactcctattggtaagcaatcatgtcatatcttgttaaattttcatgttaatttcttactctcgctgtcccagattttacggagttttcaggaaacgtttgattttgttctattctacaatttccatcgcccccaacctgtcgtgtattttctatgtgtcactctgaagaaaacaagtttagactttttaaaaatcgttttattactctaaaacttaaaagctgaaatgtcagctatagtaaaaatacata

SEQ ID NO: 55 - protein, 938 aa, Rattus norvegicus
MANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFSIPMLDLDDDDDREESLMMNRLASVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPERPPPIPPDSRLSRHFHHGESVPSRDQPMPLEAWCPRDAFGTNQVMGCRILGDGSPKPGVTANSNLNGRHSRMGSDQVLMRKHRRHDLPSEGAKVFSNGHLAPEEYDVPPRLSPPPPVTALLPSIKCTGPIANCLSEKTRDTVEEDDDEYKIPSSHPVSLNSQPSHCHNVKPPVRSCDNGHCILNGTHGTPSEMKKSNIPDLGIYLKGEDAFDALPPSLPPPPPPARHSLIEHSKPPGSSSRPSSGQDLFLLPSDPFFDPASGQVPLPPARRAPGDGVKSNRASQDYDQLPSSSDGSQAPARPPKPRPRRTAPEIHHRKPHGPEAALENVDAKIAKLMGEGYAFEEVKRALEIAQNNLEVARSILREFAFPPPVSPRLNL

SEQ ID NO: 56 - protein, 913 aa, Mus musculus
MAGNVKKSSGAGGGGSGGSGAGGLIGLMKDAFQPHHHHHHLSPHPPCTVDKKMVEKCWKLMDKVVRLCQNPKLALKNSPPYILDLLPDTYQHLRTVLSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLDAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDSLFMMKELAGAKVERPSSPFSMAPQASLPPVPPRLDLLQQRAPVPASTSVLGTASKAASGSLHKDKPLPIPPTLRDLPPPPPPDRPYSVGAETRPQRRPLPCTPGDCPSRDKLPPVPSSRPGDSWLSRPIPKVPVATPNPGDPWNGRELTNRHSLPFSLPSQMEPRADVPRLGSTFSLDTSMTMNSSPVAGPESEHPKIKPSSSANAIYSLAARPLPMPKLPPGEQGESEEDTEYMTPTSRPVGVQKPEPKRPLEATQSSRACDCDQQIDSCTYEAMYNIQSQALSVAENSASGEGNLATAHTSTGPEESENEDDGYDVPKPPVPAVLARRTLSDISNASSSFGWLSLDGDPTNFNEGSQVPERPPKPFPRRINSERKASSYQQGGGATANPVATAPSPQLSSEIERLMSQGYSYQDIQKALVIAHNNIEMAKNILREFVSISSPAHVAT

SEQ ID NO: 57 - protein, 448 aa, Drosophila
MATRGSGTRVQSQPKIFPSLLSKLHGAISEACVSQRLSTDKKTLEKTWKLMDKVVKLCQQPKMNLKNSPPFILDILPDTYQRLRLIYSKKEDQMHLLHANEHFNVFINNLMRKCKRAIKLFKEGKEKMFDENSHYRRNLTKLSLVFSHMLSELKAIFPNGVFAGDQFRITKADAADFWKSNFGNSTLVPWKIFRQELSKVHPIISGLEAMALKTTIDLTCNDFISNFEFDVFTRLFQPWVTLLRNWQILAVTHPGYVAFLTYDEVKARLQRYILKAGSYVFRLSCTRLGQWAIGYVTAEGEILQTIPQNKSLCQALLDGHREGFYLYPDGQAYNPDLSSAVQSPTEDHITVTQEQYELYCEMGSTFQLCKICAENDKDIRIEPCGHLLCTPCLTSWQVDSEGQGCPFCRAEIKGTEQIVVDAFDPRKQHNRNVTNGRQQQQEEDDTEV

SEQ ID NO: 58 - protein, 582 aa, C. elegans
MGSINTIFHRIHRFVNGTGNNARFVPSTNNSTEALTLSPRAVPSTVSLFEIPSASEMPGFCSEEDRRFLLKACKFMDQVVKSCHSPRLNLKNSPPFILDILPDTYTHLMLIFTQNNDILQDNDYLKIFLESMINKCKEIIKLFKTSAIYNDQSEERRKLTKMSLTFSHMLFEIKALFPEGIYIEDRFRMTKKEAESFWSHHFTKKNIVPWSTFFTALEKHHGSTIGKMEAAELKATIDLSGDDFISNFEFDVFTRLFYPFKTLIKNWQTLTTAHPGYCAFLTYDEVKKRLEKLTKKPGSYIFRLSCTRPGQWAIGYVAPDGKIYQTIPQNKSLIQALHEGHKEGFYIYPNGRDQDINLSKLMDVPQADRVQVTSEQYELYCEMGTTFELCKICDDNEKNIKIEPCGHLLCAKCLANWQDSDGGGNTCPFCRYEIKGTNRVIIDRFKPTPVEIEKAKNVAAAEKKLISLVPDVPPRTYVSQCSQSLLHDASNSIPSVDELPLVPPPLPPKALGTLDTLNSSQTSSSYVNIKELENVETSGEALAQVVNRQRAPSIQAPPLPPRLSASEHQPHHPYTNTNSERE

SEQ ID NO: 59 - DNA, 21 bp, Homo sapiens
uggaaggcacaguagagugtt

SEQ ID NO: 60 - DNA, 21 bp, Homo sapiens
cacucuacugugccuuccatt

SEQ ID NO: 61 - DNA, 21 bp, Homo sapiens
uuaugaucuucucaucccutt

SEQ ID NO: 62 - DNA, 21 bp, Homo sapiens
agggaugagaagaucauaatt

SEQ ID NO: 63 - DNA, 21 bp, Homo sapiens
gcaugguucuucacucaactt

SEQ ID NO: 64 - DNA, 21 bp, Homo sapiens
guugagugaagaaccaugctt